SEQUENCE LISTING (94 sequences)

SEQ ID NO: 1 - protein, 34 aa, ARTIFICIAL
  Artificial protein based on human PYY
IKPEAPGEDASPEELNRYYASLRHYLNLVTRQKY

SEQ ID NO: 2 - protein, 34 aa, ARTIFICIAL
  Artificial protein based on human PYY
IKPEAPGEDASPEELNRYYASLRHYLNLVTRKRY

SEQ ID NO: 3 - protein, 34 aa, ARTIFICIAL
  Artificial protein based on human PYY
IKPEAPGEDASPEELNRYYASLRHYLNLVTKQRY

SEQ ID NO: 4 - protein, 34 aa, ARTIFICIAL
  Artificial protein based on human PYY
IKPEAPGEDASPEELNRYYASLRHYLNLVKRQRY

SEQ ID NO: 5 - protein, 34 aa, ARTIFICIAL
  Artificial protein based on human PYY
IKPEAPGEDASPEELNRYYASLRHYLNLKTRQRY

SEQ ID NO: 6 - protein, 34 aa, ARTIFICIAL
  Artificial protein based on human PYY
IKPEAPGEDASPEELNRYYASLRHYLNKVTRQRY

SEQ ID NO: 7 - protein, 34 aa, ARTIFICIAL
  Artificial protein based on human PYY
IKPEAPGEDASPEELNRYYASLRHYLKLVTRQRY

SEQ ID NO: 8 - protein, 34 aa, ARTIFICIAL
  Artificial protein based on human PYY
IKPEAPGEDASPEELNRYYASLRHYKNLVTRQRY

SEQ ID NO: 9 - protein, 34 aa, ARTIFICIAL
  Artificial protein based on human PYY
IKPEAPGEDASPEELNRYYASLRHKLNLVTRQRY

SEQ ID NO: 10 - protein, 34 aa, ARTIFICIAL
  Artificial protein based on human PYY
IKPEAPGEDASPEELNRYYASLRKYLNLVTRQRY

SEQ ID NO: 11 - protein, 34 aa, ARTIFICIAL
  Artificial protein based on human PYY
IKPEAPGEDASPEELNRYYASLKHYLNLVTRQRY

SEQ ID NO: 12 - protein, 34 aa, ARTIFICIAL
  Artificial protein based on human PYY
IKPEAPGEDASPEELNRYYASKRHYLNLVTRQRY

SEQ ID NO: 13 - protein, 34 aa, ARTIFICIAL
  Artificial protein based on human PYY
IKPEAPGEDASPEELNRYYAKLRHYLNLVTRQRY

SEQ ID NO: 14 - protein, 34 aa, ARTIFICIAL
  Artificial protein based on human PYY
IKPEAPGEDASPEELNRYYKSLRHYLNLVTRQRY

SEQ ID NO: 15 - protein, 34 aa, ARTIFICIAL
  Artificial protein based on human PYY
IKPEAPGEDASPEELNRYKASLRHYLNLVTRQRY

SEQ ID NO: 16 - protein, 34 aa, ARTIFICIAL
  Artificial protein based on human PYY
IKPEAPGEDASPEELNRKYASLRHYLNLVTRQRY

SEQ ID NO: 17 - protein, 34 aa, ARTIFICIAL
  Artificial protein based on human PYY
IKPEAPGEDASPEELNKYYASLRHYLNLVTRQRY

SEQ ID NO: 18 - protein, 34 aa, ARTIFICIAL
  Artificial protein based on human PYY
IKPEAPGEDASPEELKRYYASLRHYLNLVTRQRY

SEQ ID NO: 19 - protein, 34 aa, ARTIFICIAL
  Artificial protein based on human PYY
IKPEAPGEDASPEEKNRYYASLRHYLNLVTRQRY

SEQ ID NO: 20 - protein, 34 aa, ARTIFICIAL
  Artificial protein based on human PYY
IKPEAPGEDASPEKLNRYYASLRHYLNLVTRQRY

SEQ ID NO: 21 - protein, 34 aa, ARTIFICIAL
  Artificial protein based on human PYY
IKPEAPGEDASPKELNRYYASLRHYLNLVTRQRY

SEQ ID NO: 22 - protein, 34 aa, ARTIFICIAL
  Artificial protein based on human PYY
IKPEAPGEDASKEELNRYYASLRHYLNLVTRQRY

SEQ ID NO: 23 - protein, 34 aa, ARTIFICIAL
  Artificial protein based on human PYY
IKPEAPGEDAKPEELNRYYASLRHYLNLVTRQRY

SEQ ID NO: 24 - protein, 34 aa, ARTIFICIAL
  Artificial protein based on human PYY
IKPEAPGEDKSPEELNRYYASLRHYLNLVTRQRY

SEQ ID NO: 25 - protein, 34 aa, ARTIFICIAL
  Artificial protein based on human PYY
IKPEAPGEKASPEELNRYYASLRHYLNLVTRQRY

SEQ ID NO: 26 - protein, 34 aa, ARTIFICIAL
  Artificial protein based on human PYY
IKPEAPGKDASPEELNRYYASLRHYLNLVTRQRY

SEQ ID NO: 27 - protein, 34 aa, ARTIFICIAL
  Artificial protein based on human PYY
IKPEAPKEDASPEELNRYYASLRHYLNLVTRQRY

SEQ ID NO: 28 - protein, 34 aa, ARTIFICIAL
  Artificial protein based on human PYY
IKPEAKGEDASPEELNRYYASLRHYLNLVTRQRY

SEQ ID NO: 29 - protein, 34 aa, ARTIFICIAL
  Artificial protein based on human PYY
IKPEKPGEDASPEELNRYYASLRHYLNLVTRQRY

SEQ ID NO: 30 - protein, 34 aa, ARTIFICIAL
  Artificial protein based on human PYY
IKPKAPGEDASPEELNRYYASLRHYLNLVTRQRY

SEQ ID NO: 31 - protein, 34 aa, ARTIFICIAL
  Artificial protein based on human PYY
IKKEAPGEDASPEELNRYYASLRHYLNLVTRQRY

SEQ ID NO: 32 - protein, 34 aa, ARTIFICIAL
  Artificial protein based on human PYY
IKPEAPGEDASPEELNRYYASLRHYLNLVTRQRY

SEQ ID NO: 33 - protein, 34 aa, ARTIFICIAL
  Artificial protein based on human PYY
KKPEAPGEDASPEELNRYYASLRHYLNLVTRQRY

SEQ ID NO: 34 - protein, 34 aa, ARTIFICIAL; identical to SEQ ID NO: 32
  Artificial protein based on human PYY

SEQ ID NO: 35 - protein, 34 aa, ARTIFICIAL
  Artificial protein based on human PYY
IRPEAPGEDASPEELNRYYASLRHYLNKVTRQRY

SEQ ID NO: 36 - protein, 32 aa, ARTIFICIAL
  Artificial protein based on human PYY
PEAPGEDASPEELNRYYASLRHYLNKVTRQRY

SEQ ID NO: 37 - protein, 34 aa, ARTIFICIAL
  Artificial protein based on human PYY
IRPEAPGEDASPEELERYYASLRHYLNKVTRQRY

SEQ ID NO: 38 - protein, 32 aa, ARTIFICIAL
  Artificial protein based on human PYY
PEAPGEDASPEELERYYASLRHYLNKVTRQRY

SEQ ID NO: 39 - protein, 34 aa, ARTIFICIAL
  Artificial protein based on human PYY
IRPEAPGEDASPEELERYYASLRHYLQKVTRQRY

SEQ ID NO: 40 - protein, 34 aa, ARTIFICIAL; identical to SEQ ID NO: 6
  Artificial protein based on human PYY

SEQ ID NO: 41 - protein, 34 aa, ARTIFICIAL; identical to SEQ ID NO: 37
  Artificial protein based on human PYY

SEQ ID NO: 42 - protein, 36 aa, ARTIFICIAL
  Artificial protein based on human PYY
KPIKPEAPGEDASPEELNRYYASLRHYLNLVTRQRY

SEQ ID NO: 43 - protein, 34 aa, ARTIFICIAL
  Artificial protein based on human PYY
ISPEAPGEDASPEELNRYYASLRHYLNKVTRQRY

SEQ ID NO: 44 - protein, 34 aa, ARTIFICIAL
  Artificial protein based on human PYY
IKPEAPGEDASPEELDRYYASLRHYLNKVTRQRY

SEQ ID NO: 45 - protein, 34 aa, ARTIFICIAL
  Artificial protein based on human PYY
IKPEAPGEDASPEELNRYYASLRHYLDKVTRQRY

SEQ ID NO: 46 - protein, 34 aa, ARTIFICIAL
  Artificial protein based on human PYY
IEPEAPGEDASPEELNRYYASLRHYLNKVTRQRY

SEQ ID NO: 47 - protein, 34 aa, ARTIFICIAL; identical to SEQ ID NO: 44
  Artificial protein based on human PYY

SEQ ID NO: 48 - protein, 34 aa, ARTIFICIAL
  Artificial protein based on human PYY
IRPEAPGEDASPEELNRYYESLRHYLNKVTRQRY

SEQ ID NO: 49 - protein, 34 aa, ARTIFICIAL
  Artificial protein based on human PYY
IRPEAPGEDASPEELERYYESLRHYLNKVTRQRY

SEQ ID NO: 50 - protein, 34 aa, ARTIFICIAL
  Artificial protein based on human PYY
IRPEAPGEDASPEELDRYYASLRHYLNKVTRQRY

SEQ ID NO: 51 - protein, 34 aa, ARTIFICIAL; identical to SEQ ID NO: 44
  Artificial protein based on human PYY

SEQ ID NO: 52 - protein, 34 aa, ARTIFICIAL
  Artificial protein based on human PYY
IKPEAPGEDASAEELNRYYASLRHYLNKVTRQRY

SEQ ID NO: 53 - protein, 34 aa, ARTIFICIAL
  Artificial protein based on human PYY
IKPEAPGEDASPEELARYYASLRHYLNKVTRQRY

SEQ ID NO: 54 - protein, 34 aa, ARTIFICIAL
  Artificial protein based on human PYY
IVPEAPGEDASPEELNRYYASLRHYLNKVTRQRY

SEQ ID NO: 55 - protein, 34 aa, ARTIFICIAL; identical to SEQ ID NO: 6
  Artificial protein based on human PYY

SEQ ID NO: 56 - protein, 34 aa, ARTIFICIAL; identical to SEQ ID NO: 6
  Artificial protein based on human PYY

SEQ ID NO: 57 - protein, 34 aa, ARTIFICIAL; identical to SEQ ID NO: 6
  Artificial protein based on human PYY

SEQ ID NO: 58 - protein, 34 aa, ARTIFICIAL; identical to SEQ ID NO: 6
  Artificial protein based on human PYY

SEQ ID NO: 59 - protein, 34 aa, ARTIFICIAL; identical to SEQ ID NO: 6
  Artificial protein based on human PYY

SEQ ID NO: 60 - protein, 34 aa, ARTIFICIAL; identical to SEQ ID NO: 6
  Artificial protein based on human PYY

SEQ ID NO: 61 - protein, 34 aa, ARTIFICIAL; identical to SEQ ID NO: 6
  Artificial protein based on human PYY

SEQ ID NO: 62 - protein, 34 aa, ARTIFICIAL; identical to SEQ ID NO: 6
  Artificial protein based on human PYY

SEQ ID NO: 63 - protein, 34 aa, ARTIFICIAL; identical to SEQ ID NO: 6
  Artificial protein based on human PYY

SEQ ID NO: 64 - protein, 34 aa, ARTIFICIAL; identical to SEQ ID NO: 6
  Artificial protein based on human PYY

SEQ ID NO: 65 - protein, 34 aa, ARTIFICIAL; identical to SEQ ID NO: 6
  Artificial protein based on human PYY

SEQ ID NO: 66 - protein, 34 aa, ARTIFICIAL; identical to SEQ ID NO: 44
  Artificial protein based on human PYY

SEQ ID NO: 67 - protein, 32 aa, ARTIFICIAL; identical to SEQ ID NO: 36
  Artificial protein based on human PYY

SEQ ID NO: 68 - protein, 34 aa, ARTIFICIAL
  Artificial protein based on human PYY
IAPEAPGEDASPEELDRYYASLRHYLNKVTRQRY

SEQ ID NO: 69 - protein, 34 aa, ARTIFICIAL; identical to SEQ ID NO: 35
  Artificial protein based on human PYY

SEQ ID NO: 70 - protein, 28 aa, ARTIFICIAL
  Artificial protein based on human PYY
GEDASPEELNRYYASLRHYLNKVTRQRY

SEQ ID NO: 71 - protein, 34 aa, ARTIFICIAL
  Artificial protein based on human PYY
IGPEAPGEDASPEELNRYYASLRHYLNKVTRQRY

SEQ ID NO: 72 - protein, 33 aa, ARTIFICIAL
  Artificial protein based on human PYY
RPEAPGEDASPEELDRYYASLRHYLNKVTRQRY

SEQ ID NO: 73 - protein, 33 aa, ARTIFICIAL; identical to SEQ ID NO: 72
  Artificial protein based on human PYY

SEQ ID NO: 74 - protein, 33 aa, ARTIFICIAL
  Artificial protein based on human PYY
RPEAPGEDASPEELNRYYASLRHYLNKVTRQRY

SEQ ID NO: 75 - protein, 34 aa, ARTIFICIAL
  Artificial protein based on human PYY
IKPEAPGEDASPEELNRYYASLRHYLNKVTRQRF

SEQ ID NO: 76 - protein, 34 aa, ARTIFICIAL
  Artificial protein based on human PYY
IKPEAPGEDASPEELNRYYASLRHYLNKVTRQRA

SEQ ID NO: 77 - protein, 34 aa, ARTIFICIAL
  Artificial protein based on human PYY
IKPEAPGEDASPEELNEYYRSLRHYLNKVTRQRY

SEQ ID NO: 78 - protein, 34 aa, ARTIFICIAL; identical to SEQ ID NO: 35
  Artificial protein based on human PYY

SEQ ID NO: 79 - protein, 34 aa, ARTIFICIAL; identical to SEQ ID NO: 71
  Artificial protein based on human PYY

SEQ ID NO: 80 - protein, 34 aa, ARTIFICIAL; identical to SEQ ID NO: 35
  Artificial protein based on human PYY

SEQ ID NO: 81 - protein, 34 aa, ARTIFICIAL; identical to SEQ ID NO: 50
  Artificial protein based on human PYY

SEQ ID NO: 82 - protein, 34 aa, ARTIFICIAL; identical to SEQ ID NO: 6
  Artificial protein based on human PYY

SEQ ID NO: 83 - protein, 34 aa, ARTIFICIAL; identical to SEQ ID NO: 68
  Artificial protein based on human PYY

SEQ ID NO: 84 - protein, 34 aa, ARTIFICIAL; identical to SEQ ID NO: 50
  Artificial protein based on human PYY

SEQ ID NO: 85 - protein, 34 aa, ARTIFICIAL; identical to SEQ ID NO: 35
  Artificial protein based on human PYY

SEQ ID NO: 86 - protein, 34 aa, ARTIFICIAL; identical to SEQ ID NO: 35
  Artificial protein based on human PYY

SEQ ID NO: 87 - protein, 34 aa, ARTIFICIAL
  Artificial protein based on human PYY
IAPEAPGEDASPEELNRYYASLRHYLNKVTRQRY

SEQ ID NO: 88 - protein, 34 aa, ARTIFICIAL; identical to SEQ ID NO: 6
  Artificial protein based on human PYY

SEQ ID NO: 89 - protein, 34 aa, ARTIFICIAL; identical to SEQ ID NO: 87
  Artificial protein based on human PYY

SEQ ID NO: 90 - protein, 34 aa, ARTIFICIAL; identical to SEQ ID NO: 35
  Artificial protein based on human PYY

SEQ ID NO: 91 - protein, 34 aa, ARTIFICIAL
  Artificial protein based on human PYY
IKPEAEGEDASPEELNRYYASLRHYLNKVTRQRY

SEQ ID NO: 92 - protein, 34 aa, ARTIFICIAL
  Artificial protein based on human PYY
IKPEAPEEDASPEELNRYYASLRHYLNKVTRQRY

SEQ ID NO: 93 - protein, 34 aa, ARTIFICIAL; identical to SEQ ID NO: 87
  Artificial protein based on human PYY

SEQ ID NO: 94 - protein, 34 aa, Homo sapiens; identical to SEQ ID NO: 32
  MISC_FEATURE (1)..(1): This position is referred to as position 3 in the description

(19) United States
(12) Patent Application Publication
(10) Pub. No.: US 2013/0096055 A1
(43) Pub. Date: Apr. 18, 2013

(54) LONG-ACTING Y2 RECEPTOR AGONISTS

(71) Applicants: Jacob Kofoed, Vaerloese (DK); Lars Ynddal, Jyllinge (DK); Soeren Oestergaard, Broenshoej (DK); Rasmus Joergensen, Maaloev (DK); Flemming Sejer Nielsen, Roskilde (DK); Cecilia Nilsson, Uppsala (SE); Jens Kaalby Thomsen, Hillerup (DK)

(72) Inventors: Jacob Kofoed, Vaerloese (DK); Lars Ynddal, Jyllinge (DK); Soeren Oestergaard, Broenshoej (DK); Rasmus Joergensen, Maaloev (DK); Flemming Sejer Nielsen, Roskilde (DK); Cecilia Nilsson, Uppsala (SE); Jens Kaalby Thomsen, Hillerup (DK)

(73) Assignee: Novo Nordisk A/S, Bagsværd (DK)

(21) Appl. No.: 13/508,433

(22) PCT Filed: Nov. 15, 2010
(86) PCT No.: PCT/EP2010/067454

§ 371 Date: Jul. 12, 2012

Related U.S. Application Data
(60) Provisional application No. 61/261,771, filed on Nov. 17, 2009.

(30) Foreign Application Priority Data
Nov. 13, 2009 (EP) 09175895.3

(51) Int. Cl. C07K 14/575

(52) U.S. Cl. CPC C07K 14/575
USPC 514/5.2; 530/324; 514/9.7; 514/6.9

(57) ABSTRACT

The present invention relates to PYY derivatives comprising a serum albumin binding side chain, wherein said derivative has a half-life of at least 7 hours as determined by Assay (IV) described herein as well as compositions comprising said derivative and its use in therapy.

### FIELD OF THE INVENTION

This invention relates to the field of therapeutic peptides, i.e. to new protracted PYY peptides derivatives comprising a serum albumin binding side chain, and their use in therapy.

### BACKGROUND OF THE INVENTION

Peptide Tyrosine-Tyrosine (PYY) is released during a meal from L-cells in the distal small intestine and the colon. PYY is known to have peripheral effects in the GI-tract and also act centrally as a satiety signal. PYY is released as PYY(1-36) but is cleaved to PYY(3-36) which constitutes approximately 50% of the circulating PYY. The enzyme responsible for the degradation is dipeptidyl peptidase IV (DPP IV). PYY(3-36) is rapidly eliminated by proteases and other clearance mechanisms. The half-life of PYY(3-36) has been reported to be <30 minutes in pigs (Ito T et al, Journal of Endocrinology (2006), 191, pp 113-119). Thus, PYY display suboptimal pharmacokinetic properties and have to be administered at least once-daily. PYY(3-36) signals through the Y2 receptor and the determinants for specificity of PYY towards the Y2 receptor are mainly located in the C-terminal part of the peptide. Whereas PYY1-36 activates Y1, Y2, and Y5 receptors with very little selectivity the DPPIV processed PYY3-36 display increased selectivity for the Y2 receptor. Y2 receptor activation is known to decrease appetite and food-intake whereas Y1 and Y5 receptor activation lead to an increase in appetite and food-intake. Furthermore, Y1 and Y5 receptor activation can increase blood pressure.

Bioactive peptides need to be inactivated after stimuli of their respective receptors. In-activation routes for circulating peptides comprise degradation by cell-surface or soluble plasma peptidases as well as different excretion routes. The former is accomplished by a panel of proteases, including both highly specialized peptidases which regulate receptor selectivity of peptides as well as broad specificity peptidases responsible for complete peptide inactivation.

Among the types of clearance mechanisms, renal clearance is the most important for the removal of PYY. The removal of peptides and proteins depends on the hydrodynamic radius of the molecule but also other factors such as charge, protein binding, polarity and reabsorbtion play some roles. In most cases peptides and proteins with a molecular weight below 20 kDa will pass freely into the glomerular basal membrane and accumulates in the proximal tubule after which degradation will find place and finally excretion into the urine. Larger proteins are degraded in the kidneys by membrane bound enzymes and the degradation kinetics is very different across different proteins.

### SUMMARY OF THE INVENTION

In one embodiment the invention relates to a PYY derivative comprising a serum albumin binding side chain, wherein said serum albumin binding side chain comprises an alkyl chain with at least 14 carbon atoms and wherein said derivative has a half-life of at least 7 hours as determined by Assay (IV).

In one embodiment the invention relates to a PYY derivative comprising a serum albumin binding side chain, wherein
said derivative has a half-life of at least 7 hours as determined by Assay (IV), and wherein
said serum albumin binding side chain comprises an alkyl chain of at least 14 carbon atoms, and wherein
said alkyl chain comprises a distal carboxylic acid group or a distal tetrazole group, and wherein
said serum albumin binding side chain is attached to the N-terminal amino group or an amino acid in a position selected from the group consisting of position 1, 3, 6, 7, 9, 10, 11, 12, 14, 15, 17, 18, 19, 21, 22, 23 and 30, wherein said position is relative to hPYY(1-36).

In one embodiment the invention relates to a composition comprising the PYY derivative as defined herein and at least one pharmaceutical excipient. In one embodiment the invention relates to use of the PYY derivative in medicine.

In one embodiment the invention relates to a method of treatment of a condition responsive to Y receptor modulation, such as obesity or obesity-related diseases, by administration of the PYY derivative as defined herein.

### DESCRIPTION OF THE INVENTION

The present invention relates to certain PYY derivatives, compositions thereof and their use in medicine. In one embodiment the PYY derivative has protracted pharmacokinetic properties. In one embodiment the PYY derivative is an agonist of the Y2 receptor and has protracted pharmacokinetic properties. In one embodiment the present invention provides PYY derivatives with an improved half-life determined according to Assay (IV) as described herein compared to hPYY(3-36). In one embodiment the improved half-life is caused by covalent attachment of an albumin binding side chain to the PYY compound. In one embodiment the improved half-life is caused by the type of acylation and/or the position of acylation in the peptide sequence of the PYY derivative. Surprisingly, the present inventors have found that the half-life of the PYY derivatives depend on the position of acylation.

The beneficial effects of PYY(3-36) is believed to be mediated through the Y2 receptor while activation of the Y1 and Y5 receptors can lead to adverse effects or abolish the therapeutic effect. Thus, it is desirable to develop PYY derivatives thereof with increased selectivity for the Y2 receptor relative to the Y1 and/or Y5 receptors. An increase in Y2 receptor selectivity is intended to mean a relative decrease in Y1 and/or Y5 receptor potency compared to Y2 receptor potency or a relative increase in Y2 receptor potency compared to Y1 and/or Y5 receptor potency. Thus a PYY(3-36) derivative which, compared to hPYY(3-36), displays a relatively larger decrease in potency on the Y1 and/or Y5 receptor than on the Y2 receptor as determined by Assay (II) and/or (III) and (I), respectively, has an increased Y2 receptor selectivity. In one embodiment the PYY derivative has increased selectivity for the Y2 receptor relative to the Y1 and/or Y5 receptor compared to hPYY(3-36). In one embodiment the PYY derivative has increased selectivity for the Y2 receptor relative to the Y1 receptor, i.e. a higher Y1/Y2 receptor potency ratio, compared to hPYY(3-36). In one embodiment the PYY derivative has increased selectivity for the Y2 receptor relative to the Y5 receptor, i.e. a higher Y5/Y2 receptor potency ratio, compared to hPYY(3-36). In one embodiment the PYY derivative has increased selectivity for the Y2 receptor relative to the Y1 and/or the Y5 receptor and has protracted pharmacokinetic properties, such as a longer half-life. In one embodiment the PYY derivative has an improved Y2 receptor potency, i.e a lower EC50 value. In one embodiment administration of the PYY derivative results in reduced food intake. In one embodiment food intake is determined according to Assay (V) or Assay (VII) described herein. In one embodiment administration of the PYY derivative results in reduced body weight. In one embodiment body weight is determined according to Assay (VI) described herein.

The term “agonist” means any compound that activates the target receptor and elicits at least one of the in vivo or in vitro effects elicited by the endogenous agonist for said receptor.

“Protracted properties” of a peptide is prolonged duration of action of the peptide which results in a dosing regime with lower frequency, e.g., once-daily, every other day, once-weekly or less than once-weekly. In one embodiment the protracted properties of the PYY derivative is shown as prolonged half-life in plasma or prolonged biological activity compared to hPYY(1-36) or hPYY(3-36). In one embodiment the protraction of the PYY derivative is determined by monitoring the concentration thereof in plasma after administration to animals, such as healthy pigs, using methods as described herein, such as Assay (IV) described herein, PK i.v. mini-pig. In one embodiment the protraction of the PYY derivative is determined by monitoring the duration of effect of said derivative in at least one biological assay, such as Assay (IV), Assay (V), Assay (VI), Assay (VII), Assay (VIII) or Assay (IX) described herein.

The terms “human PYY” and “hPYY” are intended to mean hPYY(1-36) or hPYY(3-36). In one embodiment hPYY(1-36) is YPIKPEAPGEDASPEELNRYYASLRHYLNLVTRQRY. In one embodiment hPYY(3-36) is hPYY(1-36), wherein the N-terminal Tyr and Pro are deleted. In one embodiment the term PYY is intended to refer to human PYY. In one embodiment a combination of at least two of the effects mentioned herein is achieved. The terms “peptide backbone” and “amino acid sequence of the PYY derivative” are used interchangeably to describe the peptide part of the PYY derivative, i.e. the PYY derivative without the serum albumin binding side chain.

### PYY Derivative

In one embodiment the PYY derivative comprises a serum albumin binding side chain which forms attachment of said derivative to serum albumin in vivo. In one embodiment said attachment is covalent or non-covalent. In one embodiment said attachment is non-covalent. In one embodiment the serum albumin binding side chain comprises an alkyl chain with at least 14 carbon atoms and the PYY derivative binds non-covalently to albumin. In one embodiment the PYY derivative has an improved duration of action. In one embodiment the PYY derivative can be dosed less frequently, such as once-daily or more rarely, than hPYY(3-36). In one embodiment the PYY derivative comprises attachment of at least one side chain which binds to albumin, i.e. a serum albumin binding side chain. In one embodiment the serum albumin binding side chain comprises a carboxy group of a carboxylic acid or of a dicarboxylic acid acylated to a nitrogen atom.

In one embodiment the serum albumin binding group comprises an alkyl chain with at least 14 carbon atoms. In one embodiment the serum albumin binding side chain comprises at least 14 carbon atoms, such as 16, 18 or 20 carbon atoms. In one embodiment the serum albumin binding group comprises an alkyl chain with at least 16 carbon atoms. In one embodiment the serum albumin binding group comprises an alkyl chain with at least 18 carbon atoms. In one embodiment the serum albumin binding group comprises an alkyl chain with at least 20 carbon atoms. In one embodiment the serum albumin binding side chain comprises an amide. In one embodiment the serum albumin binding side chain comprises a distal carboxylic acid group or a distal tetrazole group. In one embodiment the serum albumin binding side chain comprises an alkyl chain with at least 14 carbon atoms comprising a distal carboxylic acid or a distal tetrazole group. In one embodiment the serum albumin binding side chain comprises a C18 dicarboxylic acid or a C16 dicarboxylic acid. In one embodiment the serum albumin binding side chain comprises a C16 carboxylic acid.

In one embodiment the serum albumin binding side chain comprises a distal carboxylic group, wherein said carboxylic group is bound to the first end of an alkyl chain with at least 14 carbon atoms, wherein the second end of said alkyl chain is bound to the carbonyl group of an amide.

In one embodiment the serum albumin binding side chain comprises an alkyl chain with at least 14 carbon atoms, wherein said alkyl chain comprises a distal carboxylic acid or a distal tetrazole group and wherein said alkyl chain comprises a proximal carbonyl group. In one embodiment the serum albumin binding side chain comprises formula (X)

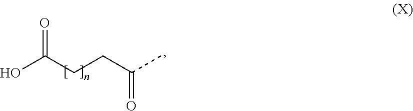

wherein n is at least 13, such as n is 13, 14, 15, 16, 17, 18 or 19. In one embodiment the serum albumin binding side chain comprises formula (X), wherein n is in the range of 13 to 19, such as in the range of 13 to 17. In one embodiment the serum albumin binding side chain comprises formula (X), wherein n is 13, 15 or 17. In one embodiment the serum albumin binding side chain comprises formula (X), wherein n is 13. In one embodiment the serum albumin binding side chain comprises formula (X), wherein n is 15. In one embodiment the serum albumin binding side chain comprises formula (X), wherein n is 17.

The term “derivative” and the related terms “derivatised” and “derivatisation” as used herein in relation to a peptide means a chemically altered peptide, wherein at least one substituent is not present in the non-altered peptide, i.e. a peptide which has been covalently altered. Typical alterations are amides, carbohydrates, alkyl groups, acyl groups, esters and the like. In one embodiment the serum albumin binding side chain is present at the N-terminus, C-terminus or at the side chain of an amino acid residue in the sequence of the PYY derivative. In one embodiment additional sites for derivatisation are provided by substitution of at least one amino acid with lysine, aspartic acid, glutamic acid or cysteine. In one embodiment the PYY derivative is conjugated to one, two or three serum albumin binding side chain molecules. In one embodiment the serum albumin binding side chain is attached to an amine group or a carboxylic acid group of the amino acid sequence of the PYY derivative. In one embodiment the serum albumin binding side chain is attached to an amine group or a carboxylic acid group of an amino acid side chain in the PYY derivative.

In one embodiment any amino acid residue in the PYY derivative may be derivatised. In one embodiment the amino acid residue which is derivatised comprises an amino group. In one embodiment the amino acid residue which is derivatised comprises a primary amino group in a side chain. In one embodiment the amino acid residue which is derivatised is lysine. In one embodiment the amino acid residue which is derivatised is cysteine. In one embodiment the PYY derivative is only derivatised in one position, e.g. only one amino acid residue is derivatised. In one embodiment position 1 in the PYY derivative is derivatised. In one embodiment position 2 in the PYY derivative is derivatised.

In one embodiment position 3 in the PYY derivative is derivatised. In one embodiment position 4 in the PYY derivative is derivatised. In one embodiment position 5 in the PYY derivative is derivatised. In one embodiment position 6 in the PYY derivative is derivatised. In one embodiment position 7 in the PYY derivative is derivatised. In one embodiment position 8 in the PYY derivative is derivatised. In one embodiment position 9 in the PYY derivative is derivatised. In one embodiment position 10 in the PYY derivative is derivatised. In one embodiment position 11 in the PYY derivative is derivatised. In one embodiment position 12 in the PYY derivative is derivatised. In one embodiment position 13 in the PYY derivative is derivatised. In one embodiment position 14 in the PYY derivative is derivatised. In one embodiment position 15 in the PYY derivative is derivatised. In one embodiment position 16 in the PYY derivative is derivatised. In one embodiment position 17 in the PYY derivative is derivatised. In one embodiment position 18 in the PYY derivative is derivatised. In one embodiment position 19 in the PYY derivative is derivatised. In one embodiment position 20 in the PYY derivative is derivatised. In one embodiment position 21 in the PYY derivative is derivatised. In one embodiment position 22 in the PYY derivative is derivatised. In one embodiment position 23 in the PYY derivative is derivatised. In one embodiment position 24 in the PYY derivative is derivatised. In one embodiment position 25 in the PYY derivative is derivatised. In one embodiment position 26 in the PYY derivative is derivatised. In one embodiment position 27 in the PYY derivative is derivatised. In one embodiment position 28 in the PYY derivative is derivatised. In one embodiment position 29 in the PYY derivative is derivatised. In one embodiment position 30 in the PYY derivative is derivatised. In one embodiment position 31 in the PYY derivative is derivatised. In one embodiment position 32 in the PYY derivative is derivatised. In one embodiment position 33 in the PYY derivative is derivatised. In one embodiment position 34 in the PYY derivative is derivatised. In one embodiment position 35 in the PYY derivative is derivatised. In one embodiment position 36 in the PYY derivative is derivatised.

In one embodiment the serum albumin binding side chain is attached to the N-terminal amino group or an amino acid in a position selected from the group consisting of position 1, 3, 6, 7, 9, 10, 11, 12, 14, 15, 17, 18, 19, 21, 22, 23 and 30. In one embodiment the serum albumin binding side chain is attached to the N-terminal amino group or an amino acid in a position selected from the group consisting of position 3, 6, 7, 10, 11, 14, 17, 18, 19, 21, 22 and 30. In one embodiment the serum albumin binding side chain is attached to an amino acid in a position selected from the group consisting of position 7, 10, 21, 22 and 30. In one embodiment the serum albumin binding side chain is attached to an amino acid in a position selected from the group consisting of position 10, 11, 14, 17, 19, 21 and 30. In one embodiment the serum albumin binding side chain is attached to an amino acid in a position selected from the group consisting of position 10, 21 and 30, such as in position 30. In one embodiment the serum albumin binding side chain is attached to the N-terminal amino group or an amino acid in a position selected from the group consisting of position 3, 6, 7, 10, 11, 14, 17, 18, 19, 21, 22 and 30. In one embodiment the serum albumin binding side chain is attached to the amino acid in position 30.

In one embodiment the serum albumin binding side chain is not attached to the amino acid in positions 18, 19, 22 or 23. In one embodiment the serum albumin binding side chain is not attached to the N-terminal or C-terminal amino group.

In one embodiment the PYY derivative has improved half-life as determined by Assay (IV) as described herein; such as a half-life of at least 7 h, such as at least 35 h. In one embodiment the PYY derivative does not have a serum albumin binding side chain attached to the side chain of the amino acid in position 8. In one embodiment the PYY derivative has a serum albumin binding side chain attached to the side chain of the amino acid in a position selected from the group consisting of position 17, 22, 24, 25, 26, 27, 28, 29, 30, 31, 32, 33 and 35.

In one embodiment the PYY derivative maintains a sufficient or has improved Y2 receptor potency as determined by Assay (I) as described herein; such as a Y2 receptor potency of less than 18 nM, such as less than 9 nM. In one embodiment the PYY derivative has a serum albumin binding side chain attached to the N-terminal amino group or to the side chain of the amino acid in a position selected from the group consisting of 1, 3, 6, 7, 10, 11, 14, 17, 18, 19, 21, 22, 23 and 30. In one embodiment the PYY derivative has a serum albumin binding side chain attached to the side chain of the amino acid in a position selected from the group consisting of 6, 7, 10, 11, 14, 17, 18, 19, 21, 22 and 30.

In one embodiment the PYY derivative maintains a sufficient or has improved Y1/Y2 receptor potency ratio as determined by Assay (I) and Assay (II), respectively, as described herein; such as a Y1/Y2 receptor potency ratio of at least 22, such as at least 44. In one embodiment the PYY derivative has a serum albumin binding side chain attached to the side chain of the amino acid in a position selected from the group consisting of 7, 10, 20, 21, 22, 28 and 30; such as position 22 or 30.

In one embodiment the PYY derivative maintains a sufficient or has improved Y5/Y2 receptor potency ratio as determined by Assay (I) and Assay (III), respectively, as described herein; such as a Y5/Y2 receptor potency ratio of at least 10, such as at least 19. In one embodiment the PYY derivative has a serum albumin binding side chain attached to the side chain of the amino acid in a position selected from the group consisting of 8, 10, 11, 13, 14, 16, 17, 19, 20, 21, 30 and 32; such as a position selected from the group consisting of 8, 11, 13 and 30.

In one embodiment the N-terminal position of the PYY derivative is derivatised. In one embodiment the N-terminal position of the PYY derivative is acylated. In one embodiment the N-terminal position of the PYY derivative is derivatised with a serum albumin binding side chain comprising CH₃(CH₂)ᵣCO—, wherein r is at least 12, such as 14, 16 or 18. In one embodiment the N-terminal position of hPYY(3-36) is derivatised with a serum albumin binding side chain comprising CH₃(CH₂)ᵣCO—, wherein r is at least 12, such as 14, 16 or 18.

Examples of amino acid residues comprising an amino group is lysine, ornithine, Epsilon-N-alkylated lysine such as Epsilon-N methyllysine, O-aminoethylserine, O-aminopropylserine or longer O-alkylated serines containing a primary or secondary amino group in the side chain. In one embodiment the derivatised amino acid residue comprises a primary amino group in a side chain. Examples of amino acid residues comprising a primary amino group is lysine, ornithine, O-aminoethylserine, O-aminopropylserine or longer O alkylated serines containing a primary amino group in the side chain. In one embodiment the term “serum albumin binding side chain” is intended to mean a moiety which has serum albumin binding properties. In one embodiment the term “serum albumin binding” refers to an inherent ability of a compound to bind to circulating serum albumin and said binding is optionally non-covalent. Various compounds exhibit different ability to bind to albumin. A high ability of a compound to bind to serum albumin results in a high fraction of compound bound to albumin while the corresponding fraction of compound, which is not bound to albumin, in serum will be low.

An example of a method for determination of albumin binding is as follows: Serum albumin binding can be measured by using columns with immobilised serum albumin from human or other species. The affinity of a given peptide can be measured by an altered elution time from the column and the relative affinities between different albumin binding peptides can be established by comparing the elution time profiles. In one method serum albumin peptides can be biotinylated and the binding of the peptide can be determined by enzyme linked immuno assay (ELISA) technique using microtiter plate with immobilised albumin. The visualisation of the binding is done by using avidin or streptavidin conjugated to either horseradish peroxidise or alkaline phosphatase. The relative affinities of different albumin binding peptides can be measured. Other affinity experiments that may be used in the measurement of albumin binding comprise Biacore analysis and microcalorimetry.

In one embodiment the serum albumin binding side chain is lipophilic. In one embodiment the serum albumin binding side chain is attached to a lysine residue optionally via a spacer by conjugation chemistry such as by alkylation, acylation, ester formation or amide formation or to a cysteine residue by maleimide coupling. The term “spacer” as used herein means a molecular unit separates a peptide and a serum albumin binding side chain. In one embodiment the term “spacer” as used herein means a spacer that separates a peptide and the serum albumin binding side chain with a chemical moiety which comprises at least 5 non-hydrogen atoms where 30-50% of these are either N or O.

In one embodiment the serum albumin binding side chain is negatively charged at physiological pH. In one embodiment the serum albumin binding side chain comprises a group which can be negatively charged. In one embodiment the serum albumin binding side chain comprises a carboxylic acid group.

In one embodiment the serum albumin binding side chain is selected from the group consisting of a straight chain alkyl group, a branched alkyl group, a group which has an co-carboxylic acid group, and a partially or completely hydrogenated cyclopentanophenanthrene skeleton. In one embodiment the serum albumin binding side chain is a cibacronyl residue. In one embodiment the serum albumin binding side chain has from 6 to 40 carbon atoms, from 8 to 26 carbon atoms or from 8 to 20 carbon atoms. In one embodiment the serum albumin binding side chain is an acyl group selected from the group comprising CH₃(CH₂)ᵣCO—, wherein r is an integer from 4 to 38, specifically an integer from 4 to 24, more preferred selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—. In one embodiment the serum albumin binding side chain is an acyl group of a straight-chain or branched alkane α,ω-dicarboxylic acid. In one embodiment the serum albumin binding side chain is A-B-C-D- or A-C-D- or A-B-C- or A-C-,

wherein A- is

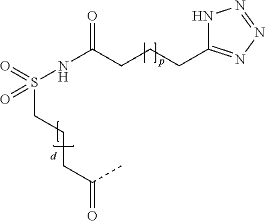

wherein p is selected from the group consisting of 10, 11, 12, 13 and 14, and d is selected from the group consisting of 0, 1, 2, 3, 4 and 5, and

-B- is selected from the group consisting of

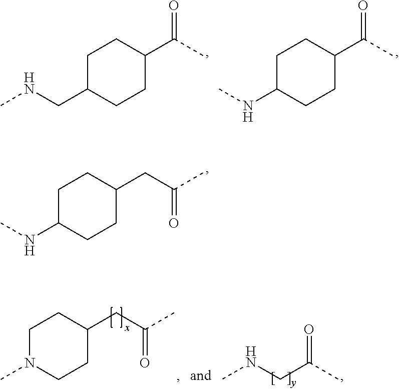

wherein x is selected from the group consisting of 0, 1, 2, 3 and 4, and y is selected from the group consisting of 1, 2, 3, 4, 5, 6, 7, 8, 9, 10, 11 and 12,

or A- is

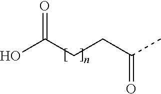

wherein n is selected from the group consisting of 12, 13, 14, 15, 16 17, 18 and 19, and B is selected from the group consisting of

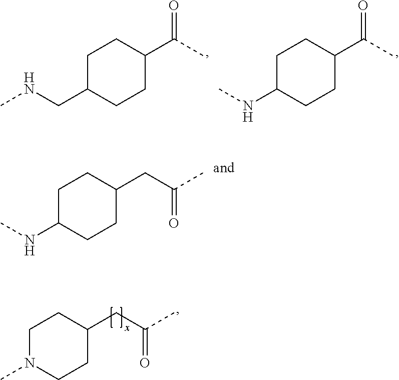

wherein x is selected from the group consisting of 0, 1, 2, 3 and 4, and

-C- is selected from the group consisting of

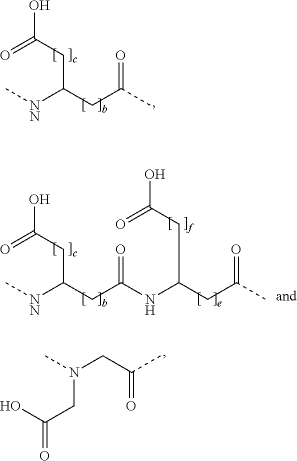

wherein b and e are each independently selected from the group consisting of 0, 1 and 2, and c and f are each independently selected from the group consisting of 0, 1 and 2 with the proviso that b is 1 or 2 when c is 0, or b is 0 when c is 1 or 2, and e is 1 or 2 when f is 0, or e is 0 when f is 1 or 2, and

-D- is attached to said amino acid residue and is a spacer. In one embodiment the serum albumin binding side chain is A-B-C-D- or A-C-D- or A-B-C- or A-C-,

wherein A- is

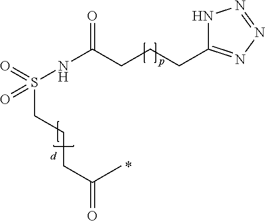

wherein p is selected from the group consisting of 10, 11, 12, 13 and 14, and d is selected from the group consisting of 0, 1, 2, 3, 4 and 5, and

-B- is selected from the group consisting of

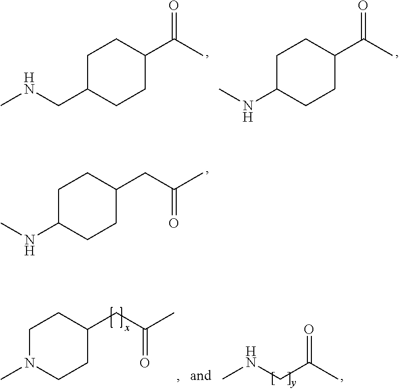

wherein x is selected from the group consisting of 0, 1, 2, 3 and 4, and y is selected from the group consisting of 1, 2, 3, 4, 5, 6, 7, 8, 9, 10, 11 and 12,

or A- is

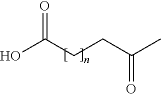

wherein n is selected from the group consisting of 12, 13, 14, 15, 16 17, 18 and 19, and B is selected from the group consisting of

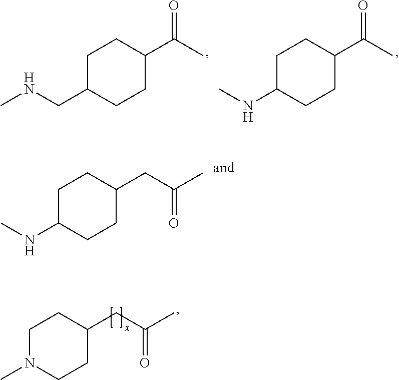

wherein x is selected from the group consisting of 0, 1, 2, 3 and 4, and

-C- is selected from the group consisting of

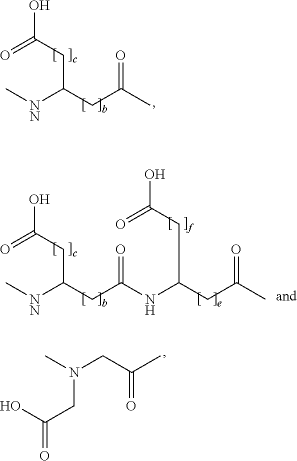

wherein b and e are each independently selected from the group consisting of 0, 1 and 2, and c and f are each independently selected from the group consisting of 0, 1 and 2 with the proviso that b is 1 or 2 when c is 0, or b is 0 when c is 1 or 2, and e is 1 or 2 when f is 0, or e is 0 when f is 1 or 2, and

-D- is attached to said amino acid residue and is a spacer.

In one embodiment the serum albumin binding side chain is attached to the N-terminal amino group, the amino group of the amidated C-terminal or the side chain of an amino acid, such as the side chain of 2,3-diaminopropionic acid, 2,4-diaminobutyric acid, ornithine or Lys.

In one embodiment the serum albumin binding side chain comprises at least one dicarboxylic acid, such as hexadecanedioic acid, octadecanedioic acid or dodecanedioic acid. It is believed that the negative charge in the distal end of the dicarboxylic acid increases affinity of the PYY derivative to serum albumin. In one embodiment the fatty diacid or tetrazole may be attached to a spacer, such as a negatively charged amino acid, e.g., L-gamma-glutamate. In one embodiment the alkyl chain, optionally comprising a dicarboxylic acid or a tetrazole group, may be attached to a hydrophobic spacer, such as tranexamic acid and isonipecotinic acid. In one embodiment the combined alkyl chain, optionally comprising a dicarboxylic acid or a tetrazole group, and the negatively charged amino acid may be separated with a spacer, such as 8-amino-3,6-dioxaoctanoic acid (Oeg) or several Oeg moelcules.

In one embodiment the serum albumin binding side chain is 2-(2-{2-[2-(2-{2-[(S)-4-Carboxy-4-({trans-4-[(19-carboxynonadecanoylamino)methyl]cyclohexanecarbonyl}amino)butyrylamino]-ethoxy}-ethoxy)acetylamino]ethoxy}ethoxy)acetyl. In one embodiment the serum albumin binding side chain is 2-(2-{2-[2-(2-{2-[(S)-4-Carboxy-4-(19-carboxynonadecanoylamino)butyrylamino]ethoxy}ethoxy)acetyl-amino]ethoxy}ethoxy)acetyl. In one embodiment the serum albumin binding side chain is 2-(2-{2-[2-(2-{2-(19-carboxynonadecanoylamino)ethoxy}ethoxy)-acetylamino]ethoxy}ethoxy)acetyl. In one embodiment the serum albumin binding side chain is [2-(2-{2-[2-(2-{2-((S)-4-carboxy-[(S)-4-Carboxy-4-(17-carboxyheptadecanoylamino)butyrylamino]butyrylamino)ethoxy}ethoxy)-acetylamino]ethoxy}ethoxy)acetyl. In one embodiment the serum albumin binding side chain is 2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl. In one embodiment the serum albumin binding side chain is (S)-4-Carboxy-4-(17-carboxyheptadecanoylamino)butyrylamino. In one embodiment the serum albumin binding side chain is (S)-4-Carboxy-4-(17-carboxyheptadecanoylamino)butyrylamino]-Ser-Ser-Gly-Ser-Ser-Gly.

In one embodiment the serum albumin binding side chain is 2-(2-{2-[2-(2-{2-[(S)-4-Carboxy-4-(15-carboxypentadecanoylamino)butyrylamino]ethoxy}ethoxy)acetylamino]ethoxy}ethoxy)acetyl. In one embodiment the serum albumin binding side chain is 2-(2-{2-[2-(2-{2-[(S)-4-Carboxy-4-(hexadecanoylamino) butyrylamino]ethoxy}ethoxy)acetyl-amino]ethoxy}ethoxy)acetyl. In one embodiment the serum albumin binding side chain is 4-(16-(1H-Tetrazol-5-yl)hexadecanoylsulfamoyl)butyryl]ethoxy}ethoxy)acetylamino]ethoxy}ethoxy)acetyl. In one embodiment the serum albumin binding side chain is 2-(2-{2-[2-(2-{2-[(4-(16-(1H-Tetrazol-5-yl)hexadecanoylsulfamoyl)butyryl]-ethoxy}ethoxy)-acetylamino]ethoxy}ethoxy)acetyl.

In one embodiment the serum albumin binding side chain is [2-(2-{2-[2-(2-{2-(11-carboxyundecanoylamino)-ethoxy}ethoxy)-acetylamino]ethoxy}ethoxy)acetyl]. In one embodiment the serum albumin binding side chain is [2-(2-{2-[2-(2-{2-(13-carboxytridecanoylamino)-ethoxy}ethoxy)-acetylamino]ethoxy}ethoxy)acetyl]. In one embodiment the serum albumin binding side chain is [2-(2-{2-[2-(2-{2-(15-carboxypentadecanoylamino)-ethoxy}ethoxy)-acetylamino]ethoxy}ethoxy)acetyl]. In one embodiment the serum albumin binding side chain is [2-(2-{2-[2-(2-{2-(17-carboxyheptadecanoylamino)-ethoxy}ethoxy)-acetylamino]ethoxy}ethoxy)acetyl]. In one embodiment the serum albumin binding side chain is [2-(2-{2-[2-(2-{2-[(S)-4-Carboxy-4-(11-carboxyundecanoylamino)butyrylamino]ethoxy}ethoxy)-acetylamino]ethoxy}ethoxy)acetyl]. In one embodiment the serum albumin binding side chain is [2-(2-{2-[2-(2-{2-[(S)-4-Carboxy-4-(13-carboxytridecanoylamino)butyrylamino]ethoxy}ethoxy)-acetylamino]ethoxy}ethoxy)acetyl].

In one embodiment -D- is a spacer providing distance of the serum albumin binding side chain to the peptide and may be selected from the group consisting of at least one PEG molecule, such as at least two consecutive PEG molecules, at least one glycine, such as at least two consecutive glycines, or other small polar residues. In one embodiment said spacer may be at least one 8-amino-3,6-dioxaoctanoic acid (Oeg) molecule, such as at least two consecutive Oeg molecules, or other spacers of the PEG type. In one embodiment said spacer may be a peptide and may comprise or consist of at least two consecutive Gly molecules forming a glycine polymer. In one embodiment the spacer is composed of several polar or hydrophilic amino acids, such as (Ser-Gly)ₙ where n is an integer and n=1-20, 1-10 or 1-5. In one embodiment the spacer may comprise non-alpha-amino acids, such as beta-alanine or 8-amino-caprylic acid or combinations thereof.

In one embodiment -D- is

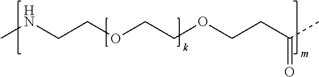

wherein k is selected from the group consisting of 0, 1, 2, 3, 4, 5, 11 and 27, and m is selected from the group consisting of 0, 1, 2, 3, 4, 5 and 6.

In one embodiment A-B-C-D- is selected and combined from any one of:

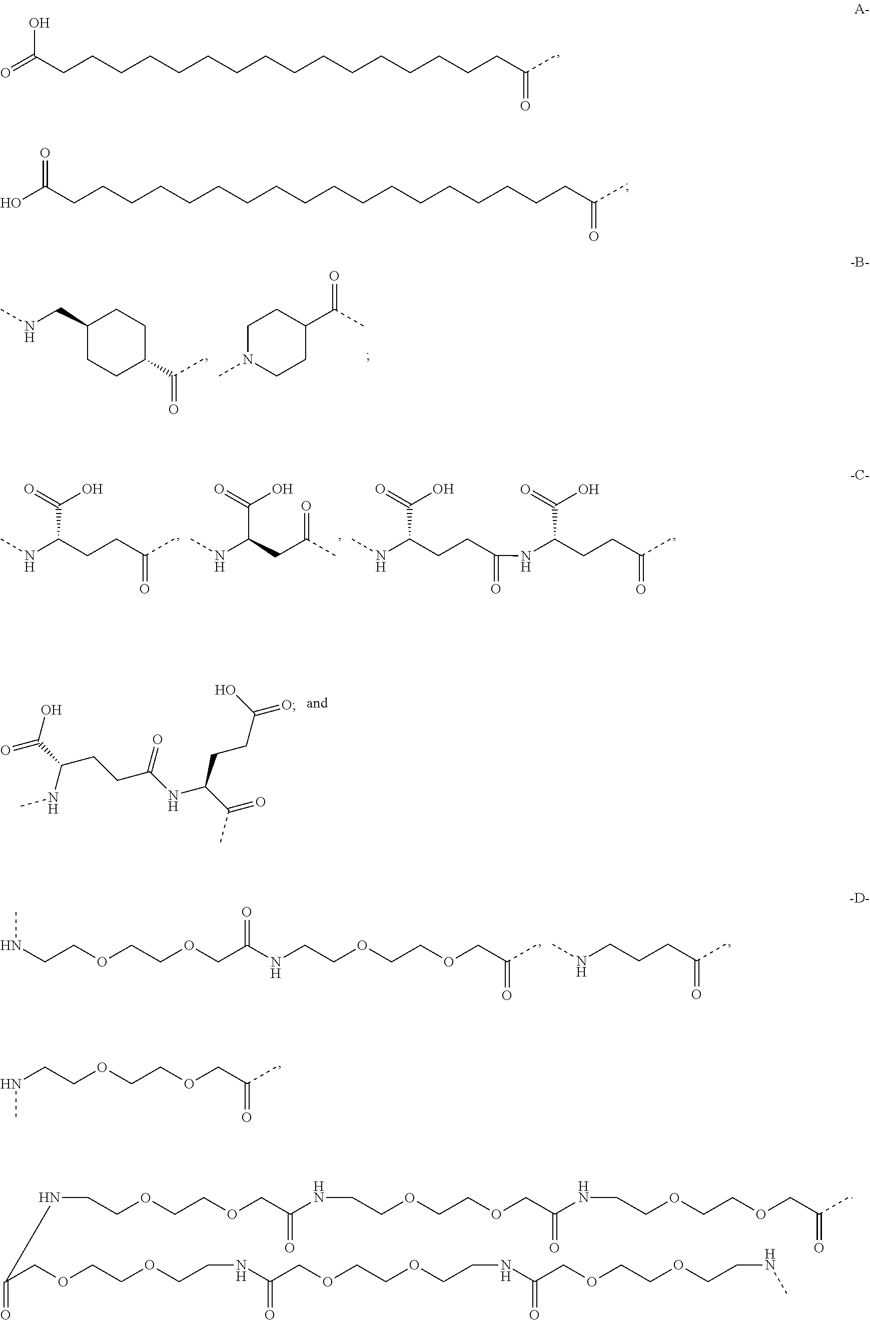

In one embodiment A-B-C-D- is selected and combined from any one of

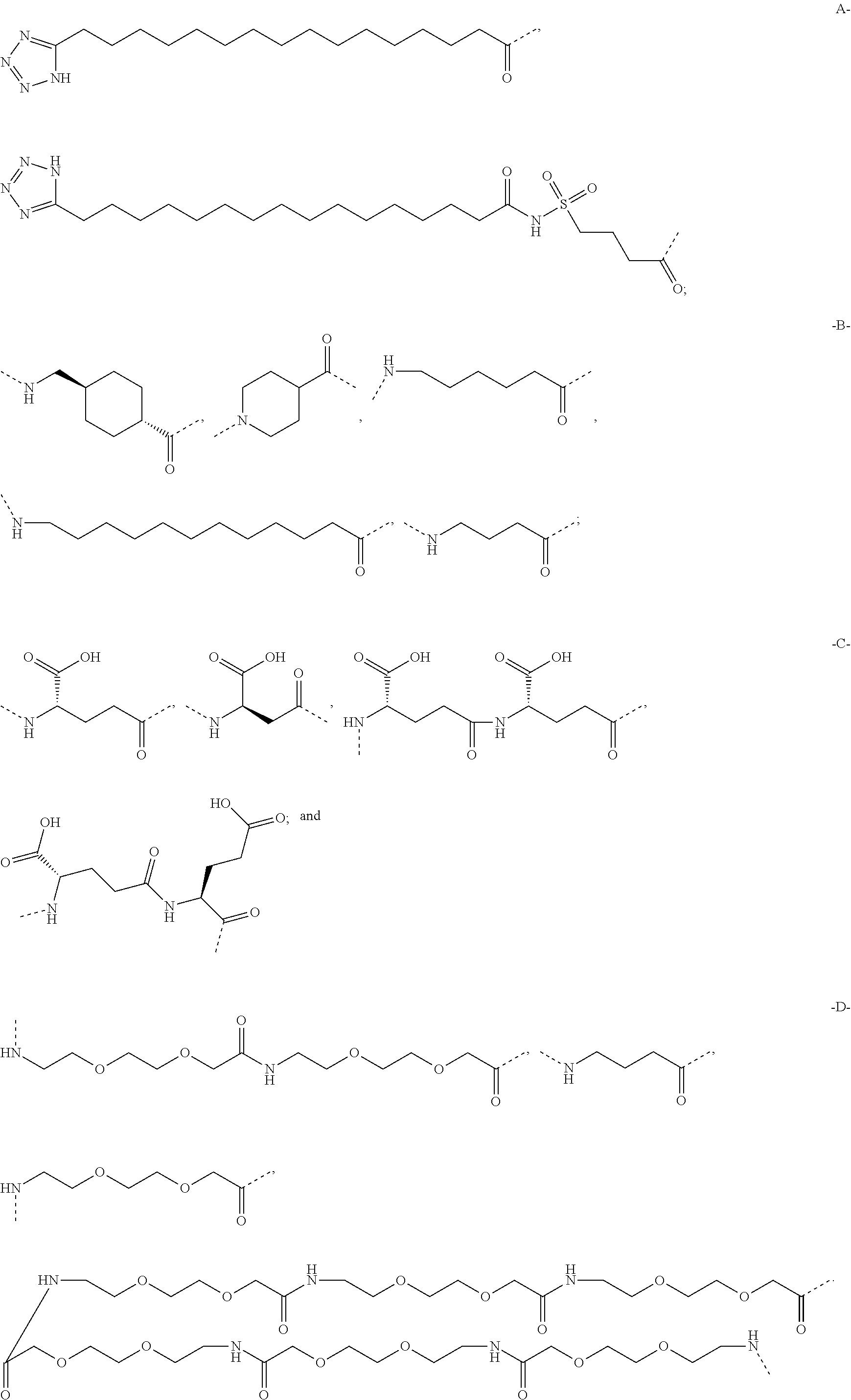

In one embodiment A-B-C-D- is selected from the group consisting of

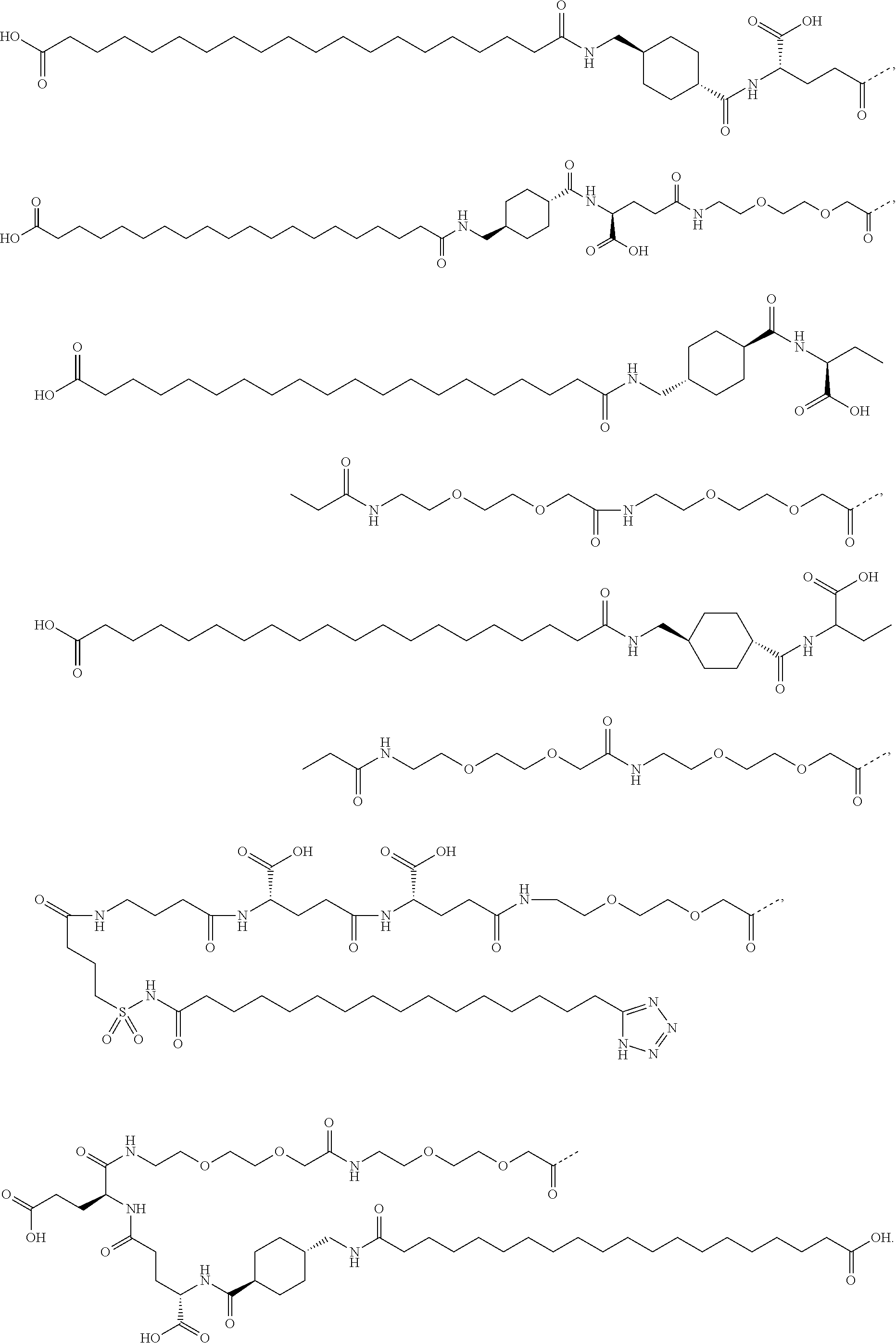

In one embodiment A-B-C-D comprises a serum albumin binding fragment A-B-C- and a hydrophilic spacer, D. In one embodiment the serum albumin binding side chain may be linked to an amino, carboxyl or thiol group, and may be linked by N or C termini or at the side chains of lysine, aspartic acid, glutamic acid or cysteine. In one embodiment the serum albumin binding side chain may be linked with diamine and dicarboxylic groups. In one embodiment the serum albumin binding side chain is a linear or branched lipophilic moiety containing 4-40 carbon atoms having a distal acidic group. In one embodiment the terminal dashed bonds from the attached groups A, B, C and D shown in the formulas herein are to be regarded as attachment bonds and not ending in methylene groups unless stated. In one embodiment the groups A, B, C and/or D are attached to each other by amide bonds.

### PYY Analogue

In one embodiment the PYY derivative comprises hPYY(1-36). In one embodiment the PYY derivative comprises hPYY(3-36). In one embodiment the PYY derivative comprises hPYY(5-36). In one embodiment the PYY derivative comprises an analogue of PYY. In one embodiment the PYY derivative comprises human PYY (hPYY), such as hPYY(3-36), or an analogue thereof. In one embodiment the PYY derivative comprises alterations selected from the group consisting of substitutions, deletions and modifications into the PYY peptide, wherein the PYY peptide may be hPYY(1-36) or hPYY(3-36). The term “analogue” as used herein referring to a peptide means a peptide wherein at least one amino acid residue of the peptide has been substituted with another amino acid residue and/or wherein at least one amino acid residue has been deleted from the peptide and/or wherein at least one amino acid residue has been added to the peptide and/or wherein at least one amino acid residue of the peptide has been modified. Such addition or deletion of amino acid residues can take place at the N-terminal of the peptide and/or at the C-terminal of the peptide. A simple nomenclature is used to describe the peptides according to the invention, e.g., [Ala25] hPYY(3-36) designates an analogue of the human PYY wherein the naturally occurring arginine in position 25 has been substituted with alanine and the naturally occurring tyrosine and proline in position 1 and 2, respectively, have been deleted. In one embodiment the PYY derivative comprises a maximum of twelve, such as a maximum of 10, 8 or 6, amino acids which have been alterered, e.g., by substitution, deletion, insertion and/or modification, as compared to hPYY(1-36).

In one embodiment references herein to positions in a peptide or the PYY derivative refers to positions in hPYY(1-36) or hPYY(3-36). In one embodiment the N-terminal position in PYY(3-36) is referred to as position 3. In one embodiment the PYY derivative may be derived from vertebrates, such as a mammal, including human, mouse, sheep, goat, cow or horse.

The term “peptide” as used herein means a compound composed of at least five constituent amino acids connected by peptide bonds. In one embodiment the N-terminus of the peptide is an amino group and/or said C-terminus is a carboxylic acid group. In one embodiment all amino acids in the PYY derivative for which the optical isomer is not stated is to be understood to mean the L-isomer. In one embodiment at least one of the amino acids in the PYY derivative is a D-amino acid. In one embodiment the constituent amino acids of the PYY derivative may be selected from at least one of the group of the proteinogenic amino acids encoded by the genetic code and the non-proteinogenic amino acids, such as natural amino acids which are not encoded by the genetic code and synthetic amino acids. In one embodiment the proteinogenic amino acids comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, selenocysteine, and pyrrolysine. Non-proteinogenic amino acids comprise natural amino acids which are not encoded by the genetic code, e.g. D-isomers of the amino acids encoded by the genetic code such as but not limited to D-alanine, D-leucine or D-glutamine; other natural amino acids such as but not limited to γ-carboxyglutamate, ornithine, hydroxyproline or phosphoserine; synthetic chemically manufactured amino acids such as the beta analogues of amino acids such as but not limited to β-alanine; C-alpha methylated amino acid such as but not limited to Aib (α-aminoisobutyric acid), C-alpha-methyl Phe or C-alpha-methyl Tyr; N-methyl amino acids, such as but not limited to N-methyl Asn, N-methyl His, N-methyl Arg or N-methyl Tyr; homo amino acids such as but not limited to homohistidine, homoglutamine, homoarginine (2-Amino-6-guanidino-hexanoic acid, HomoArg), homoleucine or homophenylalanine, beta-homo amino acid such as but not limited to (3-homo Gln; nor amino acids such as but not limited to norleucine, diamino acids such as but not limited to diaminopropionic acid, 2,4-diaminobutyric acid or ornithine; N-substituted glycines such as but not limited to (2-Carbamoyl-ethylamino)-acetic acid (NGln) or (3-Guanidinopropylamino)-acetic acid (NArg); amino acids with methylated side chain functional groups such as but not limited to N-epsilon-methyllysine, (N-epsilon, N-epsilon)dimethyllysine, (N-epsilon, N-epsilon, N-epsilon)trimethyllysine, N-omega-methylarginine or (N-omega, N-omega)-dimethylarginine; amino acids with shortened sidechains such as but not limited to 2-amino-3-guanidino-propionic acid (Agp), 2-amino-4-guanidino-butyric acid (Agb); other arginine mimicking amino acids such as but not limited to (2-Guanidino-ethylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or amino-(1-carbamimidoyl-piperidin-4-yl)-acetic acid; or other synthetic amino acids such as but not limited to 3-pyridylalanine, (4-Hydroxy-benzylamino)-acetic acid, Abu (α-aminobutyric acid), Tle (tert-butylglycine), 3-aminomethyl benzoic acid, anthranilic acid, pyridylalanine, 2-pyridylalanine, 4-pyridylalanine, (1-aminocyclopropyl)carboxylic acid, (1-aminocyclobutyl)carboxylic acid, (1-aminocyclopentyl)carboxylic acid, (1-aminocyclohexyl)carboxylic acid, (1-aminocycloheptyl)carboxylic acid, (1-aminocyclooctyl)carboxylic acid, thiotyrosine, 3-mercaptophenylalanine, 3- or 4-aminophenylalanine, 3- or 4-acetylphenylalanine, 2- or 3- hydroxyphenylalanine (o- or m-tyrosine), hydroxymethylglycine, aminoethylglycine, 1-methyl-1-mercaptoethylglycine, aminoethylthioethylglycine, 2-amino-histidine, p-hydroxy-histidine or mercaptoethylglycine etc. Many of the non-proteinogenic amino acids useful in the present invention are commercially available. Others may be prepared by methods known in the art. In one embodiment a non-proteinogenic amino acid is a moiety which can be incorporated into a peptide via peptide bonds but is not a proteogenic amino acid. In one embodiment the constituent amino acids of the PYY derivative according to the invention may be selected from at least one of the group of the amino acids encoded by the genetic code, proteinogenic amino acids which are not encoded by the genetic code and synthetic amino acids.

In one embodiment the invention involves the use of N-substituted glycines, which are a specific subclass of peptidomimetics. The N-substituted glycines are closely related to proteinogenic or non-proteinogenic amino acid counterpart, but differ chemically in that their side chains are appended to nitrogen atoms along the backbone of the molecule, rather than to the α-carbons (as they are in amino acids). For convenience, we have given the monomers of N-substituted glycines designations corresponding to the amino acids with similar functionality, with the prefix N. As examples NArg is intented to mean N-substituted glycine with an arginine sidechain and NGln is intented to mean N-substituted glycine with a glutamine sidechain, see formulas for NArg and NGln below.

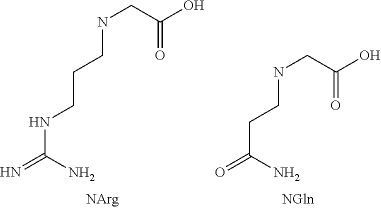 [SMILES: N=C(N)NCCCNCC(=O)O ; NC(=O)CCNCC(=O)O]

In one embodiment the invention involves the use of arginine mimics, which are closely related to proteinogenic or non-proteinogenic amino acid counterpart, but differ chemically in that their side chains are either elongated or truncated. For convenience, we have given the monomers of arginine mimics the following designations: Agp is intented to mean 2-amino-3-guanidino-propionic acid in which the guanidino group containing sidechain has been shortened by two carbon atoms; Agb is intented to mean 2-amino-4-guanidino-butyric acid in which the guanidino group containing sidechain has been shortened by one carbon atom; and homoarginine or HomoArg is intented to mean 2-Amino-6-guanidino-hexanoic acid in which the guanidino group containing sidechain has been elongated by one carbon atom.

In one embodiment the PYY derivative does not comprise non-proteinogenic amino acid residues. In one embodiment the PYY derivative comprises only L-amino acid residues and/or modified proteinogenic L-amino acid residues.

In one embodiment the PYY derivative comprises at least one amino acid residue which is substituted with proteinogenic and non-proteinogenic amino acids including but not limited to (both the D- and L-configuration are contemplated, however, for convenience only the L-configuration is shown):

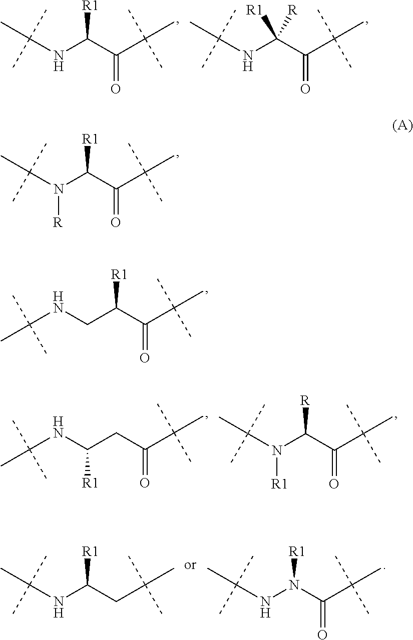

wherein, R1 is intented to mean the functional amino acid side chain selected from the group consisting of

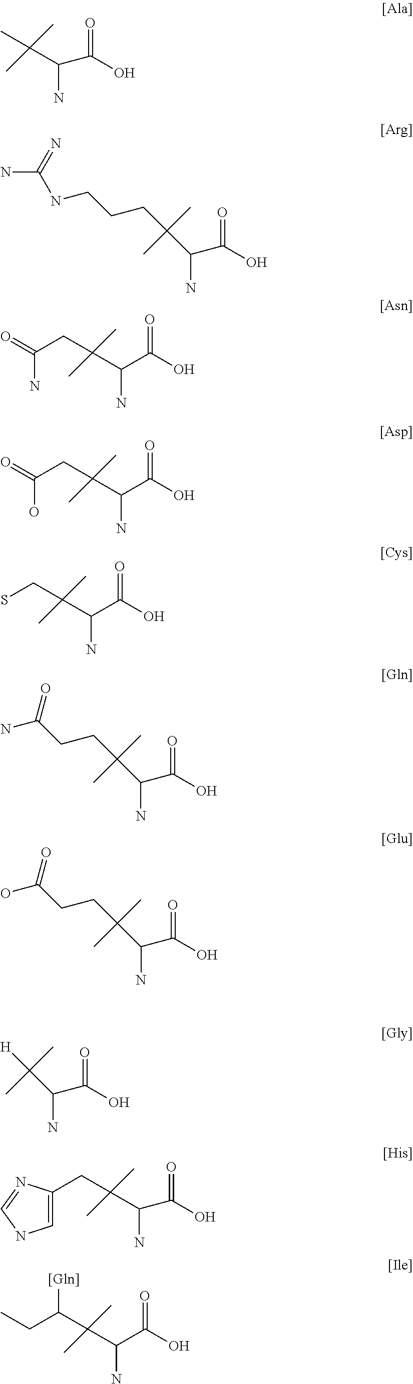

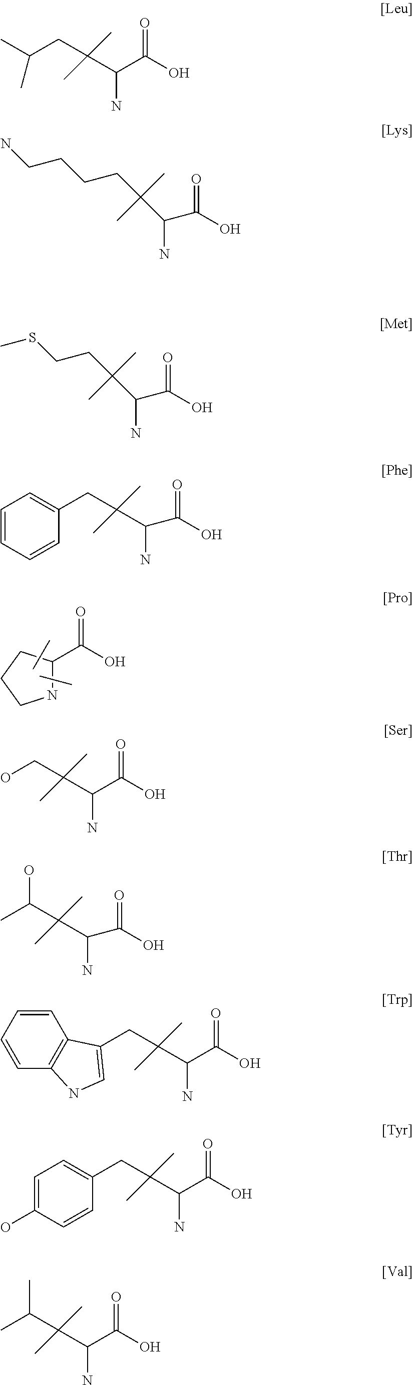

wherein the dashed line represents the disconnection between the functional sidechain and the amino acid backbone; and
R is selected from the group comprising H and C1-C12 alkyl.

In one embodiment the PYY derivative comprises the amino acid residue represented by formula (A) in at least one position of the peptide backbone of said derivative. In one embodiment the amino acid residue of formula (A) is found at least one position selected from the group consisting of position 33, 34, 35 and 36. In one embodiment the amino acid residue of formula (A) is found in position 35. In one embodiment when the amino acid residue of formula (A) is found in position 35 then R is the amino acid side chain of Arg and R is selected from the group consisting of H, alkyl (such as C1-C12 alkyl), benzyl or phenyl.

In one embodiment the PYY derivative comprises at least one peptide bond which is altered to a reduced peptide bond or a peptide bond isoster selected from at least one from the group consisting of a tetrazole, a sulphoneamide and an azide.

In one embodiment a maximum of 8 amino acids have been substituted, deleted, inserted and/or modified in the PYY derivative as compared to hPYY(1-36). In one embodiment a maximum of 7 amino acids have been substituted, deleted, inserted and/or modified in the PYY derivative as compared to hPYY(1-36). In one embodiment a maximum of 6 amino acids have been substituted, deleted, inserted and/or modified in the PYY derivative as compared to hPYY(1-36). In one embodiment a maximum of 5 amino acids have been substituted, deleted, inserted and/or modified in the PYY derivative as compared to hPYY(1-36). In one embodiment a maximum of 4 amino acids have been substituted, deleted, inserted and/or modified in the PYY derivative as compared to hPYY(1-36). In one embodiment a maximum of 3 amino acids have been substituted, deleted, inserted and/or modified in the PYY derivative as compared to hPYY(1-36). In one embodiment a maximum of 2 amino acids have been substituted, deleted, inserted and/or modified in the PYY derivative as compared to hPYY(1-36). In one embodiment 1 amino acid has been substituted, deleted, inserted and/or modified in the PYY derivative as compared to hPYY(1-36).

In one embodiment the PYY derivative exhibits at least 60%, 65%, 70%, 80% or 90% sequence identity to PYY(1-36) or PYY(3-36) over the entire length of the PYY(1-36) or PYY(3-36), respectively. As an example of a method for determination of sequence identity between two analogues the two peptides [Ala34]PYY(1-36) and PYY(1-36) are aligned. The sequence identity of Ala34 analogue relative to PYY(1-36) is given by the number of aligned identical residues minus the number of different residues divided by the total number of residues in PYY(1-36). Accordingly, in said example the sequence identity is (36-1)/36.

In one embodiment the PYY derivative comprises at least one alteration, such as at least one of substitution, insertion, deletion and modification. In one embodiment the PYY derivative comprises at least one substitution, insertion, deletion and modification of a “non-essential” amino acid residue. A “non-essential” amino acid residue is intended to mean a residue that can be altered, i.e., deleted or substituted, in the sequence of the peptide without abolishing or substantially reducing the activity of said peptide. In one embodiment “activity” of the PYY derivative of the invention is Y2 receptor potency as is determined by a Y2 receptor potency assay, such as Assay (I) described herein, Y2 receptor ACTOne assay. The term “substitution” is intended to mean the change of one amino acid in the native sequence with another amino acid. The term “deletion” is intended to mean the removal of one or more amino acids from the native sequence. The term “insertion” is intended to mean the addition of one or more amino acid into the native sequence. The term “modification” is intended to mean alterations covalently attached to the side chain of one or more amino acids or the alpha nitrogen atom of one or more amino acid in the native peptide sequence.

In one embodiment the C-terminal of the derivative according to the invention may be terminated as either an acid or amide. In one embodiment the C-terminal of the derivative of the invention is an amide.

Substitutions.

In one embodiment the PYY derivative has at least one substitution in the amino acid sequence compared to hPYY(1-36), alone or in combination with at least one insertion or deletion. In one embodiment the substitution does not abolish or substantially reduce activity of the PYY derivative. In one embodiment the PYY derivative has a single substitution or a consecutive or non-consecutive substitution of more than one amino acid residues compared to the amino acid sequence of hPYY(1-36). In one embodiment the PYY derivative comprises one, two or three amino acid substitutions compared to the amino acid sequence of hPYY(1-36).

In one embodiment the amino acid residues of at the helical C-terminus region of PYY (e.g., residues 20, 24, 25, 27 and 29), the tail end residues (32-36), and/or the N-terminus prolines at position 5 and 8 are not substituted. In one embodiment amino acid residues are not substituted at positions 32 through 36 of PYY. In one embodiment amino acid residues of PYY are not substituted at at least one amino acid sequence position selected from: 5, 7, 8, 20, 24, 25, 27, 29, 32, 33, 34, 35, 36 and any combination thereof.

In one embodiment amino acids are substituted by conservative substitution. The term “conservative substitution” as used herein denotes that at least one amino acid is replaced by at least one biologically similar residue. Examples comprise substitution of amino acid residues with similar characteristics, e.g., small amino acids, acidic amino acids, polar amino acids, basic amino acids, hydrophobic amino acids and aromatic amino acids. For example, in one embodiment Met residues are substituted with norleucine (Nle) or with leucine, isoleucine or valine, which—as opposed to Met—are not readily oxidised. Another example of a conservative substitution with a residue normally not found in endogenous, mammalian peptides and proteins would be the conservative substitution of Arg or Lys with for example, ornithine, canavanine, aminoethylcysteine or another basic amino acid. For further information concerning phenotypically silent substitutions in peptides and proteins, see, e.g., Bowie et.al. Science 247, 1306-1310, 1990. Conservatively substituted analogues of the invention may have, e.g., up to 10 conservative substitutions, such as up to 5 or such as 3 or fewer conservative substitutions.

In one embodiment the PYY derivative comprises substitutions of at least one non-proteinogenic and/or non-amino acid, e.g., amino acid mimetics, into the sequence of PYY. In one embodiment the non-amino acids inserted into the sequence of PYY are beta-turn mimetics or molecules, such as —NH—X—CO—, wherein X═(CH2)n (where n can be 2-20) or —NH—CH2CH2(—O—CH2CH2—O-)m-CH2-CO— (where m=1-5). In one embodiment molecules inserted into the sequence of PYY may be selected from aminocaproyl (“Aca”), beta-alanyl, and 8-amino-3,6-dioxaoctanoyl. beta-turn mimetics are available commercially (BioQuadrant Inc, Quebec, Canada).

Ease of Manufacture.

In one embodiment the PYY derivative has improved properties for manufacturing. In one embodiment less side products are formed when coupling the serum albumin binding side chain to PYY. Substituting the lysine in position 4 avoids formation of side products, such as double acylated compounds. In one embodiment the PYY derivative does not comprise an unsubstituted lysine side chain.

In one embodiment the serum albumin binding side chain is attached to the amino acid sequence of the PYY derivative after synthesis and purification of said amino acid sequence. In one embodiment, if the serum albumin binding side chain is to be attached to a Lys of said amino acid sequence, which is different from Lys in position 4, then it is an advantage to substitute or delete the Lys in position 4. In one embodiment said substitution or deletion of the Lys in position 4 would provide improved ease of manufacture of the PYY derivative. In one embodiment said substitution of Lys in position 4 is with Arg (see e.g. SEQ ID NO: 35) or Glu (see, e.g. SEQ ID NO: 38). In one embodiment said deletion is a deletion of Pro3 and Lys4 (see, e.g., SEQ ID NO: 36 and 38).

In one embodiment the Asn in position 18 and position 29 are substituted with another amino acid. Said substitution of Asn provides the advantage of abolishing any risk of deamidation of said Asn amino acid residues. In one embodiment Asn is substituted with Glu (see, e.g., SEQ ID NO: 37, 38 and 39). In one embodiment position 29 is Asn.

Deletions.

In one embodiment the PYY derivative has at least one amino acid residue deleted from the amino acid sequence of hPYY(1-36), alone or in combination with at least one insertion or substitution. In one embodiment the PYY derivative has at least one amino acid residue deleted at amino acid positions 4 through 35 of PYY. Such deletions may comprise at least one consecutive or non-consecutive deletion at amino acid positions 4 through 35 of PYY. In one embodiment the amino acid residues at positions 24 through 36 of PYY are not deleted.

In one embodiment the PYY derivative comprises N-terminal or C-terminal truncations or internal deletions at amino acid positions 4 to 35 as long as at least one biological activity of hPYY(1-36) is retained. In one embodiment positions 1 and 2 of PYY are deleted. In one embodiment the amino acid residues at positions 5 through 8 and 24 through 36, more specifically positions 5 through 8 and positions 32 through 35 of PYY are not deleted.

Insertions.

In one embodiment the PYY derivative has at least one amino acid residue inserted into the amino acid sequence of hPYY(1-36), alone or in combination with at least one deletion and/or substitution. In one embodiment the PYY derivative has a single insertion or consecutive or non-consecutive insertions of more than one amino acid residues into the amino acid sequence of hPYY(1-36). In one embodiment at least one amino acid is inserted at the N-terminal or C-terminal end of the PYY derivative. In one embodiment amino acid residues are not inserted at positions 24 through 36 of PYY.

In one embodiment the PYY derivative comprises chemical alterations to at least one amino acid residue. Such chemical alterations comprise amidation, glycosylation, acylation, sulfation, phosphorylation, acetylation and/or cyclization. The chemical alterations may occur singularly at the N- or C-terminus or at the side chains of amino acid residues within the sequence of the PYY derivative. In one embodiment the C-terminus of these peptides may have a free —OH or —NH₂ group. In one embodiment the N-terminal end of the peptides may be capped with an isobutyloxycarbonyl group, an isopropyloxycarbonyl group, an n-butyloxycarbonyl group, an ethoxycarbonyl group, an isocaproyl group (isocap), an octanyl group, an octyl glycine group (G(Oct)), an 8-aminooctanic acid group or a Fmoc group. In one embodiment there are multiple sites of chemical alteration along the peptide of the PYY derivative.

In one embodiment the PYY derivative comprises insertions of at least one non-proteinogenic amino acid and/or non-amino acid into the sequence of hPYY(1-36). In one embodiment the non-proteinogenic amino acids inserted into the sequence of hPYY(1-36) may be beta-turn mimetics or molecules inserted into the sequence of PYY. Examples of molecules inserted into the sequence of PYY comprise aminocaproyl (“Aca”), beta-alanyl and 8-amino-3,6-dioxaoctanoyl.

In one embodiment the PYY derivative comprises combinations of two or more changes selected from the group consisting of deletion, insertion, and substitution. In one embodiment the PYY derivative comprises one, two or three amino acid substitutions. In one embodiment the PYY derivative comprises one, two or three amino acid modifications.

In one embodiment the PYY derivative comprises an N-terminal acetyl or sucvinyl group.

In one embodiment the PYY derivative has an improved enzymatic stability compared to hPYY(3-36). In one embodiment improved enzymatic stability results in improved half-life, which may be determined by Assay (IV) as described herein.

In one embodiment the PYY derivative comprises the amino acid sequence of formula (I):

Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅-Xaa₂₆-Xaa₂₇-Xaa₂₈-Xaa₂₉-Xaa₃₀-Xaa₃₁-Xaa₃₂-Xaa₃₃-Xaa₃₄-Xaa₃₅-Xaa₃₆ Formula (I)

wherein
Xaa₁ is Tyr, Phe, Ala, 2,3-diaminopropionic acid, 2,4-diaminobutyric acid, ornitine, Lys or absent;
Xaa₂ is Pro, Ala, Leu, Phe, hydroxyproline, 2,3-diaminopropionic acid, 2,4-diaminobutyric acid, ornitine, Lys or absent;
Xaa₃ is Ile, Val, Leu (1-aminocyclopentyl)carboxylic acid, (1-aminocyclohexyl)carboxylic acid, 1-aminobutyric acid, 2,3-diaminopropionic acid, 2,4-diaminobutyric acid, ornitine, Lys, D-Ile, D-allo-Ile or absent;
Xaa₄ is Lys, Arg, Glu, 2,3-diaminopropionic acid, 2,4-diaminobutyric acid, ornitine, Lys, absent, Ala, Val, Ser or Gly;
Xaa₅ is Pro, 2,3-diaminopropionic acid, 2,4-diaminobutyric acid, ornitine, absent or Lys;
Xaa₆ is Glu, 2,3-diaminopropionic acid, 2,4-diaminobutyric acid, ornithine, absent or Lys;
Xaa₇ is Ala, 2,3-diaminopropionic acid, 2,4-diaminobutyric acid, ornitine, absent or Lys;
Xaa₈ is Pro, Ala, 2,3-diaminopropionic acid, 2,4-diaminobutyric acid, ornitine, absent, Glu or Lys;
Xaa₉ is Gly, 2,3-diaminopropionic acid, 2,4-diaminobutyric acid, ornitine, absent, Glu or Lys;
Xaa₁₀ is Glu, 2,3-diaminopropionic acid, 2,4-diaminobutyric acid, ornitine or Lys;
Xaa₁₁ is Asp, Ala, 2,3-diaminopropionic acid, 2,4-diaminobutyric acid, ornitine or Lys;
Xaa₁₂ is Ala, 2,3-diaminopropionic acid, 2,4-diaminobutyric acid, ornitine or Lys;
Xaa₁₃ is Ser, 2,3-diaminopropionic acid, 2,4-diaminobutyric acid, ornithine, Lys;
Xaa₁₄ is Pro, hydroxyproline or Ala;
Xaa₁₅ is Glu, 2,3-diaminopropionic acid, 2,4-diaminobutyric acid, ornitine or Lys;
Xaa₁₆ is Glu, 2,3-diaminopropionic acid, 2,4-diaminobutyric acid, ornitine or Lys;
Xaa₁₇ is Leu, Val, Ile, homoleucine, norleucine, (1-aminocyclopentyl)carboxylic acid, (1-aminocyclohexyl)carboxylic acid or 1-aminobutyric acid;
Xaa₁₈ is Asn, Ala, Glu, 2,3-diaminopropionic acid, 2,4-diaminobutyric acid, ornitine, Lys, Gln, Asp, D-Asp, IsoAsp or D-IsoAsp;
Xaa₁₉ is Arg, Ala, 2,3-diaminopropionic acid, 2,4-diaminobutyric acid, ornithine, Glu or Lys;
Xaa₂₀ is Tyr, Ala, Phe, 3-pyridylalaine, 2,3-diaminopropionic acid, 2,4-diaminobutyric acid, ornitine or Lys;
Xaa₂₁ is Tyr, Ala, Phe, 3-pyridylalaine, 2,3-diaminopropionic acid, 2,4-diaminobutyric acid, ornitine or Lys;
Xaa₂₂ is Ala, 2,3-diaminopropionic acid, 2,4-diaminobutyric acid, ornithine, Arg, Glu or Lys;
Xaa₂₃ is Ser, 2,3-diaminopropionic acid, 2,4-diaminobutyric acid, ornitine or Lys;
Xaa₂₄ is Leu, Ile, Val, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₂₅ is Arg, Ala, His, aminoisobutyric acid, 2,3-diaminopropionic acid, 2,4-diaminobutyric acid, ornitine or Lys;
Xaa₂₆ is His, Arg, 2,3-diaminopropionic acid, 2,4-diaminobutyric acid, ornitine or Lys;
Xaa₂₇ is Tyr, Ala, Phe, homoPhe or 3-pyridylalanine, 2,3-diaminopropionic acid, 2,4-diaminobutyric acid, ornitine or Lys;
Xaa₂₈ is Leu, Ile, Val, homoleucine, norleucine, (1-aminocyclopentyl)carboxylic acid, (1-aminocyclohexyl)carboxylic acid, aminoisobutyric acid, 1-aminobutyric acid, 2,3-diaminopropionic acid, 2,4-diaminobutyric acid, ornitine or Lys;
Xaa₂₉ is Asn, Gln, 2,3-diaminopropionic acid, 2,4-diaminobutyric acid, ornithine, D-IsoAsp or Lys;
Xaa₃₀ is Leu, Met, Val, Ile, homoleucine, aminoisobutyric acid, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₁ is Val, Leu, Ile, aminoisobutyric acid, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₂ is Thr, Ser, 2,3-diaminopropionic acid, 2,4-diaminobutyric acid, ornitine or Lys;
Xaa₃₃ is Arg, N-methyl Arg, methyllysine, dimethyllysine, trimethyllysine, 2-amino-3-guanidino-propionic acid, 2-amino-4-guanidino-butyric acid or monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid;
Xaa₃₄ is Gln, Asn, His, Pro, N-methyl Gln, β-homo Gln, (2-Carbamoyl-ethylamino)-acetic acid, N-methyl Asn or N-methyl His;
Xaa₃₅ is Arg, N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid; and
Xaa₃₆ is Tyr, Phe, N-methyl Tyr, C-methyl Phe, 3-pyridylalanine or (4-Hydroxybenzylamino)-acetic acid, 4-fluorophenylalanine or 4-pyridylalanine.

In one embodiment the PYY derivative comprises the amino acid sequence of formula (I):

Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅-Xaa₂₆-Xaa₂₇-Xaa₂₈-Xaa₂₉-Xaa₃₀-Xaa₃₁-Xaa₃₂-Xaa₃₃-Xaa₃₄-Xaa₃₅-Xaa₃₆ Formula (I)

wherein
Xaa₁ is Tyr, Phe, Ala, 2,3-diaminopropionic acid, 2,4-diaminobutyric acid, ornithine, Lys or absent;
Xaa₂ is Pro, Ala, Leu, Phe, hydroxyproline, 2,3-diaminopropionic acid, 2,4-diaminobutyric acid, ornithine, Lys or absent;
Xaa₃ is Ile, Val, Leu (1-aminocyclopentyl)carboxylic acid, (1-aminocyclohexyl)carboxylic acid, 1-aminobutyric acid, 2,3-diaminopropionic acid, 2,4-diaminobutyric acid, ornithine, Lys or absent;
Xaa₄ is Lys, Glu, 2,3-diaminopropionic acid, 2,4-diaminobutyric acid, ornithine, Lys or absent;
Xaa₅ is Pro, 2,3-diaminopropionic acid, 2,4-diaminobutyric acid, ornithine or Lys;
Xaa₆ is Glu, 2,3-diaminopropionic acid, 2,4-diaminobutyric acid, ornithine or Lys;
Xaa₇ is Ala, 2,3-diaminopropionic acid, 2,4-diaminobutyric acid, ornithine or Lys;
Xaa₈ is Pro, Ala, 2,3-diaminopropionic acid, 2,4-diaminobutyric acid, ornithine or Lys;
Xaa₉ is Gly, 2,3-diaminopropionic acid, 2,4-diaminobutyric acid, ornithine or Lys;
Xaa₁₀ is Glu, 2,3-diaminopropionic acid, 2,4-diaminobutyric acid, ornithine or Lys;
Xaa₁₁ is Asp, Ala, 2,3-diaminopropionic acid, 2,4-diaminobutyric acid, ornithine or Lys;
Xaa₁₂ is Ala, 2,3-diaminopropionic acid, 2,4-diaminobutyric acid, ornithine or Lys;
Xaa₁₃ is Ser, 2,3-diaminopropionic acid, 2,4-diaminobutyric acid, ornithine or Lys;
Xaa₁₄ is Pro or hydroxyproline;
Xaa₁₅ is Glu, 2,3-diaminopropionic acid, 2,4-diaminobutyric acid, ornithine or Lys;
Xaa₁₆ is Glu, 2,3-diaminopropionic acid, 2,4-diaminobutyric acid, ornithine or Lys;
Xaa₁₇ is Leu, Val, Ile, homoleucine, norleucine, (1-aminocyclopentyl)carboxylic acid, (1-aminocyclohexyl)carboxylic acid or 1-aminobutyric acid;
Xaa₁₈ is Asn, 2,3-diaminopropionic acid, 2,4-diaminobutyric acid, ornithine or Lys;
Xaa₁₉ is Arg, Ala, 2,3-diaminopropionic acid, 2,4-diaminobutyric acid, ornithine or Lys;
Xaa₂₀ is Tyr, Ala, Phe, 3-pyridylalaine, 2,3-diaminopropionic acid, 2,4-diaminobutyric acid, ornithine or Lys;
Xaa₂₁ is Tyr, Ala, Phe, 3-pyridylalaine, 2,3-diaminopropionic acid, 2,4-diaminobutyric acid, ornithine or Lys;
Xaa₂₂ is Ala, 2,3-diaminopropionic acid, 2,4-diaminobutyric acid, ornithine or Lys;
Xaa₂₃ is Ser, 2,3-diaminopropionic acid, 2,4-diaminobutyric acid, ornithine or Lys;
Xaa₂₄ is Leu, Ile, Val, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornithine or Lys;
Xaa₂₅ is Arg, Ala, His, aminoisobutyric acid, 2,3-diaminopropionic acid, 2,4-diaminobutyric acid, ornithine or Lys;
Xaa₂₆ is His, Arg, 2,3-diaminopropionic acid, 2,4-diaminobutyric acid, ornithine or Lys;
Xaa₂₇ is Tyr, Ala, Phe, homoPhe or 3-pyridylalanine, 2,3-diaminopropionic acid, 2,4-diaminobutyric acid, ornithine or Lys;
Xaa₂₈ is Ile, Val, Leu, homoleucine, norleucine, (1-aminocyclopentyl)carboxylic acid, (1-aminocyclohexyl)carboxylic acid, aminoisobutyric acid, 1-aminobutyric acid, 2,3-diaminopropionic acid, 2,4-diaminobutyric acid, ornithine or Lys;
Xaa₂₉ is Asn, Gln, 2,3-diaminopropionic acid, 2,4-diaminobutyric acid, ornithine or Lys;
Xaa₃₀ is Met, Leu, Val, Ile, homoleucine, aminoisobutyric acid, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornithine or Lys;
Xaa₃₁ is Leu, Val, Ile, aminoisobutyric acid, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornithine or Lys;
Xaa₃₂ is Ser, Thr, 2,3-diaminopropionic acid, 2,4-diaminobutyric acid, ornithine or Lys;
Xaa₃₃ is Arg, N-methyl Arg, methyllysine, dimethyllysine, trimethyllysine, 2-amino-3-guanidino-propionic acid (Apg), 2-amino-4-guanidino-butyric acid (Abg), monomethyllarginine, dimethylarginine, (2-Guanidino-ethylamino)-acetic acid, (3-Guanidinopropylamino)-acetic acid (NArg), (4-Guanidino-butylamino)-acetic acid, 2-Amino-3-(1-carbamimidoyl-pyrrolidin-2-yl)-propionic acid, 2-Amino-4-(2-amino-pyrimidin-4-yl)butyric acid, 2-Amino-3-(4-guanidino-phenyl)-propionic acid or Amino-(1-carbamimidoyl-piperidin-4-yl)-acetic acid;
Xaa₃₄ is Gln, Asn, His, Pro, N-methyl Gln, β-homo Gln, (2-Carbamoyl-ethylamino)-acetic acid (NGln), N-methyl Asn or N-methyl His;
Xaa₃₅ is Arg, N-methyl Arg, methyllysine, dimethyllysine, trimethyllysine, 2-amino-3-guanidino-propionic acid (Agp), 2-amino-4-guanidino-butyric acid (Agb), homoarginine (2-Amino-6-guanidino-hexanoic acid, HomoArg) monomethylarginine, dimethylarginine, (2-Guanidino-ethylamino)-acetic acid, (3-Guanidino-propylamino)-acetic acid (NArg), (4-Guanidino-butylamino)-acetic acid, 2-Amino-3-(1-carbamimidoyl-pyrrolidin-2-yl)propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid;
Xaa₃₆ is Tyr, Phe, N-methyl Tyr, C-α-methyl Phe, 3-pyridylalanine, (4-Hydroxybenzylamino)-acetic acid.

In one embodiment Xaa₁ is Tyr. In one embodiment Xaa₁ is Lys.

In one embodiment Xaa₂ is Pro.

In one embodiment Xaa₃ is Ile. In one embodiment Xaa₃ is Lys. In one embodiment Xaa₃ is Val. In one embodiment Xaa₃ is absent.

In one embodiment Xaa₄ is Lys. In one embodiment Xaa₄ is Lys. In one embodiment Xaa₄ is Arg. In one embodiment Xaa₄ is Asp. In one embodiment Xaa₄ is Glu. In one embodiment Xaa₄ is Val. In one embodiment Xaa₄ is Ala. In one embodiment Xaa₄ is Ser. In one embodiment Xaa₄ is Gly. In one embodiment Xaa₄ is not Glu or not Lys. In one embodiment Xaa₄ is absent.

In one embodiment Xaa₅ is Pro. In one embodiment Xaa₅ is Lys.

In one embodiment Xaa₆ is Glu. In one embodiment Xaa₆ is Lys.

In one embodiment Xaa₇ is Ala. In one embodiment Xaa₇ is Lys.

In one embodiment Xaa₈ is Pro. In one embodiment Xaa₈ is Lys. In one embodiment Xaa₈ is not Glu.

In one embodiment Xaa₉ is Gly. In one embodiment Xaa₉ is Glu. In one embodiment Xaa₉ is Lys.

In one embodiment Xaa₁₀ is Glu. In one embodiment Xaa₁₀ is Lys.

In one embodiment Xaa₁₁ is Asp. In one embodiment Xaa₁₁ is Lys. In one embodiment Xaa₁₁ is Glu.

In one embodiment Xaa₁₂ is Ala. In one embodiment Xaa₁₂ is Lys.

In one embodiment Xaa₁₃ is Ser. In one embodiment Xaa₁₃ is Lys.

In one embodiment Xaa₁₄ is Pro. In one embodiment Xaa₁₄ is Ala. In one embodiment Xaa₁₄ is Lys.

In one embodiment Xaa₁₅ is Glu. In one embodiment Xaa₁₅ is Lys.

In one embodiment Xaa₁₆ is Glu. In one embodiment Xaa₁₆ is Lys.

In one embodiment Xaa₁₇ is Leu. In one embodiment Xaa₁₇ is Lys.

In one embodiment Xaa₁₈ is Ala. In one embodiment Xaa₁₈ is Lys. In one embodiment Xaa₁₈ is Asp. In one embodiment Xaa₁₈ is IsoAsp. In one embodiment Xaa₁₈ is Glu. In one embodiment Xaa₁₈ is Gln. In one embodiment Xaa₁₈ is not D-IsoAsp.

In one embodiment Xaa₁₉ is Arg. In one embodiment Xaa₁₉ is Glu. In one embodiment Xaa₁₉ is Lys.

In one embodiment Xaa₂₀ is Tyr. In one embodiment Xaa₂₀ is Lys. In one embodiment Xaa₂₀ is Ala.

In one embodiment Xaa₂₁ is Tyr. In one embodiment Xaa₂₁ is Lys.

In one embodiment Xaa₂₂ is Ala. In one embodiment Xaa₂₂ is Lys. In one embodiment Xaa₂₂ is Asp. In one embodiment Xaa₂₂ is Glu. In one embodiment Xaa₂₂ is Arg.

In one embodiment Xaa₂₂ is not Glu or not D-Arg.

In one embodiment Xaa₂₃ is Ser. In one embodiment Xaa₂₃ is Lys.

In one embodiment Xaa₂₄ is Leu. In one embodiment Xaa₂₄ is Lys.

In one embodiment Xaa₂₅ is Arg. In one embodiment Xaa₂₅ is Lys. In one embodiment Xaa₂₅ is His. In one embodiment Xaa₂₅ is Aib. In one embodiment Xaa₂₅ is Tyr.

In one embodiment Xaa₂₆ is His. In one embodiment Xaa₂₆ is Lys.

In one embodiment Xaa₂₇ is Tyr. In one embodiment Xaa₂₇ is Lys. In one embodiment Xaa₂₇ is Ala.

In one embodiment Xaa₂₈ is Leu. In one embodiment Xaa₂₈ is Lys.

In one embodiment Xaa₂₉ is Asn. In one embodiment Xaa₂₉ is Lys. In one embodiment Xaa₂₉ is Gln. In one embodiment Xaa₂₉ is not Gln or not D-IsoAsp.

In one embodiment Xaa₃₀ is Leu. In one embodiment Xaa₃₀ is Lys.

In one embodiment Xaa₃₁ is Val. In one embodiment Xaa₃₁ is Lys.

In one embodiment Xaa₃₂ is Thr. In one embodiment Xaa₃₂ is Lys.

In one embodiment Xaa₃₃ is Arg. In one embodiment Xaa₃₃ is Lys. In one embodiment Xaa₃₃ is N-methyl Arg.

In one embodiment Xaa₃₄ is Gln. In one embodiment Xaa₃₄ is Lys. In one embodiment Xaa₃₄ is N-methyl Gln. In one embodiment Xaa₃₄ is β-homo Gln.

In one embodiment Xaa₃₅ is Arg. In one embodiment Xaa₃₅ is Lys. In one embodiment Xaa₃₅ is N-methyl Arg.

In one embodiment Xaa₃₆ is Tyr. In one embodiment Xaa₃₆ is C-α-methyl Phe. In one embodiment Xaa₃₆ is not 4-pyridylalanine.

In one embodiment Xaa₄ is Arg and Xaa₁₈ is Asp. In one embodiment Xaa₄ is Ala and Xaa₁₈ is Asp.

In one embodiment Xaa₁ and Xaa₂ are absent. In one embodiment Xaa₁, Xaa₂, Xaa₃ and Xaa₄ are absent.

In one embodiment the PYY derivative is selected from the group consisting of

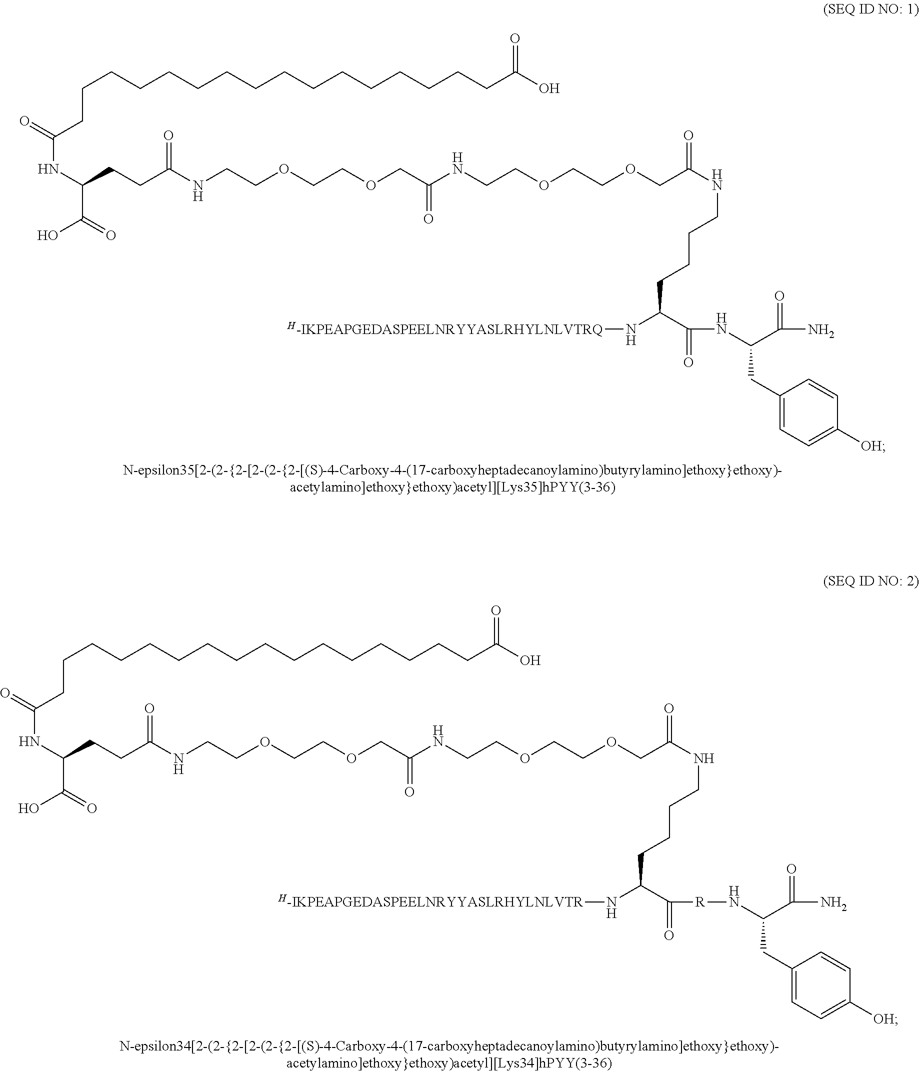

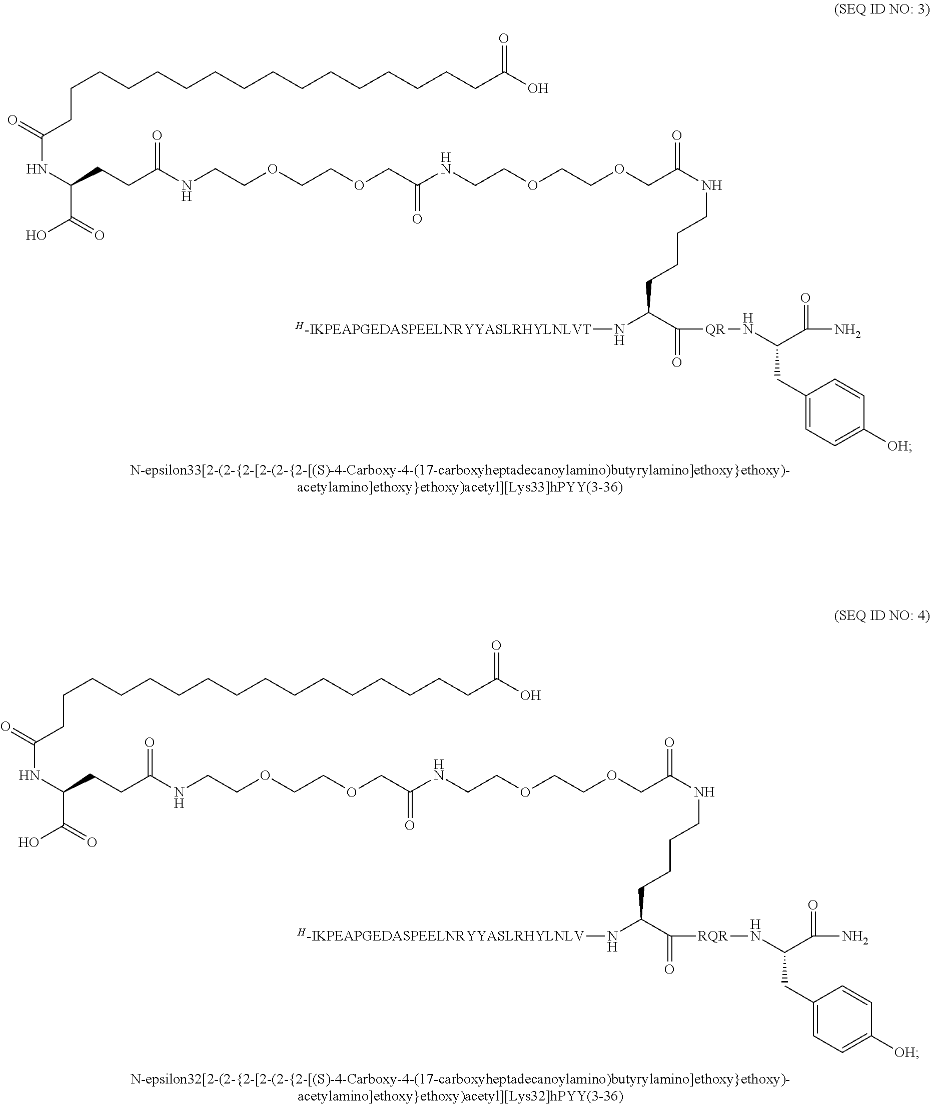

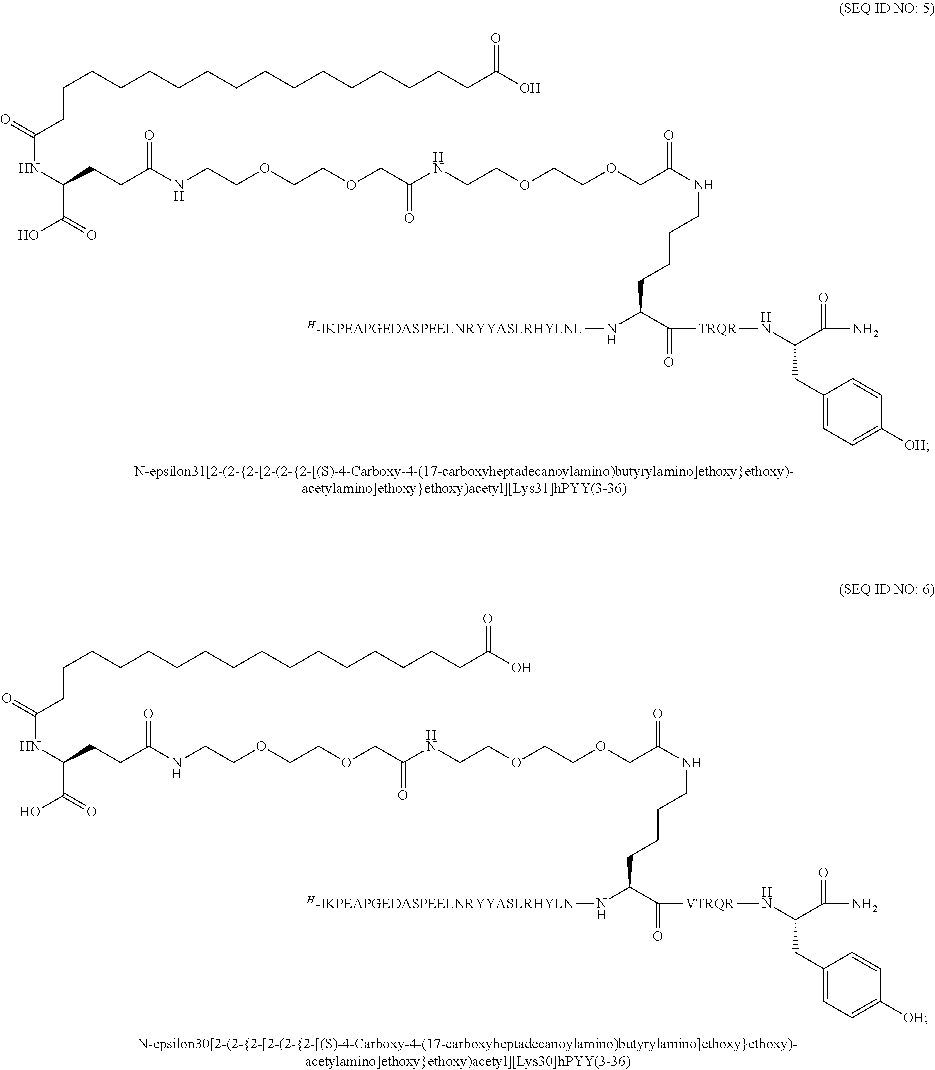

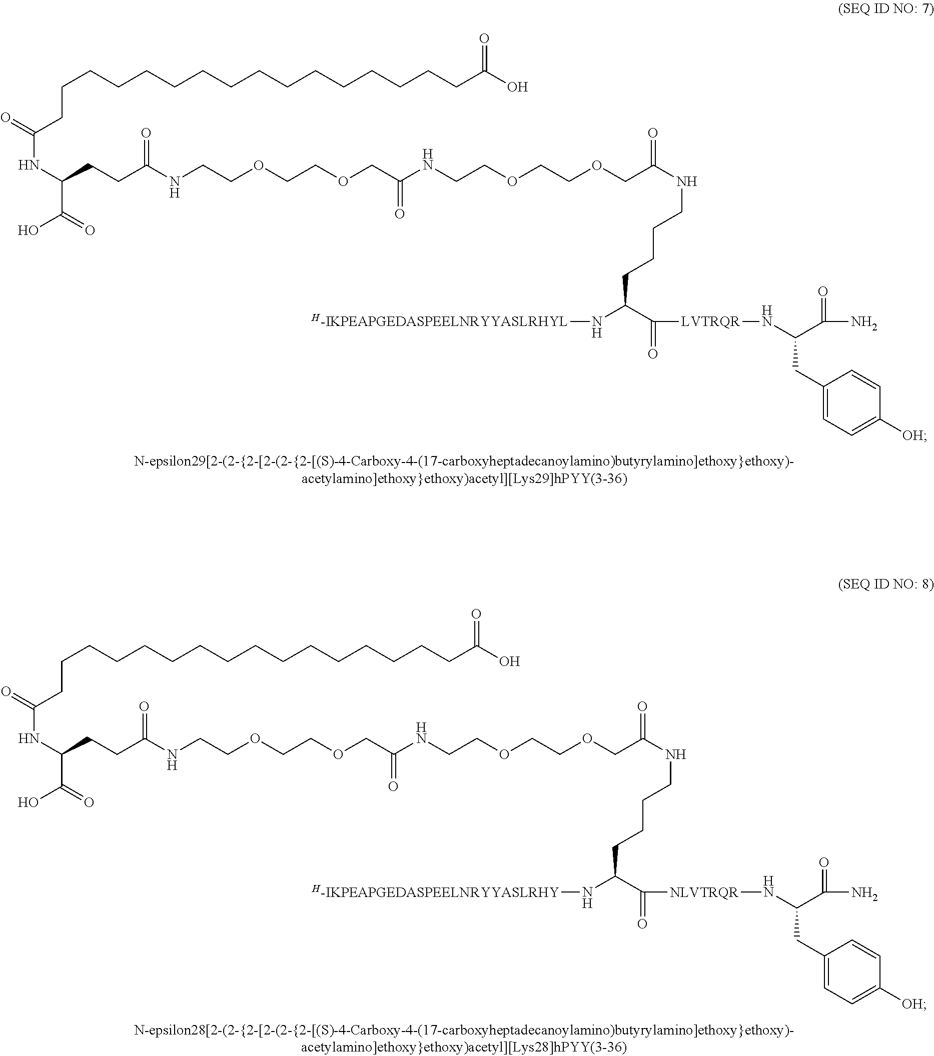

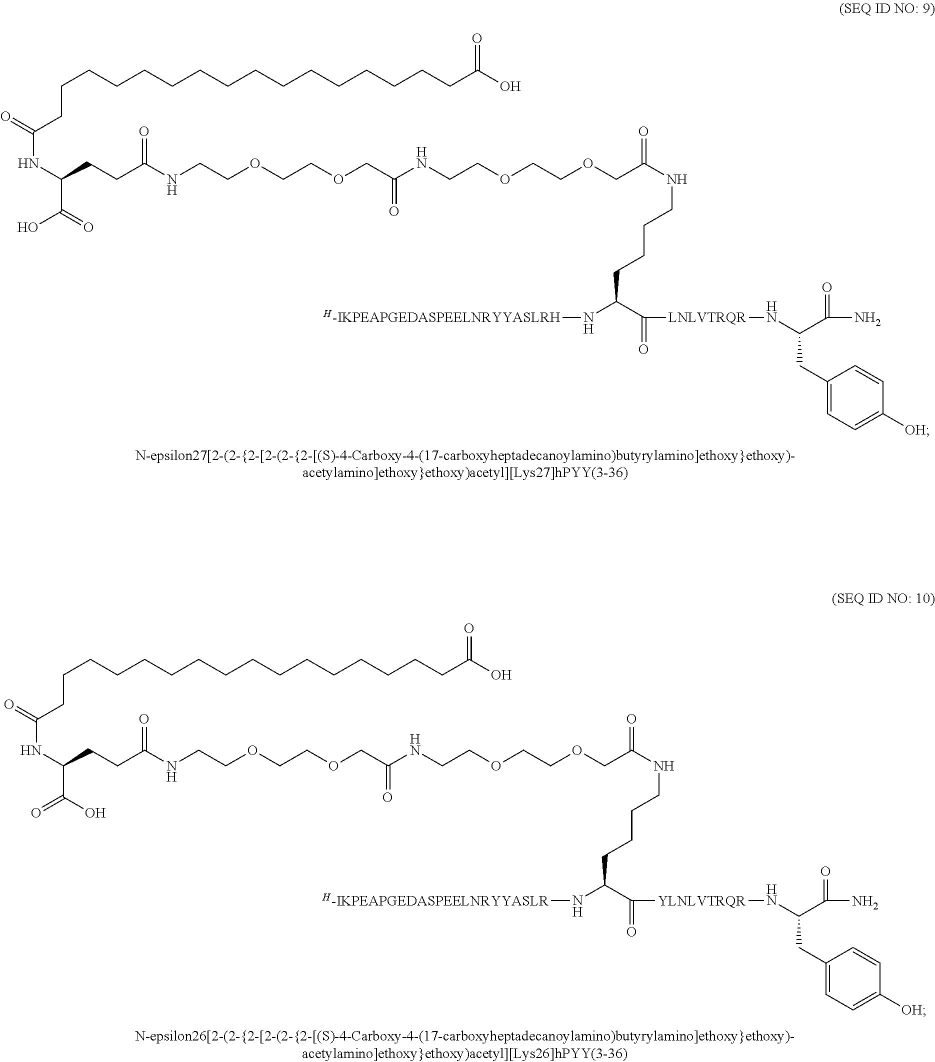

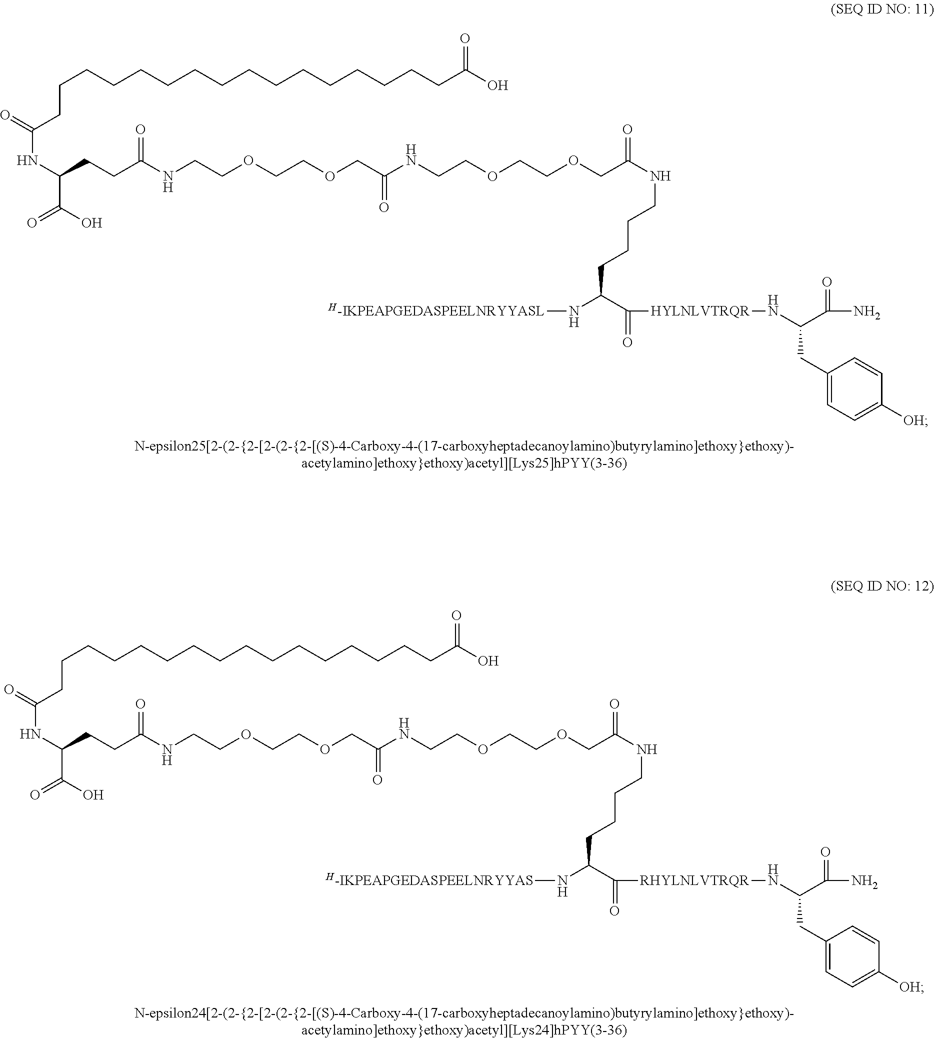

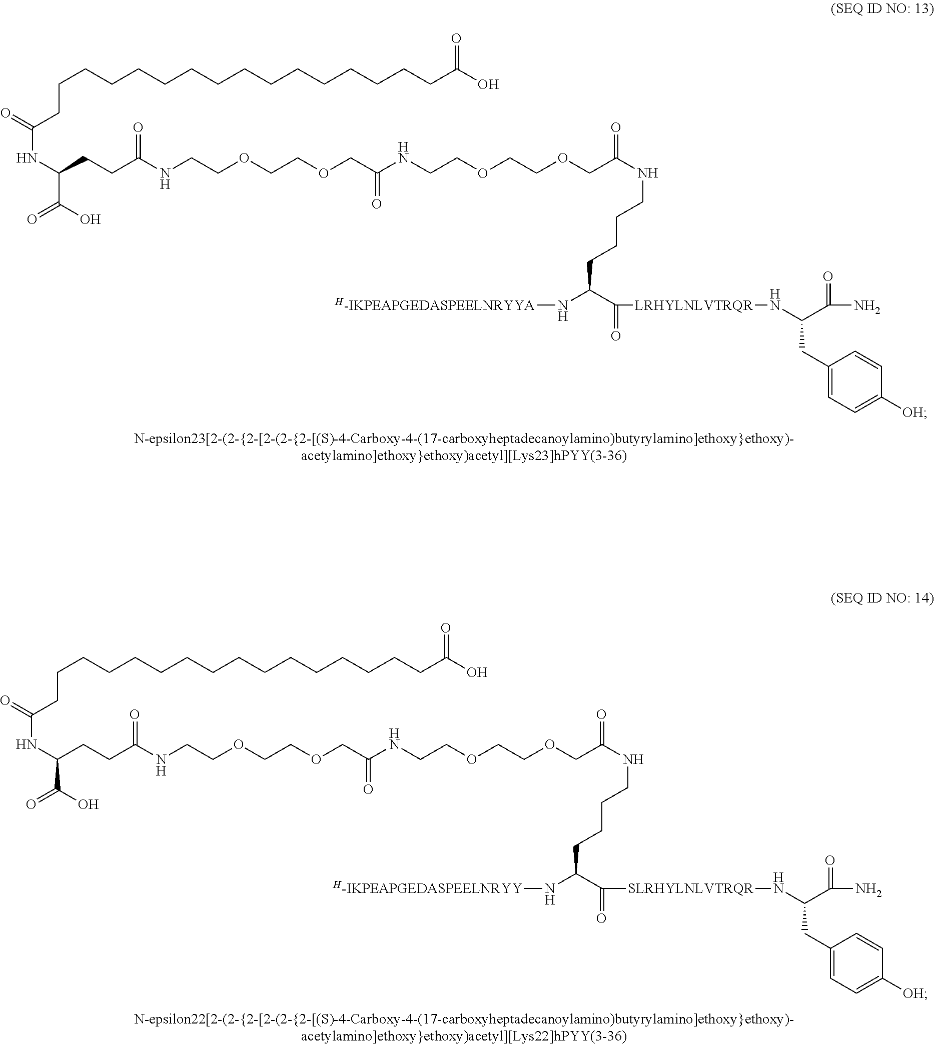

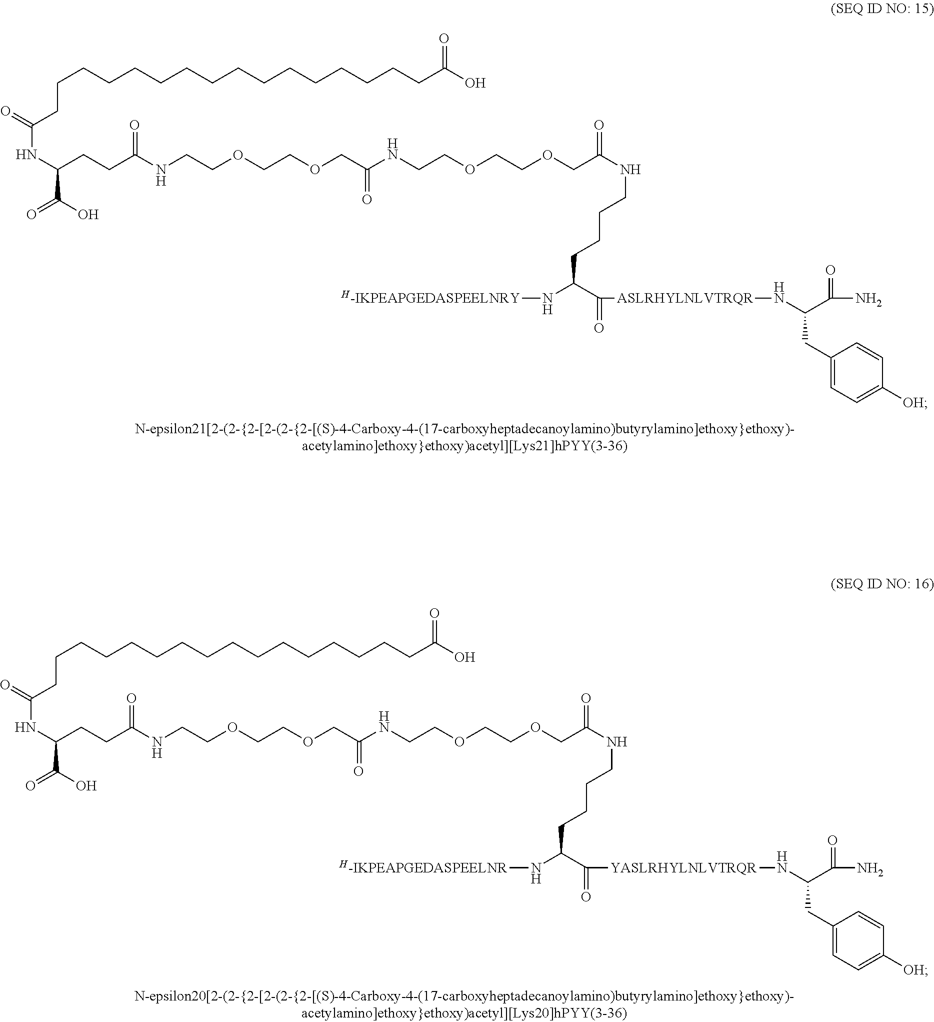

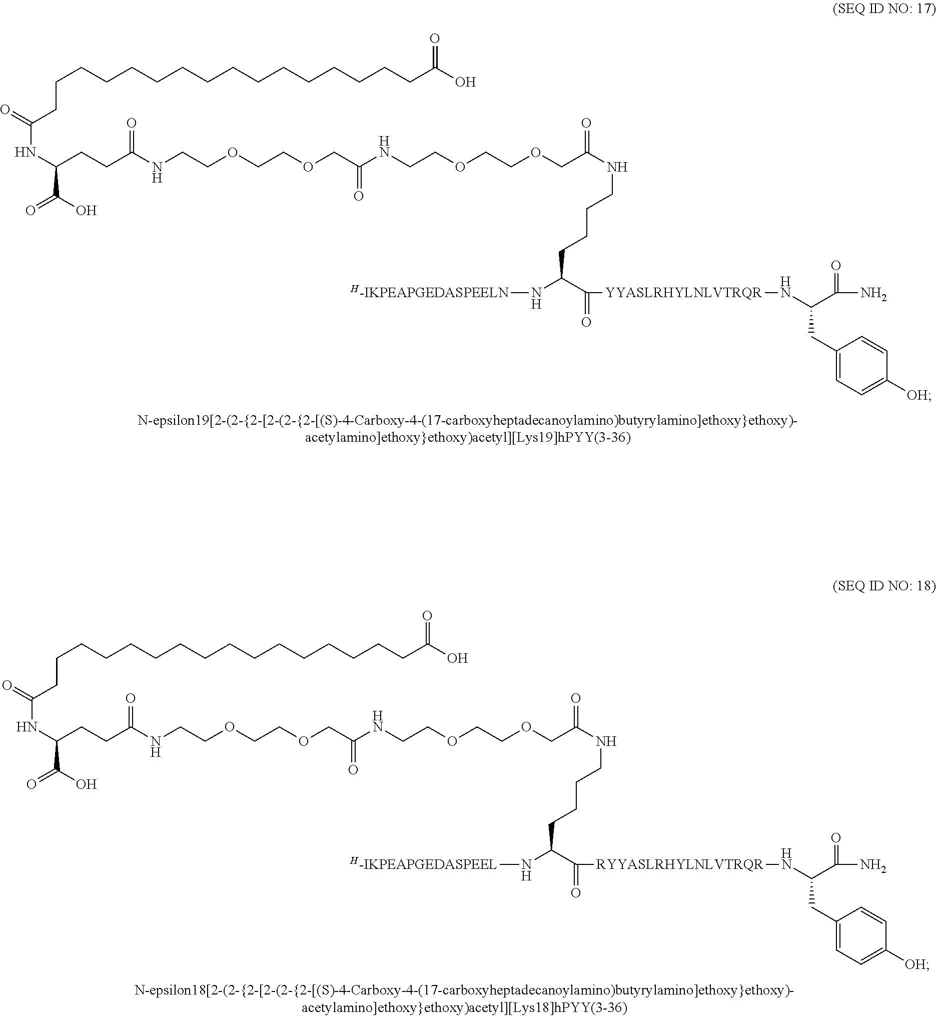

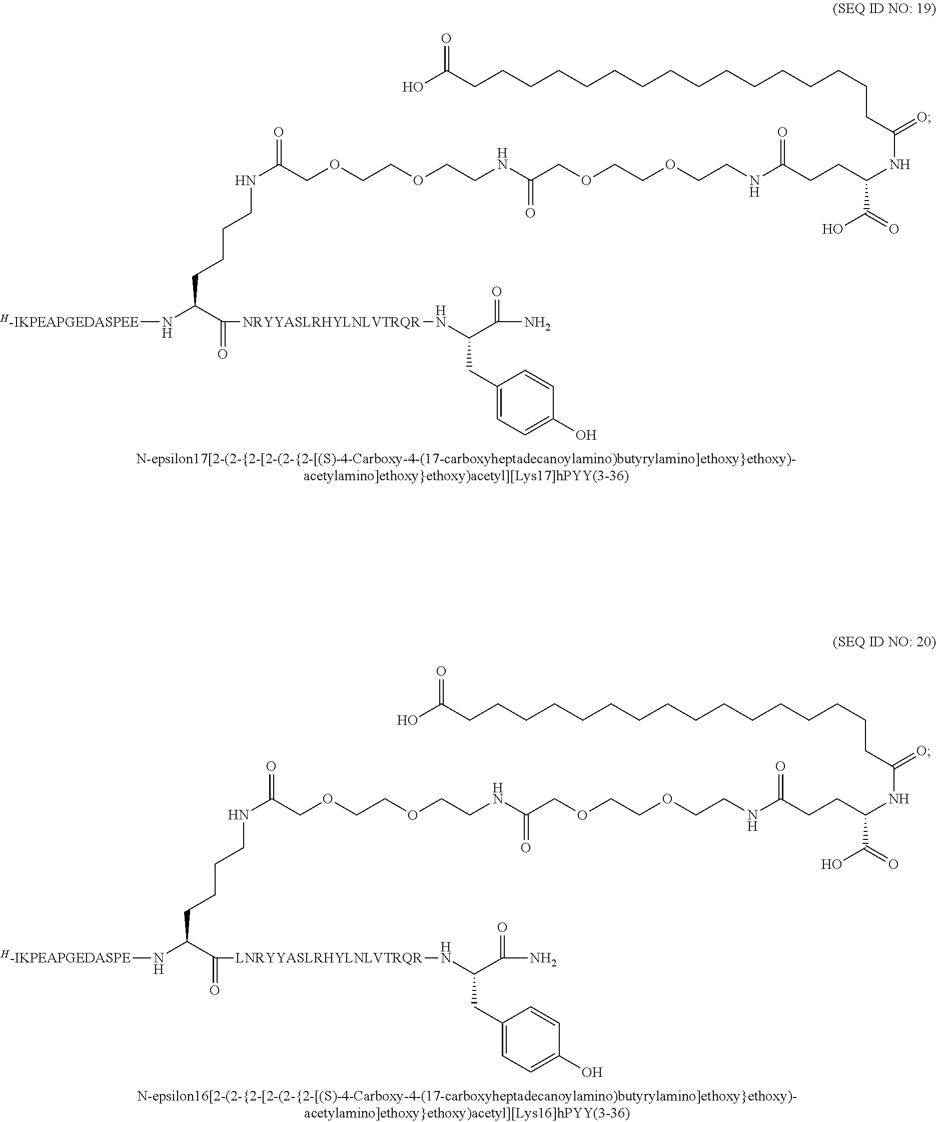

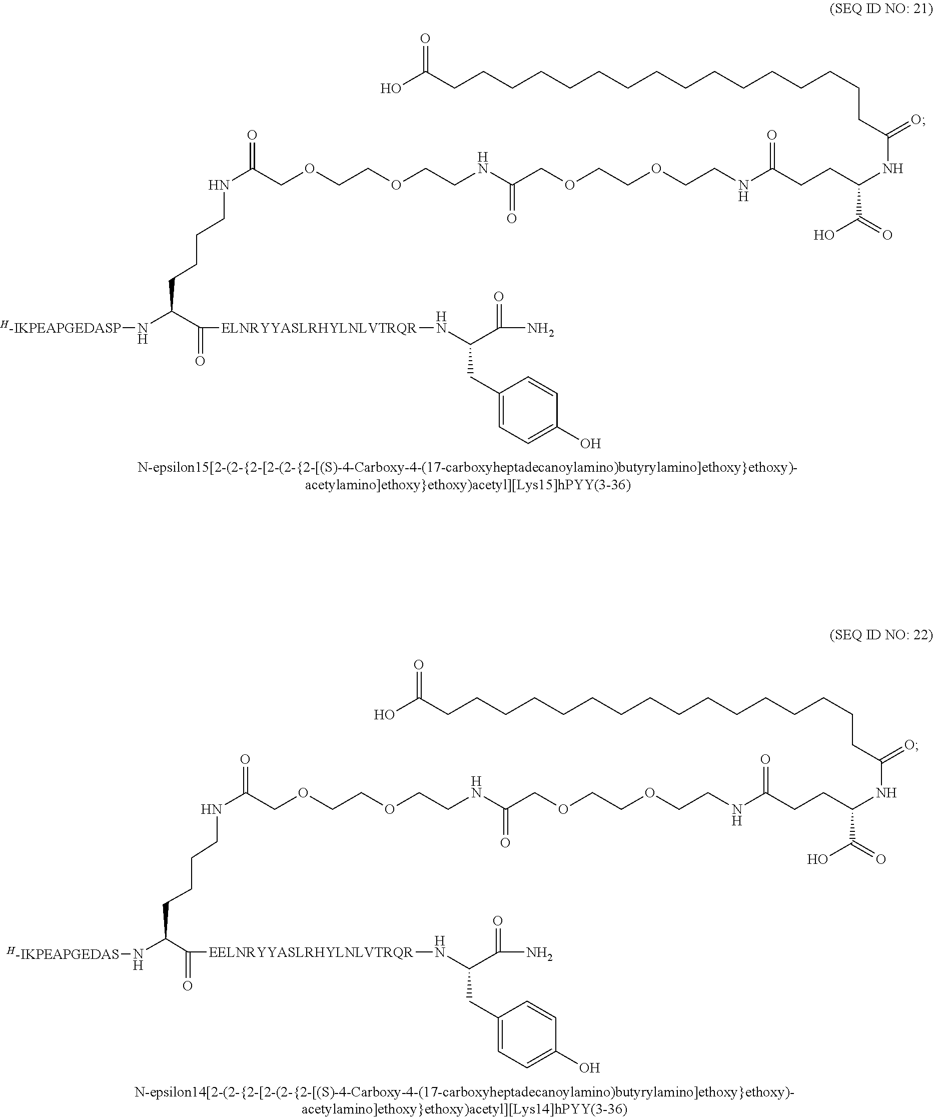

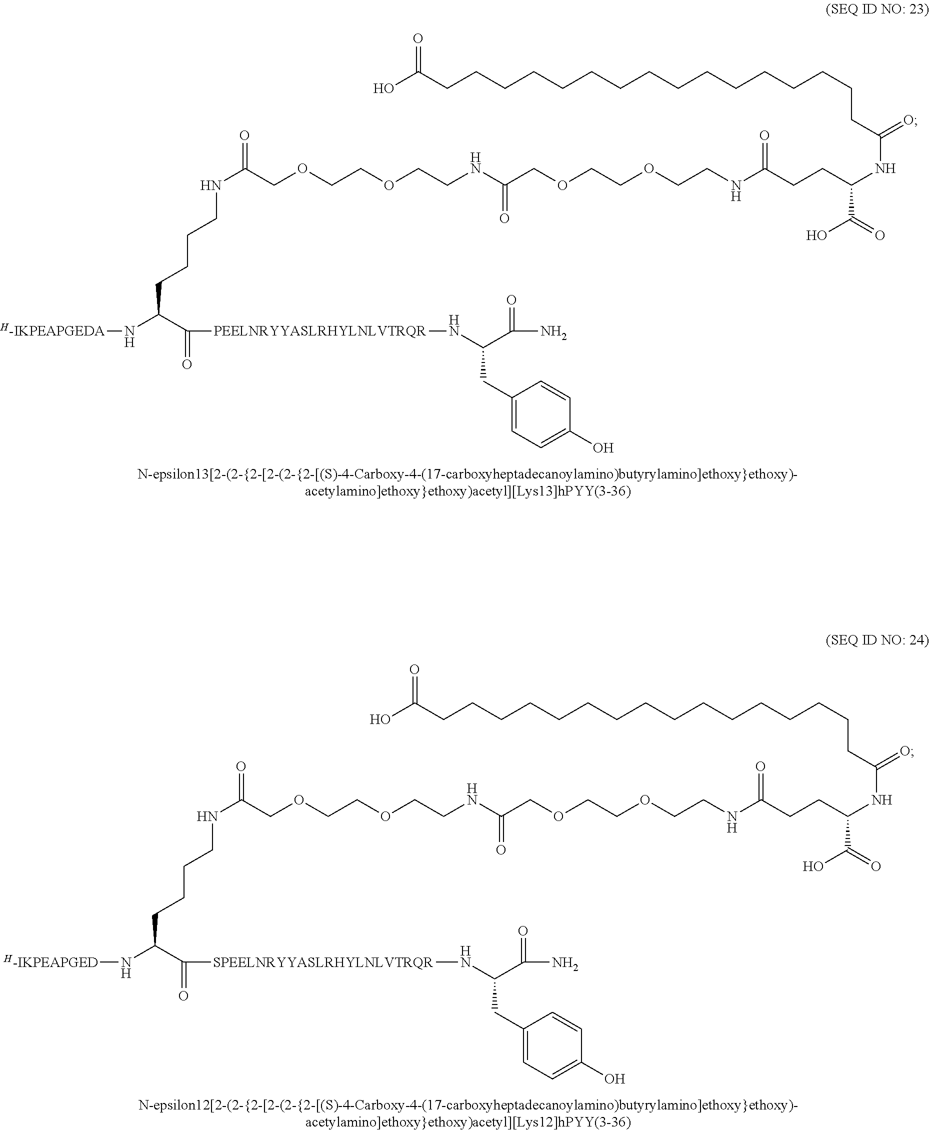

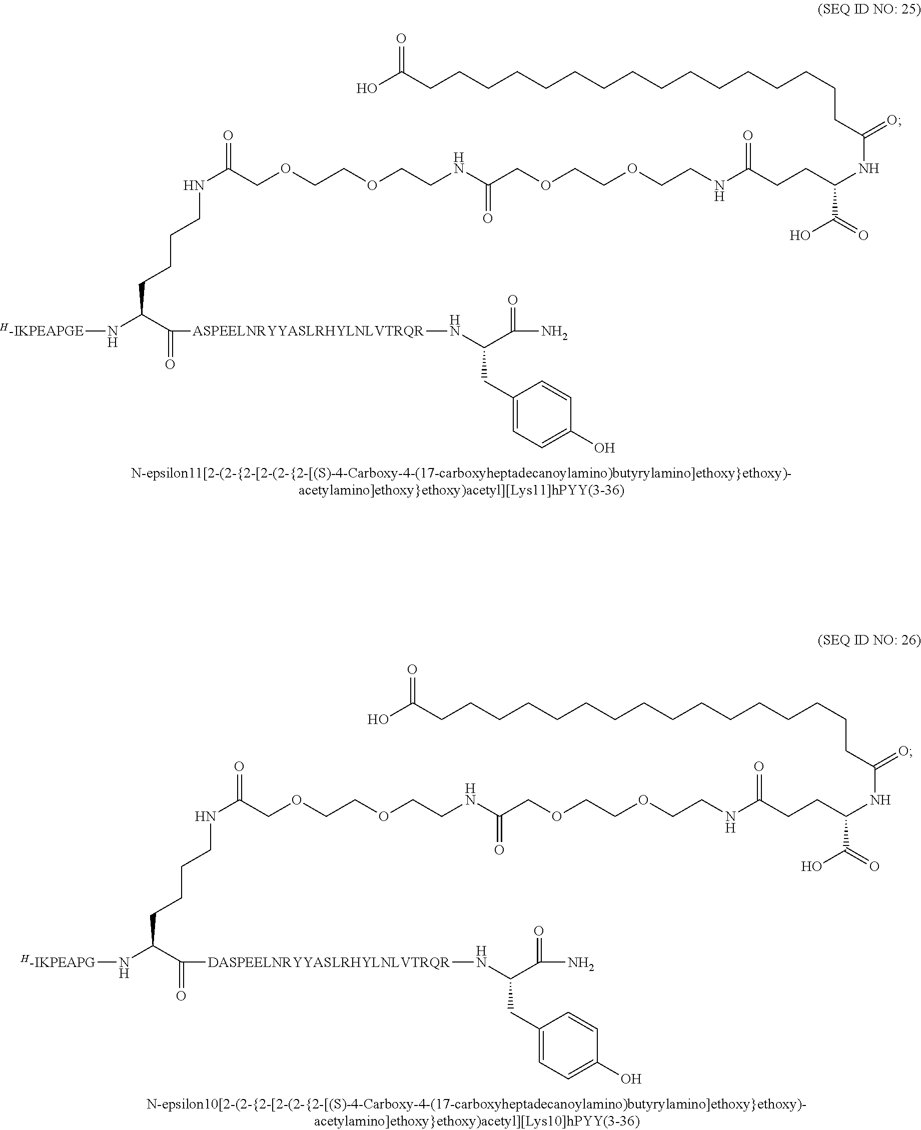

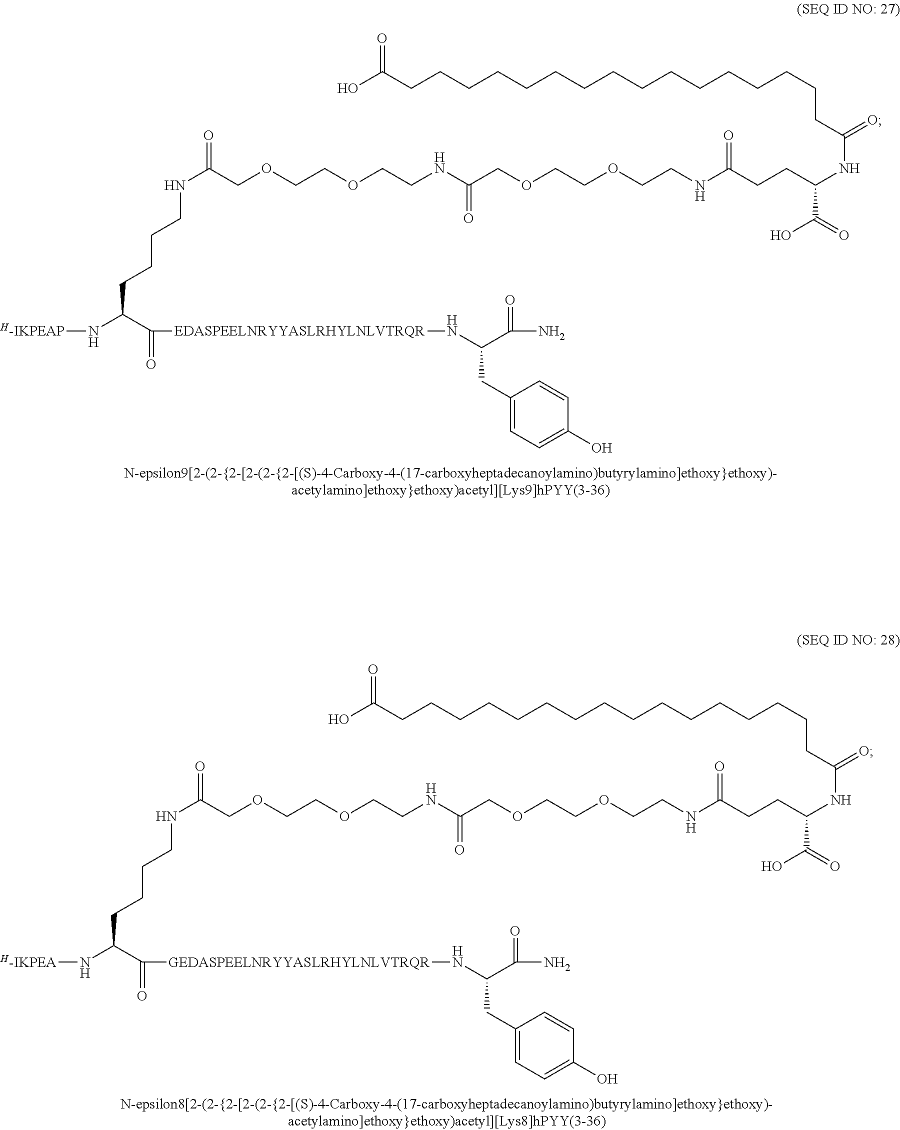

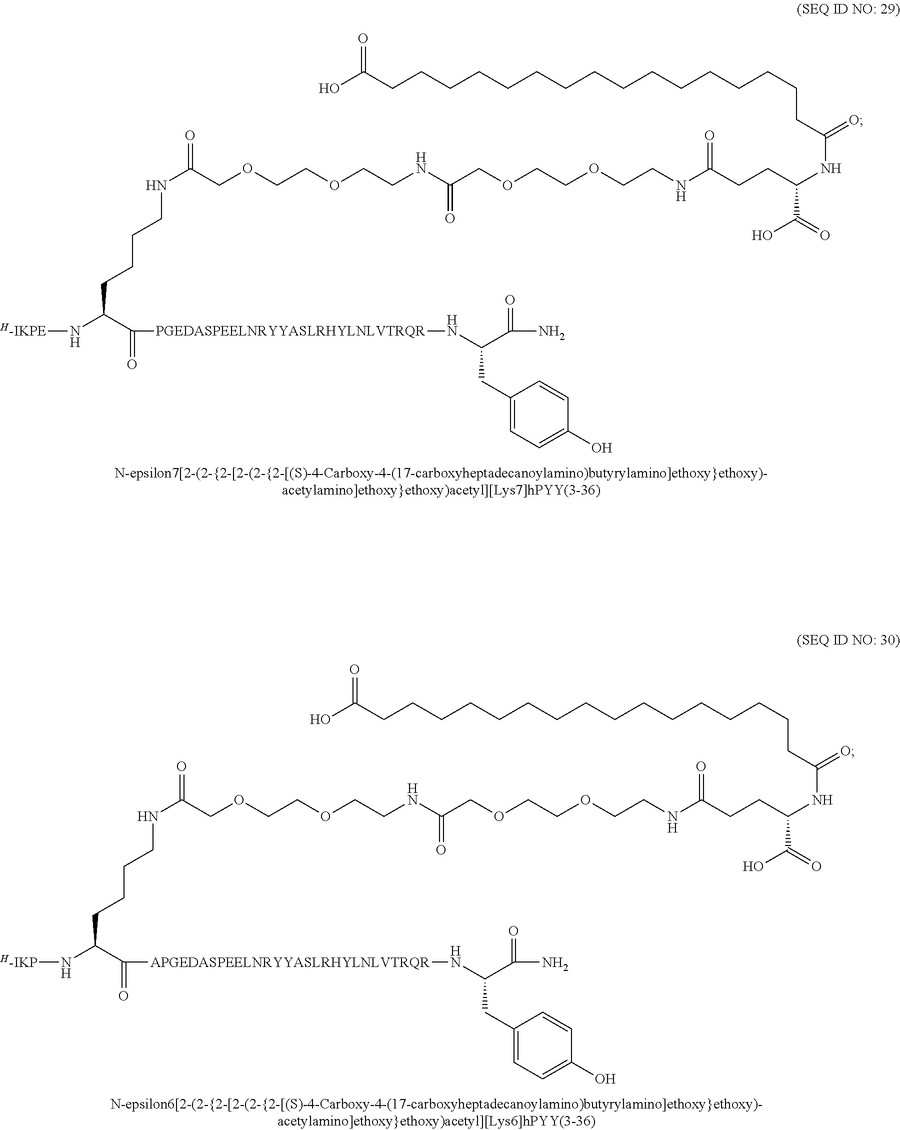

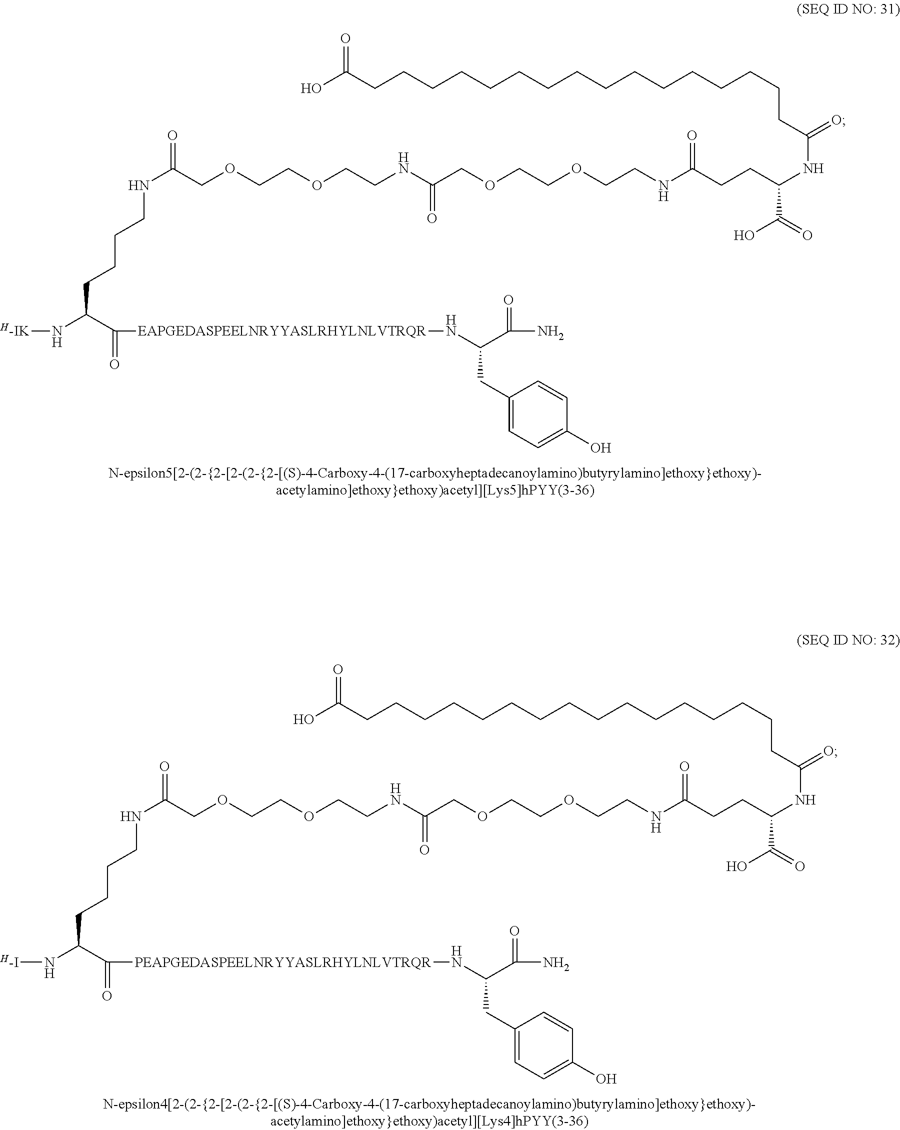

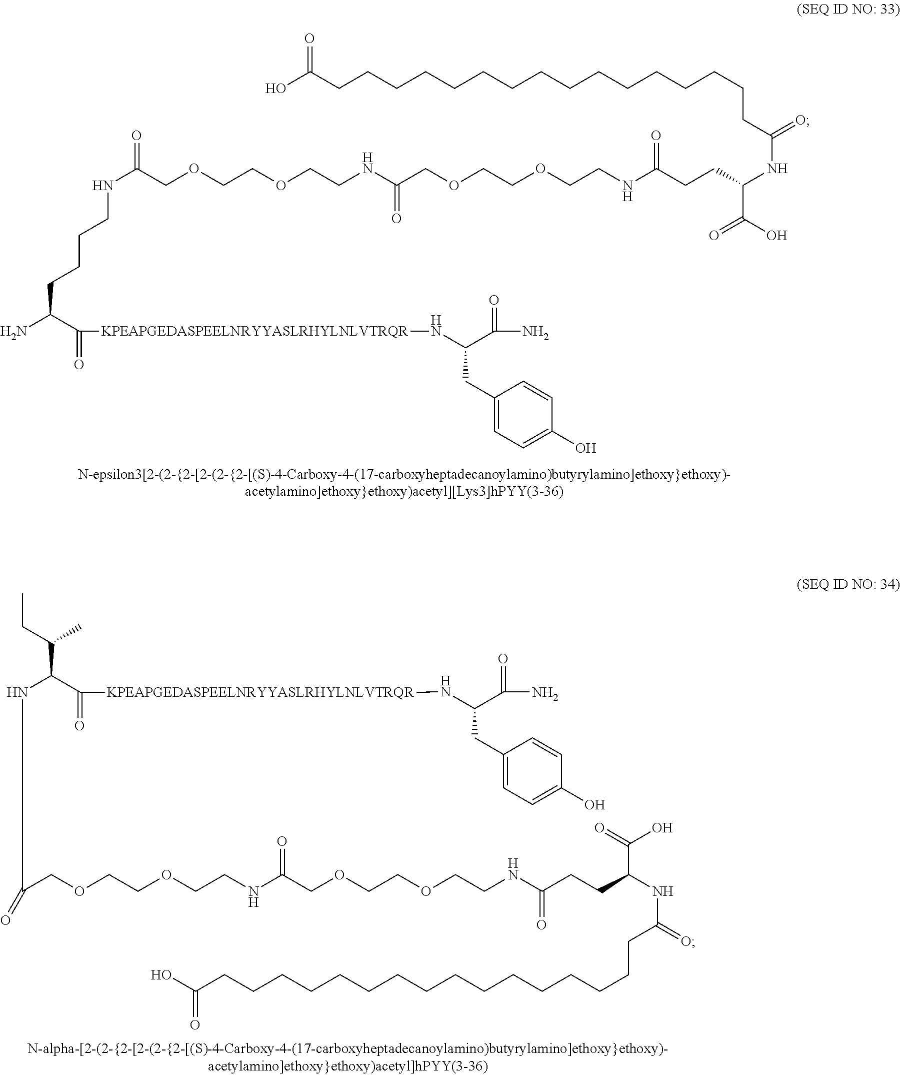

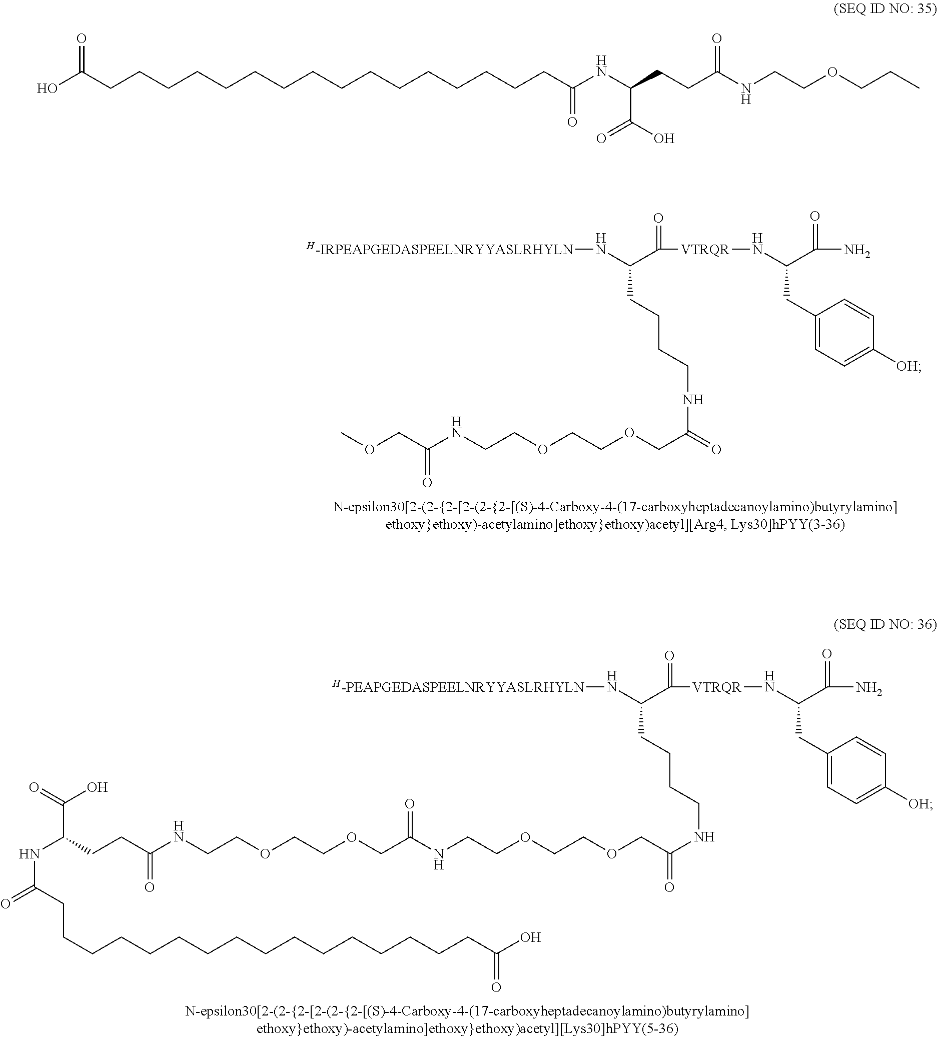

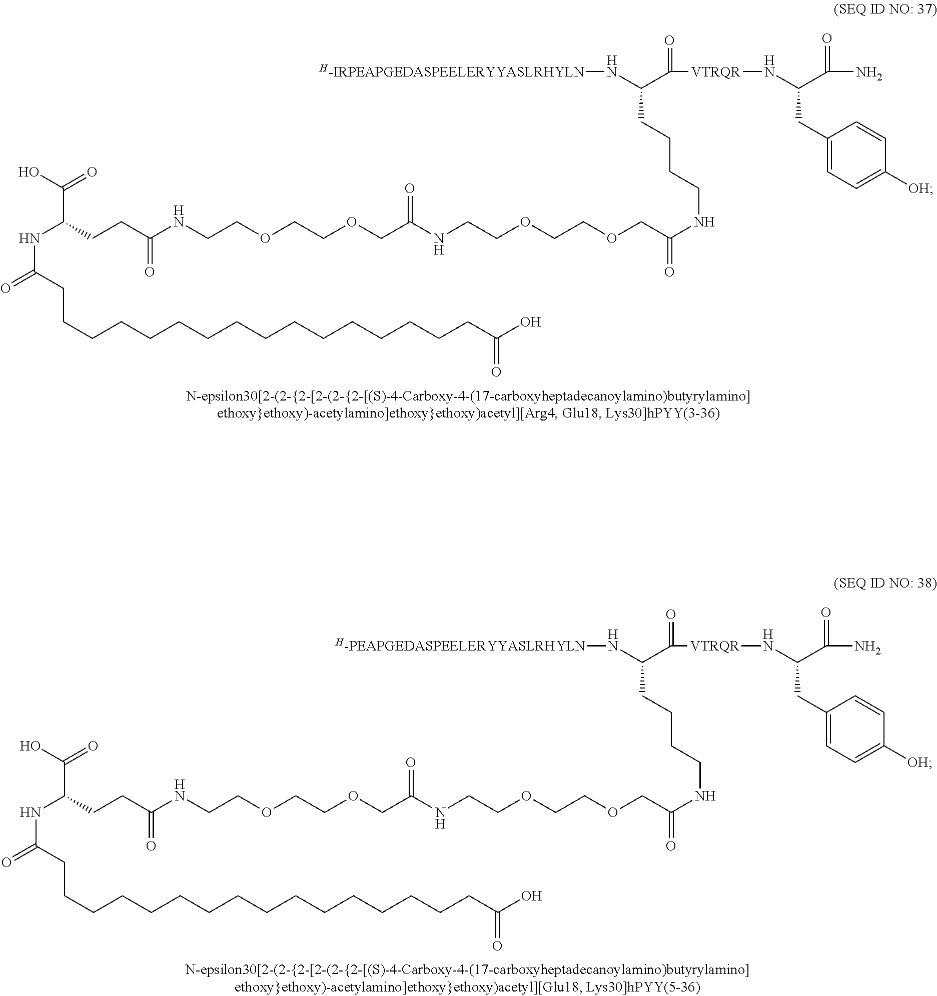

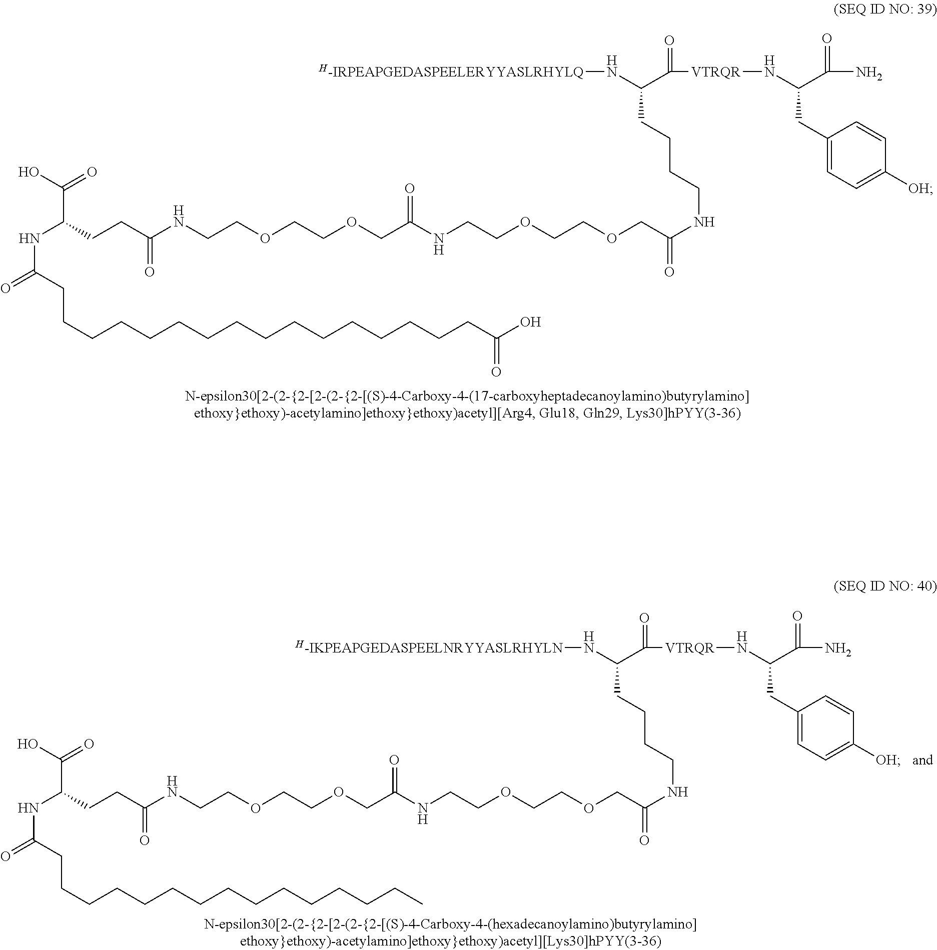

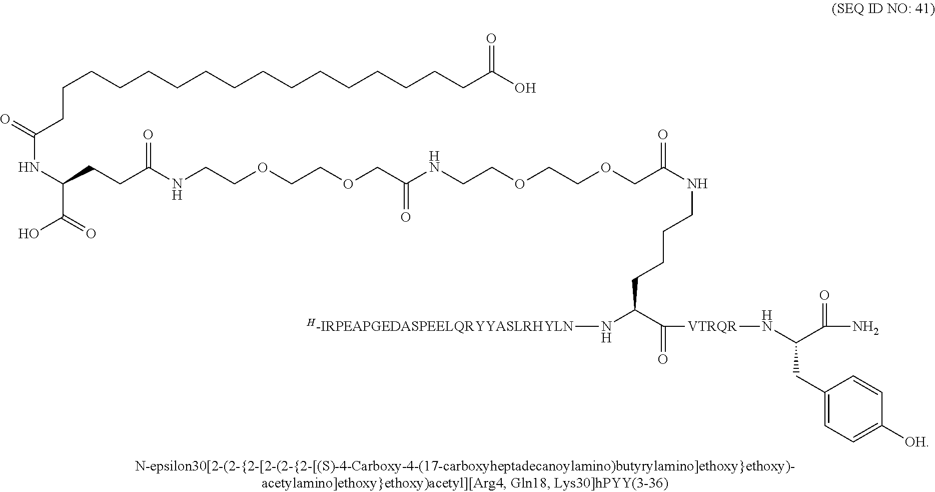

In one embodiment the PYY derivative is

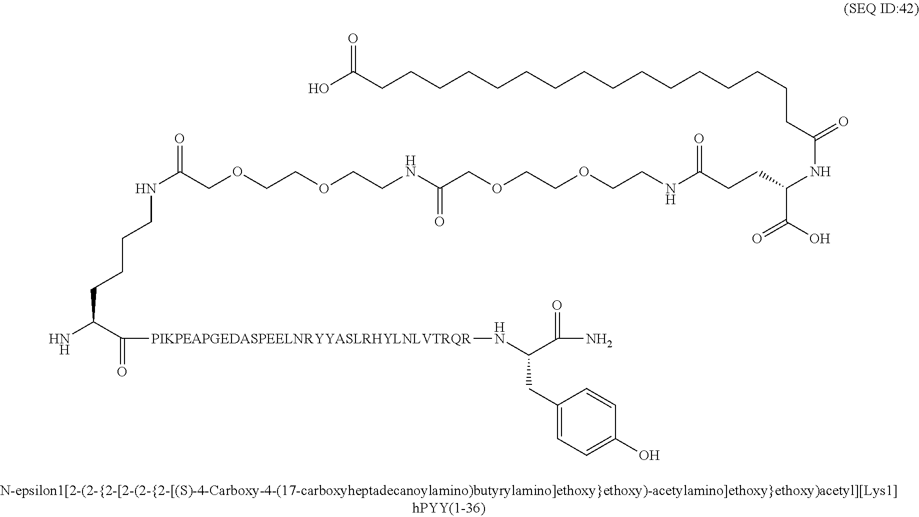

In one embodiment the PYY derivative of the invention is selected from the group consisting of

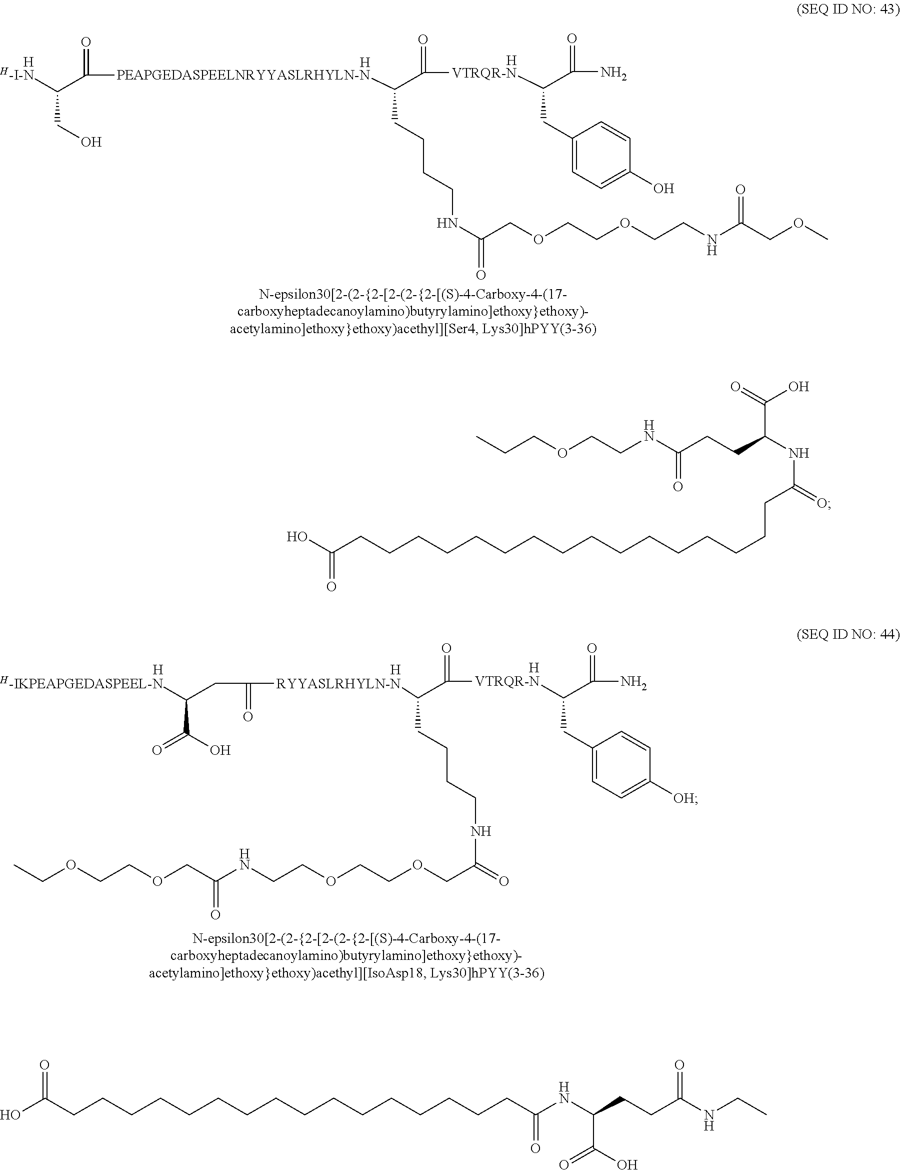

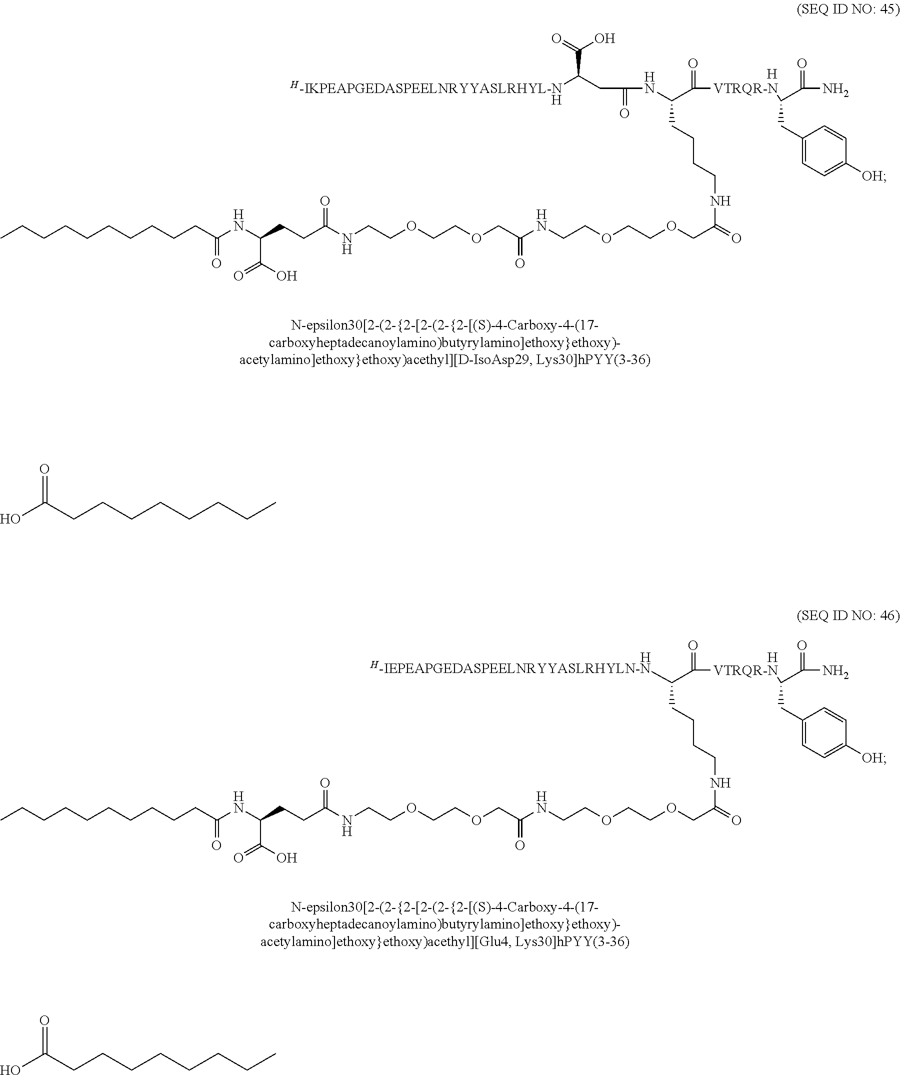

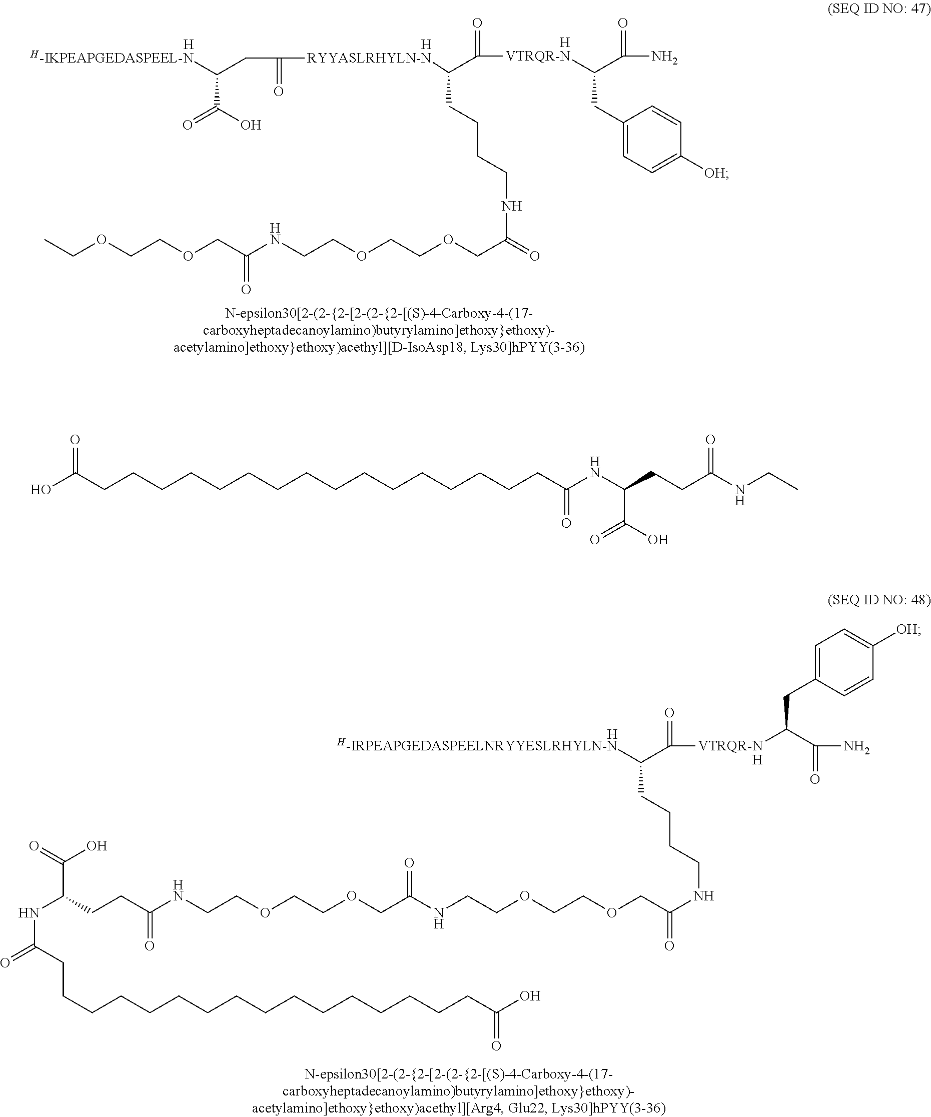

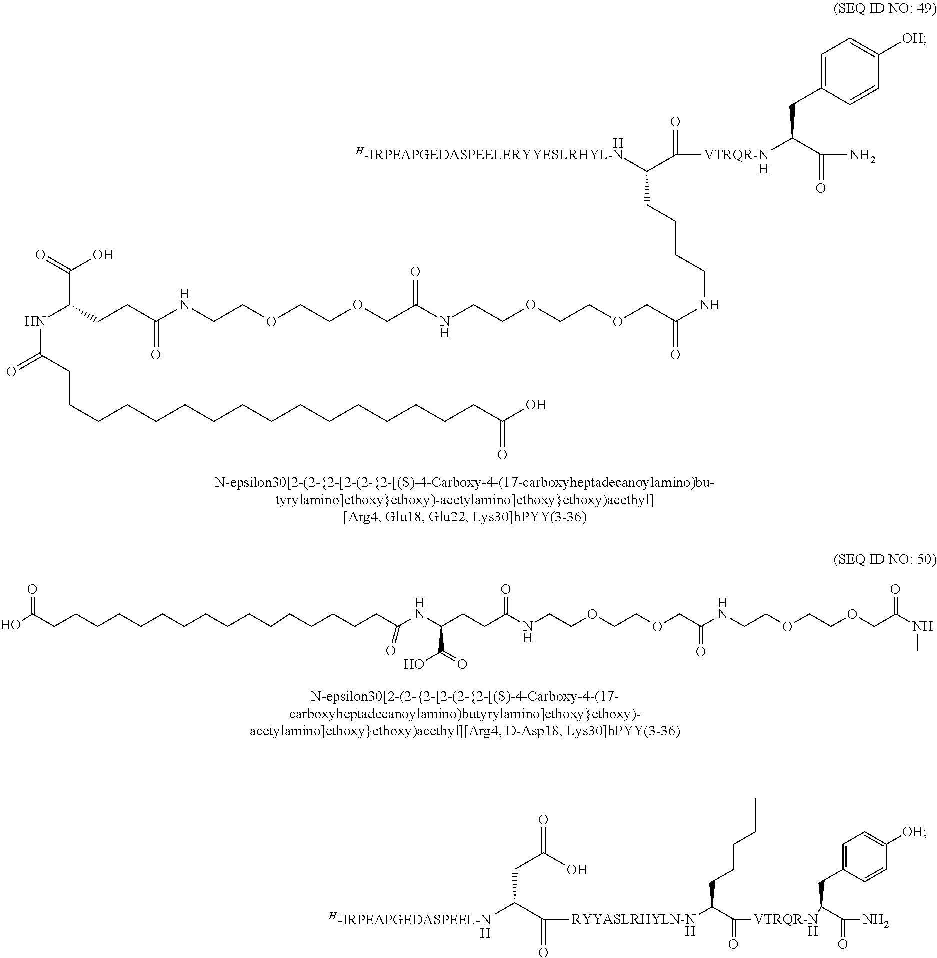

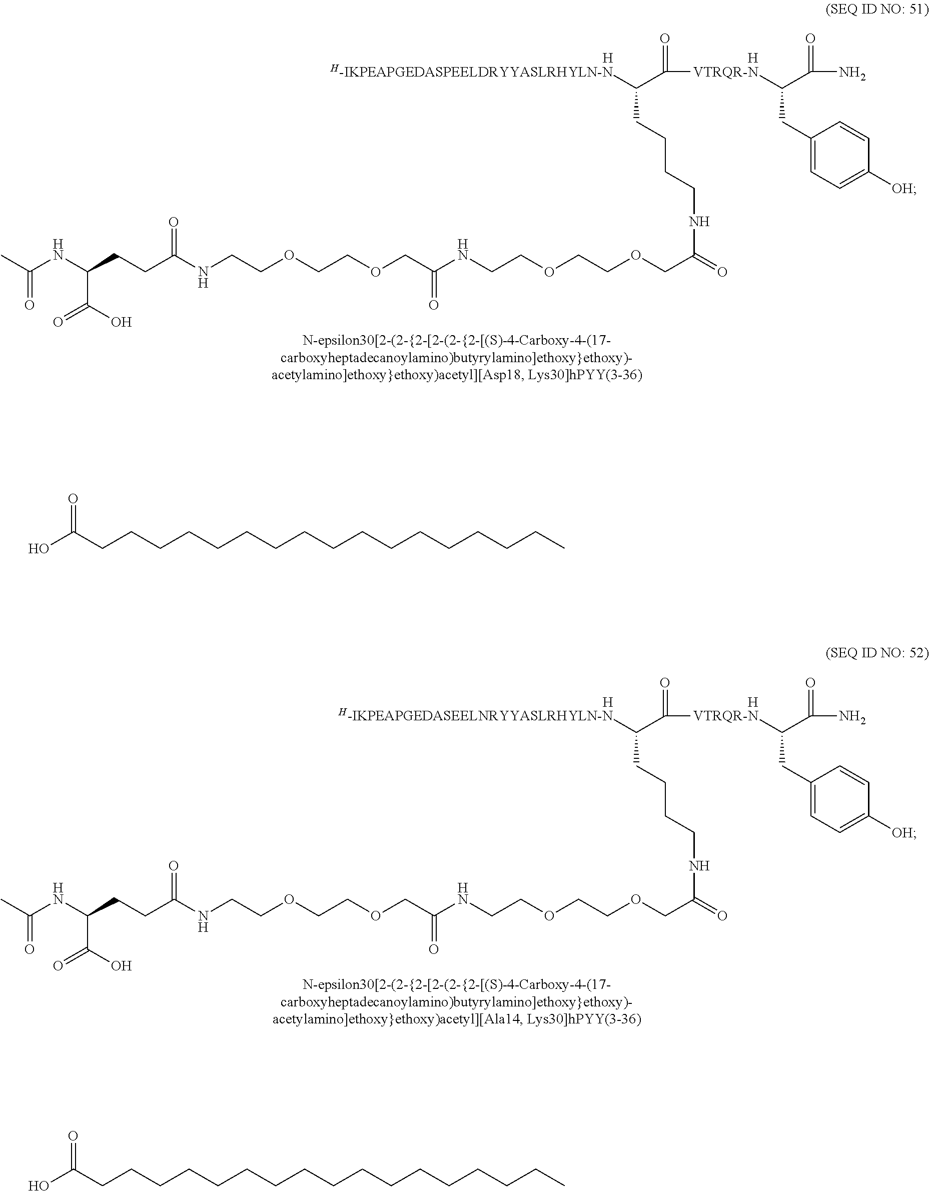

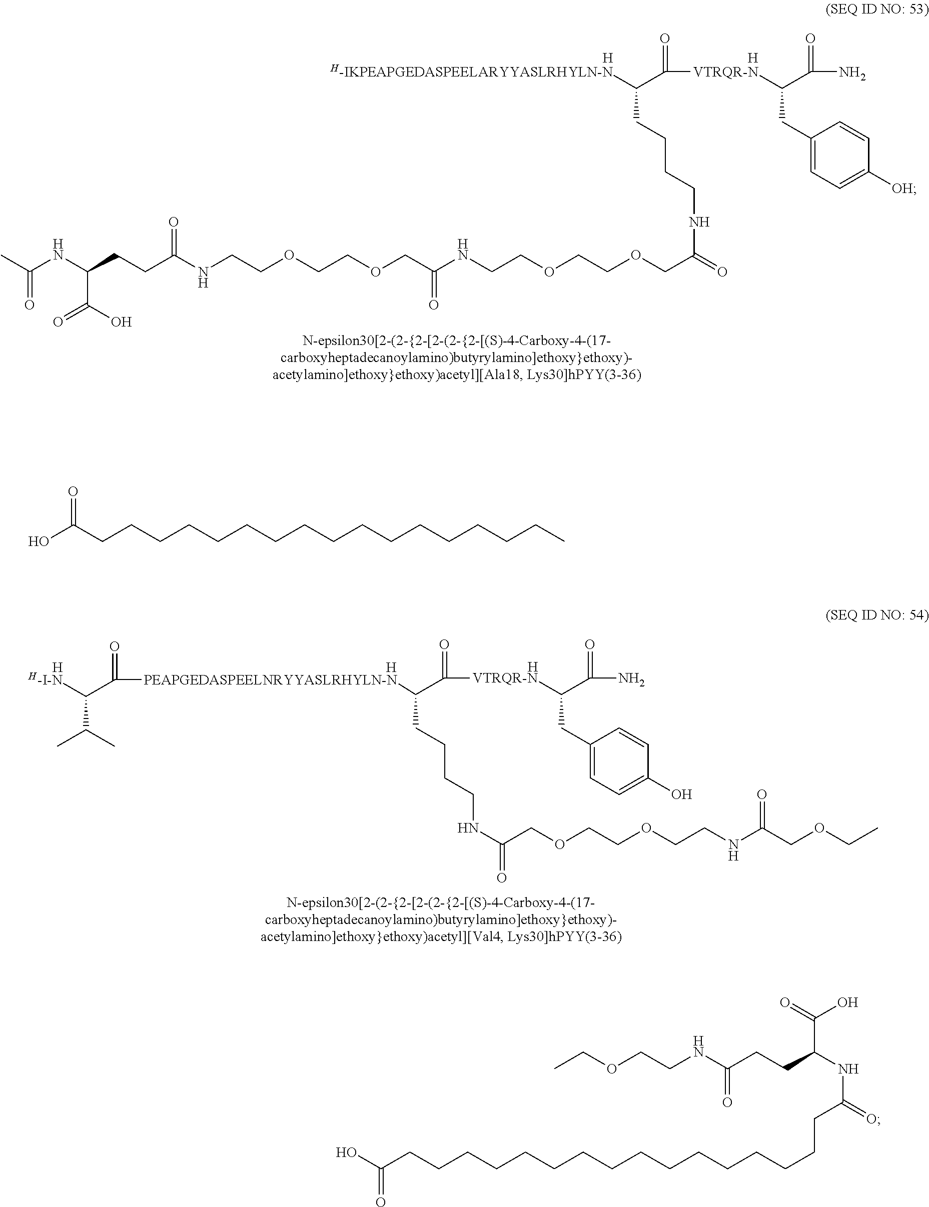

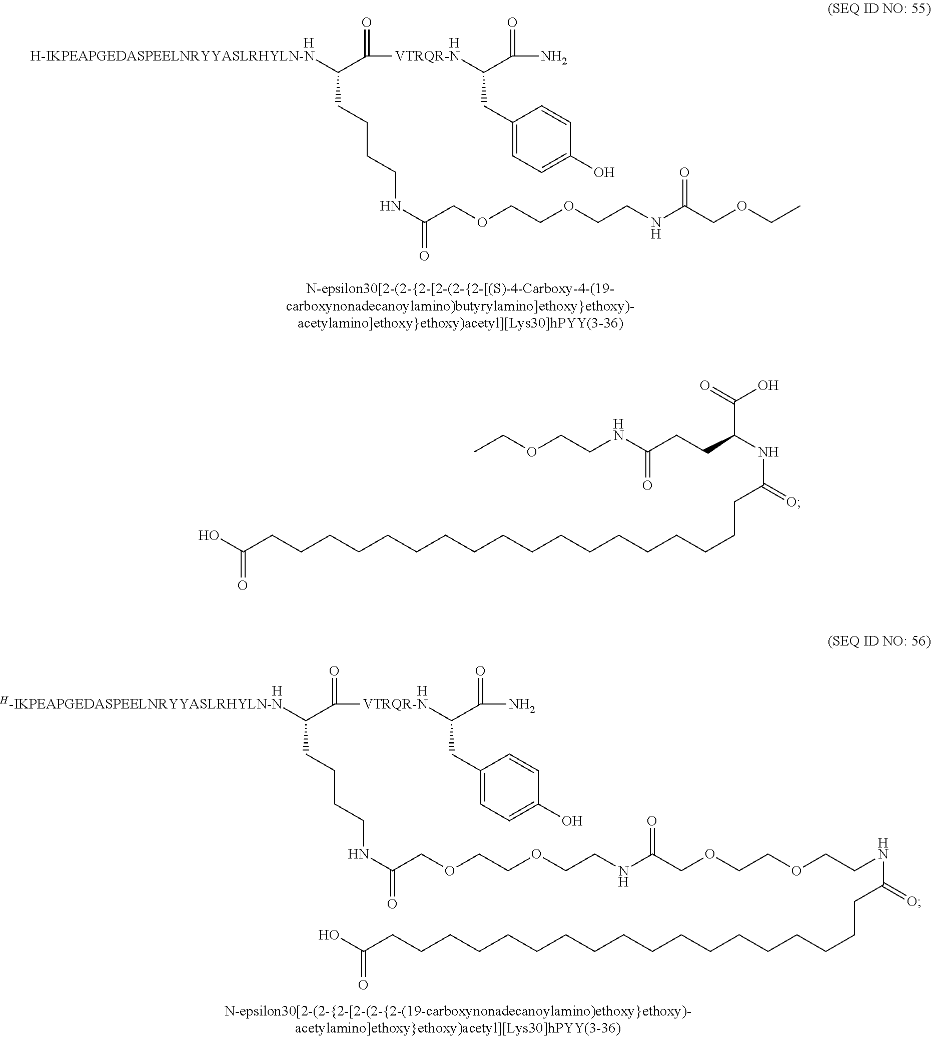

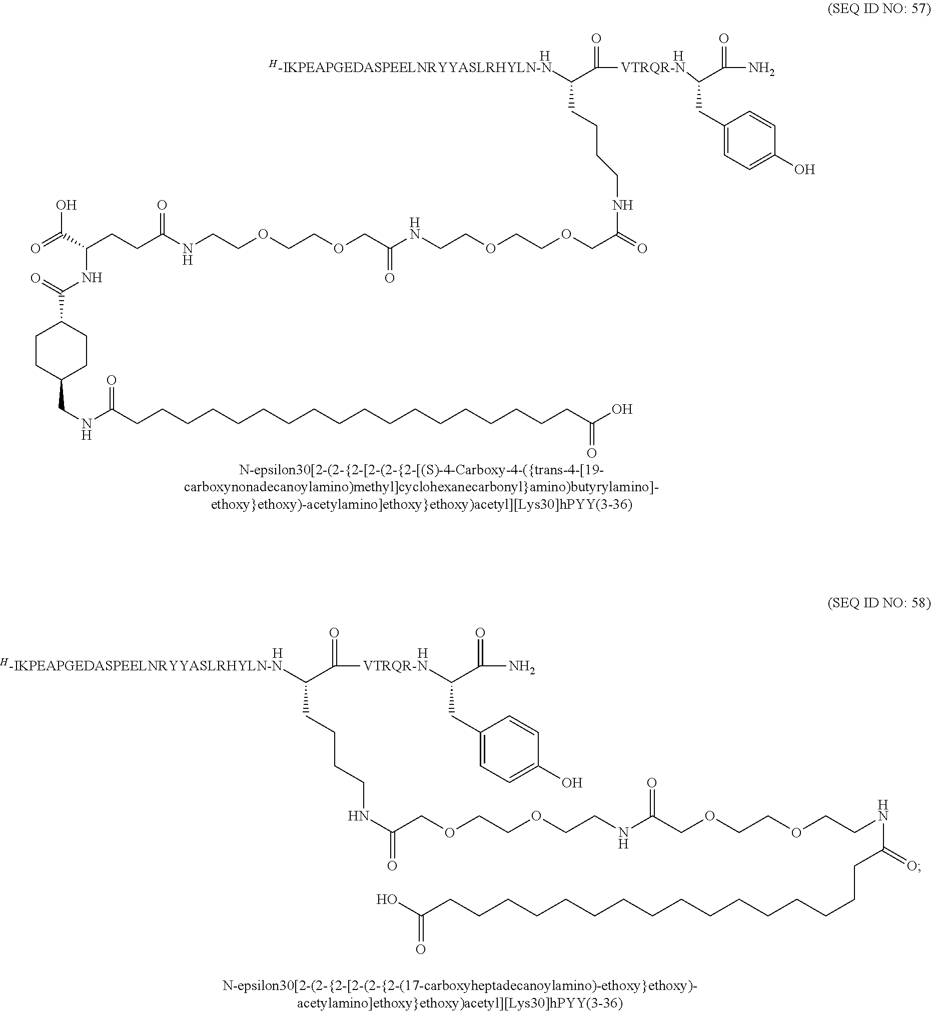

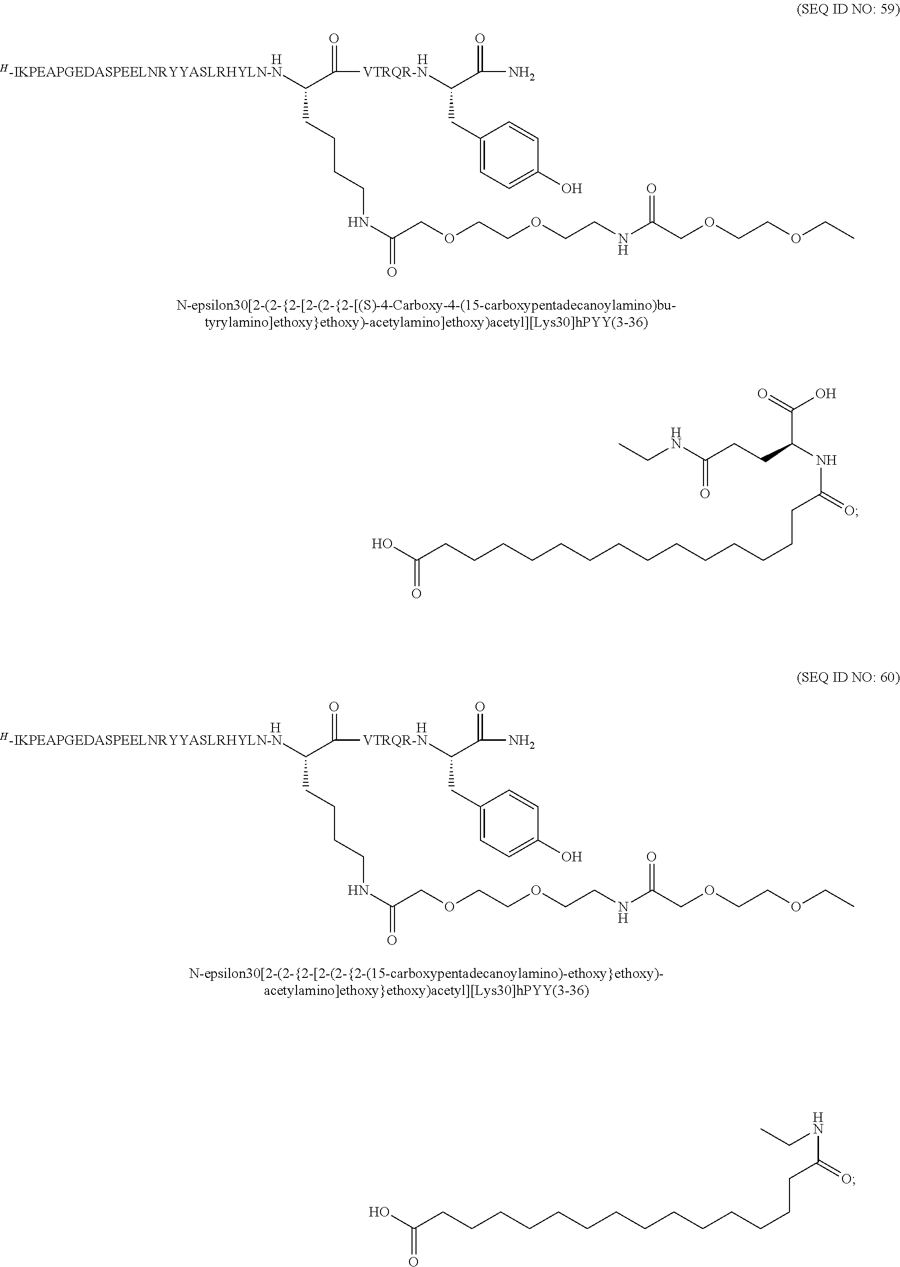

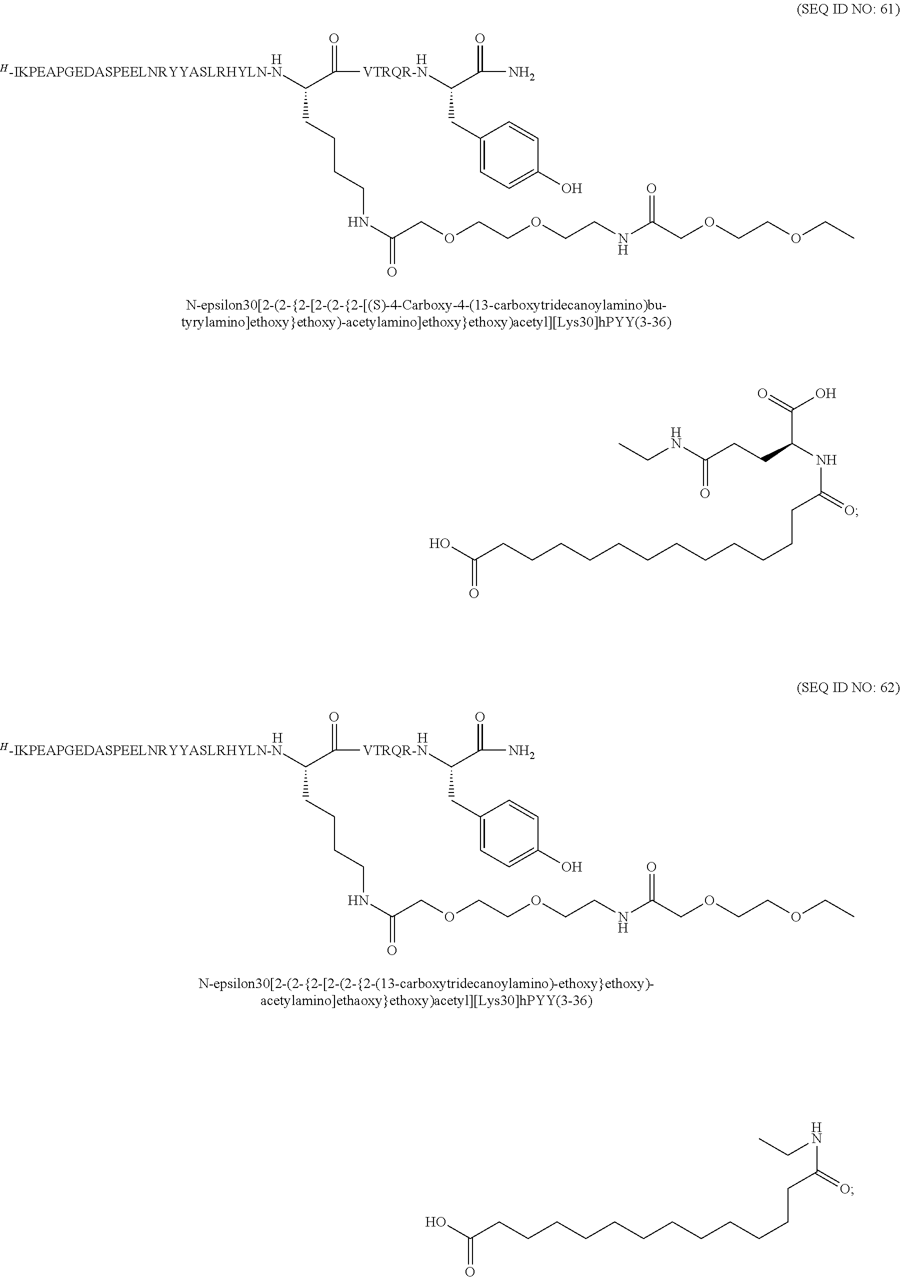

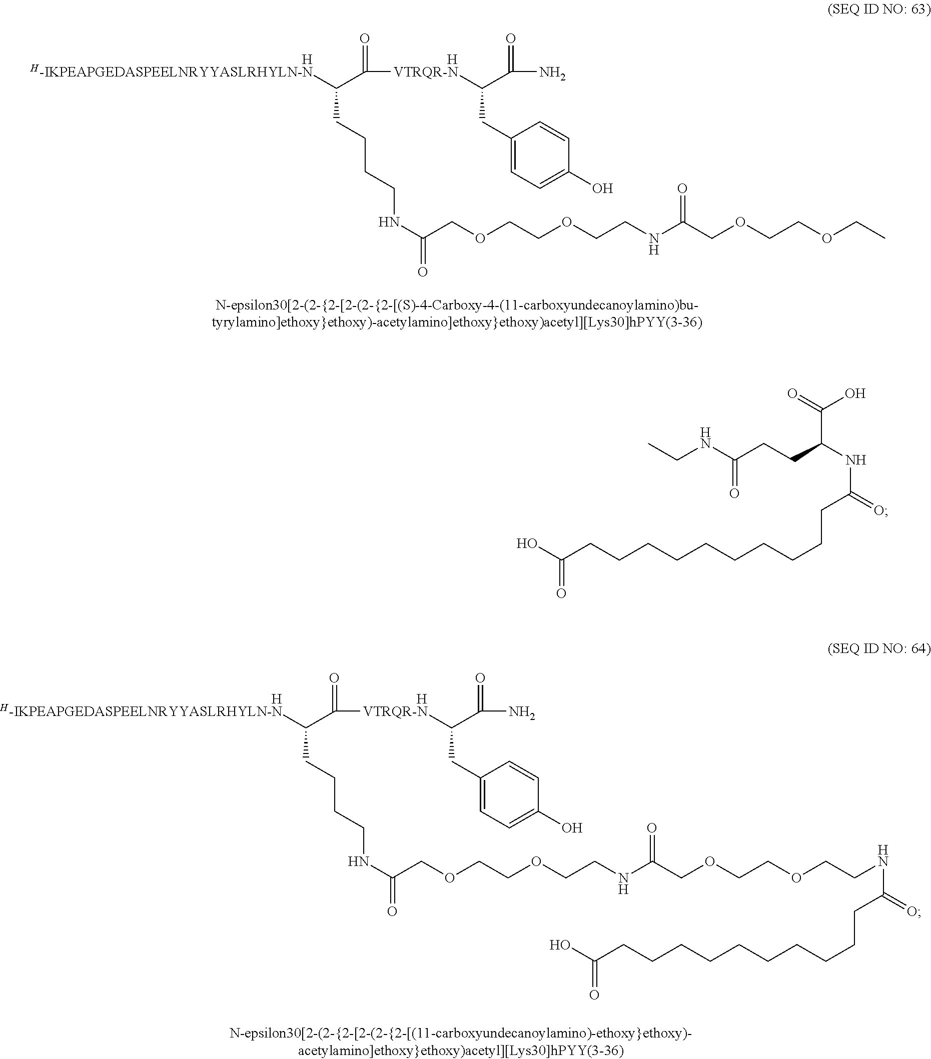

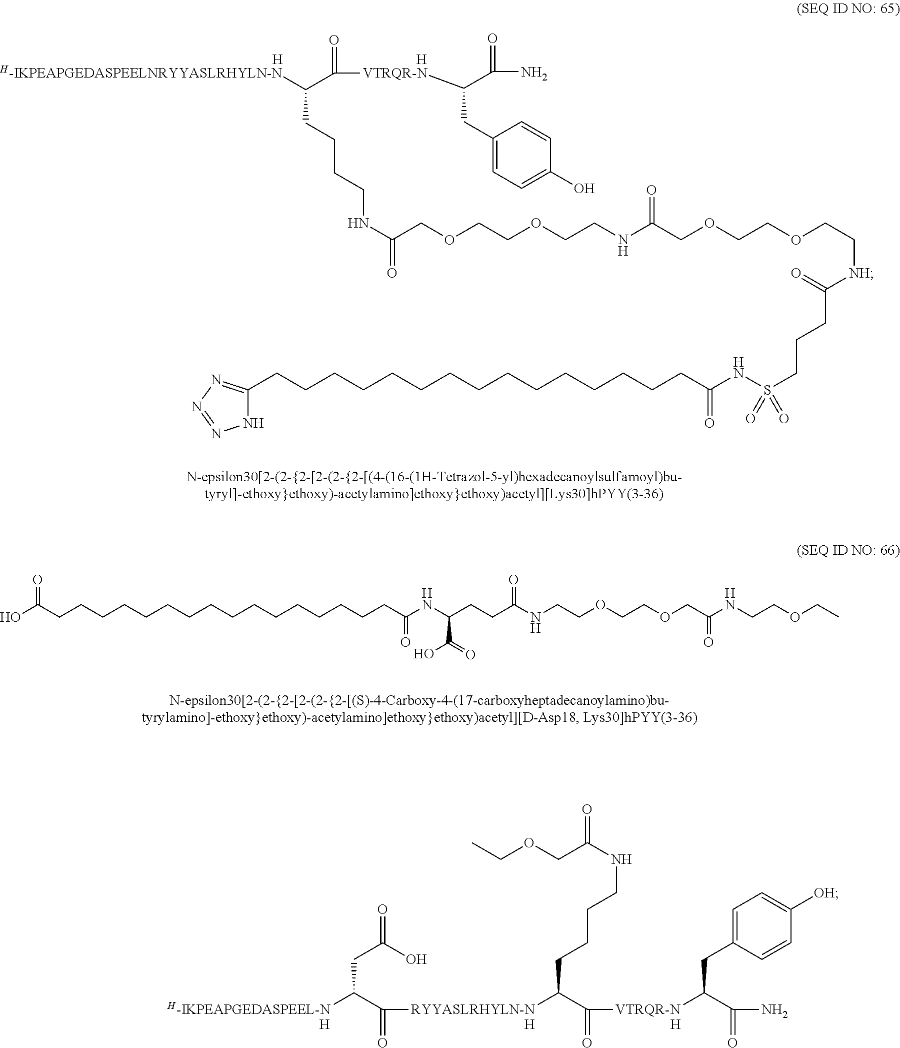

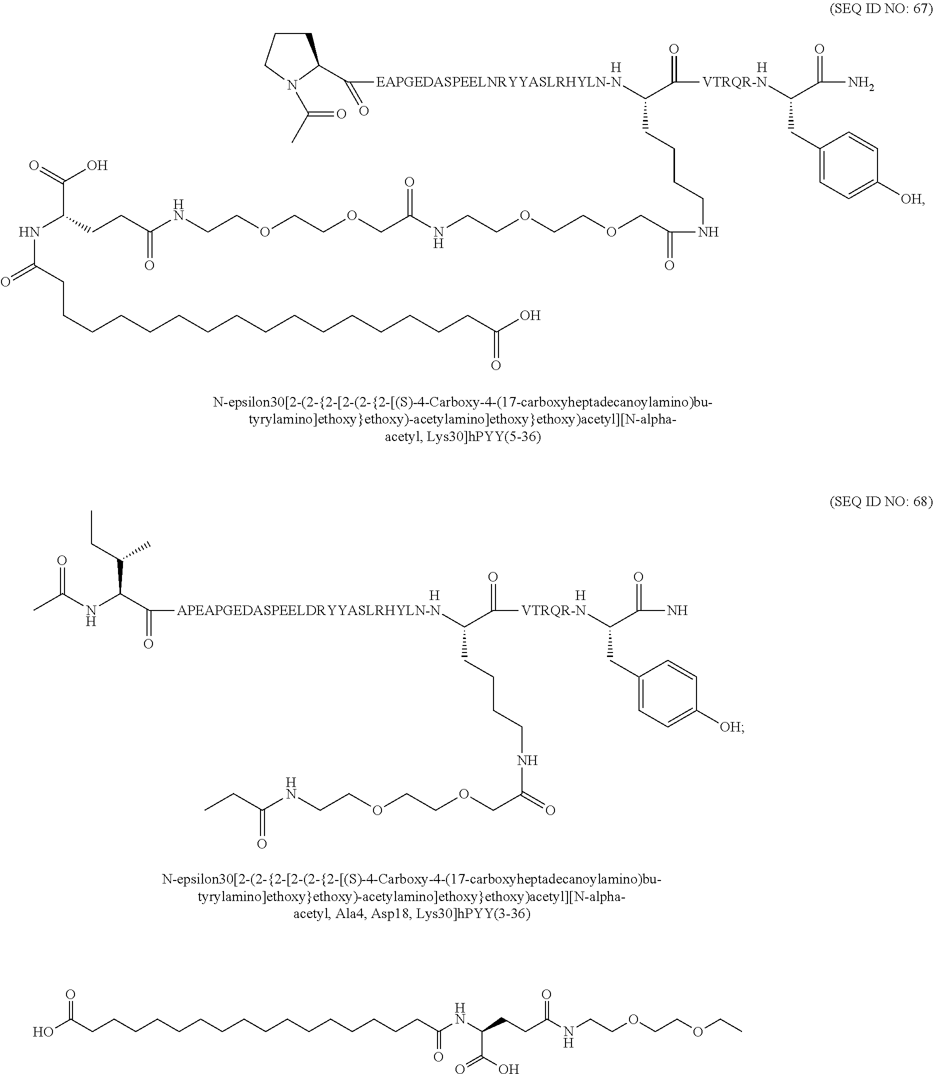

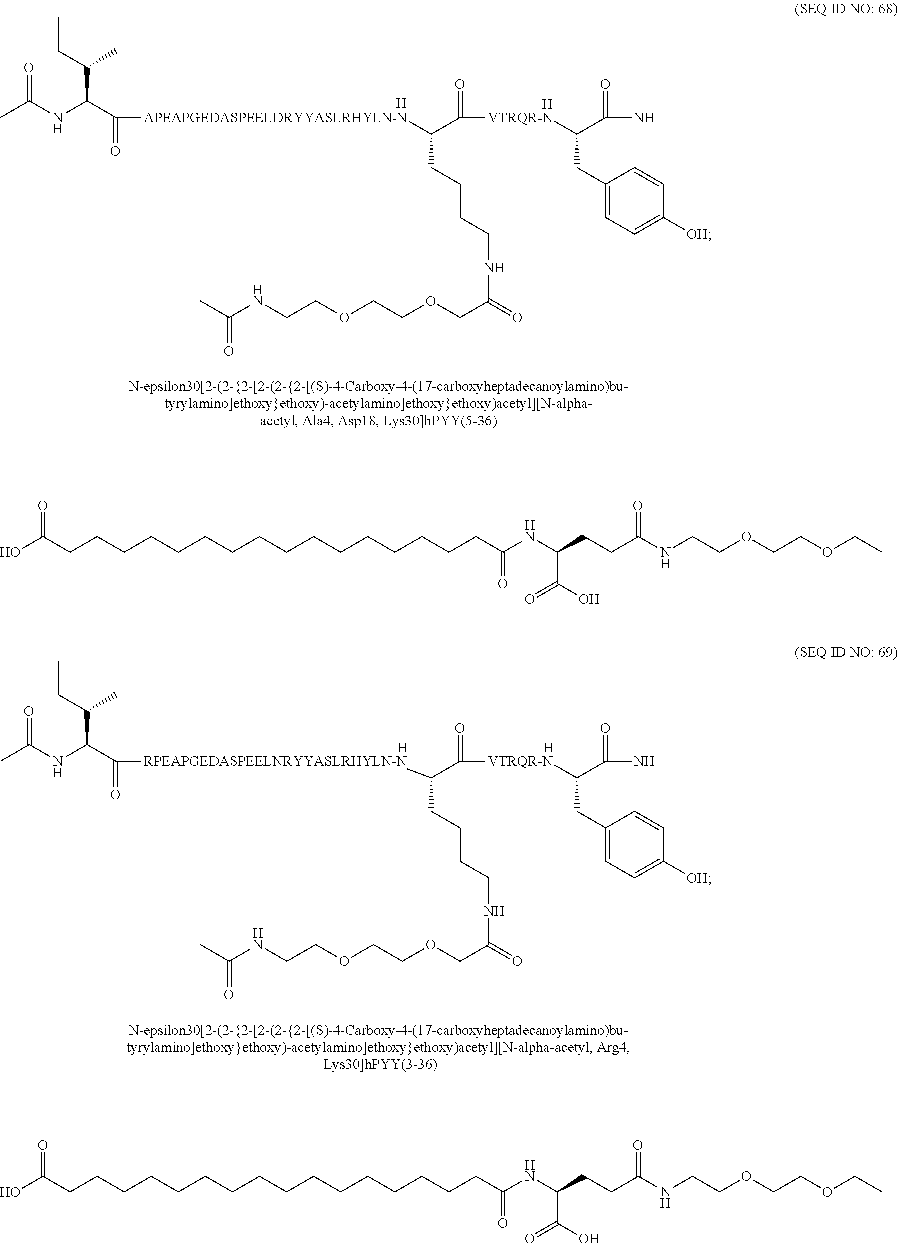

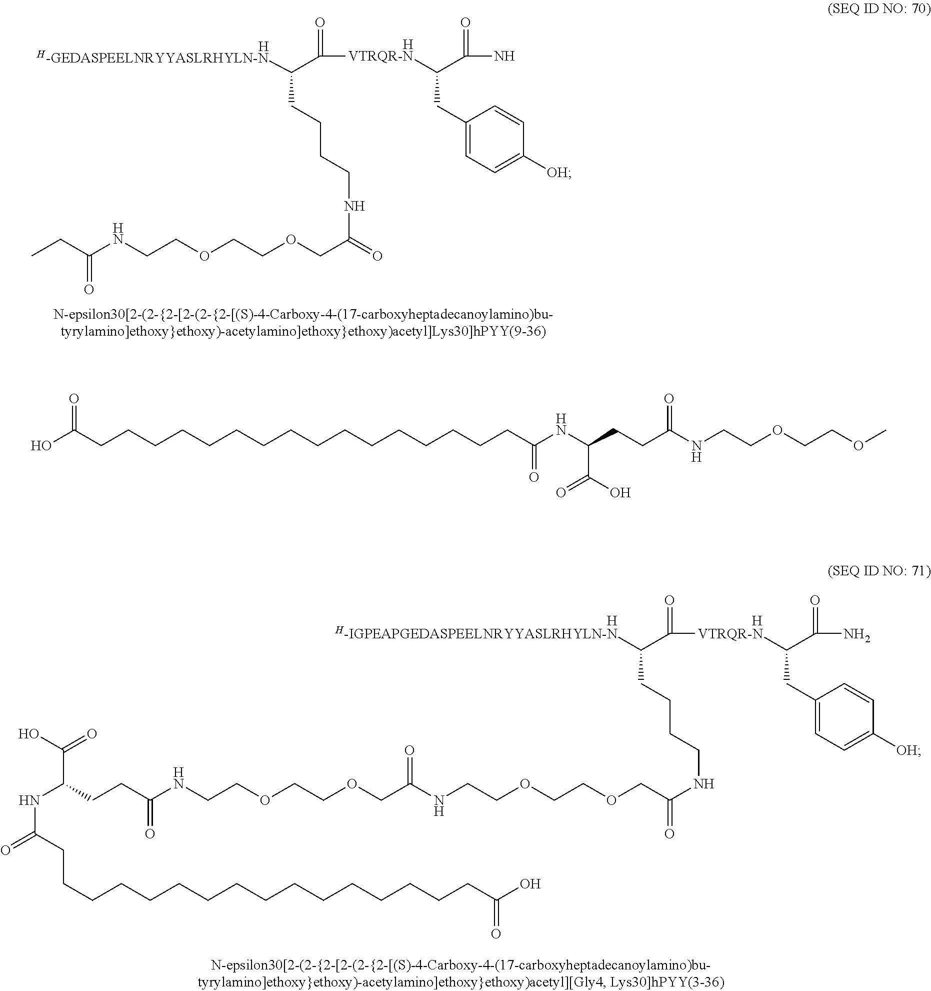

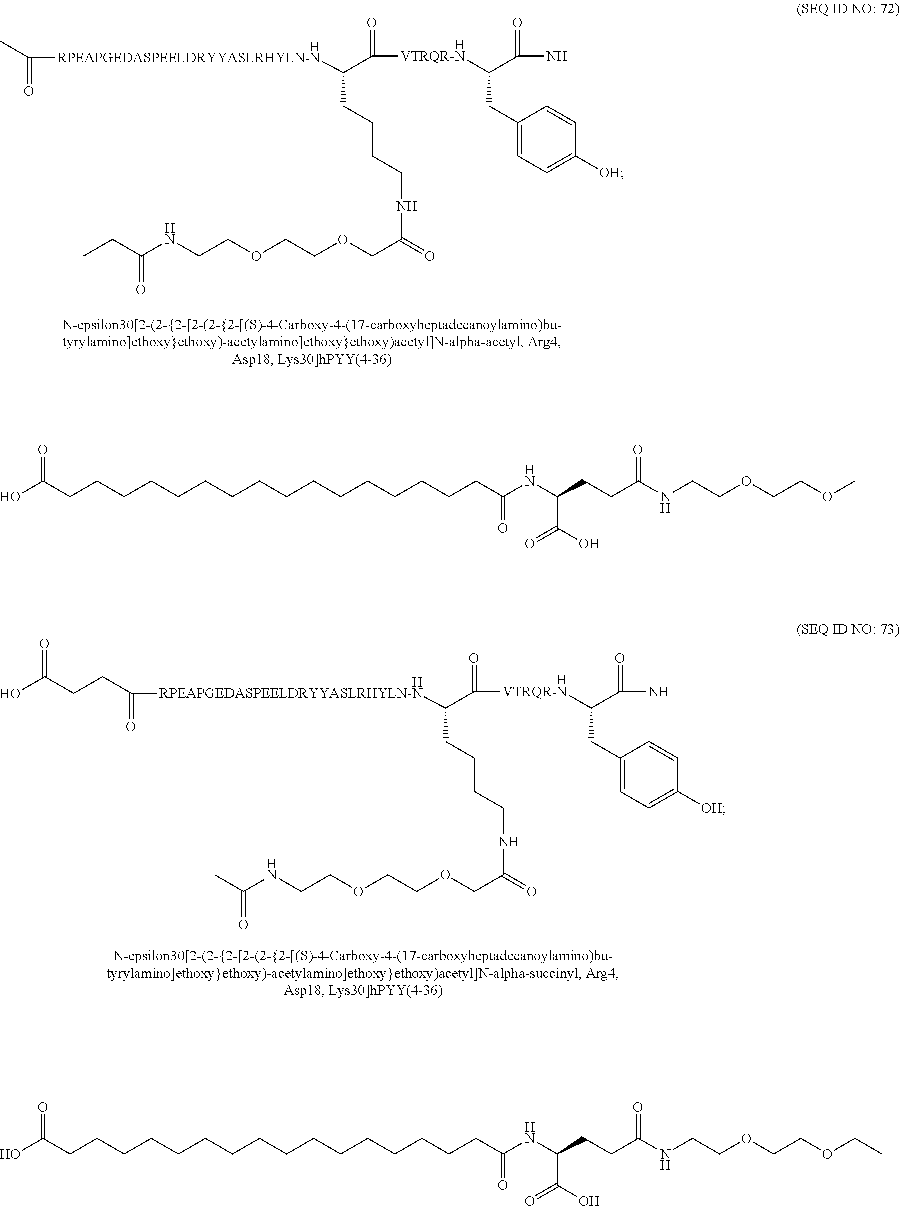

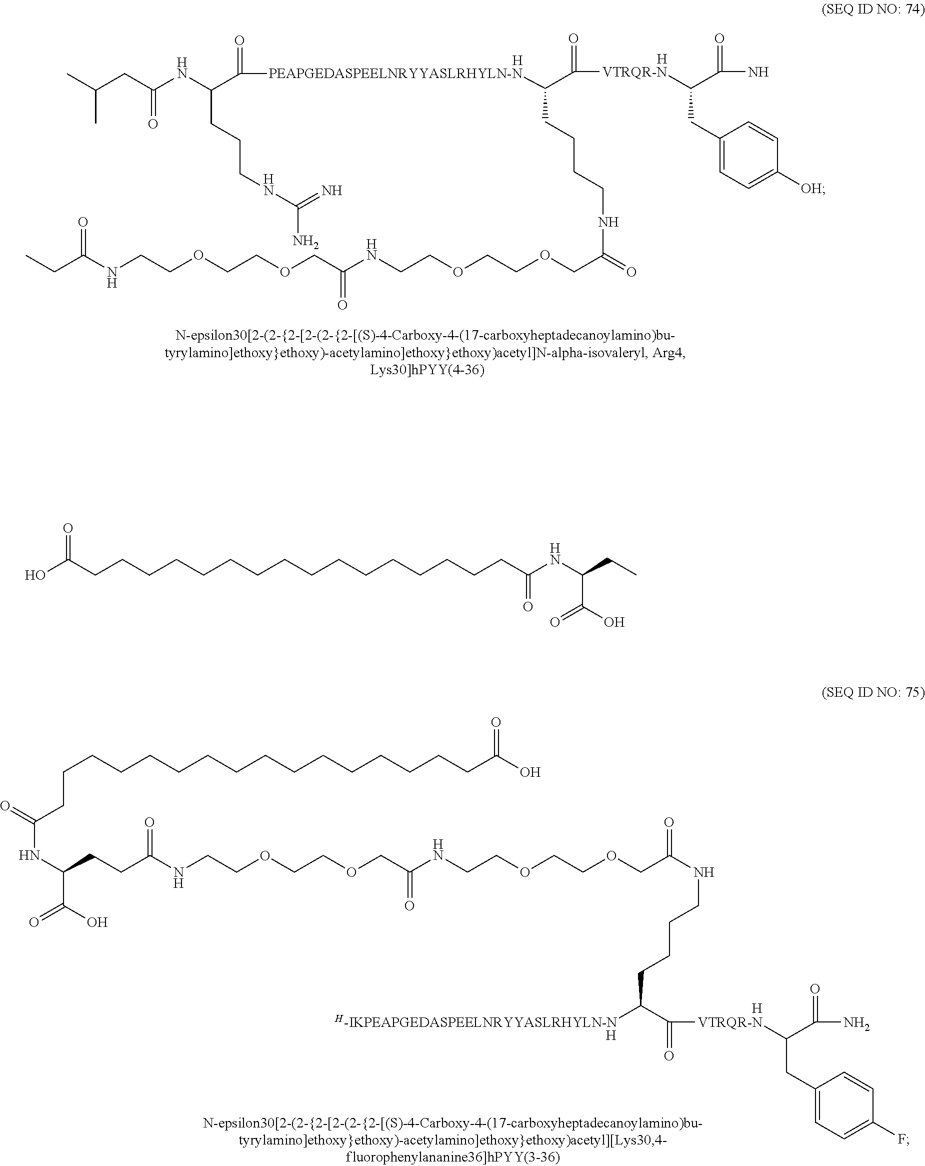

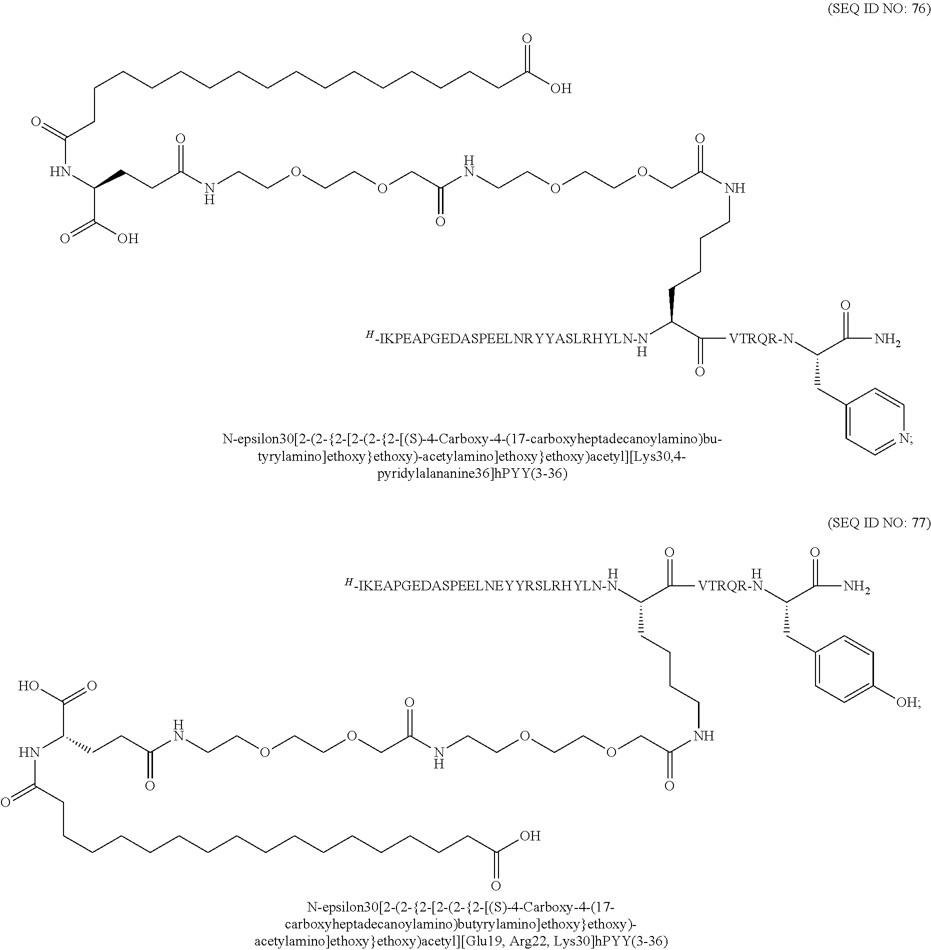

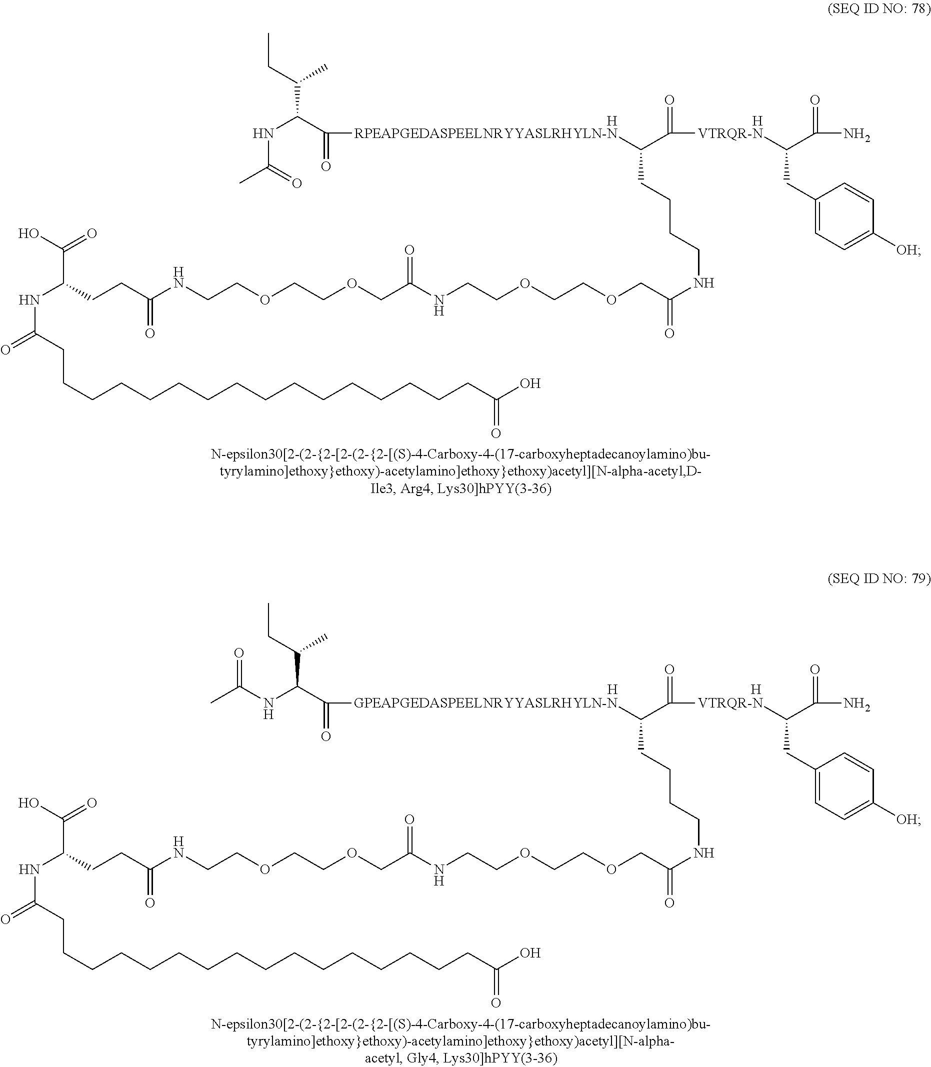

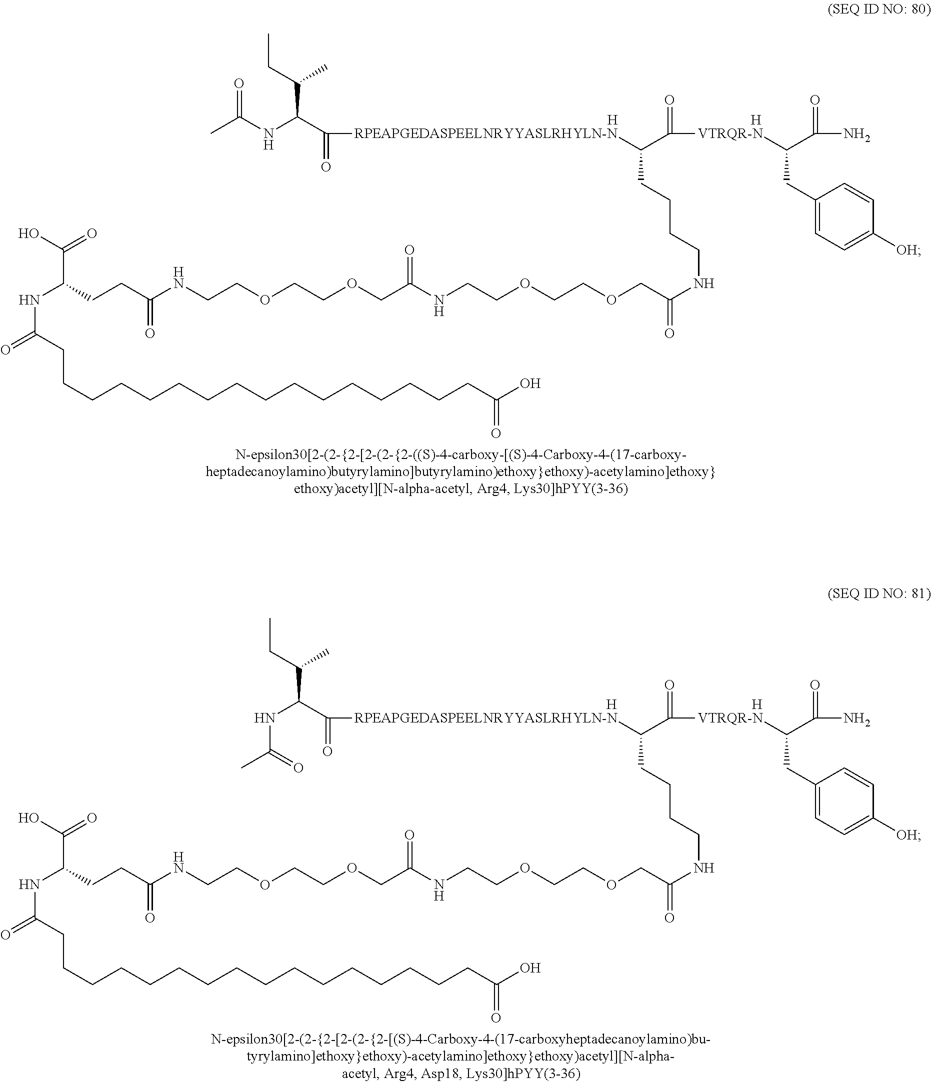

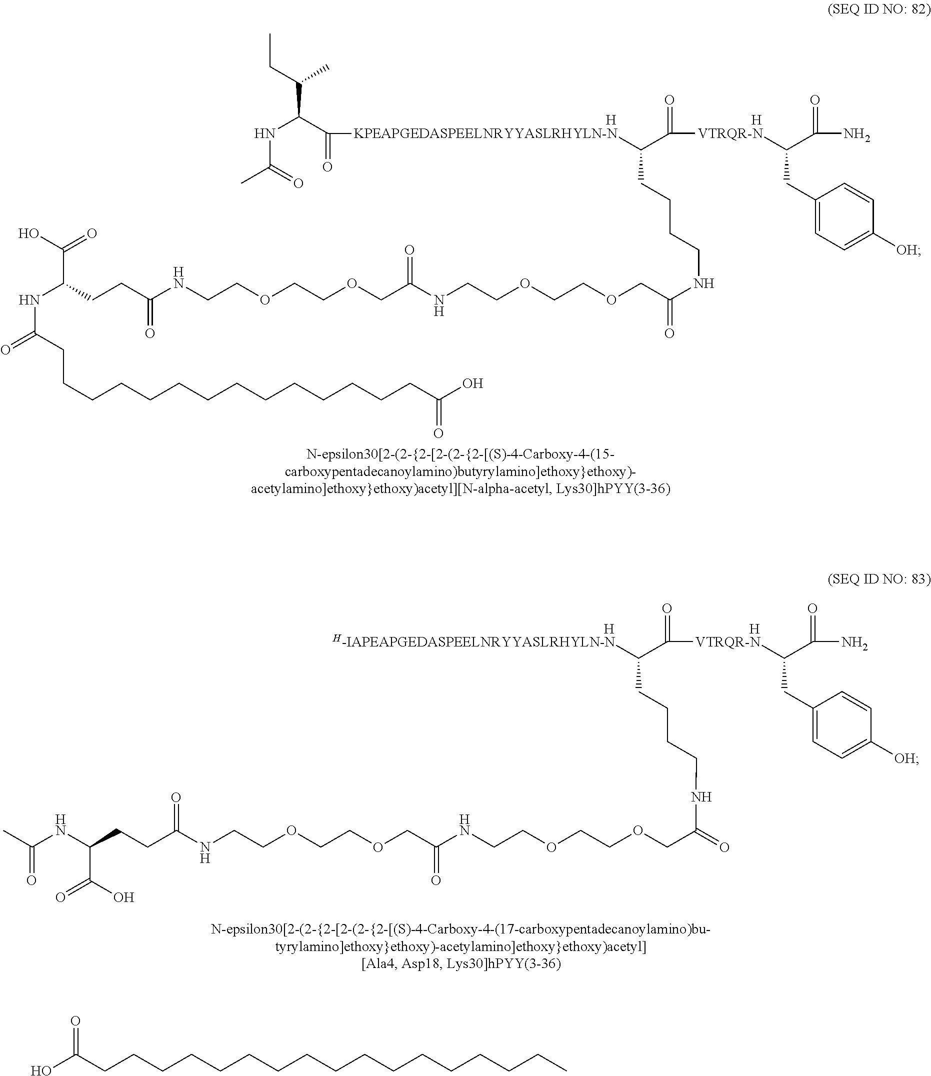

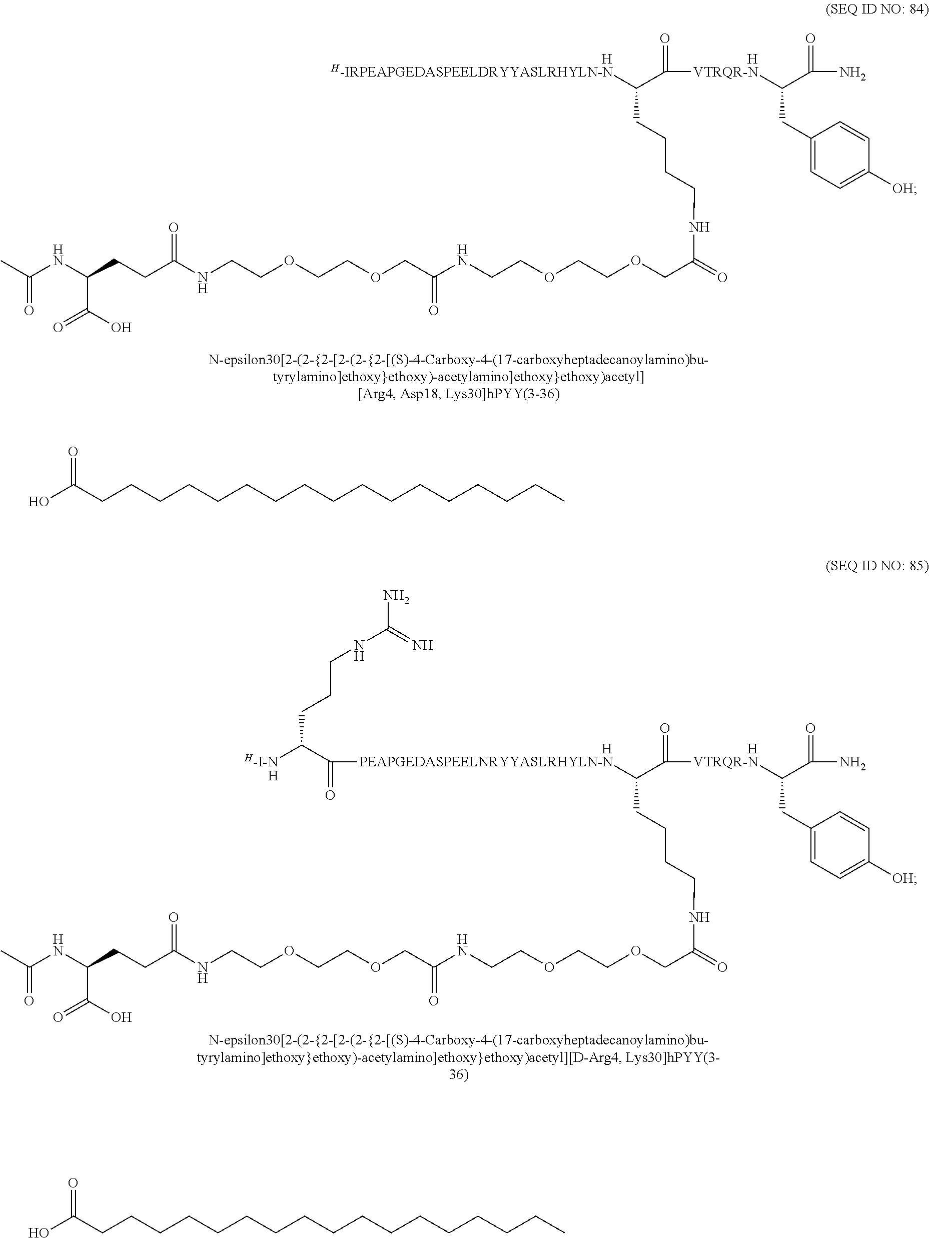

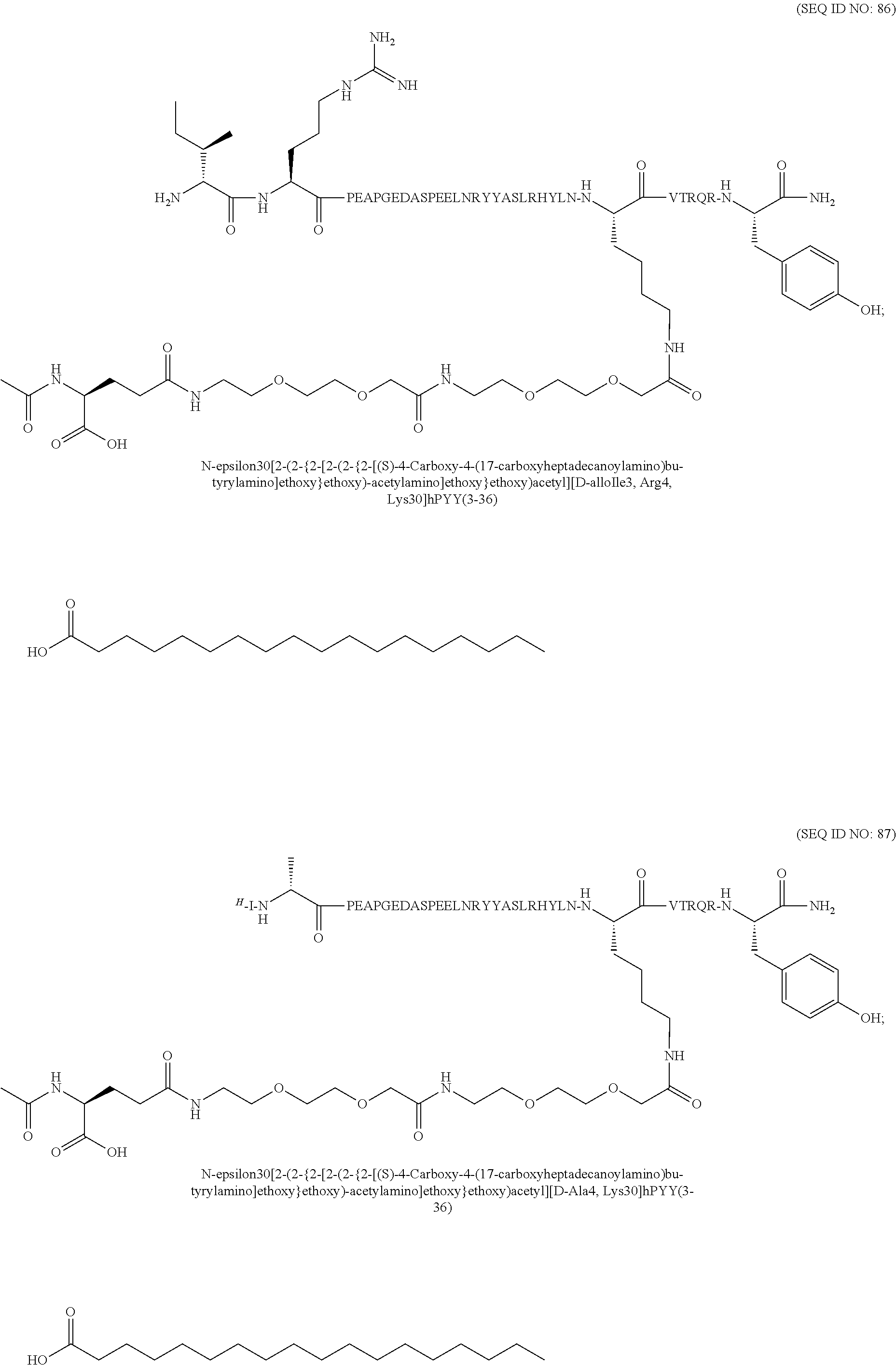

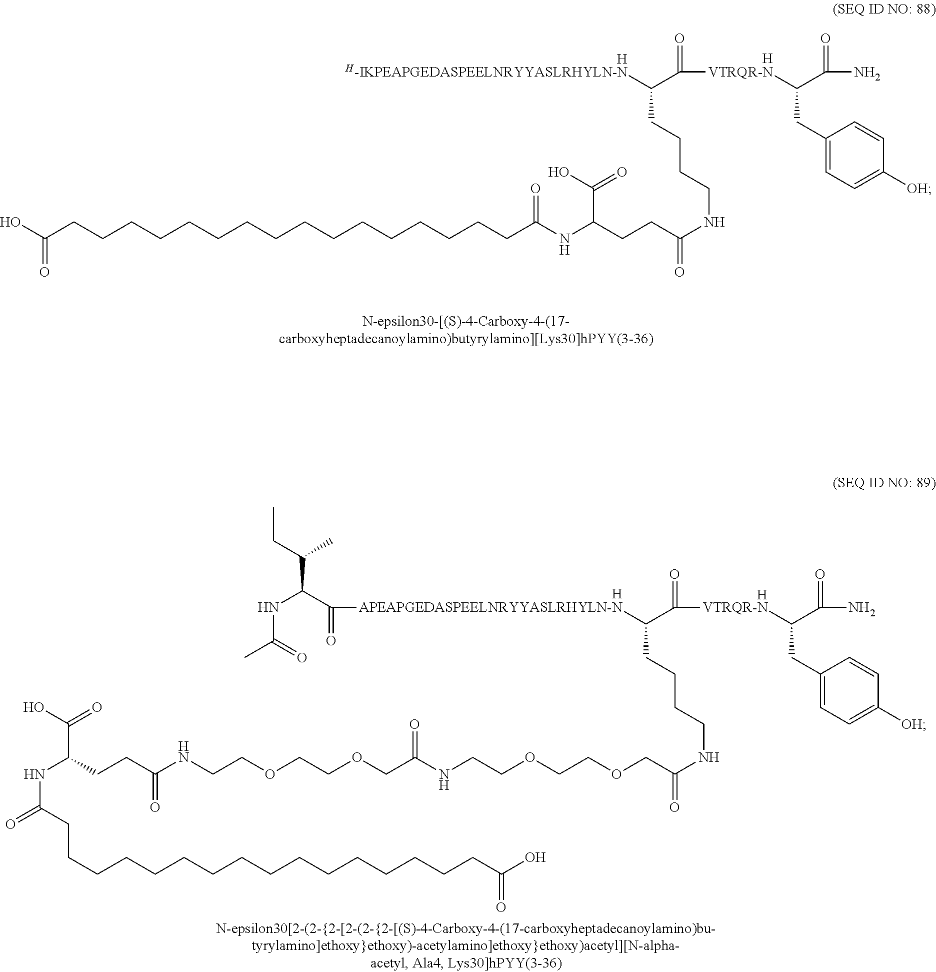

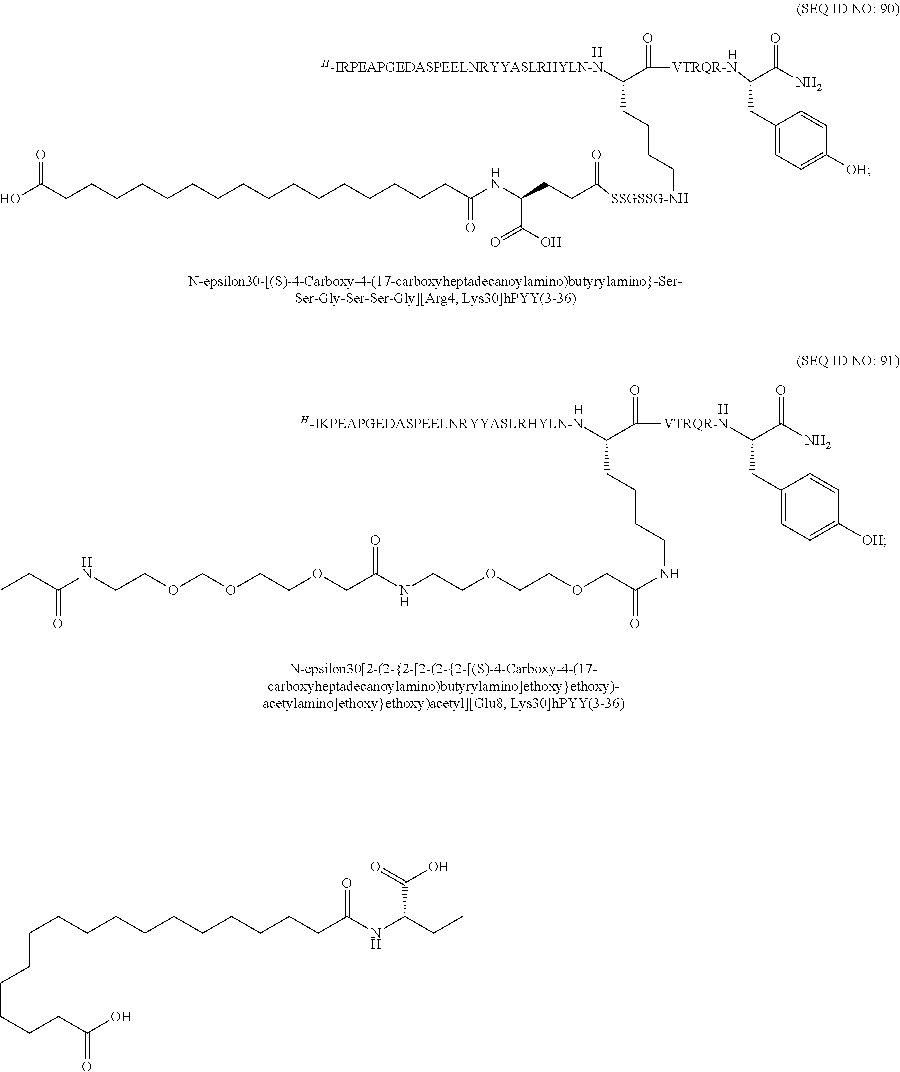

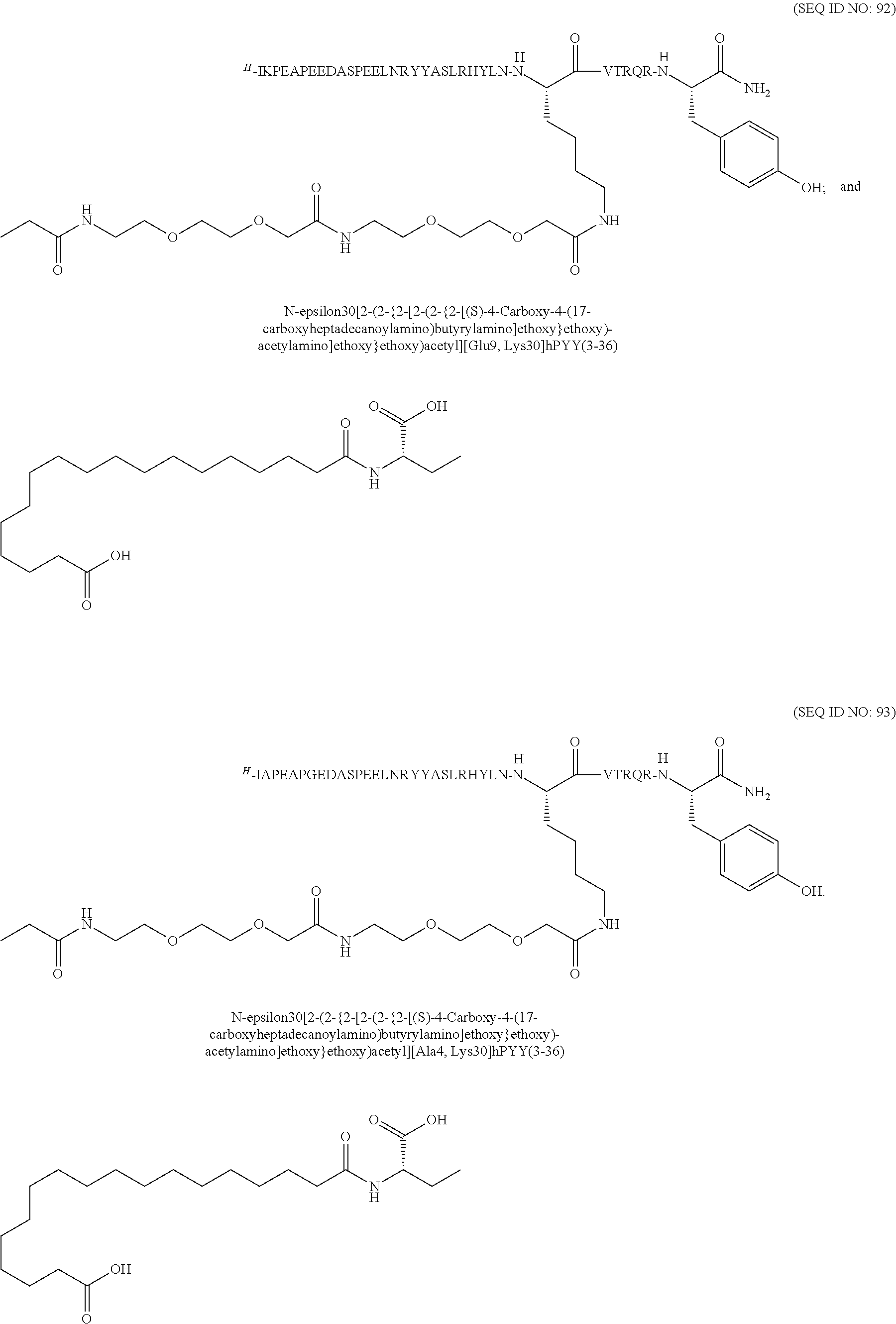

In one embodiment the PYY derivative is SEQ ID NO: 1. In one embodiment the PYY derivative is SEQ ID NO: 2. In one embodiment the PYY derivative is SEQ ID NO: 3. In one embodiment the PYY derivative is SEQ ID NO: 4. In one embodiment the PYY derivative is SEQ ID NO: 5. In one embodiment the PYY derivative is SEQ ID NO: 6. In one embodiment the PYY derivative is SEQ ID NO: 7. In one embodiment the PYY derivative is SEQ ID NO: 8. In one embodiment the PYY derivative is SEQ ID NO: 9. In one embodiment the PYY derivative is SEQ ID NO: 10. In one embodiment the PYY derivative is SEQ ID NO: 11. In one embodiment the PYY derivative is SEQ ID NO: 12. In one embodiment the PYY derivative is SEQ ID NO: 13. In one embodiment the PYY derivative is SEQ ID NO: 14. In one embodiment the PYY derivative is SEQ ID NO: 15. In one embodiment the PYY derivative is SEQ ID NO: 16. In one embodiment the PYY derivative is SEQ ID NO: 17. In one embodiment the PYY derivative is SEQ ID NO: 18. In one embodiment the PYY derivative is SEQ ID NO: 19. In one embodiment the PYY derivative is SEQ ID NO: 20. In one embodiment the PYY derivative is SEQ ID NO: 21. In one embodiment the PYY derivative is SEQ ID NO: 22. In one embodiment the PYY derivative is SEQ ID NO: 23. In one embodiment the PYY derivative is SEQ ID NO: 24. In one embodiment the PYY derivative is SEQ ID NO: 25. In one embodiment the PYY derivative is SEQ ID NO: 26. In one embodiment the PYY derivative is SEQ ID NO: 27. In one embodiment the PYY derivative is SEQ ID NO: 28. In one embodiment the PYY derivative is SEQ ID NO: 29. In one embodiment the PYY derivative is SEQ ID NO: 30. In one embodiment the PYY derivative is SEQ ID NO: 31. In one embodiment the PYY derivative is SEQ ID NO: 32. In one embodiment the PYY derivative is SEQ ID NO: 33. In one embodiment the PYY derivative is SEQ ID NO: 34. In one embodiment the PYY derivative is SEQ ID NO: 35. In one embodiment the PYY derivative is SEQ ID NO: 36. In one embodiment the PYY derivative is SEQ ID NO: 37. In one embodiment the PYY derivative is SEQ ID NO: 38. In one embodiment the PYY derivative is SEQ ID NO: 39. In one embodiment the PYY derivative is SEQ ID NO: 40. In one embodiment the PYY derivative is SEQ ID NO: 41. In one embodiment the PYY derivative is SEQ ID NO: 42. In one embodiment the PYY derivative is SEQ ID NO: 43. In one embodiment the PYY derivative is SEQ ID NO: 44. In one embodiment the PYY derivative is SEQ ID NO: 45. In one embodiment the PYY derivative is SEQ ID NO: 46. In one embodiment the PYY derivative is SEQ ID NO: 47. In one embodiment the PYY derivative is SEQ ID NO: 48. In one embodiment the PYY derivative is SEQ ID NO: 49. In one embodiment the PYY derivative is SEQ ID NO: 50. In one embodiment the PYY derivative is SEQ ID NO: 51. In one embodiment the PYY derivative is SEQ ID NO: 52. In one embodiment the PYY derivative is SEQ ID NO: 53. In one embodiment the PYY derivative is SEQ ID NO: 54. In one embodiment the PYY derivative is SEQ ID NO: 55. In one embodiment the PYY derivative is SEQ ID NO: 56. In one embodiment the PYY derivative is SEQ ID NO: 57. In one embodiment the PYY derivative is SEQ ID NO: 58. In one embodiment the PYY derivative is SEQ ID NO: 59. In one embodiment the PYY derivative is SEQ ID NO: 60. In one embodiment the PYY derivative is SEQ ID NO: 61. In one embodiment the PYY derivative is SEQ ID NO: 62. In one embodiment the PYY derivative is SEQ ID NO: 63. In one embodiment the PYY derivative is SEQ ID NO: 64. In one embodiment the PYY derivative is SEQ ID NO: 65. In one embodiment the PYY derivative is SEQ ID NO: 66. In one embodiment the PYY derivative is SEQ ID NO: 67. In one embodiment the PYY derivative is SEQ ID NO: 68. In one embodiment the PYY derivative is SEQ ID NO: 69. In one embodiment the PYY derivative is SEQ ID NO: 70. In one embodiment the PYY derivative is SEQ ID NO: 71. In one embodiment the PYY derivative is SEQ ID NO: 72. In one embodiment the PYY derivative is SEQ ID NO: 73. In one embodiment the PYY derivative is SEQ ID NO: 74. In one embodiment the PYY derivative is SEQ ID NO: 75. In one embodiment the PYY derivative is SEQ ID NO: 76. In one embodiment the PYY derivative is SEQ ID NO: 77. In one embodiment the PYY derivative is SEQ ID NO: 78. In one embodiment the PYY derivative is SEQ ID NO: 79. In one embodiment the PYY derivative is SEQ ID NO: 80. In one embodiment the PYY derivative is SEQ ID NO: 81. In one embodiment the PYY derivative is SEQ ID NO: 82. In one embodiment the PYY derivative is SEQ ID NO: 83. In one embodiment the PYY derivative is SEQ ID NO: 84. In one embodiment the PYY derivative is SEQ ID NO: 85. In one embodiment the PYY derivative is SEQ ID NO: 86. In one embodiment the PYY derivative is SEQ ID NO: 87. In one embodiment the PYY derivative is SEQ ID NO: 88. In one embodiment the PYY derivative is SEQ ID NO: 89. In one embodiment the PYY derivative is SEQ ID NO: 90. In one embodiment the PYY derivative is SEQ ID NO: 91. In one embodiment the PYY derivative is SEQ ID NO: 92. In one embodiment the PYY derivative is SEQ ID NO: 93.

In one embodiment the PYY derivative is not
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY3-36,
N-epsilon13-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys13]hPYY3-36,
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY3-36,
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys11]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY2-36,
N-epsilon4-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY3-36,
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Arg26]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Leu3,Glu4,Val6,Tyr7,Lys13,Arg26] hPYY1-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4, Lys13,Arg26]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4,Tyr7,Lys13,Arg26]hPYY1-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY3-36,
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys4]hPYY3-36,
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys11]hPYY3-36,
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY3-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY18-36,
N-epsilon25-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys25]hPYY3-36,
N-epsilon24-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys24]hPYY3-36,
N-epsilon19-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys19]hPYY3-36,
N-epsilon 13-[4-(16-(1H-Tetrazol-5-yl)hexadecanoylsulfamoyl)butyryl]ethoxy}ethoxy)acetylamino]ethoxy}ethoxy)acetyl][Lys13]PYY3-36,
N-epsilon25-[4-(16-(1H-Tetrazol-5-yl)hexadecanoylsulfamoyl)butyryl]ethoxy}ethoxy)acetylamino]ethoxy}ethoxy)acetyl][Lys25]PYY3-36,
N-alpha-[4-(16-(1H-Tetrazol-5-yl)hexadecanoylsulfamoyl)butyryl]ethoxy}ethoxy)acetylamino]ethoxy}ethoxy)acetyl]PYY3-36,
N-alpha-[4-(16-(1H-Tetrazol-5-yl)hexadecanoylsulfamoyl)butyryl]ethoxy}ethoxy)acetylamino]ethoxy}ethoxy)acetyl]PYY3-36,
N-epsilon18-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys18]PYY3-36,
N-epsilon22-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys22]PYY3-36,
N-epsilon26-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys26]PYY3-36,
N-epsilon29-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys29]PYY3-36,
N-epsilon36-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys36]PYY3-36,
N-epsilon21-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys21]PYY3-36,
N-epsilon30-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys30]PYY3-36,
N-epsilon31-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys31]PYY3-36,
N-epsilon14-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys14]PYY3-36,
N-epsilon15-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys15]PYY3-36,
N-epsilon16-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys16]PYY3-36,
N-epsilon20-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys20]PYY3-36,
N-epsilon28-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys28]PYY3-36 or
N-epsilon32-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys32]PYY3-36.

In one embodiment the PYY derivative is not
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}-ethoxy)acetylamino]ethoxy}ethoxy)acetyl] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][N-Methyl Arg35] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][His25,N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Aib25,N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Tyr25,N-Methyl Gln34] PYY(3-36),
N-alpha-{2-(2-{2-[2-(2-{2-[4-Carboxy-4-(17-carboxyheptadecanoylamino)butyrylamino]ethoxy}-ethoxy)acetylamino]-ethoxy}ethoxy)acetyl}[Ala20] PYY(3-36),
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[N-methyl Arg35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Ala27] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Agp35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Agb35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[HomoArg35] PYY(3-36),
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[NArg35] PYY(3-36) or
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[NGln34] PYY(3-36).

In one embodiment the PYY derivative is not
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,N-methyl Tyr36] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Cα-methyl Tyr36] PYY(3-36),
[N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl] PYY(3-36),
[N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl][NTyr36]]PYY3-36, or
N-alpha-Acetyl[N-epsilon10-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}][Lys10,N-methyl Arg35]]PYY(3-36).

In one embodiment the PYY derivative is not SEQ ID NO: 4. In one embodiment the PYY derivative is not N-epsilon32[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys32]hPYY(3-36). In one embodiment the PYY derivative is not SEQ ID NO: 5. In one embodiment the PYY derivative is not N-epsilon31[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl [Lys31]hPYY(3-36). In one embodiment the PYY derivative is not SEQ ID NO: 6. In one embodiment the PYY derivative is not N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(3-36). In one embodiment the PYY derivative is not SEQ ID NO: 8. In one embodiment the PYY derivative is not N-epsilon28[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys28]hPYY(3-36). In one embodiment the PYY derivative is not SEQ ID NO: 15. In one embodiment the PYY derivative is not N-epsilon21[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys21]hPYY(3-36). In one embodiment the PYY derivative is not SEQ ID NO: 16. In one embodiment the PYY derivative is not N-epsilon20[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys20]hPYY(3-36). In one embodiment the PYY derivative is not SEQ ID NO: 20. In one embodiment the PYY derivative is not N-epsilon16[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys16]hPYY(3-36). In one embodiment the PYY derivative is not SEQ ID NO: 21. In one embodiment the PYY derivative is not N-epsilon15[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys15]hPYY(3-36). In one embodiment the PYY derivative is not SEQ ID NO: 22. In one embodiment the PYY derivative is not N-epsilon14[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl][Lys14]hPYY(3-36). In one embodiment the PYY derivative is not SEQ ID NO: 23. In one embodiment the PYY derivative is not N-epsilon13[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys13]hPYY(3-36). In one embodiment the PYY derivative is not SEQ ID NO: 25. In one embodiment the PYY derivative is not N-epsilon11[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys11]hPYY(3-36). In one embodiment the PYY derivative is not SEQ ID NO: 32. In one embodiment the PYY derivative is not N-epsilon4[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Lys4]hPYY(3-36).

### Biological Activity

In one embodiment the PYY derivative has at least 25%, specifically 30%, 40%, 50%, 60%, 70%, 80%, 90%, 95%, 98% or 99% percent of the biological activity of hPYY(1-36). In one embodiment the term “biological activity” of PYY is intended to mean the ability to induce an effect in an in vivo model, such as the assay for acute food-intake, e.g. in mice, described herein as Assay (V). In one embodiment “biological activity” of PYY is intended to mean reduction of food intake, effect on body weight, gastric emptying, change in respiratory quotient and/or effect on intestinal electrolyte secretion. Methods for determination of said biological effects are described herein, e.g., in Assay (IV), Assay (V), Assay (VI), Assay (VII), Assay (VIII) and Assay (IX). In one embodiment the PYY derivative exhibits improved biological activity compared to hPYY(1-36) administered at the same dose and dosing frequency. In one embodiment the PYY derivative has at least 110%, 125%, 130%, 140%, 150%, 200% or more of the biological activity of hPYY(1-36) or hPYY(3-36) administered at the same dose and dosing frequency. In one embodiment the PYY derivative has an effect in at least one of the assays described herein, such as food intake, effect on body weight, gastric emptying, appetite, change in respiratory quotient, effect on intestinal electrolyte secretion, Assay (IV), Assay (V), Assay (VI), Assay (VII), Assay (VIII) and Assay (IX), which is equal to or greater than the potency of hPYY(1-36) or hPYY(3-36) in the same assay. In one embodiment the PYY derivative exhibits improved ease of manufacture, stability and/or ease of formulation compared to hPYY(1-36) or hPYY(3-36).

In one embodiment the PYY derivative has improved pharmacokinetic profile compared to hPYY(1-36) or hPYY(3-36). In one embodiment the PYY derivative comprising a serum albumin binding side chain according to the invention displays protracted properties that make them suitable for administration once daily or with lower frequency than once-daily, such as in a once-weekly, every other day, twice-monthly or once-monthly dosing regime. In one embodiment said pharmacokinetic profile or said protracted properties is determined by measuring the half-life of the PYY derivative.

In one embodiment the invention provides the PYY derivative with high affinity serum albumin binding effect. In one embodiment high affinity serum albumin binding effect is defined as at least 10 times, such as at least 20 times, at least 50 times or at least 100 times higher serum albumin binding of the PYY derivative according to the invention relative to hPYY(1-36) or hPYY(3-36).

In one embodiment the PYY derivative has substantially improved half-life relative to hPYY(1-36) or hPYY(3-36). In one embodiment the half-life of the PYY derivative in a rodent or in a non-rodent model is improved at least 3 fold, such as at least 6 fold, 10 fold or 50 fold, relative to hPYY(1-36) or hPYY(3-36). In one embodiment the PYY derivative shows an improvement of half-life compared to hPYY(3-36) in the range of 5-500, such as 10-500, 20-500, 50-500, 10-400, 20-400, 50-400, 100-500, 100-400 or 200-500 fold determined in vivo using a non-rodent model. In one embodiment the PYY derivative has a substantially improved half-life in a non-rodent model relative to hPYY(1-36) or hPYY(3-36).

In one embodiment the half-life of the PYY derivative is at least 5 h, such as at least 7 hours determined by Assay (IV) described herein. In one embodiment the half-life of the PYY derivative is at least 8 h, such as at least 15 hours or at least 30 hours, determined by Assay (IV) described herein. In one embodiment the half-life of the PYY derivative is at least 40 h, such as at least 50 hours or at least 60 hours, determined by Assay (IV) described herein. In one embodiment the PYY derivative has a half-life of at least 10 h, such as at least 20 h, at least 30 h, at least 40 h, at least 50 h, at least 100 h, at least 150 h, at least 200 h, at least 250 h, at least 300 h or at least 350 h determined by Assay (IV) described herein. In one embodiment the half-life of the PYY derivative is at least 80 h determined by Assay (IV) described herein.

In one embodiment the half-life of the PYY derivative is longer than the half-life of hPYY(3-36). In one embodiment the half-life of the PYY derivative is at least 10 times, such as at least 20, at least 40, at least 60, at least 75, at least 100, at least 150, at least 200, at least 250, at least 300, at least 350 or at least 400 times the half-life of hPYY(3-36). In one embodiment half-life is determined by Assay (IV) as described herein, PK i.v. minipigs.

In one embodiment the Y2 receptor potency of the PYY derivative is less than 2 times the Y2 receptor potency of hPYY(3-36). In one embodiment the Y2 receptor potency of the PYY derivative is less than 5 times, such as less than 10, 20, 50, 100 or 150 times the Y2 receptor potency of hPYY(3-36). In one embodiment the Y2 receptor potency of the PYY derivative is less than the Y2 receptor potency of hPYY(3-36). In one embodiment the Y2 receptor potency of the PYY derivative is reduced less than 2 times compared to the Y2 receptor potency of hPYY(3-36). In one embodiment the Y2 receptor potency of the PYY derivative is reduced less than 5 times, such as less than 10, 20, 50, 100 or 150 times compared to the Y2 receptor potency of hPYY(3-36). In one embodiment the Y2 receptor potency of the PYY derivative is less than the Y2 receptor potency of hPYY(3-36). In one embodiment the Y2 receptor potency of the PYY derivative is between 0.5 and 5 times the Y2 receptor potency of hPYY(3-36). In one embodiment the Y2 receptor potency of the PYY derivative is between 0.1 and 10 times the Y2 receptor potency of hPYY(3-36). In one embodiment the Y2 receptor potency is determined by Assay (I), Y2 receptor ACTOne assay. In one embodiment the Y2 receptor potency of the PYY derivative is up to 20 nM determined by Assay (I) described herein. In one embodiment the Y2 receptor potency of the PYY derivative is up to 10 nM determined by Assay (I) described herein.

In one embodiment the PYY derivative has a Y1 receptor potency which is lower (e.g. the EC50 value higher) than the Y1 receptor potency of hPYY(3-36) as determined by Assay (II).

In one embodiment the PYY derivative has a Y5 receptor potency which is lower (e.g. the EC50 value higher) than the Y5 receptor potency of hPYY(3-36) as determined by Assay (III).

In one embodiment the PYY derivative has a Y5/Y2 receptor potency ratio which is at least 5 as determined by Assay (III) and Assay (I), respectively. In one embodiment the PYY derivative has a Y5/Y2 receptor potency ratio which is at least equal to or higher than the Y5/Y2 receptor potency ratio of hPYY(3-36) as determined by Assay (III) and Assay (I), respectively. In one embodiment the PYY derivative has a Y5/Y2 receptor potency ratio which at least 15 or at least 20 as determined by Assay (III) and Assay (I), respectively.

In one embodiment the PYY derivative has a Y1/Y2 receptor potency ratio which is at least 2 as determined by Assay (II) and Assay (I), respectively. In one embodiment the PYY derivative has a Y1/Y2 receptor potency ratio which is higher than the Y1/Y2 receptor potency ratio of hPYY(3-36) as determined by Assay (II) and Assay (I), respectively. In one embodiment the PYY derivative has a Y1/Y2 receptor potency ratio which is at least 15 or at least 20 as determined by Assay (II) and Assay (I), respectively. In one embodiment the PYY derivative has a Y1/Y2 receptor potency ratio which is at least 30 or at least 50 as determined by Assay (II) and Assay (I), respectively.

In one embodiment the PYY derivative has a half-life of at least 8 hours as determined by Assay (IV) and a Y2 receptor potency of less than 10 nM as determined by Assay (I).

In one embodiment the PYY derivative has a Y2 receptor potency of less than 20 nM as determined by Assay (I), and (a) a Y1/Y2 receptor potency ratio which is higher than the Y1/Y2 receptor potency ratio of hPYY(3-36), wherein the Y1 receptor potency and the Y2 receptor potency is determined by Assay (II) and Assay (I), respectively; and/or (b) a Y5/Y2 receptor potency ratio which is higher than the Y5/Y2 receptor potency ratio of hPYY(3-36), and wherein the Y5 receptor potency and the Y2 receptor potency is determined by Assay (III) and Assay (I), respectively.

In one embodiment the PYY derivative has a Y1/Y2 receptor potency ratio which is higher than the Y1/Y2 receptor potency ratio of hPYY(3-36), wherein the Y1 receptor potency and the Y2 receptor potency is determined by Assay (II) and Assay (I), respectively.

In one embodiment the PYY derivative has a Y5/Y2 receptor potency ratio which is higher than the Y5/Y2 receptor potency ratio of hPYY(3-36), and wherein the Y5 receptor potency and the Y2 receptor potency is determined by Assay (III) and Assay (I), respectively.

In one embodiment the PYY derivative has a half-life of at least 8 hours as determined by Assay (IV), a Y2 receptor potency of less than 10 nM as determined by Assay (I), and a Y5/Y2 receptor potency ratio of at least 5 as determined by Assay (III) and Assay (I), respectively. In one embodiment the PYY derivative has a half-life of at least 8 hours as determined by Assay (IV), a Y2 receptor potency of less than 10 nM as determined by Assay (I), and a Y1/Y2 receptor potency ratio of at least 2 as determined by Assay (III) and Assay (I), respectively.

In one embodiment the PYY derivative has a half-life of at least 7 hours, such as at least 8 or at least 20 hours, as determined by Assay (IV), a Y2 receptor potency of less than 20 nM, such as less than 10 nM, as determined by Assay (I), a Y5/Y2 receptor potency ratio which is higher than the Y5/Y2 receptor potency ratio of hPYY(3-36) as determined by Assay (III) and Assay (I), respectively and/or a Y1/Y2 receptor potency ratio which is higher than the Y1/Y2 receptor potency ratio of hPYY(3-36) as determined by Assay (III) and Assay (I), respectively.

In one embodiment effects of the PYY derivative described herein is determined relative to hPYY(3-36) with a serum albumin binding side chain identical to that of said derivative.

Y2 receptor selectivity is intended to mean the ability to selectively activate the Y2 receptor relative to the Y1 and/or the Y5 receptor. The selectivity for the Y2 receptor relative to the Y1 or Y5 receptor is determined by the ratio of Y1/Y2 potency or Y5/Y2 potency ratio, respectively. The Y2, Y1, and Y5 receptor potency is determined by Assay (I), Assay (II), and Assay (III), respectively. If the ratio Y1/Y2 and/or Y5/Y2 is higher than observed for hPYY(3-36) the Y2 receptor selectivity is increased.

### Physical Stability

In one embodiment the PYY derivative has improved physical stability. In solution at neutral pH (i.e. pH 7-8) certain PYY compounds, such as SEQ ID NO: 6, have poor physical stability. Poor physical stability may lead to precipitation or amyloid fibril formation. In one embodiment physical stability includes long term storage under quiescent conditions. In one embodiment physical stability can be defined as the ability to withstand physical stress, such as increased temperature and/or shaking. In one embodiment physical stability may be determined using the method described in Example 49 herein. In one embodiment the PYY derivative has a physical stability of at least 90%, such as at least 95% peptide recovery as determined by the method described in Example 49 herein.

In one embodiment the PYY derivative has improved physical stability and comprises one or more of:
a) removing N-terminal positive charge, such as by N-terminal acetylation;
b) introducing a negatively charged or a non-charged amino acid in position 4; and
c) introducing one or more negatively charged amino acids at other positions than position 4.

In one embodiment the PYY derivative comprises an N-terminal acetylation.

In one embodiment the PYY derivative comprises a negatively charged amino acid, such as Glu, in position 4. In one embodiment the PYY derivative comprises a non-charged amino acid, such as Val or Ala, in position 4. In one embodiment the PYY derivative does not comprise Arg in position 4 as the sole substitution. In one embodiment the PYY derivative is not SEQ ID NO: 35.

In one embodiment the PYY derivative comprises a deletion of the amino acid in position 3 or position 3 and 4.

In one embodiment the PYY derivative comprises Glu in position 18 and/or position 22.

### Pharmaceutical Compositions

One object of the present invention is to provide a pharmaceutical formulation comprising the PYY derivative which is present in a concentration from 0.1 mg/ml to 25 mg/ml, and wherein said formulation has a pH in the range of 3.0 to 9.0. The formulation may further comprise at least one selected from the group consisting of a buffer system, preservative(s), tonicity agent(s), chelating agent(s), stabilizer(s) and surfactant(s). The term “pharmaceutical composition” as used herein means a product comprising an active analogue or derivative according to the invention together with pharmaceutical excipients selecting from the group consisting of a buffer, a preservative, and optionally a tonicity modifier and/or a stabilizer.

In one embodiment the invention relates to the use of the PYY derivative for the preparation of a medicament. In one embodiment the invention relates to the use of the PYY derivative in the manufacture of a medicament for therapeutic applications mentioned herein.

### Indications

In one embodiment the invention relates to the use of at least one PYY derivative for the preparation of a medicament. In one embodiment a method of treating a disease, condition or disorder modulated by a Y2 receptor agonist using the PYY derivative is provided. In one embodiment the PYY derivative is administered peripherally, such as i.v, s.c. or orally. In one embodiment the PYY derivative is administered by the buccal or sublingual route. In one embodiment the subject to be treated by a method of the invention is a mammal, such as a human, a cat or a dog. In one embodiment a therapeutically effective amount of the PYY derivative is used. The PYY derivative may be used alone or in combination with at least one additional pharmaceutical agent that is useful in the treatment of the disease, condition or disorder or a co-morbidity of the disease, condition or disorder. In one embodiment, diseases, conditions or disorders modulated by a Y2 receptor agonist in mammals comprise obesity and being overweight. Co-morbidities of such diseases, conditions or disorders would likely be incidentally improved by treatment of such diseases, conditions or disorders. In one embodiment a method of treating obesity using the PYY derivative is provided. In one embodiment a method of treating diabetes, e.g. type 2 diabetes, using the PYY derivative is provided.

As used herein, the term “therapeutically effective amount” of a compound refers to an amount sufficient to cure, alleviate or partially arrest the clinical manifestations of a given disease and/or its complications with respect to appropriate control values determined prior to treatment or in a vehicle-treated group. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. In one embodiment the terms are intended to comprise the full spectrum of treatments for a given condition from which the patient is suffering, such as administration of the PYY derivative in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition or disorder, and comprises the administration of the PYY derivative in question to prevent the onset of symptoms or complications. The terms “treating”, “treat” or “treatment” embrace both preventative, i.e., prophylactic, and palliative treatment. In one embodiment the invention relates to a method of reducing weight or promoting weight loss (including preventing or inhibiting weight gain) in a mammal which comprises peripherally administering to the mammal a weight-controlling or weight-reducing amount of the PYY derivative.

In one embodiment the invention relates to a method of reducing food intake by administration of the PYY derivative. In one embodiment the invention relates to a method of inducing satiety in a subject by administration of the PYY derivative. In one embodiment the invention relates to a method of reducing caloric intake in a subject by administration of the PYY derivative.

In one embodiment the invention relates to a method of reducing nutrient availability by administration of the PYY derivative. In one embodiment the invention relates to a method of inhibition of food intake, slowing of gastric emptying, inhibition of gastric acid secretion, and inhibition of pancreatic enzyme secretion by administration of the PYY derivative. In one embodiment the invention relates to a method of treating or preventing metabolic diseases, such as type 1 diabetes, type 2 diabetes, gestational diabetes mellitus, obesity and other manifestations of insulin-resistance syndrome (Syndrome X) by administration of the PYY derivative.

In one embodiment the invention relates to a method for altering energy metabolism in a subject by administration of the PYY derivative. In one embodiment the method for altering energy metabolism in a subject comprises administration of the PYY derivative. In one embodiment the invention relates a method of increasing energy expenditure and decreasing efficiency of calorie utilization in a subject by administration of the PYY derivative. In one embodiment the invention relates to a method of increasing energy expenditure by administration of the PYY derivative.

In one embodiment the invention relates to a method for treating and/or preventing obesity, wherein the method comprises administering a therapeutically or prophylactically effective amount of the PYY derivative to a subject in need thereof. In one embodiment the subject is an obese or overweight subject. In one embodiment, while “obesity” is generally defined as a body mass index over 30, for purposes of this disclosure, any subject, including those with a body mass index of less than 30, who needs or wishes to reduce body weight is comprised in the scope of “obese”. Subjects who are insulin resistant, glucose intolerant, or have any form of diabetes, such as type 1 diabetes, type 2 diabetes or gestational diabetes, can benefit from the methods disclosed herein. In one embodiment the invention relates to methods of reducing food intake, reducing nutrient availability, causing weight loss, affecting body composition, and altering body energy content or increasing energy expenditure, treating diabetes mellitus and/or improving lipid profile (including reducing LDL cholesterol and triglyceride levels and/or changing HDL cholesterol levels), wherein the method comprises administration of the PYY derivative. In one embodiment the methods of the invention are used to treat or prevent conditions or disorders which can be alleviated by reducing nutrient availability in a subject comprising administration to said subject of the PYY derivative, such conditions and disorders comprise, but are not limited to, hypertension, dyslipidemia, cardiovascular disease, eating disorders, insulin-resistance, obesity, and diabetes mellitus of any kind.

In one embodiment the invention relates to a method for treating and/or preventing obesity-related diseases, such as reduction of food intake, Syndrome X (metabolic syndrome), diabetes, such as type 1 diabetes or type 2 diabetes, or Non Insulin Dependent Diabetes Mellitus (NIDDM), hyperglycemia, insulin resistance, impaired glucose tolerance, cardiovascular disease, hypertension, atherosclerosis, coronary artery disease, myocardial infarction, peripheral vascular disease, stroke, thromboembolic diseases, hypercholesterolemia, hyperlipidemia, gallbladder disease, osteoarthritis, sleep apnea, reproductive disorders such as polycystic ovary syndrome (PCOS) or cancer, such as breast, prostate or colon cancer, by administration of the PYY derivative.

In one embodiment the PYY derivative provides a reduction of food intake of at least 5%, such as at least 10%, 15%, 20%, 25% or 30%, compared to vehicle. In one embodiment the PYY derivative provides a reduction of food intake in the range of 5-30%, such as at least 5-20%, 5-15% or 10-20%, compared to vehicle. In one embodiment the PYY derivative provides a reduction of body weight of at least 5%, such as at least 10%, 15%, 20%, 25% or 30%, compared to vehicle. In one embodiment the PYY derivative provides a reduction of body weight in the range of 5-30%, such as at least 5-20%, 5-15% or 10-20%, compared to vehicle.

### Gastro-Intestinal-Related Indications

In one embodiment the invention relates to a method for treating and/or preventing a disease associated with excess intestinal electrolyte and water secretion, decreased absorption or intestinal inflammatory condition, e.g., infectious diarrhoea, inflammatory diarrhoea, short bowel syndrome or the diarrhoea which typically occurs following surgical procedures, e.g., ileostomy. Examples of infectious diarrhoea comprise, without limitation, acute viral diarrhoea, acute bacterial diarrhoea (e.g., salmonella, Campylobacter, and Clostridium or due to protozoal infections) or traveller's diarrhoea (e.g., Norwalk virus or rotavirus). Examples of inflammatory diarrhoea comprise, without limitation, malabsorption syndrome, tropical sprue, chronic pancreatitis, ulcerative colitis, Crohn's disease, diarrhoea, and irritable bowel syndrome by administration of the PYY derivative.

The PYY derivative exhibits a broad range of biological activities, some related to their antisecretory and antimotility properties. The PYY derivative may suppress gastrointestinal secretions by direct interaction with epithelial cells or, optionally, by inhibiting secretion of hormones or neurotransmitters which stimulate intestinal secretion. Anti-secretory properties comprise inhibition of gastric and/or pancreatic secretions and can be useful in the treatment or prevention of diseases and disorders including gastritis, acute pancreatitis, Barrett's esophagus, and Gastroesophageal Reflux Disease.

The PYY derivative may be useful in the treatment of any number of gastrointestinal disorders (see e.g., Harrison's Principles of Internal Medicine, McGraw-Hill Inco, New York, 12th Ed.) that are associated with excess intestinal electrolyte and water secretion as well as decreased absorption. A method of measuring intestinal electrolyte secretion is described on page 1250 of (Eto B et al., Comparison of the antisecretory effect of endogenous forms of peptide YY on fed and fasted rat jejunum, Peptides, 1997; 18(8): 1249-55).

In one embodiment the invention relates to a method for treating and/or preventing a condition characterized by damage to the intestine (see WO 03/105763, incorporated herein by reference in its entirety) such as chemotherapy-induced diarrhoea, ulcerative colitis, inflammatory bowel disease, bowel atrophy, loss bowel mucosa and/or loss of bowel mucosal function by administration of the PYY derivative. In one embodiment assays for said damage to the intestine described in WO 03/105763 may be used, said assays comprise 11 week old male HSD rats, ranging 250-300 grams housed in a 12:12 light-dark cycle, and allowed ad libitum access to a standard rodent diet (Teklad LM 485, Madison, Wis.) and water, wherein the animals were fasted for 24 hours before the experiment. In one embodiment the simple and reproducible rat model of chronic colonic inflammation described by Morris G P, et al., “Hapten- induced model of chronic inflammation and ulceration in the rat colon”, Gastroenterology, 1989; 96:795-803, may be used which exhibits a relatively long duration of inflammation and ulceration, affording an opportunity to study the pathophysiology of colonic inflammatory disease in a specifically controlled fashion, and to evaluate new treatments potentially applicable to inflammatory bowel disease in humans. In said model of chronic colonic inflammation rats are anesthetized with 3% isofluorane and placed on a regulated heating pad set at 37° C. A gavage needle is inserted rectally into the colon 7 cm. The hapten trinitrobenzenesulfonic acid (TNBS) dissolved in 50% ethanol (v/v) is delivered into the lumen of the colon through the gavage needle at a dose of 30 mg/kg, in a total volume of 0.4-0.6 mL, as described in Mazelin, et al., “Protective role of vagal afferents in experimentally-induced colitis in rats”, Juton Nery Syst, 1998; 73:38 45. Control groups receive saline solution (NaCl 0.9%) intracolonically. Four days after induction of colitis, the colon is resected from anesthetized rats, which is then euthanized by decapitation. Weights of excised colon and spleen are measured, and the colons photographed for scoring of gross morphologic damage. Inflammation is defined as regions of hyperemia and bowel wall thickening.

Further indications in which the PYY derivative may be used as well as methods of determination of effects of said derivative in relation to said indication are described in the international application no. PCT/EP2009/055989. Specifically, the PYY derivative may be useful for treatment and/or prevention of indications selected from the group consisting of potentiating, inducing, enhancing or restoring glucose responsiveness in pancreatic islets or cells, treating or preventing conditions associated with metabolic disorders, anxiety, hypotension, rhinitis, promoting wound healing, decreasing time of recreation after surgery, promoting arteriogenesis as described in the international application no. PCT/EP2009/055989, wherein methods of determining the effect of said derivative in said indications are also described. In one embodiment the invention relates to a method for treating and/or preventing osteoporosis.

In one embodiment an acute test may be performed where the PYY derivative is administered to ensure that said derivative have the intended effect in the subject to be treated before a chronic treatment is started, whereby it is ensured that only subjects who are susceptible to treatment with the PYY derivative are treated with said derivative.

### EMBODIMENTS OF THE INVENTION

1. A PYY derivative comprising a serum albumin binding side chain, wherein said derivative has a half-life of at least 7 hours as determined by Assay (IV), provided that the PYY derivative is not
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY3-36,
N-epsilon13-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys13]hPYY3-36,
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY3-36,
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys11]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY2-36,
N-epsilon4-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY3-36,
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Arg26]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Leu3,Glu4,Val6,Tyr7, Lys13,Arg26]hPYY1-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4, Lys13,Arg26]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4,Tyr7,Lys13,Arg26]hPYY1-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY3-36,
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys4]hPYY3-36,
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys11]hPYY3-36,
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY3-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY18-36
N-epsilon25-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys25]hPYY3-36,
N-epsilon24-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys24]hPYY3-36,
N-epsilon19-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys19]hPYY3-36,
N-epsilon13-[4-(16-(1H-Tetrazol-5-yl)hexadecanoylsulfamoyl)butyryl]ethoxy}ethoxy)acetylamino]ethoxy}ethoxy)acetyl][Lys13]PYY3-36,
N-epsilon25-[4-(16-(1H-Tetrazol-5-yl)hexadecanoylsulfamoyl)butyryl]ethoxy}ethoxy)acetylamino]ethoxy}ethoxy)acetyl][Lys25]PYY3-36,
N-alpha-[4-(16-(1H-Tetrazol-5-yl)hexadecanoylsulfamoyl)butyryl]ethoxy}ethoxy)acetylamino]ethoxy}ethoxy)acetyl]PYY3-36,
N-alpha-[4-(16-(1H-Tetrazol-5-yl)hexadecanoylsulfamoyl)butyryl]ethoxy}ethoxy)acetylamino]ethoxy}ethoxy)acetyl]PYY3-36,
N-epsilon18-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys18]PYY3-36,
N-epsilon22-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys22]PYY3-36,
N-epsilon26-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys26]PYY3-36,
N-epsilon29-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys29]PYY3-36,
N-epsilon36-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys36]PYY3-36,
N-epsilon21-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys21]PYY3-36,
N-epsilon30-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys30]PYY3-36,
N-epsilon31-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys31]PYY3-36,
N-epsilon14-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys14]PYY3-36,
N-epsilon15-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys15]PYY3-36,
N-epsilon16-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys16]PYY3-36,
N-epsilon20-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys20]PYY3-36,
N-epsilon28-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys28]PYY3-36,
N-epsilon32-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys32]PYY3-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}-ethoxy)acetylamino]ethoxy}ethoxy)acetyl] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][N-Methyl Arg35] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][His25,N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Aib25,N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Tyr25,N-Methyl Gln34] PYY(3-36),
N-alpha-{2-(2-{2-[2-(2-{2-[4-Carboxy-4-(17-carboxyheptadecanoylamino)butyrylamino]ethoxy}-ethoxy)acetylamino]-ethoxy}ethoxy)acetyl}[Ala20] PYY(3-36),
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[N-methyl Arg35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Ala27] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Agp35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Agb35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[HomoArg35] PYY(3-36),
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[NArg35] PYY(3-36) or
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[NGln34] PYY(3-36).
   2. The PYY derivative according to any one of the preceding embodiments, wherein said derivative has a half-life of at least 8 hours, such as at least 15 hours or at least 30 hours, as determined by Assay (IV).
   3. The PYY derivative according to any one of the preceding embodiments, wherein said half-life is at least 40 h, such as at least 50 h or at least 60 h, as determined by Assay (IV).
   4. The PYY derivative according to any one of the preceding embodiments, wherein said serum albumin binding side chain is attached to the side chain of an amino acid in a position selected from the group consisting of position 12 and 16-31.
   5. The PYY derivative according to any one of the preceding embodiments, wherein said serum albumin binding side chain is attached to the side chain of an amino acid in a position selected from the group consisting of position 17, 20-22 and 24-31.
   6. The PYY derivative according to any one of the preceding embodiments, wherein said serum albumin binding side chain is attached to the side chain of an amino acid in a position selected from the group consisting of position 24, 25, 27, 28, 30 and 31.
   7. The PYY derivative according to any one of the preceding embodiments, wherein said serum albumin binding side chain is attached the side chain of the amino acid in position 17.
   8. The PYY derivative according to any one of the preceding embodiments, wherein said serum albumin binding side chain is attached the side chain of the amino acid in position 21.
   9. The PYY derivative according to any one of the preceding embodiments, wherein said serum albumin binding side chain is attached the side chain of the amino acid in position 30.
   10. The PYY derivative according to any one of the preceding embodiments, wherein said serum albumin binding side chain is attached the side chain of the amino acid in position 31.
   11. The PYY derivative according to any one of the preceding embodiments, wherein said derivative has a Y2 receptor potency of up to 20 nM as determined by Assay (I) as described herein.
   12. The PYY derivative according to any one of the preceding embodiments, wherein said derivative has a Y2 receptor potency of up to 10 nM as determined by Assay (I).
   13. The PYY derivative according to any one of the preceding embodiments, wherein said derivative has a Y5/Y2 receptor potency ratio which is at least 5 as determined by Assay (III) and Assay (I), respectively.
   14. The PYY derivative according to any one of the preceding embodiments, wherein said derivative has a Y5/Y2 receptor potency ratio which is at least equal to or higher than the Y5/Y2 receptor potency ratio of hPYY(3-36) as determined by Assay (III) and Assay (I), respectively.
   15. The PYY derivative according to any one of the preceding embodiments, wherein said derivative has a Y5/Y2 receptor potency ratio which at least 15 or at least 20 as determined by Assay (III) and Assay (I), respectively.
   16. The PYY derivative according to any one of the preceding embodiments, wherein said derivative has a Y1/Y2 receptor potency ratio which is at least 2 as determined by Assay (II) and Assay (I), respectively.
   17. The PYY derivative according to any one of the preceding embodiments, wherein said derivative has a Y1/Y2 receptor potency ratio which is higher than the Y1/Y2 receptor potency ratio of hPYY(3-36) as determined by Assay (II) and Assay (I), respectively.
   18. The PYY derivative according to any one of the preceding embodiments, wherein said derivative has a Y1/Y2 receptor potency ratio which is at least 15 or at least 20, such as at least 30 or at least 50, as determined by Assay (II) and Assay (I), respectively.
   19. The PYY derivative according to any one of the preceding embodiments, wherein said derivative has a half-life of at least 8 hours as determined by Assay (IV) and a Y2 receptor potency of less than 10 nM as determined by Assay (I).
   20. The PYY derivative according to any one of the preceding embodiments, wherein said derivative has a half-life of at least 8 hours as determined by Assay (IV), a Y2 receptor potency of less than 10 nM as determined by Assay (I), and a Y5/Y2 receptor potency ratio of at least 5 as determined by Assay (III) and Assay (I), respectively.
   21. The PYY derivative according to any one of the preceding embodiments, wherein said serum albumin binding side chain comprises a distal carboxylic acid or a distal tetrazole group.
   22. The PYY derivative according to any one of the preceding embodiments, wherein said serum albumin binding side chain comprises an alkyl chain with at least 14 carbon atoms.
   23. The PYY derivative according to any one of the preceding embodiments, wherein said serum albumin binding side chain comprises an alkyl chain with at least 14 carbon atoms comprising a distal carboxylic acid or a distal tetrazole group.
   24. The PYY derivative according to any one of the preceding embodiments, wherein said serum albumin binding side chain comprises a C18 dicarboxylic acid or a C16 dicarboxylic acid.
   25. The PYY derivative according to any one of the preceding embodiments, wherein said serum albumin binding side chain comprises a C16 carboxylic acid.
   26. The PYY derivative according to any one of the preceding embodiments, wherein said serum albumin binding side chain is selected from the group consisting of
2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl,
2-(2-{2-[2-(2-{2-[(S)-4-Carboxy-4-(19-carboxynonadecanoylamino)butyrylamino]ethoxy}ethoxy)acetyl-amino]ethoxy}ethoxy)acetyl,
2-(2-{2-[2-(2-{2-[(S)-4-Carboxy-4-(17-carboxyheptadecanoylamino)butyrylamino]ethoxy}ethoxy)acetyl-amino]ethoxy}ethoxy)acetyl,
2-(2-{2-[2-(2-{2-[(S)-4-Carboxy-4-(15-carboxypentadecanoylamino)butyrylamino]ethoxy}ethoxy)acetyl-amino]ethoxy}ethoxy)acetyl and
2-(2-{2-[2-(2-{2-[(S)-4-Carboxy-4-(hexadecanoylamino)butyrylamino]ethoxy}ethoxy)acetyl-amino]ethoxy}ethoxy)acetyl.
   27. The PYY derivative according to any one of the preceding embodiments, wherein said serum albumin binding side chain is [4-(16-(1H-Tetrazol-5-yl)hexadecanoylsulfamoyl)butyryl]ethoxy}ethoxy)acetylamino]ethoxy}ethoxy)acetyl].
   28. The PYY derivative according to any one of the preceding embodiments, wherein said serum albumin binding side chain is attached to the side chain of either 2,3-diaminopropionic acid, 2,4-diaminobutyric acid, ornitine or Lys.
   29. The PYY derivative according to any one of the preceding embodiments, wherein in said derivative two amino acid residues are cross-linked using e.g. a disulfide, lactame or tetrazole linkage.
   30. The PYY derivative according to any of the preceding embodiments, wherein said derivative comprises formula (I):

Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅-Xaa₂₆-Xaa₂₇-Xaa₂₈-Xaa₂₉-Xaa₃₀-Xaa₃₁-Xaa₃₂-Xaa₃₃-Xaa₃₄-Xaa₃₅-Xaa₃₆ Formula (I)

wherein
Xaa₁ is Tyr, Phe, Ala, 2,3-diaminopropionic acid, 2,4-diaminobutyric acid, ornitine, Lys or absent;
Xaa₂ is Pro, Ala, Leu, Phe, hydroxyproline, 2,3-diaminopropionic acid, 2,4-diaminobutyric acid, ornitine, Lys or absent;
Xaa₃ is Ile, Val, Leu (1-aminocyclopentyl)carboxylic acid, (1-aminocyclohexyl)carboxylic acid, 1-aminobutyric acid, 2,3-diaminopropionic acid, 2,4-diaminobutyric acid, ornitine, Lys or absent;
Xaa₄ is Lys, Arg, Glu, 2,3-diaminopropionic acid, 2,4-diaminobutyric acid, ornitine, Lys or absent;
Xaa₅ is Pro, 2,3-diaminopropionic acid, 2,4-diaminobutyric acid, ornitine or Lys;
Xaa₆ is Glu, 2,3-diaminopropionic acid, 2,4-diaminobutyric acid, ornithine or Lys;
Xaa₇ is Ala, 2,3-diaminopropionic acid, 2,4-diaminobutyric acid, ornitine or Lys;
Xaa₈ is Pro, Ala, 2,3-diaminopropionic acid, 2,4-diaminobutyric acid, ornitine or Lys;
Xaa₉ is Gly, 2,3-diaminopropionic acid, 2,4-diaminobutyric acid, ornitine or Lys;
Xaa₁₀ is Glu, 2,3-diaminopropionic acid, 2,4-diaminobutyric acid, ornitine or Lys;
Xaa₁₁ is Asp, Ala, 2,3-diaminopropionic acid, 2,4-diaminobutyric acid, ornitine or Lys;
Xaa₁₂ is Ala, 2,3-diaminopropionic acid, 2,4-diaminobutyric acid, ornitine or Lys;
Xaa₁₃ is Ser, 2,3-diaminopropionic acid, 2,4-diaminobutyric acid, ornithine, Lys;
Xaa₁₄ is Pro or hydroxyproline;
Xaa₁₅ is Glu, 2,3-diaminopropionic acid, 2,4-diaminobutyric acid, ornitine or Lys;
Xaa₁₆ is Glu, 2,3-diaminopropionic acid, 2,4-diaminobutyric acid, ornitine or Lys;
Xaa₁₇ is Leu, Val, Ile, homoleucine, norleucine, (1-aminocyclopentyl)carboxylic acid, (1-aminocyclohexyl)carboxylic acid or 1-aminobutyric acid;
Xaa₁₈ is Asn, Ala, Glu, 2,3-diaminopropionic acid, 2,4-diaminobutyric acid, ornitine or Lys;
Xaa₁₉ is Arg, Ala, 2,3-diaminopropionic acid, 2,4-diaminobutyric acid, ornitine or Lys;
Xaa₂₀ is Tyr, Ala, Phe, 3-pyridylalaine, 2,3-diaminopropionic acid, 2,4-diaminobutyric acid, ornitine or Lys;
Xaa₂₁ is Tyr, Ala, Phe, 3-pyridylalaine, 2,3-diaminopropionic acid, 2,4-diaminobutyric acid, ornitine or Lys;
Xaa₂₂ is Ala, 2,3-diaminopropionic acid, 2,4-diaminobutyric acid, ornitine or Lys;
Xaa₂₃ is Ser, 2,3-diaminopropionic acid, 2,4-diaminobutyric acid, ornitine or Lys;
Xaa₂₄ is Leu, Ile, Val, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₂₅ is Arg, Ala, His, aminoisobutyric acid, 2,3-diaminopropionic acid, 2,4-diaminobutyric acid, ornitine or Lys;
Xaa₂₆ is His, Arg, 2,3-diaminopropionic acid, 2,4-diaminobutyric acid, ornitine or Lys;
Xaa₂₇ is Tyr, Ala, Phe, homoPhe or 3-pyridylalanine, 2,3-diaminopropionic acid, 2,4-diaminobutyric acid, ornitine or Lys;
Xaa₂₈ is Leu, Ile, Val, homoleucine, norleucine, (1-aminocyclopentyl)carboxylic acid, (1-aminocyclohexyl)carboxylic acid, aminoisobutyric acid, 1-aminobutyric acid, 2,3-diaminopropionic acid, 2,4-diaminobutyric acid, ornitine or Lys;
Xaa₂₉ is Asn, Gln, 2,3-diaminopropionic acid, 2,4-diaminobutyric acid, ornitine or Lys;
Xaa₃₀ is Leu, Met, Val, Ile, homoleucine, aminoisobutyric acid, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₁ is Val, Leu, Ile, aminoisobutyric acid, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₂ is Thr, Ser, 2,3-diaminopropionic acid, 2,4-diaminobutyric acid, ornitine or Lys;
Xaa₃₃ is Arg, N-methyl Arg, methyllysine, dimethyllysine, trimethyllysine, 2-amino-3-guanidino-propionic acid, 2-amino-4-guanidino-butyric acid or monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid;
Xaa₃₄ is Gln, Asn, His, Pro, N-methyl Gln, β-homo Gln, (2-Carbamoyl-ethylamino)-acetic acid, N-methyl Asn or N-methyl His;
Xaa₃₅ is Arg, N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid; and
Xaa₃₆ is Tyr, Phe, N-methyl Tyr, C-methyl Phe, 3-pyridylalanine or (4-Hydroxybenzylamino)-acetic acid.
31. The PYY derivative according to any of the preceding embodiments, wherein Xaa₁ and Xaa₂ or Xaa₁, Xaa₂, Xaa₃ and Xaa₄ are absent.
32. The PYY derivative according to any of the preceding embodiments, wherein Xaa₄ is Arg.
33. The PYY derivative according to any of the preceding embodiments, wherein Xaa₄ is Asp or Glu.
34. The PYY derivative according to any of the preceding embodiments, wherein Xaa₁₈ is Ala, Glu or Gln.
35. The PYY derivative according to any of the preceding embodiments, wherein Xaa₂₂ is Asp or Glu.
36. The PYY derivative according to any of the preceding embodiments, wherein Xaa₁, Xaa₂, Xaa₃ and Xaa₄ are absent.
37. The PYY derivative according to any of the preceding embodiments, wherein said serum albumin binding side chain is selected from the group consisting of A-B-C-D-, A-C-D-, A-B-C- and A-C-,
wherein A- is

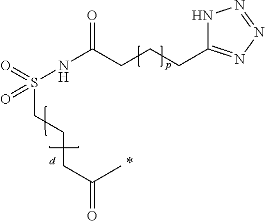

wherein p is selected from the group consisting of 10, 11, 12, 13 and 14, and d is selected from the group consisting of 0, 1, 2, 3, 4 and 5, and
-B- is selected from the group consisting of

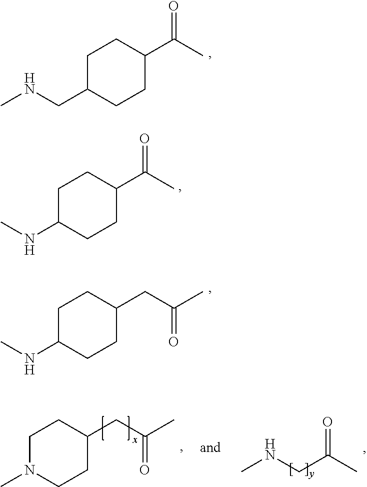

wherein x is selected from the group consisting of 0, 1, 2, 3 and 4, and y is selected from the group consisting of 1, 2, 3, 4, 5, 6, 7, 8, 9, 10, 11 and 12,

### or A- is

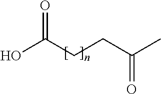

wherein n is selected from the group consisting of 12, 13, 14, 15, 16 17, 18 and 19, and B is selected from the group consisting of

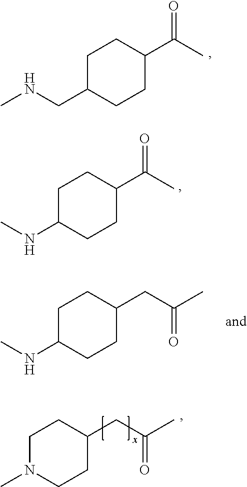

wherein x is selected from the group consisting of 0, 1, 2, 3 and 4, and
-C- is selected from the group consisting of

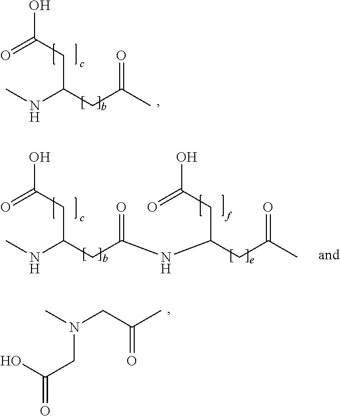

wherein b and e are each independently selected from the group consisting of 0, 1 and 2, and c and f are each independently selected from the group consisting of 0, 1 and 2 with the proviso that b is 1 or 2 when c is 0, or b is 0 when c is 1 or 2, and e is 1 or 2 when f is 0, or e is 0 when f is 1 or 2, and
-D- is attached to said amino acid residue and is a spacer, such as at least one 8-amino-3,6-dioxaoctanoic acid (Oeg) molecule.
38. The PYY derivative according to any one of the preceding embodiments, wherein said spacer comprises two Oeg molecules.
39. The PYY derivative according to any one of the preceding embodiments, wherein said derivative is selected from the group consisting of
N-epsilon35[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys35]hPYY(3-36) (SEQ ID NO: 1);
N-epsilon34[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys34]hPYY(3-36) (SEQ ID NO: 2);
N-epsilon33[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys33]hPYY(3-36) (SEQ ID NO: 3);
N-epsilon32[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys32]hPYY(3-36) (SEQ ID NO: 4);
N-epsilon31[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys31]hPYY(3-36) (SEQ ID NO: 5);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(3-36) (SEQ ID NO: 6);
N-epsilon29[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys29]hPYY(3-36) (SEQ ID NO: 7);
N-epsilon28[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys28]hPYY(3-36) (SEQ ID NO: 8);
N-epsilon27[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys27]hPYY(3-36) (SEQ ID NO: 9);
N-epsilon26[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys26]hPYY(3-36) (SEQ ID NO: 10);
N-epsilon25[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys25]hPYY(3-36) (SEQ ID NO: 11);
N-epsilon24[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys24]hPYY(3-36) (SEQ ID NO: 12);
N-epsilon23[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys23]hPYY(3-36) (SEQ ID NO: 13);
N-epsilon22[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys22]hPYY(3-36) (SEQ ID NO: 14);
N-epsilon21[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys21]hPYY(3-36) (SEQ ID NO: 15);
N-epsilon20[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys20]hPYY(3-36) (SEQ ID NO: 16);
N-epsilon19[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys19]hPYY(3-36) (SEQ ID NO: 17);
N-epsilon18[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys18]hPYY(3-36) (SEQ ID NO: 18);
N-epsilon17[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys17]hPYY(3-36) (SEQ ID NO: 19);
N-epsilon16[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys16]hPYY(3-36) (SEQ ID NO: 20);
N-epsilon15[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys15]hPYY(3-36) (SEQ ID NO: 21);
N-epsilon14[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys14]hPYY(3-36) (SEQ ID NO: 22);
N-epsilon13[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys13]hPYY(3-36) (SEQ ID NO: 23);
N-epsilon12[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys12]hPYY(3-36) (SEQ ID NO: 24);
N-epsilon11[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys11]hPYY(3-36) (SEQ ID NO: 25);
N-epsilon10[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys10]hPYY(3-36) (SEQ ID NO: 26);
N-epsilon9[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys9]hPYY(3-36) (SEQ ID NO: 27);
N-epsilon8[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys8]hPYY(3-36) (SEQ ID NO: 28);
N-epsilon7[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys7]hPYY(3-36) (SEQ ID NO: 29);
N-epsilon6[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys6]hPYY(3-36) (SEQ ID NO: 30);
N-epsilon5[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys5]hPYY(3-36) (SEQ ID NO: 31);
N-epsilon4[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys4]hPYY(3-36) (SEQ ID NO: 32);
N-epsilon3[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys3]hPYY(3-36) (SEQ ID NO: 33);
N-alpha-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl]hPYY(3-36) (SEQ ID NO: 34);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Lys30]hPYY(3-36) (SEQ ID NO: 35);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(5-36) (SEQ ID NO: 36);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4, Glu18,Lys30]hPYY(3-36) (SEQ ID NO: 37);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu18,Lys30]hPYY(5-36) (SEQ ID NO: 38);
N-epsilon30[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl][Arg4,Glu18,Gln29,Lys30]hPYY(3-36) (SEQ ID NO: 39);
N-epsilon30[2-(2-{2-[2-(2-{2-[(S)-4-Carboxy-4-(hexadecanoylamino)butyrylamino]ethoxy}ethoxy)-acetylamino]ethoxy}ethoxy)acetyl][Lys30]hPYY(3-36) (SEQ ID NO: 40);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Gln18,Lys30]hPYY(3-36) (SEQ ID NO: 41); and
N-epsilon1[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys1]hPYY(1-36) (SEQ ID NO: 42).
   40. The PYY derivative according to any of the preceding embodiments, wherein said derivative is derived from a vertebrate such as a mammal, e.g., a human.
   41. The PYY derivative according to any one of the preceding embodiments, wherein said derivative comprises at least one amino acid residue substituted into a proteinogenic or non-proteinogenic amino acid residue selected from the group consisting of

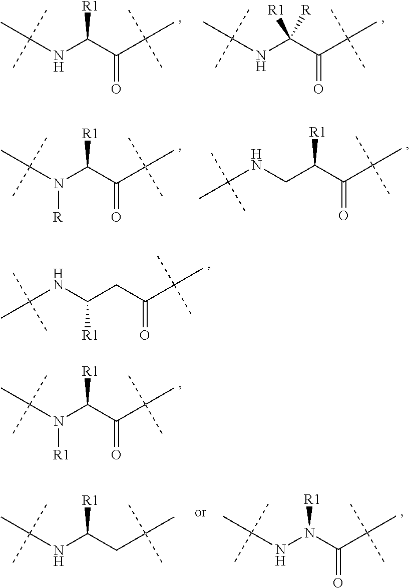

wherein R1 is side a chain of an amino acid; and R is H or C1-C12 alkyl.
42. The PYY derivative according to any one of the preceding embodiments, wherein said derivative comprises the amino acid residue represented by formula (A)

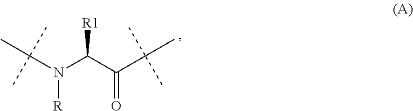

wherein R1 is a side chain of an amino acid and R is selected from the group consisting of alkyl, benzyl or phenyl.
43. The PYY derivative according to any of the preceding embodiments, wherein said derivative is suitable for administration in a dosing regime selected from group consisting of a once-daily, an every other day, a once-weekly, a twice-monthly and a once-monthly dosing regime.
44. A composition comprising the PYY derivative as defined in any of the preceding embodiments and at least one pharmaceutical excipient.
45. The PYY derivative according to any of the preceding embodiments for use in the treatment of a condition responsive to Y receptor modulation.
46. The PYY derivative according to embodiment 45, wherein the condition responsive to Y receptor modulation is obesity.
47. The PYY derivative according to embodiment 45 or 46, wherein said derivative is administered once-daily, every other day, twice-weekly or once-weekly.
48. Use of the PYY derivative as defined in any of embodiments 1-43 for the preparation of a medicament for the treatment of a condition responsive to Y receptor modulation, such as obesity or obesity-related diseases.
49. Use of the PYY derivative as defined in any of embodiments 1-43 for administration in a mammal, wherein said derivative shows protracted properties compared to human PYY(3-36).
50. A method of treatment of a condition responsive to Y receptor modulation by administration of the PYY derivative as defined in any of embodiments 1-43.
51. A method according to embodiment 50, wherein said condition responsive to Y receptor modulation is obesity.
52. A method according to embodiment 50 or 51, wherein said derivative is administered once-daily, every other day, twice-weekly or once-weekly.
53. A method of treatment according to any one of embodiments 50-52, wherein the condition responsive to Y receptor modulation is obesity-related diseases, such as reduction of food intake, Syndrome X (metabolic syndrome), diabetes, type 2 diabetes mellitus or Non Insulin Dependent Diabetes Mellitus (NIDDM), hyperglycemia, insulin resistance, polycystic ovary syndrome (PCOS) or impaired glucose tolerance.
54. A method of treatment according to any one of embodiments 50-53, wherein the condition responsive to Y receptor modulation is an obesity-related cardiovascular disease such as hypertension, atherosclerosis, coronary artery disease, myocardial infarction, peripheral vascular disease, stroke, thromboembolic diseases, hypercholesterolemia or hyperlipidemia.
55. A method of treatment according to any one of embodiments 50-54, wherein the condition responsive to Y receptor modulation is diarrhoea such as infectious diarrhoea, inflammatory diarrhoea, chemotherapy-induced diarrhoea, short bowel syndrome or the diarrhoea which typically occurs following surgical procedures, e.g., ileostomy.
56. A method of treatment according to any one of embodiments 50-55, wherein the condition responsive to Y receptor modulation is a condition characterized by damage to the intestine such as chemotherapy-induced diarrhoea, ulcerative colitis, Crohns disease, bowel atrophy, loss of bowel mucosa, and/or loss of bowel mucosal function.
57. A method of treatment according to any one of embodiments 50-56, wherein the condition responsive to Y receptor modulation is an intestinal inflammatory condition such as ulcerative colitis or Crohn's disease.
58. A method of treatment according to any one of embodiments 50-57, wherein the condition responsive to Y receptor modulation is allergic or non-allergic rhinitis.
59. A method of treatment according to any one of embodiments 50-58, wherein the condition responsive to Y receptor modulation is anxiety.

Further embodiments of the invention are:

1. A PYY derivative comprising a serum albumin binding side chain, wherein said derivative said derivative has a half-life of at least 7 hours as determined by Assay (IV).
2. The PYY derivative according to any one of the preceding embodiments, wherein said serum albumin binding side chain comprises an alkyl chain of at least 14 carbon atoms.
3. The PYY derivative according to any one of the preceding embodiments, wherein said serum albumin binding side chain or said alkyl chain comprises a distal carboxylic acid group or a distal tetrazole group.
4. The PYY derivative according to any one of the preceding embodiments, wherein said derivative comprises human PYY (hPYY), such as hPYY(3-36), or an analogue thereof.
5. The PYY derivative comprising a serum albumin binding side chain, wherein said serum albumin binding side chain is attached to the N-terminal amino group or an amino acid in a position selected from the group consisting of position 1, 3, 6, 7, 9, 10, 11, 12, 14, 15, 17, 18, 19, 21, 22, 23 and 30.
6. The PYY derivative according to any one of the preceding embodiments, wherein said distal carboxylic acid group has the formula (X)

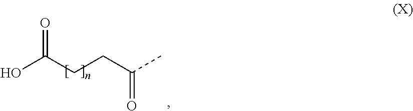

wherein n is at least 13.
7. The PYY derivative according to any one of the preceding embodiments, wherein said derivative has a Y2 receptor potency of less than 20 nM as determined by Assay (I), and
a Y1/Y2 receptor potency ratio which is higher than the Y1/Y2 receptor potency ratio of hPYY(3-36), wherein the Y1 receptor potency and the Y2 receptor potency is determined by Assay (II) and Assay (I), respectively; and/or
a Y5/Y2 receptor potency ratio which is higher than the Y5/Y2 receptor potency ratio of hPYY(3-36), and wherein the Y5 receptor potency and the Y2 receptor potency is determined by Assay (III) and Assay (I), respectively.
   8. The PYY derivative according to any one of the preceding embodiments, wherein said serum albumin binding side chain is attached to the N-terminal amino group or an amino acid in a position selected from the group consisting of position 3, 6, 7, 10, 11, 14, 17, 18, 19, 21, 22 and 30.
   9. The PYY derivative according to any one of the preceding embodiments, wherein said serum albumin binding side chain is attached to an amino acid in a position selected from the group consisting of position 7, 10, 21, 22 and 30.
   10. The PYY derivative according to any one of the preceding embodiments, wherein said serum albumin binding side chain is attached to an amino acid in a position selected from the group consisting of position 10, 11, 14, 17, 19, 21 and 30.
   11. The PYY derivative according to any one of the preceding embodiments, wherein said serum albumin binding side chain is attached to an amino acid in a position selected from the group consisting of position 10, 21 and 30, such as in position 30.
   12. The PYY derivative according to any one of the preceding embodiments, wherein said serum albumin binding side chain is attached to the N-terminal amino group or an amino acid in a position selected from the group consisting of position 3, 6, 7, 10, 11, 14, 17, 18, 19, 21, 22 and 30.
   13. The PYY derivative according to any one of the preceding embodiments, wherein said serum albumin binding side chain is attached to the amino acid in position 30.
   14. The PYY derivative according to any one of the preceding embodiments, wherein said serum albumin binding side chain is not attached to the amino acid in positions 18, 19, 22 or 23 and/or not attached to N-terminal or C-terminal amino group.
   15. The PYY derivative according to any one of the preceding embodiments, wherein the amino acid in position 29 is not Gln or not D-IsoAsp.
   16. The PYY derivative according to any one of the preceding embodiments, wherein the amino acid in position 29 is Asn.
   17. The PYY derivative according to any one of the preceding embodiments, wherein the amino acid in position 4 is not Glu or not Lys.
   18. The PYY derivative according to any one of the preceding embodiments, wherein the amino acid in position 4 is Arg.
   19. The PYY derivative according to any one of the preceding embodiments, wherein the amino acid in position 18 is not D-IsoAsp.
   20. The PYY derivative according to any one of the preceding embodiments, wherein the amino acid in position 18 is Asp.
   21. The PYY derivative according to any one of the preceding embodiments, wherein the amino acid in position 22 is not Glu or not D-Arg.
   22. The PYY derivative according to any one of the preceding embodiments, wherein the amino acid in position 36 is not 4-pyridylalanine.
   23. The PYY derivative according to any one of the preceding embodiments, wherein said position is relative to hPYY(1-36).
   24. The PYY derivative according to any one of the preceding embodiments, wherein said deriveative has a Y1/Y2 receptor potency ratio which is higher than the Y1/Y2 receptor potency ratio of hPYY(3-36), wherein the Y1 receptor potency and the Y2 receptor potency is determined by Assay (II) and Assay (I), respectively.
   25. The PYY derivative according to any one of the preceding embodiments, wherein said deriveative has a Y5/Y2 receptor potency ratio which is higher than the Y5/Y2 receptor potency ratio of hPYY(3-36), and wherein the Y5 receptor potency and the Y2 receptor potency is determined by Assay (III) and Assay (I), respectively.
   26. The PYY derivative according to any one of the preceding embodiments, wherein said derivative is not
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY3-36,
N-epsilon13-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys13]hPYY3-36,
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY3-36,
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys11]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY2-36,
N-epsilon4-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY3-36,
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Arg26]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Leu3,Glu4,Val6,Tyr7, Lys13,Arg26]hPYY1-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4, Lys13,Arg26]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4,Tyr7,Lys13,Arg26]hPYY1-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY3-36,
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys4]hPYY3-36,
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys11]hPYY3-36,
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY3-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY18-36
N-epsilon25-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys25]hPYY3-36,
N-epsilon24-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys24]hPYY3-36,
N-epsilon19-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys19]hPYY3-36,
N-epsilon13-[4-(16-(1H-Tetrazol-5-yl)hexadecanoylsulfamoyl)butyryl]ethoxy}ethoxy)acetylamino]ethoxy}ethoxy)acetyl][Lys13]PYY3-36,
N-epsilon25-[4-(16-(1H-Tetrazol-5-yl)hexadecanoylsulfamoyl)butyryl]ethoxy}ethoxy)acetylamino]ethoxy}ethoxy)acetyl][Lys25]PYY3-36,
N-alpha-[4-(16-(1H-Tetrazol-5-yl)hexadecanoylsulfamoyl)butyryl]ethoxy}ethoxy)acetylamino]ethoxy}ethoxy)acetyl]PYY3-36,
N-alpha-[4-(16-(1H-Tetrazol-5-yl)hexadecanoylsulfamoyl)butyryl]ethoxy}ethoxy)acetylamino]ethoxy}ethoxy)acetyl]PYY3-36,
N-epsilon18-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys18]PYY3-36,
N-epsilon22-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys22]PYY3-36,
N-epsilon26-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys26]PYY3-36,
N-epsilon29-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys29]PYY3-36,
N-epsilon36-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys36]PYY3-36,
N-epsilon21-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys21]PYY3-36,
N-epsilon30-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys30]PYY3-36,
N-epsilon31-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys31]PYY3-36,
N-epsilon14-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys14]PYY3-36,
N-epsilon15-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys15]PYY3-36,
N-epsilon16-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys16]PYY3-36,
N-epsilon20-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys20]PYY3-36,
N-epsilon28-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys28]PYY3-36,
N-epsilon32-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys32]PYY3-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}-ethoxy)acetylamino]ethoxy}ethoxy)acetyl] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][N-Methyl Arg35] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][His25,N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Aib25,N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Tyr25,N-Methyl Gln34] PYY(3-36),
N-alpha-{2-(2-{2-[2-(2-{2-[4-Carboxy-4-(17-carboxyheptadecanoylamino)butyrylamino]ethoxy}-ethoxy)acetylamino]-ethoxy}ethoxy)acetyl}[Ala20] PYY(3-36),
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[N-methyl Arg35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Ala27] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Agp35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Agb35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[HomoArg35] PYY(3-36),
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[NArg35] PYY(3-36) or
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[NGln34] PYY(3-36).
   27. The PYY derivative according to any one of the preceding embodiments, wherein said derivative is not
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,N-methyl Tyr36] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Cα-methyl Tyr36] PYY(3-36),
[N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl] PYY(3-36),
[N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl][NTyr36]]PYY3-36, or
N-alpha-Acetyl[N-epsilon10-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}][Lys10,N-methyl Arg35]] PYY(3-36).
   28. The PYY derivative according to any one of the preceding embodiments, wherein said derivative has a half-life of at least 8 hours, such as at least 15 hours or at least 30 hours, as determined by Assay (IV).
   29. The PYY derivative according to any one of the preceding embodiments, wherein said half-life is at least 40 h, such as at least 50 h or at least 60 h, as determined by Assay (IV).
   30. The PYY derivative according to any one of the preceding embodiments, wherein said serum albumin binding side chain is attached to the side chain of an amino acid in a position selected from the group consisting of position 12 and 16-31.
   31. The PYY derivative according to any one of the preceding embodiments, wherein said serum albumin binding side chain is attached to the side chain of an amino acid in a position selected from the group consisting of position 17, 20-22 and 24-31.
   32. The PYY derivative according to any one of the preceding embodiments, wherein said serum albumin binding side chain is attached to the side chain of an amino acid in a position selected from the group consisting of position 24, 25, 27, 28, 30 and 31.
   33. The PYY derivative according to any one of the preceding embodiments, wherein said serum albumin binding side chain is attached the side chain of the amino acid in position 17.
   34. The PYY derivative according to any one of the preceding embodiments, wherein said serum albumin binding side chain is attached the side chain of the amino acid in position 21.
   35. The PYY derivative according to any one of the preceding embodiments, wherein said serum albumin binding side chain is attached the side chain of the amino acid in position 30.
   36. The PYY derivative according to any one of the preceding embodiments, wherein said serum albumin binding side chain is attached the side chain of the amino acid in position 31.
   37. The PYY derivative according to any one of the preceding embodiments, wherein said derivative has a Y2 receptor potency of up to 20 nM as determined by Assay (I) as described herein.
   38. The PYY derivative according to any one of the preceding embodiments, wherein said derivative has a Y2 receptor potency of up to 10 nM as determined by Assay (I).
   39. The PYY derivative according to any one of the preceding embodiments, wherein said derivative has a Y5/Y2 receptor potency ratio which is at least 5 as determined by Assay (III) and Assay (I), respectively.
   40. The PYY derivative according to any one of the preceding embodiments, wherein said derivative has a Y5/Y2 receptor potency ratio which is at least equal to or higher than the Y5/Y2 receptor potency ratio of hPYY(3-36) as determined by Assay (III) and Assay (I), respectively.
   41. The PYY derivative according to any one of the preceding embodiments, wherein said derivative has a Y5/Y2 receptor potency ratio which at least 15 or at least 20 as determined by Assay (III) and Assay (I), respectively.
   42. The PYY derivative according to any one of the preceding embodiments, wherein said derivative has a Y1/Y2 receptor potency ratio which is at least 2 as determined by Assay (II) and Assay (I), respectively.
   43. The PYY derivative according to any one of the preceding embodiments, wherein said derivative has a Y1/Y2 receptor potency ratio which is higher than the Y1/Y2 receptor potency ratio of hPYY(3-36) as determined by Assay (II) and Assay (I), respectively.
   44. The PYY derivative according to any one of the preceding embodiments, wherein said derivative has a Y1/Y2 receptor potency ratio which is at least 15 or at least 20, such as at least 30 or at least 50, as determined by Assay (II) and Assay (I), respectively.
   45. The PYY derivative according to any one of the preceding embodiments, wherein said derivative has a half-life of at least 8 hours as determined by Assay (IV) and a Y2 receptor potency of less than 10 nM as determined by Assay (I).
   46. The PYY derivative according to any one of the preceding embodiments, wherein said derivative has a half-life of at least 8 hours as determined by Assay (IV), a Y2 receptor potency of less than 10 nM as determined by Assay (I), and a Y5/Y2 receptor potency ratio of at least 5 as determined by Assay (III) and Assay (I), respectively.
   47. The PYY derivative according to any one of the preceding embodiments, wherein said serum albumin binding side chain comprises a distal carboxylic acid or a distal tetrazole group.
   48. The PYY derivative according to any one of the preceding embodiments, wherein said serum albumin binding side chain comprises an alkyl chain with at least 14 carbon atoms.
   49. The PYY derivative according to any one of the preceding embodiments, wherein said serum albumin binding side chain comprises an alkyl chain with at least 14 carbon atoms comprising a distal carboxylic acid or a distal tetrazole group.
   50. The PYY derivative according to any one of the preceding embodiments, wherein said serum albumin binding side chain comprises a C18 dicarboxylic acid or a C16 dicarboxylic acid.
   51. The PYY derivative according to any one of the preceding embodiments, wherein said serum albumin binding side chain comprises a C16 carboxylic acid.
   52. The PYY derivative according to any one of the preceding embodiments, wherein said serum albumin binding side chain is selected from the group consisting of
2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl,
2-(2-{2-[2-(2-{2-[(S)-4-Carboxy-4-(19-carboxynonadecanoylamino)butyrylamino]ethoxy}ethoxy)acetyl-amino]ethoxy}ethoxy)acetyl,
2-(2-{2-[2-(2-{2-[(S)-4-Carboxy-4-(17-carboxyheptadecanoylamino)butyrylamino]ethoxy}ethoxy)acetyl-amino]ethoxy}ethoxy)acetyl,
2-(2-{2-[2-(2-{2-[(S)-4-Carboxy-4-(15-carboxypentadecanoylamino)butyrylamino]ethoxy}ethoxy)acetyl-amino]ethoxy}ethoxy)acetyl and 2-(2-{2-[2-(2-{2-[(S)-4-Carboxy-4-(hexadecanoylamino)butyrylamino]ethoxy}ethoxy)acetyl-amino]ethoxy}ethoxy)acetyl.
   53. The PYY derivative according to any one of the preceding embodiments, wherein said serum albumin binding side chain is [4-(16-(1H-Tetrazol-5-yl)hexadecanoylsulfamoyl)butyryl]ethoxy}ethoxy)acetylamino]ethoxy}ethoxy)acetyl].
   54. The PYY derivative according to any one of the preceding embodiments, wherein said serum albumin binding side chain is selected from the group consisting of
[2-(2-{2-[2-(2-{2-((S)-4-carboxy-[(S)-4-Carboxy-4-(17-carboxyheptacanoylamino)butyrylamino/butyrylamino)ethoxy]ethoxy)acetylamino]ethoxy}ethoxy)acetyl],
[2-(2-{2-[2-(2-{2-(15-carboxypentadecanoylamino)-ethoxy}ethoxy)acetylamino]ethoxy}ethoxy)acetyl],
[2-(2-{2-[2-(2-{2-(17-carboxyheptadecanoylamino)-ethoxy}ethoxy)acetylamino]ethoxy}ethoxy)acetyl]
[2-(2-{2-[2-(2-{2-(19-carboxynonadecanoylamino)ethoxy}ethoxy)acetylamino]ethoxy}ethoxy)acetyl],
[(S)-4-Carboxy-4-(17-carboxyheptadecanoylamino)butyrylamino], and
[(S)-4-Carboxy-4-(17-carboxyheptadecanoylamino)butyrylamino]-Ser-Ser-Gly-Ser-Ser-Gly].
   55. The PYY derivative according to any one of the preceding embodiments, wherein said serum albumin binding side chain is attached to the side chain of either 2,3-diaminopropionic acid, 2,4-diaminobutyric acid, ornitine or Lys.
   56. The PYY derivative according to any one of the preceding embodiments, wherein in said derivative two amino acid residues are cross-linked using e.g. a disulfide, lactame or tetrazole linkage.
   57. The PYY derivative according to any of the preceding embodiments, wherein said derivative comprises formula (I):

Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅-Xaa₂₆-Xaa₂₇-Xaa₂₈-Xaa₂₉-Xaa₃₀-Xaa₃₁-Xaa₃₂-Xaa₃₃-Xaa₃₄-Xaa₃₅-Xaa₃₆ Formula (I)

wherein
Xaa₁ is Tyr, Phe, Ala, 2,3-diaminopropionic acid, 2,4-diaminobutyric acid, ornitine, Lys or absent;
Xaa₂ is Pro, Ala, Leu, Phe, hydroxyproline, 2,3-diaminopropionic acid, 2,4-diaminobutyric acid, ornitine, Lys or absent;
Xaa₃ is Ile, Val, Leu (1-aminocyclopentyl)carboxylic acid, (1-aminocyclohexyl)carboxylic acid, 1-aminobutyric acid, 2,3-diaminopropionic acid, 2,4-diaminobutyric acid, ornitine, Lys, D-Ile, D-allo-Ile or absent;
Xaa₄ is Lys, Arg, Glu, 2,3-diaminopropionic acid, 2,4-diaminobutyric acid, ornitine, Lys, absent, Ala, Val, Ser or Gly;
Xaa₅ is Pro, 2,3-diaminopropionic acid, 2,4-diaminobutyric acid, ornitine, absent or Lys;
Xaa₆ is Glu, 2,3-diaminopropionic acid, 2,4-diaminobutyric acid, ornithine, absent or Lys;
Xaa₇ is Ala, 2,3-diaminopropionic acid, 2,4-diaminobutyric acid, ornitine, absent or Lys;
Xaa₈ is Pro, Ala, 2,3-diaminopropionic acid, 2,4-diaminobutyric acid, ornitine, absent, Glu or Lys;
Xaa₉ is Gly, 2,3-diaminopropionic acid, 2,4-diaminobutyric acid, ornitine, absent, Glu or Lys;
Xaa₁₀ is Glu, 2,3-diaminopropionic acid, 2,4-diaminobutyric acid, ornitine or Lys;
Xaa₁₁ is Asp, Ala, 2,3-diaminopropionic acid, 2,4-diaminobutyric acid, ornitine or Lys;
Xaa₁₂ is Ala, 2,3-diaminopropionic acid, 2,4-diaminobutyric acid, ornitine or Lys;
Xaa₁₃ is Ser, 2,3-diaminopropionic acid, 2,4-diaminobutyric acid, ornithine, Lys;
Xaa₁₄ is Pro, hydroxyproline or Ala;
Xaa₁₅ is Glu, 2,3-diaminopropionic acid, 2,4-diaminobutyric acid, ornitine or Lys;
Xaa₁₆ is Glu, 2,3-diaminopropionic acid, 2,4-diaminobutyric acid, ornitine or Lys;
Xaa₁₇ is Leu, Val, Ile, homoleucine, norleucine, (1-aminocyclopentyl)carboxylic acid, (1-aminocyclohexyl)carboxylic acid or 1-aminobutyric acid;
Xaa₁₈ is Asn, Ala, Glu, 2,3-diaminopropionic acid, 2,4-diaminobutyric acid, ornitine, Lys, Gln, Asp, D-Asp, IsoAsp or D-IsoAsp;
Xaa₁₉ is Arg, Ala, 2,3-diaminopropionic acid, 2,4-diaminobutyric acid, ornithine, Glu or Lys;
Xaa₂₀ is Tyr, Ala, Phe, 3-pyridylalaine, 2,3-diaminopropionic acid, 2,4-diaminobutyric acid, ornitine or Lys;
Xaa₂₁ is Tyr, Ala, Phe, 3-pyridylalaine, 2,3-diaminopropionic acid, 2,4-diaminobutyric acid, ornitine or Lys;
Xaa₂₂ is Ala, 2,3-diaminopropionic acid, 2,4-diaminobutyric acid, ornithine, Arg, Glu or Lys;
Xaa₂₃ is Ser, 2,3-diaminopropionic acid, 2,4-diaminobutyric acid, ornitine or Lys;
Xaa₂₄ is Leu, Ile, Val, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₂₅ is Arg, Ala, His, aminoisobutyric acid, 2,3-diaminopropionic acid, 2,4-diaminobutyric acid, ornitine or Lys;
Xaa₂₆ is His, Arg, 2,3-diaminopropionic acid, 2,4-diaminobutyric acid, ornitine or Lys;
Xaa₂₇ is Tyr, Ala, Phe, homoPhe or 3-pyridylalanine, 2,3-diaminopropionic acid, 2,4-diaminobutyric acid, ornitine or Lys;
Xaa₂₈ is Leu, Ile, Val, homoleucine, norleucine, (1-aminocyclopentyl)carboxylic acid, (1-aminocyclohexyl)carboxylic acid, aminoisobutyric acid, 1-aminobutyric acid, 2,3-diaminopropionic acid, 2,4-diaminobutyric acid, ornitine or Lys;
Xaa₂₉ is Asn, Gln, 2,3-diaminopropionic acid, 2,4-diaminobutyric acid, ornithine, D-IsoAsp or Lys;
Xaa₃₀ is Leu, Met, Val, Ile, homoleucine, aminoisobutyric acid, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₁ is Val, Leu, Ile, aminoisobutyric acid, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₂ is Thr, Ser, 2,3-diaminopropionic acid, 2,4-diaminobutyric acid, ornitine or Lys;
Xaa₃₃ is Arg, N-methyl Arg, methyllysine, dimethyllysine, trimethyllysine, 2-amino-3-guanidino-propionic acid, 2-amino-4-guanidino-butyric acid or monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid;
Xaa₃₄ is Gln, Asn, His, Pro, N-methyl Gln, 8-homo Gln, (2-Carbamoyl-ethylamino)-acetic acid, N-methyl Asn or N-methyl His;
Xaa₃₅ is Arg, N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid; and
Xaa₃₆ is Tyr, Phe, N-methyl Tyr, C-methyl Phe, 3-pyridylalanine or (4-Hydroxybenzylamino)-acetic acid, 4-fluorophenylalanine or 4-pyridylalanine.
58. The PYY derivative according to any of the preceding embodiments, wherein said derivative comprises formula (I):

Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅-Xaa₂₆-Xaa₂₇-Xaa₂₈-Xaa₂₉-Xaa₃₀-Xaa₃₁-Xaa₃₂-Xaa₃₃-Xaa₃₄-Xaa₃₅-Xaa₃₆ Formula (I)

wherein
Xaa₁ is Tyr, Phe, Ala, 2,3-diaminopropionic acid, 2,4-diaminobutyric acid, ornitine, Lys or absent;
Xaa₂ is Pro, Ala, Leu, Phe, hydroxyproline, 2,3-diaminopropionic acid, 2,4-diaminobutyric acid, ornitine, Lys or absent;
Xaa₃ is Ile, Val, Leu (1-aminocyclopentyl)carboxylic acid, (1-aminocyclohexyl)carboxylic acid, 1-aminobutyric acid, 2,3-diaminopropionic acid, 2,4-diaminobutyric acid, ornitine, Lys or absent;
Xaa₄ is Lys, Arg, Glu, 2,3-diaminopropionic acid, 2,4-diaminobutyric acid, ornitine, Lys or absent;
Xaa₅ is Pro, 2,3-diaminopropionic acid, 2,4-diaminobutyric acid, ornitine or Lys;
Xaa₆ is Glu, 2,3-diaminopropionic acid, 2,4-diaminobutyric acid, ornithine or Lys;
Xaa₇ is Ala, 2,3-diaminopropionic acid, 2,4-diaminobutyric acid, ornitine or Lys;
Xaa₈ is Pro, Ala, 2,3-diaminopropionic acid, 2,4-diaminobutyric acid, ornitine or Lys;
Xaa₉ is Gly, 2,3-diaminopropionic acid, 2,4-diaminobutyric acid, ornitine or Lys;
Xaa₁₀ is Glu, 2,3-diaminopropionic acid, 2,4-diaminobutyric acid, ornitine or Lys;
Xaa₁₁ is Asp, Ala, 2,3-diaminopropionic acid, 2,4-diaminobutyric acid, ornitine or Lys;
Xaa₁₂ is Ala, 2,3-diaminopropionic acid, 2,4-diaminobutyric acid, ornitine or Lys;
Xaa₁₃ is Ser, 2,3-diaminopropionic acid, 2,4-diaminobutyric acid, ornithine, Lys;
Xaa₁₄ is Pro or hydroxyproline;
Xaa₁₅ is Glu, 2,3-diaminopropionic acid, 2,4-diaminobutyric acid, ornitine or Lys;
Xaa₁₆ is Glu, 2,3-diaminopropionic acid, 2,4-diaminobutyric acid, ornitine or Lys;
Xaa₁₇ is Leu, Val, Ile, homoleucine, norleucine, (1-aminocyclopentyl)carboxylic acid, (1-aminocyclohexyl)carboxylic acid or 1-aminobutyric acid;
Xaa₁₈ is Asn, Ala, Glu, 2,3-diaminopropionic acid, 2,4-diaminobutyric acid, ornitine or Lys;
Xaa₁₉ is Arg, Ala, 2,3-diaminopropionic acid, 2,4-diaminobutyric acid, ornitine or Lys;
Xaa₂₀ is Tyr, Ala, Phe, 3-pyridylalaine, 2,3-diaminopropionic acid, 2,4-diaminobutyric acid, ornitine or Lys;
Xaa₂₁ is Tyr, Ala, Phe, 3-pyridylalaine, 2,3-diaminopropionic acid, 2,4-diaminobutyric acid, ornitine or Lys;
Xaa₂₂ is Ala, 2,3-diaminopropionic acid, 2,4-diaminobutyric acid, ornitine or Lys;
Xaa₂₃ is Ser, 2,3-diaminopropionic acid, 2,4-diaminobutyric acid, ornitine or Lys;
Xaa₂₄ is Leu, Ile, Val, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₂₅ is Arg, Ala, His, aminoisobutyric acid, 2,3-diaminopropionic acid, 2,4-diaminobutyric acid, ornitine or Lys;
Xaa₂₆ is His, Arg, 2,3-diaminopropionic acid, 2,4-diaminobutyric acid, ornitine or Lys;
Xaa₂₇ is Tyr, Ala, Phe, homoPhe or 3-pyridylalanine, 2,3-diaminopropionic acid, 2,4-diaminobutyric acid, ornitine or Lys;
Xaa₂₈ is Leu, Ile, Val, homoleucine, norleucine, (1-aminocyclopentyl)carboxylic acid, (1-aminocyclohexyl)carboxylic acid, aminoisobutyric acid, 1-aminobutyric acid, 2,3-diaminopropionic acid, 2,4-diaminobutyric acid, ornitine or Lys;
Xaa₂₉ is Asn, Gln, 2,3-diaminopropionic acid, 2,4-diaminobutyric acid, ornitine or Lys;
Xaa₃₀ is Leu, Met, Val, Ile, homoleucine, aminoisobutyric acid, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₁ is Val, Leu, Ile, aminoisobutyric acid, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₂ is Thr, Ser, 2,3-diaminopropionic acid, 2,4-diaminobutyric acid, ornitine or Lys;
Xaa₃₃ is Arg, N-methyl Arg, methyllysine, dimethyllysine, trimethyllysine, 2-amino-3-guanidino-propionic acid, 2-amino-4-guanidino-butyric acid or monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid;
Xaa₃₄ is Gln, Asn, His, Pro, N-methyl Gln, β-homo Gln, (2-Carbamoyl-ethylamino)-acetic acid, N-methyl Asn or N-methyl His;
Xaa₃₅ is Arg, N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)-acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)-acetic acid; and
Xaa₃₆ is Tyr, Phe, N-methyl Tyr, C-methyl Phe, 3-pyridylalanine or (4-Hydroxybenzylamino)-acetic acid.
59. The PYY derivative according to any of the preceding embodiments, wherein Xaa₁ and Xaa₂ or Xaa₁, Xaa₂, Xaa₃ and Xaa₄ are absent.
60. The PYY derivative according to any of the preceding embodiments, wherein Xaa₄ is Arg.
61. The PYY derivative according to any of the preceding embodiments, wherein Xaa₄ is Asp or Glu.
62. The PYY derivative according to any of the preceding embodiments, wherein Xaa₁₈ is Ala, Glu or Gln.
63. The PYY derivative according to any of the preceding embodiments, wherein Xaa₂₂ is Asp or Glu.
64. The PYY derivative according to any of the preceding embodiments, wherein Xaa₁, Xaa₂, Xaa₃ and Xaa₄ are absent.
65. The PYY derivative according to any of the preceding embodiments, wherein Xaa₄ is Ala.
66. The PYY derivative according to any one of the preceding embodiments, wherein Xaa₁₈ is Asp.
67. The PYY derivative according to any one of the preceding embodiments, wherein Xaa₂₉ is Asn.
68. The PYY derivative according to any of the preceding embodiments, wherein said serum albumin binding side chain is selected from the group consisting of A-B-C-D-, A-C-D-, A-B-C- and A-C-,
wherein A- is

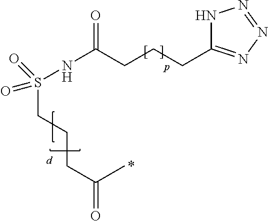

wherein p is selected from the group consisting of 10, 11, 12, 13 and 14, and d is selected from the group consisting of 0, 1, 2, 3, 4 and 5, and
-B- is selected from the group consisting of

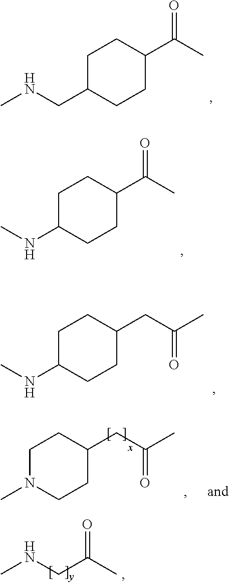

wherein x is selected from the group consisting of 0, 1, 2, 3 and 4, and y is selected from the group consisting of 1, 2, 3, 4, 5, 6, 7, 8, 9, 10, 11 and 12,

### or A- is

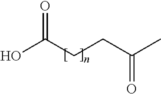

wherein n is selected from the group consisting of 12, 13, 14, 15, 16 17, 18 and 19, and
B is selected from the group consisting of

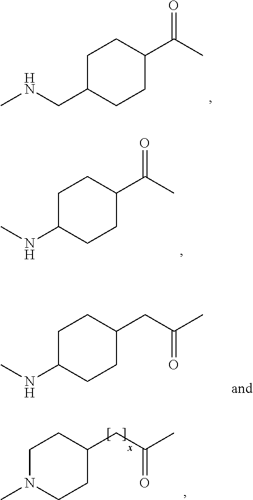

wherein x is selected from the group consisting of 0, 1, 2, 3 and 4, and
-C- is selected from the group consisting of

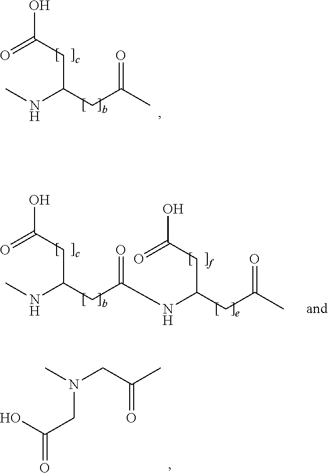

wherein b and e are each independently selected from the group consisting of 0, 1 and 2, and c and f are each independently selected from the group consisting of 0, 1 and 2 with the proviso that b is 1 or 2 when c is 0, or b is 0 when c is 1 or 2, and e is 1 or 2 when f is 0, or e is 0 when f is 1 or 2, and
-D- is attached to said amino acid residue and is a spacer, such as at least one 8-amino-3,6-dioxaoctanoic acid (Oeg) molecule.
69. The PYY derivative according to any one of the preceding embodiments, wherein said spacer comprises two Oeg molecules.
70. The PYY derivative according to any one of the preceding embodiments, wherein said derivative is selected from the group consisting of
N-epsilon35[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys35]hPYY(3-36) (SEQ ID NO: 1);
N-epsilon34[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys34]hPYY(3-36) (SEQ ID NO: 2);
N-epsilon33[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys33]hPYY(3-36) (SEQ ID NO: 3);
N-epsilon32[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys32]hPYY(3-36) (SEQ ID NO: 4);
N-epsilon31[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys31]hPYY(3-36) (SEQ ID NO: 5);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(3-36) (SEQ ID NO: 6);
N-epsilon29[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys29]hPYY(3-36) (SEQ ID NO: 7);
N-epsilon28[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys28]hPYY(3-36) (SEQ ID NO: 8);
N-epsilon27[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys27]hPYY(3-36) (SEQ ID NO: 9);
N-epsilon26[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys26]hPYY(3-36) (SEQ ID NO: 10);
N-epsilon25[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys25]hPYY(3-36) (SEQ ID NO: 11);
N-epsilon24[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys24]hPYY(3-36) (SEQ ID NO: 12);
N-epsilon23[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys23]hPYY(3-36) (SEQ ID NO: 13);
N-epsilon22[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys22]hPYY(3-36) (SEQ ID NO: 14);
N-epsilon21[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys21]hPYY(3-36) (SEQ ID NO: 15);
N-epsilon20[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys20]hPYY(3-36) (SEQ ID NO: 16);
N-epsilon19[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys19]hPYY(3-36) (SEQ ID NO: 17);
N-epsilon18[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys18]hPYY(3-36) (SEQ ID NO: 18);
N-epsilon17[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys17]hPYY(3-36) (SEQ ID NO: 19);
N-epsilon16[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys16]hPYY(3-36) (SEQ ID NO: 20);
N-epsilon15[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys15]hPYY(3-36) (SEQ ID NO: 21);
N-epsilon14[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys14]hPYY(3-36) (SEQ ID NO: 22);
N-epsilon13[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys13]hPYY(3-36) (SEQ ID NO: 23);
N-epsilon12[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys12]hPYY(3-36) (SEQ ID NO: 24);
N-epsilon11[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys11]hPYY(3-36) (SEQ ID NO: 25);
N-epsilon10[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys10]hPYY(3-36) (SEQ ID NO: 26);
N-epsilon9[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys9]hPYY(3-36) (SEQ ID NO: 27);
N-epsilon8[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys8]hPYY(3-36) (SEQ ID NO: 28);
N-epsilon7[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys7]hPYY(3-36) (SEQ ID NO: 29);
N-epsilon6[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys6]hPYY(3-36) (SEQ ID NO: 30);
N-epsilon5[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys5]hPYY(3-36) (SEQ ID NO: 31);
N-epsilon4[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys4]hPYY(3-36) (SEQ ID NO: 32);
N-epsilon3[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys3]hPYY(3-36) (SEQ ID NO: 33);
N-alpha-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl]hPYY(3-36) (SEQ ID NO: 34);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Lys30]hPYY(3-36) (SEQ ID NO: 35);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(5-36) (SEQ ID NO: 36);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4, Glu18,Lys30]hPYY(3-36) (SEQ ID NO: 37);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu18,Lys30]hPYY(5-36) (SEQ ID NO: 38);
N-epsilon30[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl][Arg4,Glu18,Gln29,Lys30]hPYY(3-36) (SEQ ID NO: 39);
N-epsilon30[2-(2-{2-[2-(2-{2-[(S)-4-Carboxy-4-(hexadecanoylamino)butyrylamino]ethoxy}ethoxy)-acetylamino]ethoxy}ethoxy)acetyl][Lys30]hPYY(3-36) (SEQ ID NO: 40);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Gln18,Lys30]hPYY(3-36) (SEQ ID NO: 41); and
N-epsilon1[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys1]hPYY(1-36) (SEQ ID NO: 42).
   71. The PYY derivative according to any one of the preceding embodiments, wherein said derivative is selected from the group consisting of
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ser4,Lys30]hPYY(3-36) (SEQ ID NO: 43);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][IsoAsp18,Lys30]hPYY(3-36) (SEQ ID NO: 44);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-IsoAsp29,Lys30]hPYY(3-36) (SEQ ID NO: 45);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu4,Lys30]hPYY(3-36) (SEQ ID NO: 46);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-IsoAsp18,Lys30]hPYY(3-36) (SEQ ID NO: 47);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Glu22,Lys30]hPYY(3-36) (SEQ ID NO: 48);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Arg4,Glu18,Glu22,Lys30]hPYY(3-36) (SEQ ID NO: 49);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,D-Asp18,Lys30]hPYY(3-36) (SEQ ID NO: 50);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Asp18,Lys30]hPYY(3-36) (SEQ ID NO: 51);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala14,Lys30]hPYY(3-36) (SEQ ID NO: 52);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala18,Lys30]hPYY(3-36) (SEQ ID NO: 53);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Val4,Lys30]hPYY(3-36) (SEQ ID NO: 54);
N-epsilon30[2-(2-{2-[2-(2-{2-[(S)-4-Carboxy-4-(19-carboxynonadecanoylamino)butyrylamino]ethoxy}ethoxy)acetylamino]ethoxy}ethoxy)acetyl][Lys30]hPYY(3-36) (SEQ ID NO: 55);
N-epsilon30[2-(2-{2-[2-(2-{2-(19-carboxynonadecanoylamino)ethoxy}ethoxy)acetylamino]ethoxy}ethoxy)acetyl][Lys30]hPYY(3-36) (SEQ ID NO: 56);
N-epsilon30[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)-acetylamino]ethoxy}ethoxy)acetyl][Lys30]hPYY(3-36) (SEQ ID NO: 57);
N-epsilon30[2-(2-{2-[2-(2-{2-(17-carboxyheptadecanoylamino)-ethoxy}ethoxy)acetylamino]ethoxy}ethoxy)acetyl][Lys30]hPYY(3-36) (SEQ ID NO: 58);
N-epsilon30[2-(2-{2-[2-(2-{2-[(S)-4-Carboxy-4-(15-carboxypentadecanoylamino) butyrylamino]ethoxy}ethoxy)-acetylamino]ethoxy}ethoxy)acetyl][Lys30]hPYY(3-36) (SEQ ID NO: 59);
N-epsilon30[2-(2-{2-[2-(2-{2-(15-carboxypentadecanoylamino)-ethoxy}ethoxy)acetylamino]ethoxy}ethoxy)acetyl][Lys30]hPYY(3-36) (SEQ ID NO: 60);
N-epsilon30[2-(2-{2-[2-(2-{2-[(S)-4-Carboxy-4-(13-carboxytridecanoylamino) butyrylamino]ethoxy}ethoxy)-acetylamino]ethoxy}ethoxy)acetyl][Lys30]hPYY(3-36) (SEQ ID NO: 61);
N-epsilon30[2-(2-{2-[2-(2-{2-(13-carboxytridecanoylamino)-ethoxy}ethoxy)acetylamino]ethoxy}ethoxy)acetyl][Lys30]hPYY(3-36) (SEQ ID NO: 62);
N-epsilon30[2-(2-{2-[2-(2-{2-[(S)-4-Carboxy-4-(11-carboxyundecanoylamino) butyrylamino]ethoxy}ethoxy)-acetylamino]ethoxy}ethoxy)acetyl][Lys30]hPYY(3-36) (SEQ ID NO: 63);
N-epsilon30[2-(2-{2-[2-(2-{2-(11-carboxyundecanoylamino)-ethoxy}ethoxy)acetylamino]ethoxy}ethoxy)acetyl][Lys30]hPYY(3-36) (SEQ ID NO: 64);
N-epsilon30[2-(2-{2-[2-(2-{2-[(4-(16-(1H-Tetrazol-5-yl)hexadecanoylsulfamoyl)butyryl]-ethoxy}ethoxy)-acetylamino]ethoxy}ethoxy)acetyl][Lys30]hPYY(3-36) (SEQ ID NO: 65);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][D-Asp18,Lys30]hPYY(3-36) (SEQ ID NO: 66);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Lys30]hPYY(5-36) (SEQ ID NO: 67);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Ala4,Asp18,Lys30]hPYY(3-36) (SEQ ID NO: 68);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Arg4, Lys30]hPYY(3-36) (SEQ ID NO: 69);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Lys30]hPYY(9-36) (SEQ ID NO: 70);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Gly4, Lys30]hPYY(3-36) (SEQ ID NO: 71);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Arg4,Asp18,Lys30]hPYY(4-36) (SEQ ID NO: 72);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-succinyl,Arg4,Asp18,Lys30]hPYY(3-36) (SEQ ID NO: 73);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-isovaleryl,Arg4,Lys30]hPYY(4-36) (SEQ ID NO: 74);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Lys30,4-fluorophenylalanine36]hPYY(3-36) (SEQ ID NO: 75);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Lys30,4-pyridylalanine36]hPYY(3-36) (SEQ ID NO: 76);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu19,Arg22,Lys30]hPYY(3-36) (SEQ ID NO: 77);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,D-Ile3,Arg4,Lys30]hPYY(3-36) (SEQ ID NO: 78);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Gly4,Lys30]hPYY(3-36) (SEQ ID NO: 79);
N-epsilon30[2-(2-{2-[2-(2-{2-((S)-4-carboxy-[(S)-4-Carboxy-4-(17-carboxyheptadecanoylamino)butyrylamino]butyrylamino)ethoxy}ethoxy)-acetylamino]ethoxy}ethoxy)acetyl][N-alpha-acetyl,Arg4,Lys30]hPYY(3-36) (SEQ ID NO: 80);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Arg4,Asp18,Lys30]hPYY(3-36) (SEQ ID NO: 81);
N-epsilon30[2-(2-{2-[2-(2-{2-[(S)-4-Carboxy-4-(15-carboxypentadecanoylamino)butyrylamino]ethoxy}ethoxy)acetylamino]ethoxy}ethoxy)acetyl][N-alpha-acetyl,Lys30]hPYY(3-36) (SEQ ID NO: 82);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Ala4,Asp18,Lys30]hPYY(3-36) (SEQ ID NO: 83);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Arg4,Asp18,Lys30]hPYY(3-36) (SEQ ID NO: 84);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][D-Arg4, Lys30]hPYY(3-36) (SEQ ID NO: 85);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][D-allo-Ile3,Arg4, Lys30]hPYY(3-36) (SEQ ID NO: 86);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][D-Ala4, Lys30]hPYY(3-36) (SEQ ID NO: 87);
N-epsilon30-[(S)-4-Carboxy-4-(17-carboxyheptadecanoylamino)butyrylamino][Lys30]hPYY(3-36) (SEQ ID NO: 88);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Ala4,Lys30]hPYY(3-36) (SEQ ID NO: 89);
N-epsilon30[{(S)-4-Carboxy-4-(17-carboxyheptadecanoylamino)butyrylamino]-Ser-Ser-Gly-Ser-Ser-Gly][Arg4,Lys30]hPYY(3-36) (SEQ ID NO: 90);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu8,Lys30]hPYY(3-36) (SEQ ID NO: 91);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu9,Lys30]hPYY(3-36) (SEQ ID NO: 92); and
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala4,Lys30]hPYY(3-36) (SEQ ID NO: 93).
   72. The PYY derivative according to any of the preceding embodiments, wherein said derivative is derived from a vertebrate such as a mammal, e.g., a human.
   73. The PYY derivative according to any one of the preceding embodiments, wherein said derivative comprises at least one amino acid residue substituted into a proteinogenic or non-proteinogenic amino acid residue selected from the group consisting of

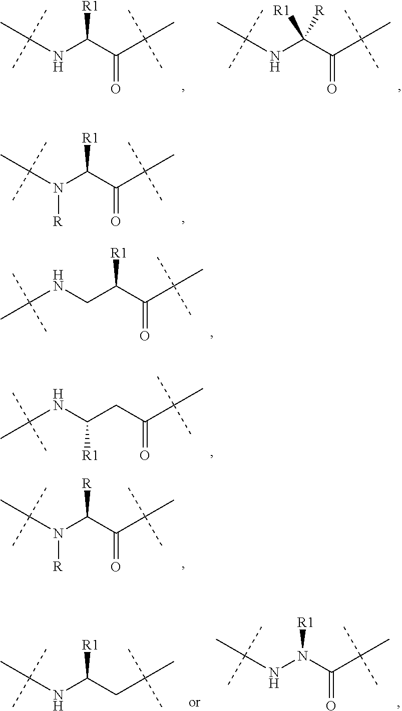

wherein R1 is side a chain of an amino acid; and R is H or C1-C12 alkyl.
74. The PYY derivative according to any one of the preceding embodiments, wherein said derivative comprises the amino acid residue represented by formula (A)

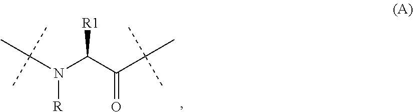

wherein R1 is a side chain of an amino acid and R is selected from the group consisting of alkyl, benzyl or phenyl.
75. The PYY derivative according to any of the preceding embodiments, wherein said derivative is suitable for administration in a dosing regime selected from group consisting of a once-daily, an every other day, a once-weekly, a twice-monthly and a once-monthly dosing regime.
76. A composition comprising the PYY derivative as defined in any of the preceding embodiments and at least one pharmaceutical excipient.
77. The PYY derivative according to any of the preceding embodiments for use as a medicament.
78. The PYY derivative according to any of the preceding embodiments for use in the treatment of a condition responsive to Y receptor modulation.
79. The PYY derivative according to embodiment 78, wherein the condition responsive to Y receptor modulation is obesity.
80. The PYY derivative according to embodiment 78 or 79, wherein said derivative is administered once-daily, every other day, twice-weekly or once-weekly.
81. Use of the PYY derivative as defined in any of embodiments 1-75 or the composition as defined in embodiment 76 for the preparation of a medicament for the treatment of a condition responsive to Y receptor modulation, such as obesity or obesity-related diseases.
82. Use of the PYY derivative as defined in any of embodiments 1-75 or the composition as defined in embodiment 76 for administration in a mammal, wherein said derivative shows protracted properties compared to human PYY(3-36).
83. A method of treatment of a condition responsive to Y receptor modulation by administration of the PYY derivative as defined in any of embodiments 1-75 or the composition as defined in embodiment 76.
84. The method according to embodiment 83, wherein said condition responsive to Y receptor modulation is obesity.
85. The method according to embodiment 83 or 84, wherein said derivative is administered once-daily, every other day, twice-weekly or once-weekly.
86. The method of treatment according to any one of embodiments 83-85, wherein the condition responsive to Y receptor modulation is obesity-related diseases, such as reduction of food intake, Syndrome X (metabolic syndrome), diabetes, type 2 diabetes mellitus or Non Insulin Dependent Diabetes Mellitus (NIDDM), hyperglycemia, insulin resistance, polycystic ovary syndrome (PCOS) or impaired glucose tolerance.
87. The method of treatment according to any one of embodiments 83-86, wherein the condition responsive to Y receptor modulation is an obesity-related cardiovascular disease such as hypertension, atherosclerosis, coronary artery disease, myocardial infarction, peripheral vascular disease, stroke, thromboembolic diseases, hypercholesterolemia or hyperlipidemia.
88. The method of treatment according to any one of embodiments 83-87, wherein the condition responsive to Y receptor modulation is diarrhoea such as infectious diarrhoea, inflammatory diarrhoea, chemotherapy-induced diarrhoea, short bowel syndrome or the diarrhoea which typically occurs following surgical procedures, e.g., ileostomy.
89. The method of treatment according to any one of embodiments 83-88, wherein the condition responsive to Y receptor modulation is a condition characterized by damage to the intestine such as chemotherapy-induced diarrhoea, ulcerative colitis, Crohns disease, bowel atrophy, loss of bowel mucosa, and/or loss of bowel mucosal function.
90. The method of treatment according to any one of embodiments 83-89, wherein the condition responsive to Y receptor modulation is an intestinal inflammatory condition such as ulcerative colitis or Crohn's disease.
91. The method of treatment according to any one of embodiments 83-91, wherein the condition responsive to Y receptor modulation is allergic or non-allergic rhinitis.
92. The method of treatment according to any one of embodiments 83-92, wherein the condition responsive to Y receptor modulation is anxiety.

### EXAMPLES

### Abbreviations Used

### Abbreviation Meaning

Boc: tert butyloxycarbonyl
CH₃CN: Acetonitrile
DCM: Dichloromethane
Dde: 1-(4,4-Dimethyl-2,6-dioxocyclohexylidene)ethyl
DIC: Diisopropylcarbodiimide
DIPEA: Diisopropylethylamine
DIPEA: Diisopropylethylamine
DMF: N,N-dimethylformamide
Et₂O: Diethylether
Fmoc: 9H-fluoren-9-ylmethoxycarbonyl
H₂O: Water
HBTU: 2-(1H-Benzotriazol-1-yl-)-1,1,3,3 tetramethyluronium hexafluorophosphate
HFIP: Hexafluoroisopropanol
HOAt: 1-Hydroxy-7-azabenzotriazole
HOBt: 1-Hydroxybenzotriazole
ivDde: 1-(4,4-Dimethyl-2,6-dioxocyclohexylidene)-3-methylbutyl
Mmt: 4-Methoxytrityl
Mtt: 4-Methyltrityl
MW: Molecular weight
NMP: 1-Methyl-pyrrolidin-2-one
Oeg: 8-Amino-3,6-dioxaoctanoic acid
OtBu: Tert butyl ester
Pmc: 2,2,5,7,8-Pentamethyl-chroman-6-sulfonyl
r.t: Room temperature
tBu: Tert butyl
TFA: Trifluoroacetic acid
TIPS: triisopropylsilane)
Trt: Triphenylmethyl
Trx: Trans-4-(aminomethyl)-cyclohexane carboxylic acid

Synthesis Method 1: Synthesis of Crude Peptides in Array Format

The PYY derivatives of the invention that were used for in vivo experiments in minipigs for determination of the plasma half-life were synthesised in array format using multipeptide synthesis equipment from Intavis, Germany. The solid phase synthesis was performed on Tentagel S RAM resin in a 96 microtiter filterplate (NUNC) with 25 mg resin in each well. Solid phase synthesis was done using the conditions described in this patent. Solid phase synthesis was a follows. Deprotection of the Fmoc-group was done by addition of 100 μl 25% piperidine in NMP for 2 min to each well, followed by another addition of 100 μl of 25% piperidine in NMP for 15 min. The wells were washed using 5×100 μl NMP followed by coupling of the Fmoc-Amino acids. The coupling was done by adding a mixture of 45 μl Fmoc-amino acid (0.3 M in 0.3 M HOAt solution) and 13 μl 1 M diisopropylcarbodiimide (DIC)+20 μl 1 M collidine in NMP to each well and allowed to couple for 5 min. The coupling was repeated with the same amount with a coupling time of 30 min followed by a third coupling with the same amount for 60 min. The N-terminal amino acid was coupled as a Boc-protected amino acid.

Removal of the Mtt protecting group of lysine was done using 75% hexafluoroisopropanol (HFIP) in dichloromethane (DCM) for 60 min. Then washed by DCM followed by a synthesis of 2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl, also called C18diacid-LgammaGlu-Oeg-Oeg, handle by conventional Fmoc-solid phase synthesis using C18 diacid monotertbutyl protected and Fmoc-Glu-OtBu (IRIS Biotech) and Fmoc-Oeg-OH (IRIS Biotech) as building blocks. The coupling was achieved by activation of 50 μl of a 0.3 M solution of building blocks in NMP containing 0.3 M HOAt with 15 μl 1 M DIC and 15 μl 1 M collidine for approx. 2 min. after which it was added to the resin in the microtiter wells and allowed to couple for 10 min. This procedure was repeated and allowed to couple for 60 min. Finally, a third coupling was performed with a coupling time of 240 min.

The final deprotection and removal of side chain protecting groups was done by successive addition of 92% TFA 5% triisopropylsilan (TIPS) and 3% thioanisol 250 μl each time. The TFA was allowed to drop into a 2 ml deepwell 96 microtiter well (NUNC). The combined TFA was reduced in volume by a stream of argon for approx. 30 min and diethylether was then added and the precipitated peptide was transferred to a solvinert deepwell filter plate with 0.45 μm frit (Waters). The precipitated peptide was washed three times with diethylether and dried by vacuum through the filter plate.

The position of the acylation was changed stepwise throughout the synthesis subsequently leading to a collection of hPYY(3-36) peptides that only differed in the position of the acylation position in the peptide backbone. The fatty acid handle that was used for the acylation was C18diacid-LgammaGlu-Oeg-Oeg.

The peptides prepared by array were not purified but analysed by MALDI-MS and UPLC analysis and were used as crude peptides when formulated in phosphate buffer pH 7.4 or pH 4 and injected into minipigs for PK experiments.

### Synthesis Method 2: Synthesis of Resin Bound Peptide

SPPS Method:

For peptides that were synthesised in larger amounts and used for in vitro characterisation the following synthesis method was used. The protected peptidyl resin was synthesized according to the Fmoc strategy on a Prelude Solid Phase Peptide Synthesizer from Protein Technologies in 0.25 mmol scale using DIC and HOAt mediated couplings in NMP. The starting resin used for the synthesis of the peptide amides was Rink-Amide resin. The protected amino acid derivatives used were standard Fmoc-amino acids (supplied from e.g. Anaspec, Bachem, Iris Biotech or Novabiochem). The epsilon amino group, e.g. of lysines, to be acylated were protected with Mtt. The synthesis of the peptides may in some cases be improved by the use of dipeptides, e.g., pseudoprolines from Novabiochem, Fmoc-Ser(tbu)-ψSer(Me,Me)-OH, see e.g. catalogue from Novobiochem 2002/2003 or newer version or W. R. Sampson (1999), J. Pep. Sci. 5, 403.

Procedure for Cleaving the Peptide Off the Resin:

After synthesis the resin was washed with DCM and dried, and the peptide was cleaved from the resin by a 2 hour treatment with TFA/TIPS/water (92.5/5/2.5) or TFA/TIPS (95/5) followed by precipitation with diethylether. The peptide was redissolved in 30% acetic acid or similar solvent and purified by standard RP-HPLC on a C18 column using acetonitrile/TFA. The identity of the peptide was confirmed by MALDI-MS.

Procedure for Removal of Mtt-Protection:

The resin was placed in a syringe and treated with hexafluoroisopropanol for 2×10 min to remove the Mtt group. The resin was then washed with DCM and NMP as described above.

Procedure for Attachment of Sidechains to Lysine Residue:

The serum albumin binding residue A-B-C-D, A-C-D, A-B-C or A-B can be attached to the peptide either by acylation to resin bound peptide or acylation in solution to the unprotected peptide using standard acylating reagent such as but not limited to DIC, HOBt/DIC, HOAt/DIC or HBTU.

Procedure for Removal of Fmoc-Protection:

The resin (0.25 mmol) was placed in a filter flask in a manual shaking apparatus and treated with N-methylpyrrolidone/methylene chloride (1:1) (2×20 ml) and with N-methylpyrrolidone (1×20 ml), a solution of 20% piperidine in N-methylpyrrolidone (3×20 ml, 10 min each). The resin was washed with N-methylpyrrolidone (2×20 ml), N-methylpyrrolidone/Methylene chloride (1:1) (2×20 ml) and methylene chloride (2×20 ml).

### Peptide Analysis and Purification

MALDI-MS:

Molecular weights of the peptides were determined using matrixassisted laser desorption time of flight mass spectroscopy (MALDI-MS), recorded on a Microflex (Bruker). A matrix of alpha-cyano-4-hydroxy cinnamic acid was used. The molecular weight of the product was calculated based on the result of MALDI-MS analysis.

Peptides were purified by reverse phase HPLC on Waters Equipment using one or more of the following methods.

UPLC (Method 04_A3_{—}1):

The UPLC-analysis was performed using a Waters Acquity UPLC system fitted with a Waters Acquity UPLC HSS T3 1.8 μm, 2.1×150 mm column. UV detections were collected at 214 and 254 nm. Oven temperature was 30 C. The following eluents were used; eluent A) 0.25M ammoniumbicarboate in water/acetonitrile (90:10) and eluent B) acetonitrile/water (70:30). The column was equilibrated with the following eluent composition: 75% eluent A, 25% eluent B. After injection, the sample was eluted at 0.4 ml/min with gradient of 25% to 55% eluent B in eluent A during 16 min.

UPLC (Method 04_A4_{—}1):

The UPLC-analysis was performed using a Waters Acquity UPLC system fitted with a Waters Acquity UPLC HSS T3 1.8 μm, 2.1×150 mm column. UV detections were collected at 214 and 254 nm. Oven temperature was 30 C. The following eluents were used; eluent A) 0.25M ammoniumbicarboate in water/acetonitrile (90:10) and eluent B) acetonitrile/water (70:30). The column was equilibrated with the following eluent composition: 65% eluent A, 35% eluent B. After injection, the sample was eluted at 0.4 ml/min with a gradient of 35% to 65% eluent B in eluent A during 16 min.

UPLC (Method: 04_A6_{—}1):

The RP-analysis was performed using a Waters UPLC system fitted with a dual band detector. UV detections at 214 nm and 254 nm were collected using an ACQUITY UPLC BEH130, C18, 130 Å, 1.7 μm, 2.1 mm×150 mm column, 40° C. The UPLC system was connected to two eluent reservoirs containing: A: 10 mM TRIS, 15 mM ammonium sulphate, 80% H₂O, 20%, pH 7.3; B: 80% CH₃CN, 20% H₂O. The following linear gradient was used: 95% A, 5% B to 10% A, 90% B over 16 minutes at a flow-rate of 0.35 ml/min.

UPLC (Method 07_B4_{—}1):

The UPLC-analysis was performed using a Waters Acquity UPLC system fitted with a Waters Acquity UPLC BEH 1.7 μm, 2.1×150 mm column. UV detections were collected at 214 and 254 nm. Oven temperature was 40 C. The following eluents were used; eluent A) 0.05% trifluoroacetic acid in water and eluent B) 0.0% trifluoroacetic acid in acetonitrile. The column was equilibrated with the following eluent composition: 95% eluent A, 5% eluent B. After injection, the sample was eluted at 0.4 ml/min with a gradient of 5% to 95% eluent B in eluent A during 16 min.

UPLC (Method 07_B2_{—}1):

The UPLC-analysis was performed using a Waters Acquity UPLC system fitted with a Waters Acquity UPLC BEH 1.7 μm, 2.1×150 mm column. UV detections were collected at 214 and 254 nm. Oven temperature was 40 C. The following eluents were used; eluent A) 0.05% trifluoroacetic acid in water and eluent B) 0.0% trifluoroacetic acid in acetonitrile. The column was equilibrated with the following eluent composition: 95% eluent A, 5% eluent B. After injection, the sample was eluted at 0.4 ml/min with a gradient of 5% to 60% eluent B in eluent A during 16 min.

UPLC (Method 07_B2_{—}2):

The UPLC-analysis was performed using a Waters Acquity UPLC system fitted with a Waters Acquity UPLC BEH 1.7 μm, 2.1×50 mm column. UV detections were collected at 214 and 254 nm. Oven temperature was 40° C. The following eluents were used; eluent A) 0.05% trifluoroacetic acid in water and eluent B) 0.0% trifluoroacetic acid in acetonitrile. The column was equilibrated with the following eluent composition: 95% eluent A, 5% eluent B. After injection, the sample was eluted at 0.4 ml/min with a gradient of 5% to 60% eluent B in eluent A during 16 min.

The retention time (RT) was calculated based on the result of the UPLC analysis.

### Example 1

SEQ ID NO: 1: N-epsilon35[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys35]hPYY(3-36)

Synthesis method: Synthesis Method 1

UPLC (Method 07_B4_{—}1): RT=8.0 min.

MALDI-MS m/z: 4050, calculated MW=4049.6.

### Example 2

SEQ ID NO: 2: N-epsilon34[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys34]hPYY(3-36)

Synthesis method: Synthesis Method 1

UPLC (Method 07_B4_{—}1): RT=7.6 min,

MALDI-MS m/z: 4764, calculated MW=Not determined.

### Example 3

SEQ ID NO: 3: N-epsilon33[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys33]hPYY(3-36)

Synthesis method: Synthesis Method 2

UPLC (Method 07_B4_{—}1): RT=6.9 min,

UPLC (Method 04_A3_{—}1): RT=5.9 min,

MALDI-MS m/z: 4737, calculated MW=Not determined.

### Example 4

SEQ ID NO: 4: N-epsilon32[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys32]hPYY(3-36)

Synthesis method: Synthesis Method 2

UPLC (Method 07_B4_{—}1): RT=7.8 min,

UPLC (Method 04_A3_{—}1): RT=9.5 min,

MALDI-MS m/z: 4792, calculated MW=4791.

### Example 5

SEQ ID NO: 5: N-epsilon31[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys31]hPYY(3-36)

Synthesis method: Synthesis Method 2

UPLC (Method 07_B4_{—}1): RT=7.0 min,

UPLC (Method 04_A3_{—}1): RT=7.8 min,

MALDI-MS m/z: 4794, calculated MW=4794.

### Example 6

SEQ ID NO: 6: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(3-36)

Synthesis method: Synthesis Method 2

UPLC (Method 07_B4_{—}1): RT=7.4 min,

UPLC (Method 04_A3_{—}1): RT=8.9 min,

MALDI-MS m/z: 4780.5, calculated MW=4780.

### Example 7

SEQ ID NO: 7: N-epsilon29[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys29]hPYY(3-36)

Synthesis method: Synthesis Method 2

UPLC (Method 07_B4_{—}1): RT=8.3 min,

MALDI-MS m/z: 4779.5, calculated MW=4782.3.

### Example 8

SEQ ID NO: 8 N-epsilon28[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys28]hPYY(3-36)

Synthesis method: Synthesis Method 2

UPLC (Method 07_B4_{—}1): RT=7.5 min,

UPLC (Method 04_A3_{—}1): RT=7.4 min,

MALDI-MS m/z: 4780.5, calculated MW=4780.

### Example 9

SEQ ID NO: 9: N-epsilon27[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys27]hPYY(3-36)

Synthesis method: Synthesis Method 2

UPLC (Method 07_B4_{—}1): RT=9.0 min,

UPLC (Method 04_A3_{—}1): RT=7.3 min,

MALDI-MS m/z: 4730.4, calculated MW=4730.

### Example 10

SEQ ID NO: 10: N-epsilon26[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys26]hPYY(3-36)

Synthesis method: Synthesis Method 2

UPLC (Method 08_B4_{—}1): RT=7.2 min,

UPLC (Method 04_A3_{—}1): RT=10.7 min,

MALDI-MS m/z: 4756.5, calculated MW=4755.

### Example 11

SEQ ID NO: 11: N-epsilon25[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys25]hPYY(3-36)

Synthesis method: Synthesis Method 2

UPLC (Method 07_B4_{—}1): RT=7.5 min,

UPLC (Method 04_A3_{—}1): RT=10.2 min,

MALDI-MS m/z: 4737.4, calculated MW=4737.

### Example 12

SEQ ID NO: 12: N-epsilon24[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys24]hPYY(3-36)

Synthesis method: Synthesis Method 2

UPLC (Method 07_B4_{—}1): RT=7.3 min,

UPLC (Method 04_A3_{—}1): RT=6.8 min,

MALDI-MS m/z: 4780.5, calculated MW=4780.

### Example 13

SEQ ID NO: 13: N-epsilon23[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys23]hPYY(3-36)

Synthesis method: Synthesis Method 2

UPLC (Method 07_B4_{—}1): RT=7.6 min,

UPLC (Method 04_A3_{—}1): RT=11.8 min,

MALDI-MS m/z: 4806.5, calculated MW=4806.

### Example 14

SEQ ID NO: 14: N-epsilon22[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys22]hPYY(3-36)

Synthesis method: Synthesis Method 2

UPLC (Method 07_B4_{—}1): RT=8.1 min,

UPLC (Method 04_A3_{—}1): RT=ND,

MALDI-MS m/z: 4822.5, calculated MW=4820.

### Example 15

SEQ ID NO: 15: N-epsilon21[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys21]hPYY(3-36)

Synthesis method: Synthesis Method 2

UPLC (Method 07_B4_{—}1): RT=7.5 min,

UPLC (Method 04_A3_{—}1): RT=10.3 min,

MALDI-MS m/z: 4730.4, calculated MW=4729.

### Example 16

SEQ ID NO: 16: N-epsilon20[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys20]hPYY(3-36)

Synthesis method: Synthesis Method 2

UPLC (Method 07_B4_{—}1): RT=7.7 min,

UPLC (Method 04_A3_{—}1): RT=9.7 min,

MALDI-MS m/z: 4730.4, calculated MW=4731.

### Example 17

SEQ ID NO: 17: N-epsilon19[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys19]hPYY(3-36)

Synthesis method: Synthesis Method 2

UPLC (Method 07_B4_{—}1): RT=9.2 min,

MALDI-MS m/z: 4737.4, calculated MW=4740.9.

### Example 18

SEQ ID NO: 18: N-epsilon18[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys18]hPYY(3-36)

Synthesis method: Synthesis Method 2

UPLC (Method 07_B4_{—}1): RT=7.5 min,

UPLC (Method 04_A3_{—}1): RT=9.9 min,

MALDI-MS m/z: 4779.5, calculated MW=4780.

### Example 19

SEQ ID NO: 19: N-epsilon17[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys17]hPYY(3-36)

Synthesis method: Synthesis Method 2

UPLC (Method 07_B4_{—}1): RT=7.5 min,

UPLC (Method 04_A3_{—}1): RT=9.9 min,

MALDI-MS m/z: 4780.5, calculated MW=4780.

### Example 20

SEQ ID NO: 20: N-epsilon16[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys16]hPYY(3-36)

Synthesis method: Synthesis Method 2

UPLC (Method 07_B4_{—}1): RT=7.4 min,

UPLC (Method 04_A3_{—}1): RT=12.8 min,

MALDI-MS m/z: 4764.5, calculated MW=not determined.

### Example 21

SEQ ID NO: 21: N-epsilon15[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys15]hPYY(3-36)

Synthesis method: Synthesis Method 2

UPLC (Method 07_B4_{—}1): RT=7.4 min,

UPLC (Method 04_A3_{—}1): RT=12.5 min,

MALDI-MS m/z: 4764.5, calculated MW=4765.

### Example 22

SEQ ID NO: 22: N-epsilon14[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys14]hPYY(3-36)

Synthesis method: Synthesis Method 2

UPLC (Method 07_B4_{—}1): RT=4.9 min,

UPLC (Method 04_A3_{—}1): RT=7.5 min,

MALDI-MS m/z: 4796.5, calculated MW=4796.

### Example 23

SEQ ID NO: 23: N-epsilon13[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys13]hPYY(3-36)

Synthesis method: Synthesis Method 2

UPLC (Method 07_B4_{—}1): RT=8.05 min,

MALDI-MS m/z: 4806.5, calculated MW=4809.

### Example 24

SEQ ID NO: 24: N-epsilon12[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys12]hPYY(3-36)

Synthesis method: Synthesis Method 2

UPLC (Method 07_B4_{—}1): RT=7.9 min,

MALDI-MS m/z: 4822.5, calculated MW=4825.5.

### Example 25

SEQ ID NO: 25: N-epsilon11[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys11]hPYY(3-36)

Synthesis method: Synthesis Method 2

UPLC (Method 07_B4_{—}1): RT=7.9 min,

UPLC (Method 04_A3_{—}1): RT=7.7 min,

MALDI-MS m/z: 4778.5, calculated MW=4778.

### Example 26

SEQ ID NO: 26: N-epsilon10[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys10]hPYY(3-36)

Synthesis method: Synthesis Method 2

UPLC (Method 07_B4_{—}1): RT=8.3 min,

UPLC (Method 04_A3_{—}1): RT=not determined,

MALDI-MS m/z: 4764.5, calculated MW=4768.

### Example 27

SEQ ID NO: 27: N-epsilon9[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys9]hPYY(3-36)

Synthesis method: Synthesis Method 2

UPLC (Method 07_B4_{—}1): RT=7.1 min,

UPLC (Method 04_A3_{—}1): RT=10.5 min,

MALDI-MS m/z: 4836.6, calculated MW=4836.

### Example 28

SEQ ID NO: 28: N-epsilon8[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys8]hPYY(3-36)

Synthesis method: Synthesis Method 2

UPLC (Method 07_B4_{—}1): RT=7.7 min,

UPLC (Method 04_A3_{—}1): RT=9.9 min,

MALDI-MS m/z: 4764.5, calculated MW=4764.

### Example 29

SEQ ID NO: 29: N-epsilon7[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys7]hPYY(3-36)

Synthesis method: Synthesis Method 2

UPLC (Method 07_B4_{—}1): RT=7.7 min,

UPLC (Method 04_A3_{—}1): RT=9.5 min,

MALDI-MS m/z: 4822.5, calculated MW=4822.

### Example 30

SEQ ID NO: 30: N-epsilon6[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys6]hPYY(3-36)

Synthesis method: Synthesis Method 2

UPLC (Method 07_B4_{—}1): RT=7.2 min,

UPLC (Method 04_A3_{—}1): RT=6.7 min,

MALDI-MS m/z: 4764.5, calculated MW=4764.

### Example 31

SEQ ID NO: 31: N-epsilon5[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys5]hPYY(3-36)

Synthesis method: Synthesis Method 2

UPLC (Method 07_B4_{—}1): RT=7.2 min,

UPLC (Method 04_A3_{—}1): RT=9.4 min,

MALDI-MS m/z: 4796.5, calculated MW=4796.

### Example 32

SEQ ID NO: 32: N-epsilon4[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys4]hPYY(3-36)

Synthesis method: Synthesis Method 2

UPLC (Method 07_B4_{—}1): RT=7.8 min,

UPLC (Method 04_A3_{—}1): RT=3.4 min,

MALDI-MS m/z: 4765.4, calculated MW=4767.

### Example 33

SEQ ID NO: 33: N-epsilon3[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys3]hPYY(3-36)

Synthesis method: Synthesis Method 2

UPLC (Method 07_B4_{—}1): RT=7.2 min,

UPLC (Method 04_A3_{—}1): RT=9.2 min,

MALDI-MS m/z: 4780.5, calculated MW=4780.

### Example 34

SEQ ID NO: 34: N-alpha-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl]hPYY(3-36)

Synthesis method: Synthesis Method 2

UPLC (Method 07_B4_{—}1): RT=8.1 min,

UPLC (Method 04_A3_{—}1): RT=9.4 min,

MALDI-MS m/z: 4765.4, calculated MW=4764.

### Example 35

SEQ ID NO: 35: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Lys30]hPYY(3-36)

Synthesis method: Synthesis Method 2

UPLC (Method 07_B4_{—}1): RT=7.6 min,

UPLC (Method 04_A3_{—}1): RT=8.7 min,

MALDI-MS m/z: 4808.5, calculated MW=4806.

### Example 36

SEQ ID NO: 36: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(5-36)

Synthesis method: Synthesis Method 2

UPLC (Method 07_B4_{—}1): RT=7.2 min,

UPLC (Method 04_A4_{—}1): RT=4.2 min,

MALDI-MS m/z: 4539.1, calculated MW=4538.

### Example 37

SEQ ID NO: 37: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4, Glu18,Lys30]hPYY(3-36)

Synthesis method: Synthesis Method 2

UPLC (Method 07_B4_{—}1): RT=7.4 min,

UPLC (Method 04_A3_{—}1): RT=8.4 min,

MALDI-MS m/z: 4823.5, calculated MW=4823.

### Example 38

SEQ ID NO: 38: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu18,Lys30]hPYY(5-36)

Synthesis method: Synthesis Method 2

UPLC (Method 07_B4_{—}1): RT=7.5 min,

UPLC (Method 04_A3_{—}1): RT=8.2 min,

MALDI-MS m/z: 4554.1, calculated MW=4554.

### Example 39

SEQ ID NO: 39: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Glu18,Gln29,Lys30]hPYY(3-36)

Synthesis method: Synthesis Method 2

UPLC (Method 07_B4_{—}1): RT=7.4 min,

UPLC (Method 04_A3_{—}1): RT=8.5 min,

MALDI-MS m/z: 4837.5, calculated MW=4837.

### Example 40

SEQ ID NO: 40: N-epsilon30[2-(2-{2-[2-(2-{2-[(S)-4-Carboxy-4-(hexadecanoylamino)butyrylamino]ethoxy}ethoxy)-acetylamino]ethoxy}ethoxy)acetyl][Lys30]hPYY(3-36)

Synthesis method: Synthesis Method 2

UPLC (Method 07_B4_{—}1): RT=not determined,

UPLC (Method 04_A3_{—}1): RT=not determined,

MALDI-MS m/z: 4722.4, calculated MW=not determined.

### Example 41

SEQ ID NO: 41: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Gln18,Lys30]hPYY(3-36)

Synthesis method: Synthesis Method 2

UPLC (Method 07_B4_{—}1): RT=3.5 min,

UPLC (Method 04_A3_{—}1): RT=7.4 min,

MALDI-MS m/z: 4822.5, calculated MW=4821.

### Example 42

SEQ ID NO: 42: N-epsilon1[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys1]hPYY(1-36)

Synthesis method: Synthesis Method 2

UPLC (Method 07_B4_{—}1): RT=7.2 min,

UPLC (Method 04_A3_{—}1): RT=9.1 min,

MALDI-MS m/z: 4990.7, calculated MW=4990.

### Example 43

SEQ ID NO: 43: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ser4,Lys30]hPYY(3-36)

Synthesis method: Synthesis Method 2

UPLC (Method 08_B4_{—}1): RT=7.6 min,

UPLC (Method 04_A4_{—}1): RT=3.6 min,

MALDI-MS m/z: 4740.5, calculated MW=4739.4.

### Example 44

SEQ ID NO: 44: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][IsoAsp18,Lys30]hPYY(3-36)

Synthesis method: Synthesis Method 2

UPLC (Method 07_B4_{—}1): RT=8.3 min,

UPLC (Method 04_A3_{—}1): RT=8.9 min,

MALDI-MS m/z: 4779, calculated MW=4781.4.

### Example 45

SEQ ID NO: 45: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-IsoAsp29,Lys30]hPYY(3-36)

Synthesis method: Synthesis Method 2

UPLC (Method 07_B4_{—}1): RT=8.3 min,

UPLC (Method 04_A4_{—}1): RT=8.9 min,

MALDI-MS m/z: 4779, calculated MW=4781.4.

### Example 46

SEQ ID NO: 46: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu4,Lys30]hPYY(3-36)

Synthesis method: Synthesis Method 2

UPLC (Method 07_B4_{—}1): RT=8.5 min,

UPLC (Method 04_A3_{—}1): RT=8.7 min,

MALDI-MS m/z: 4780, calculated MW=4781.4.

### Example 47

SEQ ID NO: 47: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-IsoAsp18,Lys30]hPYY(3-36)

Synthesis method: Synthesis Method 2

UPLC (Method 07_B4_{—}1): RT=8.4 min,

UPLC (Method 04_A3_{—}1): RT=9.3 min,

MALDI-MS m/z: 4779, calculated MW=4781.4.

### Example 48

SEQ ID NO: 48: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Glu22,Lys30]hPYY(3-36)

Synthesis method: Synthesis Method 2

UPLC (Method 07_B4_{—}1): RT=8.9 min,

UPLC (Method 04_A3_{—}1): RT=9.8 min,

MALDI-MS m/z: 4864.8, calculated MW=4866.5.

### Example 49

SEQ ID NO: 49: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Glu18,Glu22,Lys30]hPYY(3-36)

Synthesis method: Synthesis Method 2

UPLC (Method 07_B4_{—}1): RT=8.9 min,

UPLC (Method 04_A3_{—}1): RT=9.0 min,

MALDI-MS m/z: 4881.3, calculated MW=4881.5.

### Example 50

SEQ ID NO: 50: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,D-Asp18,Lys30]hPYY(3-36)

Synthesis method: Synthesis Method 2

UPLC (Method 07_B4_{—}1): RT=7.3 min,

UPLC (Method 04_A4_{—}1): RT=7.3 min,

MALDI-MS m/z: 4810, calculated MW=4810.4.

### Example 51

SEQ ID NO: 51: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Asp18,Lys30]hPYY(3-36)

Synthesis method: Synthesis Method 2

UPLC (Method 07_B4_{—}1): RT=9.1 min,

UPLC (Method 04_A7_{—}1): RT=6.6 min,

MALDI-MS m/z: 4780, calculated MW=4781.4.

### Example 52

SEQ ID NO: 52: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala14,Lys30]hPYY(3-36)

Synthesis method: Synthesis Method 2

MALDI-MS m/z: 4753, calculated MW=4754.4.

### Example 53

SEQ ID NO: 53: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala18,Lys30]hPYY(3-36)

Synthesis method: Synthesis Method 2

UPLC (Method 07_B4_{—}1): RT=9.0 min,

UPLC (Method 04_A7_{—}1): RT=7.0 min,

MALDI-MS m/z: 4736, calculated MW=4737.4.

### Example 54

SEQ ID NO: 54: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Val4,Lys30]hPYY(3-36)

Synthesis method: Synthesis Method 2

UPLC (Method 07_B4_{—}1): RT=7.6 min,

UPLC (Method 04_A4_{—}1): RT=5.1 min,

MALDI-MS m/z: 4751, calculated MW=4751.4.

### Example 55

SEQ ID NO: 55: N-epsilon30[2-(2-{2-[2-(2-{2-[(S)-4-Carboxy-4-(19-carboxynonadecanoylamino)butyrylamino]ethoxy}ethoxy)acetylamino]ethoxy}ethoxy)acetyl][Lys30]hPYY(3-36)

Synthesis method: Synthesis Method 2

UPLC (Method 07_B4_{—}1): RT=9.2 min,

MALDI-MS m/z: 4806.5, calculated MW=4808.5.

### Example 56

SEQ ID NO: 56: N-epsilon30[2-(2-{2-[2-(2-{2-(19-carboxynonadecanoylamino)ethoxy}ethoxy)acetylamino]ethoxy}ethoxy)acetyl][Lys30]hPYY(3-36)

Synthesis method: Synthesis Method 2

UPLC (Method 07_B4_{—}1): RT=9.3 min,

MALDI-MS m/z: 4677.2, calculated MW=4679.4.

### Example 57

SEQ ID NO: 57: N-epsilon30[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)-acetylamino]ethoxy}ethoxy)acetyl][Lys30]hPYY(3-36)

Synthesis method: Synthesis Method 2

UPLC (Method 07_B4_{—}1): RT=9.7 min,

MALDI-MS m/z: 4945.3, calculated MW=4947.7.

### Example 58

SEQ ID NO: 58: N-epsilon30[2-(2-{2-[2-(2-{2-(17-carboxyheptadecanoylamino)ethoxy}ethoxy)-acetylamino]ethoxy}ethoxy)acetyl][Lys30]hPYY(3-36)

Synthesis method: Synthesis Method 2

UPLC (Method 07_B4_{—}1): RT=8.8 min,

MALDI-MS m/z: 4649.2, calculated MW=4651.3.

### Example 59

SEQ ID NO: 59: N-epsilon30[2-(2-{2-[2-(2-{2-[(S)-4-Carboxy-4-(15-carboxypentadecanoylamino)butyrylamino]ethoxy}ethoxy)acetylamino]ethoxy}ethoxy)acetyl][Lys30]hPYY(3-36)

Synthesis method: Synthesis Method 2

UPLC (Method 07_B4_{—}1): RT=8.1 min,

MALDI-MS m/z: 4750.9, calculated MW=4752.4.

### Example 60

SEQ ID NO: 60: N-epsilon30[2-(2-{2-[2-(2-{2-(15-carboxypentadecanoylamino)ethoxy}ethoxy)-acetylamino]ethoxy}ethoxy)acetyl][Lys30]hPYY(3-36)

Synthesis method: Synthesis Method 2

UPLC (Method 07_B4_{—}1): RT=8.2 min,

MALDI-MS m/z: 4621.3, calculated MW=4623.3.

### Example 61

SEQ ID NO: 61: N-epsilon30[2-(2-{2-[2-(2-{2-[(S)-4-Carboxy-4-(13-carboxytridecanoylamino)butyrylamino]ethoxy}ethoxy)acetylamino]ethoxy}ethoxy)acetyl][Lys30]hPYY(3-36)

Synthesis method: Synthesis Method 2

UPLC (Method 07_B4_{—}1): RT=7.6 min,

MALDI-MS m/z: 4722.6 calculated MW=4724.3.

### Example 62

SEQ ID NO: 62: N-epsilon30[2-(2-{2-[2-(2-{2-(13-carboxytridecanoylamino)ethoxy}ethoxy)-acetylamino]ethoxy}ethoxy)acetyl][Lys30]hPYY(3-36)

Synthesis method: Synthesis Method 2

UPLC (Method 07_B4_{—}1): RT=7.8 min,

MALDI-MS m/z: 4593.3 calculated MW=4595.2.

### Example 63

SEQ ID NO: 63: N-epsilon30[2-(2-{2-[2-(2-{2-[(S)-4-Carboxy-4-(11-carboxyundecanoylamino)butyrylamino]ethoxy}ethoxy)acetylamino]ethoxy}ethoxy)acetyl][Lys30]hPYY(3-36)

Synthesis method: Synthesis Method 2

UPLC (Method 07_B4_{—}1): RT=7.3 min,

MALDI-MS m/z: 4694.9 calculated MW=4696.3.

### Example 64

SEQ ID NO: 64: N-epsilon30[2-(2-{2-[2-(2-{2-(11-carboxyundecanoylamino)ethoxy}ethoxy)-acetylamino]ethoxy}ethoxy)acetyl][Lys30]hPYY(3-36)

Synthesis method: Synthesis Method 2

UPLC (Method 07_B4_{—}1): RT=7.4 min,

MALDI-MS m/z: 4564.9 calculated MW=4567.2.

### Example 65

SEQ ID NO: 65: N-epsilon30[2-(2-{2-[2-(2-{2-[(4-(16-(1H-Tetrazol-5-yl)hexadecanoylsulfamoyl)butyryl]-ethoxy}ethoxy)acetylamino]ethoxy}ethoxy)acetyl][Lys30]hPYY(3-36)

Synthesis method: Synthesis Method 2

MALDI-MS m/z: 4808.4 ND calculated MW=4810.5.

### Example 66

SEQ ID NO: 66: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-Asp18,Lys30]hPYY(3-36)

Synthesis method: Synthesis Method 2

UPLC (Method 07_B4_{—}1): RT=7.3 min,

UPLC (Method 04_A4_{—}1): RT=5.5 min,

MALDI-MS m/z: 4781, calculated MW=4781.4.

### Example 67

SEQ ID NO: 67: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-acetyl,Lys30]hPYY(5-36)

Synthesis method: Synthesis Method 2

UPLC (Method 07_B4_{—}1): RT=8.7 min,

UPLC (Method 04_A3_{—}1): RT=9.4 min,

MALDI-MS m/z: 4582, calculated MW=4581.2.

### Example 68

SEQ ID NO: 68: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-acetyl,Ala4,Asp18,Lys30]hPYY(3-36)

Synthesis method: Synthesis Method 2

UPLC (Method 07_B4_{—}1): RT=7.8 min,

MALDI-MS m/z: 4748.4, calculated MW=4766.4.

### Example 69

SEQ ID NO: 69: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-acetyl,Arg4, Lys30]hPYY(3-36)

Synthesis method: Synthesis Method 2

UPLC (Method 07_B4_{—}1): RT=7.7 min,

UPLC (Method 04_A6_{—}1): RT=5.4 min,

MALDI-MS m/z: 4849, calculated MW=4850.5.

### Example 70

SEQ ID NO: 70: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(9-36)

Synthesis method: Synthesis Method 2

UPLC (Method 07_B4_{—}1): RT=7.4 min,

UPLC (Method 04_A6_{—}1): RT=5.5 min,

MALDI-MS m/z: 4144, calculated MW=4166.7

### Example 71

SEQ ID NO: 71: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Gly4, Lys30]hPYY(3-36)

Synthesis method: Synthesis Method 2

MALDI-MS m/z: 4709.2 calculated MW=4709.3.

### Example 72

SEQ ID NO: 72: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-acetyl,Arg4,Asp18,Lys30]hPYY(4-36)

Synthesis method: Synthesis Method 2

UPLC (Method 07_B4_{—}1): RT=10.3 min,

UPLC (Method 04_A6_{—}1): RT=5.4 min,

MALDI-MS m/z: 4735, calculated MW=4738.3.

### Example 73

SEQ ID NO: 73: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-succinyl,Arg4,Asp18,Lys30]hPYY(3-36)

Synthesis method: Synthesis Method 2

UPLC (Method 07_B4_{—}1): RT=10.3 min,

UPLC (Method 04_A6_{—}1): RT=5.3 min,

MALDI-MS m/z: 4793, calculated MW=4796.4

### Example 74

SEQ ID NO: 74: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-isovaleryl,Arg4, Lys30]hPYY(4-36

Synthesis method: Synthesis Method 2

UPLC (Method 07_B4_{—}1): RT=7.5 min,

UPLC (Method 04_A6_{—}1): RT=5.5 min,

MALDI-MS m/z: 4778, calculated MW=4779.4

### Example 75

SEQ ID NO: 75: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30,4-fluorophenylalanine36]hPYY(3-36)

Synthesis method: Synthesis Method 2

UPLC (Method 07_B4_{—}1): RT=7.6 min,

UPLC (Method 04_A6_{—}1): RT=6.4 min,

MALDI-MS m/z: 4780.6, calculated MW=4782.4.

### Example 76

SEQ ID NO: 76: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30,4-pyridylalanine-36]hPYY(3-36)

Synthesis method: Synthesis Method 2

UPLC (Method 07_B4_{—}1): RT=8.7 min,

MALDI-MS m/z: 4764, calculated MW=4838.5.

### Example 77

SEQ ID NO: 77: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu19,Arg22,Lys30]hPYY(3-36)

Synthesis method: Synthesis Method 2

UPLC (Method 07_B4_{—}1): RT=10.9 min,

UPLC (Method 04_A6_{—}1): RT=5.3 min,

MALDI-MS m/z: 4836.4, calculated MW=4838.5.

### Example 78

SEQ ID NO: 78: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-acetyl,D-Ile3,Arg4,Lys30]hPYY(3-36)

Synthesis method: Synthesis Method 2

UPLC (Method 07_B4_{—}1): RT=7.5 min,

UPLC (Method 04_A6_{—}1): RT=6.3 min,

MALDI-MS m/z: 4850.4, calculated MW=4850.5.

### Example 79

SEQ ID NO: 79: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-acetyl,Gly4,Lys30]hPYY(3-36)

Synthesis method: Synthesis Method 2

UPLC (Method 04_A6_{—}1): RT=5.6 min,

MALDI-MS m/z: 4748, calculated MW=4751.4

### Example 80

SEQ ID NO: 80: N-epsilon30[2-(2-{2-[2-(2-{2-((S)-4-carboxy-[(S)-4-Carboxy-4-(17-carboxyheptadecanoylamino)butyrylamino]butyrylamino)ethoxy}ethoxy)acetylamino]ethoxy}ethoxy)acetyl][N-alpha-acetyl,Arg4,Lys30]hPYY(3-36)

Synthesis method: Synthesis Method 2

UPLC (Method 07_B4_{—}1): RT=7.9 min,

UPLC (Method 04_A6_{—}1): RT=5.1 min,

MALDI-MS m/z: 4978.1 calculated MW=4979.6.

### Example 81

SEQ ID NO: 81: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-acetyl,Arg4,Asp18,Lys30]hPYY(3-36)

Synthesis method: Synthesis Method 2

UPLC (Method 07_B4_{—}1): RT=7.5 min,

UPLC (Method 04_A6_{—}1): RT=5.4 min,

MALDI-MS m/z: 4850 calculated MW=4851.5

### Example 82

SEQ ID NO: 82: N-epsilon30[2-(2-{2-[2-(2-{2-[(S)-4-Carboxy-4-(15-carboxypentadecanoylamino)butyrylamino]ethoxy}ethoxy)acetylamino]ethoxy}ethoxy)acetyl][N-alpha-acetyl,Lys30]hPYY(3-36)

Synthesis method: Synthesis Method 2

UPLC (Method 07_B4_{—}1): RT=7.2 min,

UPLC (Method 04_A6_{—}1): RT=4.7 min,

MALDI-MS m/z: 4793 calculated MW=4794.4

### Example 83

SEQ ID NO: 83: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala4,Asp18,Lys30]hPYY(3-36)

Synthesis method: Synthesis Method 2

UPLC (Method 07_B4_{—}1): RT=10.1 min,

UPLC (Method 04_A6_{—}1): RT=5.1 min,

MALDI-MS m/z: 4723 calculated MW=4724.3.

### Example 84

SEQ ID NO: 84: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Asp18,Lys30]hPYY(3-36)

Synthesis method: Synthesis Method 2

UPLC (Method 07_B4_{—}1): RT=9.9 min,

UPLC (Method 04_A6_{—}1): RT=5.1 min,

MALDI-MS m/z: 4810 calculated MW=4809.5.

### Example 85

SEQ ID NO: 85: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-Arg4, Lys30]hPYY(3-36)

Synthesis method: Synthesis Method 2

UPLC (Method 07_B4_{—}1): RT=7.5 min,

UPLC (Method 04_A6_{—}1): RT=6.2 min,

MALDI-MS m/z: 4811 calculated MW=4808.5.

### Example 86

SEQ ID NO: 86: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-allo-Ile3,Arg4, Lys30]hPYY(3-36)

Synthesis method: Synthesis Method 2

UPLC (Method 07_B4_{—}1): RT=7.6 min,

UPLC (Method 04_A6_{—}1): RT=5.4 min,

MALDI-MS m/z: 4805 calculated MW=4808.5.

### Example 87

SEQ ID NO: 87: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-Ala4, Lys30]hPYY(3-36)

Synthesis method: Synthesis Method 2

UPLC (Method 07_B4_{—}1): RT=8.0 min,

UPLC (Method 04_A6_{—}1): RT=5.3 min,

MALDI-MS m/z: 4724 calculated MW=4723.4.

### Example 88

SEQ ID NO: 88: N-epsilon30-[(S)-4-Carboxy-4-(17-carboxyheptadecanoylamino)butyrylamino][Lys30]hPYY(3-36)

Synthesis method: Synthesis Method 2

UPLC (Method 07_B4_{—}1): RT=9.0 min,

UPLC (Method 04_A6_{—}1): RT=12.6 min,

MALDI-MS m/z: 4490 calculated MW=4490.1.

### Example 89

SEQ ID NO: 89: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][N-alpha-acetyl,Ala4,Lys30]hPYY(3-36)

Synthesis method: Synthesis Method 2

UPLC (Method 04_A6_{—}1): RT=5.6 min,

MALDI-MS m/z: 4764 calculated MW=4765.4.

### Example 90

SEQ ID NO: 90: N-epsilon30[{(S)-4-Carboxy-4-(17-carboxyheptadecanoylamino)butyrylamino}-Ser-Ser-Gly-Ser-Ser-Gly][Arg4,Lys30]hPYY(3-36)

Synthesis method: Synthesis Method 2

UPLC (Method 07_B4_{—}1): RT=7.2 min,

UPLC (Method 04_A6_{—}1): RT=5.2 min,

MALDI-MS m/z: 4980 calculated MW=4980.6.

### Example 91

SEQ ID NO: 91: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu8,Lys30]hPYY(3-3 6)

Synthesis method: Synthesis Method 2

UPLC (Method 07_B4_{—}1): RT=7.2 min,

UPLC (Method 04_A6_{—}1): RT=5.7 min,

MALDI-MS m/z: 4812 calculated MW=4812.5

### Example 92

SEQ ID NO: 92: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu9,Lys30]hPYY(3-36)

Synthesis method: Synthesis Method 2

UPLC (Method 07_B4_{—}1): RT=7.2 min,

UPLC (Method 04_A6_{—}1): RT=5.7 min,

MALDI-MS m/z: 4853.7 calculated MW=4852.5.

### Example 93

SEQ ID NO: 93: N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala4,Lys30]hPYY(3-36)

Synthesis method: Synthesis Method 2

UPLC (Method 07_B4_{—}1): RT=7.4 min,

UPLC (Method 04_A6_{—}1): RT=5.7 min,

MALDI-MS m/z: 4724.7 calculated MW=4723.4.

### Biological Assays

The utility of the PYY derivatives thereof of the present invention as pharmaceutically active agents in the reduction of weight gain and treatment of obesity in mammals (such as humans), may be demonstrated by the activity of the agonists in conventional assays and in the in vitro and in vivo assays described below. Such assays also provide a means whereby the activities of the PYY derivatives can be compared with the activities of known PYY compounds, such as hPYY(1-36) or hPYY(3-36).

### Assay (I)—Y2 Receptor ACTOne Potency Assay

This assay provides a method for determination of in vitro effect of peptides on the Y2 receptor activity using the ACTOne based FLIPR assay. ACTOne™ is an easily scaleable cAMP biosensor HTS platform for measurement of Gs and Gi coupled 7™ receptor signalling from BD Biosciences (San Jose, Calif.). The cells express a biosensor developed around a modified rat olfactory cyclic nucleotide gated (CNG) calcium channel—a fairly non-discriminatory ion channel that responds to cAMP and cGMP. The CNG has been engineered to be cAMP selective and thus function as a cAMP responsive biosensor that signals through calcium or membrane potential responsive dyes. ACTOne HEK-293 cells expressing the Y2 receptor were obtained from BD Biosciences. The cells were loaded with a calcium responsive dye that only distributes in the cytoplasm. Probenecid, an inhibitor of the organic anion transporter was added to prevent the dye from leaving the cell. A phosphodiesterase inhibitor was added to prevent formatted cAMP from being degraded. Isoproterenol (a β1/β2 agonist) was added to activate the adenylate cyclase. When an Y2 receptor agonist was added, the adenylate cyclase was inactivated. The decreased calcium concentration in the cytoplasm was then detected as a decrease in fluorescence. Together with the test substance, isoproterenol at a concentration matching EC₈₀ was added to all wells. The assay was carried out as follows: The cells were plated out in Greiner 384-well plates. 25 μl cell suspension containing 560 cells per μl were added to each well using the Multidrop™ (384-Multidrop from Labsystems, Finland). The cell plates were then incubated in the incubator over night at 37° C. with 5% CO₂ in stacks of up to 9 plates. The cell plates were loaded with 25 μl probe from the FLIPR calcium4 kit (Molecular Devices, CA, USA) using the Multidrop™. The cell plates were returned to the incubator and incubated for 60 min at 37° C. in stacks of up to 9 plates. The cell plates were then left at room temperature for 60 min before use, without stacking the plates. The plates were covered with tinfoil to avoid light (the dye can be excited by the daylight, which results in higher baseline and variation). The FLIPR (FLIPRtetra from Molecular Devices, CA, USA) added 1 μl sample and 1 μl isoproterenol (0.05 μM final concentration) at the same time. The fluorescence signal from the wells was measured 330 seconds after sample addition on the FLIPR. The EC50 was calculated as the concentration of the Y2 receptor agonist inducing 50% decrease in fluorescence signal. A reported value of 1000 nM is intended to mean at least 1000 nM as this is the detection limit of the assay.

### Assay (II)—Y1 Receptor ACTOne Potency Assay

This assay provides a method for determination of in vitro effect of peptides on the Y1 receptor activity using the ACTOne based FLIPR assay. The assay was carried out as described for Assay (I) except that ACTOne HEK-293 cells expressing the Y1 receptor was used. A reported value of 1000 nM is intended to mean at least 1000 nM as this is the detection limit of the assay.

Assay (III)—Y5 Receptor IPOne Potency Assay The IPOne-Tb assay (Cisbio, Bagnols-sur-Cèze Cedex, France) is a homogeneous time resolved fluorescence (HTRF) assay which functions as a competitive immunoassay that measures IP1 levels using cryptate labelled anti-IP1 monoclonal antibody and d2 labelled IP1, wherein IP1 is accumulated following activation of seven transmembrane receptors that couples to the Gq pathway. In the hY5 IPOne assay a HEK293 cell line stably expressing both the human Y5 receptor and the chimeric G-protein Gqi5 was used where Gqi5 ensures Gq signalling of the Gi coupled Y5 receptor. The buffers and reagents for the assay were supplied with the IPOne-Tb kit (Cisbio, Bagnols-sur-Cèze Cedex, France). The assay was carried out as follows: on the day before the assay cells were seeded at a density of 40,000 cells/well in 20 μl in 384-well small volume white tissue culture plates, Greiner #784080, and incubated overnight at 37° C. with 5% CO₂. On the day of the assay the media was removed and 10 μl stimulation buffer supplemented with 0.005% Tween-20 was added together with 5 μl agonist serial dilution. The plates were then incubated for 1 hour at 37° C. IP1-d2 and IP1-cryptate is reconstituted in lysis buffer according to the IPOne-Tb kit protocol. 3 μl of each of the IP1-d2 and IP1-cryptate working solutions was added to each well. The plate was incubated for 1 hour at room temperature. The plate was read on a Mithras LB 940 HTRF compatible reader (Berthold Technologies, Bad Wildbad, Germany) with 665 nm and 620 nm emission filters and the signal was calculated as the fluorescence ratio 665 nm/620 nm. A reported value of 1000 nM is intended to mean at least 1000 nM as this is the detection limit of the assay.

### Assay (IV)—PK I.V. Minipig

An assay useful for measuring the pharmacokinetic (PK) profile of the PYY derivative is the following mini-pig PK assay.

Twelve male Göttingen mini-pigs weighing approximately 7 to 10 kg from Ellegaard Göttingen Minipigs A/S, Denmark were comprised in the study. The mini-pigs were dosed intra venous (i.v.) via a Venflon inserted in the ear vein. Blood was sampled from jugular vein. Test substances were dissolved in a vehicle consisting of 10 mM NaHPO4, 150 mM NaCl, 0.01% Tween80, pH 4.0 or pH 7.4 in various concentrations. For comparison a control compound, such as human PYY(1-36) or human PYY(3-36), may be administered. The pigs were usually dosed with 15 nmol test compound/kg body weight. Blood samples were taken at the following time points: pre-dose, 5 minutes, 30 minutes, 1, 2, 4, 7, 11, 24, 48, 72, 96, 120, 168, 216 and 264 hours post dosing. The blood samples were collected into test tubes containing EDTA buffer for stabilization and kept on ice for max. 20 minutes before centrifugation. The centrifugation procedure to separate plasma may be: 4° C., approx. 2500 g for 10 minutes. Plasma was collected and immediately transferred to Micronic tubes stored at −20° C. until assayed.

Quantitative Assay for Plasma Samples

The test substances were assayed in plasma by Turbulent Flow Chromatography coupled to Liquid Chromatography with subsequent Mass Spectrometric Detection (TFC/LC/MS). The selectivity of the method allows various compounds to be quantitated in one sample, e.g. cassette dosing of four compounds per animal.

The concentrations of the test substance in unknown samples were calculated using the peak area as a function of amount. Calibration graphs based on plasma samples spiked with the analyte were constructed by regression analysis. Typical dynamic range for standard assay was 1-2,000 nmol/l. The method performance was assured by co-assaying quality control (QC) samples in duplicate at three concentration levels.

Stock and working solutions of analytes were prepared in plasma and incubated by 37° C. for 1 hour.

Sample Preparation:

40.0 μl EDTA-plasma was added 160 μl 50% methanol, 1% formic acid, then vortexed and centrifuged at 14300 rpm (16457 g) at 4° C. for 20 minutes. The supernatant was transferred to a 96 well plate, then plates were incubated with 0.4% BSA, 37° C. for ½ hour. Injection volume was 25 μl.

For sample clean up a TurboFlow C8 (0.5×50 mm) or a Cyclone column (0.5×50 mm) both from Thermo Scientific, Franklin, Mass., USA, was used and the LC separation was done either on a Proteo 4 μm column (2.0×50 mm) or a Onyx C18 column (2.0×50 mm) both from Phenomenex, Torrance, Calif., USA. Eluents were isocratic and gradient combinations of methanol, acetonitril, Milli-Q water and formic acid. Selective detection was done by mass spectrometry operated in positive mode ionisation.

Non-compartmental analysis (NCA): Plasma concentration-time profiles were analyzed by non-compartmental pharmacokinetics analysis (NCA) using WinNonlin Professional 5.0 (Pharsight Inc., Mountain View, Calif., USA). NCA was performed using the individual plasma concentration-time profiles from each animal.

### Assay (V)—Mean Cumulative Food Intake

Mean cumulative food intake after single dosing in lean mice was determined in BioDaq system for automatic monitoring of food intake. 32 lean C57Bl6J mice, 8 weeks old, were used in the study. The mice were housed two per cage but with a dividing wall containing holes to separate the mice. They were housed in reversed daily rhythm and with ad libitum access to food and water. The mice were acclimatized to the system for two weeks before start of the study. The system consisted of 32 individual boxes with a scale connected to each box which automatically registered the food available to the mice. Each time the mouse ate the weight reduction of food was registered and data collected on a computer. Data was collected continuously. The collected data was analysed using GraphPadPrism software.

Before start of the study the mice were fasted for approx. 18 hours. The mice were treated s.c. (10 ml/kg) with analogues dissolved in 50 mM Na₂HPO₄, 0.145M NaCl, 0.05% Tween 80, pH 7.4. Treatment took place 30 minutes before lights go out. Data was collected up to 48 hours after dosing.

Assay (VI)—Determination of Effect on Body Weight and/or Body Composition

Additional assays useful to the invention comprise those that can determine the effect of PYY derivatives on body weight and/or body composition. An exemplary assay is the following which involves utilization of an ob/ob mouse model for metabolic disease: ob/ob mice (Taconic, Denmark) on regular diurnal rhythm and with access to a regular diet (Altromin 1324, Brogaarden, Denmark) are used. The mice were weighed on a weekly basis. Mice were used in the study when they have reached a body weight of at least 40 gram. If performing analysis of body composition then all mice are scanned for body composition (NMR scan) before starting the study. One week before starting the study the mice were weighed daily to get a stable baseline and to acclimatize them to the procedure. The mice were divided into one group (n=10) receiving s.c. dosing of vehicle and groups of n=10 animals receiving PYY derivatives by s.c. dosing of different doses or dosing intervals. At least one PYY derivative and optionally at least one control compound, such as hPYY(1-36) or hPYY(3-36), were dissolved in 50 mM NaH₂PO₄, 145 mM NaCl, pH=7.4. Dosing was performed once daily at the same time point every day, shortly before lights off. As an alternative to s.c. administration some or all of the PYY derivatives can be delivered via Alzet osmotic minipumps. The pumps can deliver PYY derivatives at, e.g., 1 μmol/kg/24 hours. The mice are dosed for 3 weeks. Body weight for all mice is recorded daily in combination with dosing. If performing analysis of body composition then after 1 week and 3 weeks of treatment, the mice are scanned for body composition using a QNMR system (Echo Medical Systems, Houston, Tex.). Thereafter the mice are euthanized with cervical dislocation. Data are analysed in Graph Pad Prism. Statistical significance is assessed by comparing the groups with ANOVA followed by Tukey's post-hoc test. A p-value <0.05 is considered statistically significant.

### Assay (VII)—Food Intake in Pigs

Female slaughter pigs, 3 months of age, weighing approx. 40 kg were used, n=4

The animals were housed in a group for 1-2 weeks during acclimatisation to the facilities. The animals were fed ad libitum with pig fodder (Svinefoder Antonio) at all times both during the acclimatisation and the experimental period. During the experimental period the animals were placed in individual pens from Monday morning to Friday afternoon for measurement of individual food intake. Food intake was monitored on line by logging the weight of fodder every minutes. The system used for this was Mpigwin, developed by Soren Ellegaard. Body weight was measured in all animals on Monday and Friday morning. The PYY derivatives were tested in 1 dose (100 nmol/kg). Animals were dosed with a single subcutaneous dose of PYY derivatives or vehicle Monday morning, and food intake was measured for 4 days after dosing. On Friday morning, 4 days after dosing, a blood sample for measurement of exposure was taken from the heart in anaesthetized animals; the animals were thereafter euthanized. Data are analysed in Graph Pad Prism. Statistical significance is assessed by comparing the groups with Two-way ANOVA followed by Bonferroni post test. A p-value <0.05 is considered statistically significant.

### Assay (VIII): Measurement of Gastric Emptying

An exemplary assay for measurement of gastric emptying is described in the materials and methods section page 1326 under the headline “Gastric emptying” in Asakawa A et al., Characterization of the effects of pancreatic polypeptide in the regulation of energy balance, Gastroenterology, 2003, 124, 1325-1336.

### Assay (IX): Measurement of Appetite

Appetite can be measured by any means known to one of skill in the art. For example, in humans, decreased appetite can be assessed by a psychological assessment. In such an embodiment, administration of the receptor agonist results in a change in perceived hunger, satiety, and/or fullness. Hunger can be assessed by any means known to one of skill in the art. In one embodiment hunger is assessed using psychological assays, such as by an assessment of hunger feelings and sensory perception using e.g. a questionnaire.

### Example 94

### Y2, Y1 and Y5 receptor potency (Assay (I), (II) and (III))

The potency of a number of PYY derivatives for the Y2, Y1 or Y5 receptors was determined according to Assay (I), Assay (II) and Assay (III), respectively. The Y1/Y2 and Y5/Y2 receptor potency ratios were calculated based on the Y1 and Y2, and Y5 and

Y2 receptor potencies, respectively. For comparison, the potency of hPYY(1-36) and hPYY(3-36) was also determined. The results are shown in Table 2.

TABLE 2
Potency of PYY derivatives, hPYY(1-36) and hPYY(3-36).

| Compound | | Receptor potency | | | | |
|---|---|---|---|---|---|---|
| (SEQ ID | Position of | Y2 | Y1 | Y1/Y2 | Y5 | Y5/Y2 |
| NO) | acylation | (nM) | (nM) | ratio | (nM) | ratio |
| hPYY(1-36) | — | 0.39 | 0.06 | 0.15 | 1.07 | 2.7 |
| hPYY(3-36) | — | 0.91 | 20 | 22 | 8.89 | 9.8 |
| 4 | 32K | 56.3 | 1000 | >17.8 | 1000 | >17.8 |
| 5 | 31K | 82 | 329 | 4.0 | 54.6 | 0.7 |
| 6 | 30K | 6.3 | 500 | 79.4 | 252 | 40.0 |
| 7 | 29K | 432 | 321 | 0.74 | 1000 | >2.3 |
| 8 | 28K | 33.5 | 780 | 23.3 | 250 | 7.5 |
| 9 | 27K | 36.7 | 345 | 9.4 | 51 | 1.4 |
| 10 | 26K | 39.7 | 337 | 8.5 | 147 | 3.7 |
| 11 | 25K | 200 | 421 | 2.1 | 800 | 4 |
| 12 | 24K | 53 | 371 | 7 | 190 | 3.6 |
| 13 | 23K | 13.7 | 70 | 5.1 | 47.5 | 3.5 |
| 14 | 22K | 3.2 | 44 | 13.8 | 7.6 | 2.4 |
| 15 | 21K | 4.8 | 120 | 25 | 78.4 | 16.3 |
| 16 | 20K | 23.8 | 740 | 31.1 | 267 | 11.2 |
| 17 | 19K | 3.3 | 55 | 16.7 | 91 | 27.5 |
| 18 | 18K | 8.1 | 61 | 7.5 | 27.7 | 3.4 |
| 19 | 17K | 5.8 | 111 | 19.1 | 99.7 | 17.2 |
| 20 | 16K | 21.5 | 180 | 8.4 | 390 | 18.1 |
| 21 | 15K | 10.1 | 63 | 6.2 | 84.3 | 8.3 |
| 22 | 14K | 5.0 | 71 | 14.2 | 63.8 | 12.7 |
| 23 | 13K | 32.7 | 291 | 8.9 | 1000 | >30.6 |
| 24 | 12K | 17 | 212 | 12.5 | 163 | 9.6 |
| 25 | 11K | 5.25 | 24 | 4.6 | 103 | 19.6 |
| 26 | 10K | 1.5 | 59 | 39 | 25.3 | 16.9 |
| 27 | 09K | 11.9 | 202 | 17.0 | 90 | 7.6 |
| 28 | 08K | 41.7 | 251 | 6.0 | 1000 | >24.0 |
| 29 | 07K | 3.1 | 71 | 22.9 | 16.7 | 5.4 |
| 30 | 06K | 7.8 | 53 | 6.8 | 32.7 | 4.2 |
| 31 | 05K | 27 | 99 | 3.7 | 22 | 0.8 |
| 32 | 04K | 27.5 | 198 | 7.2 | 16 | 0.6 |
| 33 | 03K | 9.2 | 133 | 14.5 | 19 | 2.1 |
| 34 | N-alpha | 11.9 | 27 | 2.3 | 19 | 1.6 |
| 35 | 30K | 3.55 | 365 | 103 | 300 | 84.5 |
| 36 | 30K | 12 | 356 | 29.7 | 436.5 | >36 |
| 37 | 30K | 12 | 729 | 60.8 | 1000 | >83 |
| 38 | 30K | 34 | 717 | 21.1 | 1000 | >29 |
| 39 | 30K | 274 | 1000 | >3.6 | 1000 | >3.6 |
| 40 | 30K | 0.4 | 6.7 | 17 | 0.2 | 0.5 |
| 41 | 30K | 2.7 | 326 | 120 | 180 | 67 |
| 42 | 1K | 12.7 | 5.2 | 0.41 | 2.3 | 0.18 |
| 43 | 30K | 7.4 | 334 | 45 | 1000 | >140 |
| 44 | 30K | 6 | 365 | 61 | 1000 | >170 |
| 45 | 30K | 836 | 560 | 0.67 | 1000 | >1.2 |
| 46 | 30K | 30 | 354 | 12 | 1000 | >33 |
| 47 | 30K | 61 | 393 | 6.4 | 1000 | >16 |
| 48 | 30K | 20.5 | 448 | 22 | 399 | 16 |
| 49 | 30K | 22 | 378 | 17 | 1000 | >46 |
| 50 | 30K | 4.8 | 278 | 58 | 1000 | >210 |
| 51 | 30K | 2.7 | 252 | 93 | >100 | >37 |
| 52 | 30K | 2.1 | 426 | 200 | >100 | >47 |
| 53 | 30K | 2.1 | 624 | 297 | 75 | 36 |
| 54 | 30K | 4.8 | 312 | 65 | 240 | 50 |
| 55 | 30K | 6.9 | 475 | 69 | 12 | 1.7 |
| 56 | 30K | 10 | 604 | 60 | 9 | 0.9 |
| 57 | 30K | 3.5 | 314 | 90 | 13 | 3.7 |
| 58 | 30K | 2.4 | 608 | 250 | 33 | 14 |
| 59 | 30K | 8.3 | 1000 | >120 | 213 | 26 |
| 60 | 30K | 5.1 | 1000 | >200 | 41 | 8.0 |
| 61 | 30K | 10.5 | 1000 | >95 | 286 | 27 |
| 62 | 30K | 7.1 | 1000 | >140 | 41 | >5.8 |
| 63 | 30K | 10 | 925 | 93 | 220 | 22 |
| 64 | 30K | 7.2 | 708 | 98 | 125 | 17 |
| 66 | 30K | 7.8 | 378 | 49 | 1000 | >130 |
| 67 | 30K | 13 | 347 | 27 | 1000 | >77 |
| 68 | 30K | 18 | 1000 | >56 | ND | ND |
| 69 | 30K | 11.9 | 355 | 30 | 355 | 30 |
| 70 | 30K | 18 | 289 | 16 | 1000 | >56 |
| 71 | 30K | 11 | 950 | 86 | 404 | 37 |
| 72 | 30K | 9 | 398 | 44 | 268 | 30 |
| 73 | 30K | 18 | 352 | 20 | 300 | 17 |
| 74 | 30K | 31 | 377 | 12 | 314 | 10 |
| 75 | 30K | 9.7 | 328 | 34 | 34 | 3.5 |
| 76 | 30K | 80 | 347 | 4.3 | 1000 | >13 |
| 77 | 30K | 5.8 | 864 | 150 | 114 | 20 |
| 78 | 30K | 6.8 | 1000 | >150 | 123 | 18 |
| 79 | 30K | 47 | 1000 | >21 | 295 | 6.3 |
| 80 | 30K | 18 | 1000 | >56 | 352 | 20 |
| 81 | 30K | 22 | 1000 | >45 | 112 | 5.1 |
| 82 | 30K | 29 | 1000 | >35 | 292 | 10 |
| 83 | 30K | 9.9 | 1000 | >100 | 143 | 14 |
| 84 | 30K | 5.2 | 1000 | >190 | 103 | 20 |
| 85 | 30K | 22 | 1000 | >45 | 140 | 6.4 |
| 86 | 30K | 7.7 | 780 | 100 | 104 | 14 |
| 87 | 30K | 7.2 | 1000 | >140 | 113 | 16 |
| 88 | 30K | 21 | 1000 | >48 | 102 | 4.9 |
| 89 | 30K | 17 | 1000 | >59 | 187 | 11 |
| 90 | 30K | 14 | 1000 | >71 | 103 | 7.4 |
| 91 | 30K | 37 | 1000 | >27 | 416 | 11 |
| 92 | 30K | 13 | 1000 | >77 | 165 | 13 |
| 93 | 30K | 18 | 1000 | >56 | 260 | 14 |

ND: Not determined.

### Example 95

### Plasma Half-Life (Assay (IV))

The plasma half-life in minipigs of a number of PYY derivatives was determined according to Assay (IV) as described herein. For comparison, the potency and half-life of hPYY(1-36) and hPYY(3-36) was also determined. All PYY derivatives were prepared by synthesis method 1. The results are shown in Table 3.

TABLE 3
Half-life (t½) of PYY derivatives determined in minipigs
according to Assay (IV).

| Compound | Acylation | | | Dose | t½ |
|---|---|---|---|---|---|
| (SEQ ID NO:) | position | RoA¹ | n | (nmol/kg) | (h) |
| 1 | 35 | i.v. | 3 | 18 | 67 |
| 2 | 34 | i.v. | 3 | 19 | 30 |
| 3 | 33 | i.v. | 3 | 20 | 56 |
| 4 | 32 | i.v. | 3 | 9.9 | 49 |
| 5 | 31 | i.v. | 3 | 18 | 75 |
| 6 | 30 | i.v. | 3 | 20 | 76 |
| | | i.v. | 2 | 50 | 61 |
| | | s.c. | 4 | 70 | 64 |
| 7 | 29 | i.v. | 3 | 18 | 39 |
| 8 | 28 | i.v. | 3 | 18 | 62 |
| 9 | 27 | i.v. | 3 | 20 | 52 |
| 10 | 26 | i.v. | 3 | 4.5 | 41 |
| 11 | 25 | i.v. | 3 | 19 | 55 |
| 12 | 24 | i.v. | 3 | 24 | 66 |
| 13 | 23 | i.v. | 3 | 18 | 19 |
| 14 | 22 | i.v. | 3 | 20 | 36 |
| 15 | 21 | i.v. | 3 | 20 | 34 |
| 16 | 20 | i.v. | 3 | 18 | 33 |
| 17 | 19 | i.v. | 3 | 24 | 29 |
| 18 | 18 | i.v. | 3 | 21 | 22 |
| 19 | 17 | i.v. | 3 | 18 | 39 |
| 20 | 16 | i.v. | 3 | 19 | 17 |
| 21 | 15 | i.v. | 3 | 18 | 8.8 |
| 22 | 14 | i.v. | 3 | 23 | 11 |
| 23 | 13 | i.v. | 3 | 24 | 11 |
| 24 | 12 | i.v. | 3 | 17 | 15 |
| 25 | 11 | i.v. | 3 | 17 | 11 |
| 26 | 10 | i.v. | 3 | 19 | 8.4 |
| 27 | 9 | i.v. | 3 | 24 | 12 |
| 28 | 8 | i.v. | 3 | 20 | 5.9 |
| 29 | 7 | i.v. | 3 | 26 | 11 |
| 30 | 6 | i.v. | 3 | 18 | 7.2 |
| 31 | 5 | i.v. | 3 | 16 | 8.8 |
| 32 | 4 | i.v. | 3 | 21 | 14 |
| 33 | 3 | n.a. | n.a. | n.a. | n.a. |
| 34 | N-alpha | i.v. | 2 | 50 | 16.7 |
| 35 | 30 | i.v. | 3 | 9 | 62 |
| | | s.c. | 3 | 21 | 59 |
| 36 | 30 | i.v. | 3 | 10 | 67.9 |
| 37 | 30 | i.v. | 4 | 10 | 77.5 |
| 38 | 30 | i.v. | 4 | 10 | 87.8 |
| 39 | 30 | n.a. | n.a. | n.a. | n.a. |
| 40 | 30 | i.v. | 3 | 15 | 0.35 |
| 41 | 30 | n.a. | n.a. | n.a | n.a. |
| 42 | 1 | n.a. | n.a. | n.a. | n.a. |
| 55 | 30 | i.v. | 2 | 15 | 98.9 |
| 56 | 30 | i.v. | 3 | 15 | 19.5 |
| 57 | 30 | i.v. | 3 | 15 | 64.0 |
| 58 | 30 | i.v. | 3 | 15 | 13 |
| 59 | 30 | i.v. | 3 | 15 | 28.2 |
| 60 | 30 | i.v. | 2 | 50 | 4.0 |
| 61 | 30 | i.v. | 3 | 15 | 4.3 |
| 62 | 30 | i.v. | 3 | 15 | 1.7 |
| 63 | 30 | i.v. | 3 | 15 | 1.1 |
| 64 | 30 | i.v. | 3 | 15 | 0.8 |
| 65 | 30 | i.v. | 3 | 15 | 65.3 |
| 68 | 30 | i.v. | 4 | 15 | 114.7 |
| 69 | 30 | i.v. | 4 | 15 | 119.7 |
| 77 | 30 | i.v. | 4 | 15 | 89.4 |
| 80 | 30 | i.v. | 4 | 15 | 102.0 |
| 81 | 30 | i.v. | 4 | 15 | 95.1 |
| 83 | 30 | i.v. | 4 | 15 | 85.4 |
| 84 | 30 | i.v. | 4 | 15 | 84.3 |
| 88 | 30 | i.v. | 4 | 15 | 97.4 |
| 89 | 30 | i.v. | 4 | 15 | 115.7 |
| 93 | 30 | i.v. | 4 | 15 | 84.1 |

1) RoA: route of administration. n.a.: not analysed.

### Example 96

### Mean Cumulative Food Intake (Assay (V))

Mean cumulative food intake were determined for a number of PYY derivatives according to Assay (V) as described herein. The results are shown in Table 4, 5 and 6.

TABLE 4
Mean cumulative food intake (n = 7-8 per group)

| | Mean cumulative food intake [g] | | |
|---|---|---|---|
| Time after | | SEQ ID NO: 34 | SEQ ID NO: 6 |
| injection | vehicle | 1 μmol/kg | 1 μmol/kg |
| 1 hour | 0.49 | 0.58 | 0.47 |
| 2 hours | 0.99 | 0.92 | 0.80 |
| 3 hours | 1.28 | 1.10 | 1.10 |
| 4 hours | 1.65 | 1.29 | 1.36 |
| 6 hours | 2.13 | 1.61* | 1.80 |
| 8 hours | 2.74 | 2.07* | 2.28 |
| 12 hours | 3.61 | 2.60** | 2.79* |
| 24 hours | 4.46 | 2.88*** | 3.11** |
| 36 hours | 8.51 | 6.41** | 6.65* |
| 48 hours | 9.23 | 6.88** | 6.93* |

*p < 0.05, **p < 0.01, ***p < 0.001 (ANOVA, Dunnetts post hoc)

TABLE 5
Mean cumulative food intake (n = 6-8 per group)

| | Mean cumulative food intake [g] | | | |
|---|---|---|---|---|
| Time after | | SEQ ID NO: 22 | SEQ ID NO: 21 | SEQ ID NO: 13 |
| injection | vehicle | 1.0 μmol/kg | 1.0 μmol/kg | 1.0 μmol/kg |
| 1 hour | 0.51 | 0.37 | 0.54 | 0.36 |
| 2 hours | 0.89 | 0.77 | 0.90 | 0.71 |
| 3 hours | 1.28 | 0.96 | 1.26 | 1.04 |
| 4 hours | 1.64 | 1.24 | 1.54 | 1.28 |
| 6 hours | 2.25 | 1.52* | 1.85 | 1.70 |
| 8 hours | 2.76 | 1.91* | 2.15 | 2.00* |
| 12 hours | 3.53 | 2.42*** | 2.82* | 2.49*** |
| 24 hours | 4.30 | 2.85*** | 3.37** | 3.07*** |
| 36 hours | 8.04 | 6.24*** | 7.17 | 6.76* |
| 48 hours | 8.43 | 7.02* | 7.88 | 7.72 |

*p < 0.05, **p < 0.01, ***p < 0.001 (ANOVA, Dunnetts post hoc)

TABLE 6
Mean cumulative food intake (n = 7-8 per group)

| | Mean cumulative food intake [g] | | |
|---|---|---|---|
| Time after | | SEQ ID NO: 6 | SEQ ID NO: 35 |
| injection | vehicle | 1 μmol/kg | 1 μmol/kg |
| 1 hour | 0.61 | 0.52 | 0.49 |
| 2 hours | 1.07 | 0.87 | 0.72** |
| 3 hours | 1.44 | 1.05* | 0.89** |
| 4 hours | 1.79 | 1.18** | 1.04*** |
| 6 hours | 2.29 | 1.48*** | 1.29*** |
| 8 hours | 2.67 | 1.81*** | 1.59*** |
| 12 hours | 3.50 | 2.35*** | 2.12*** |
| 24 hours | 4.11 | 2.93** | 2.86*** |
| 36 hours | 7.76 | 5.99* | 6.05* |
| 48 hours | 8.12 | 6.23* | 6.39* |

*p < 0.05, **p < 0.01, ***p < 0.001 (ANOVA, Dunnetts post hoc)

### Example 97

### Mean Cumulative Food Intake (Assay (V))

Mean cumulative food intake were determined for a number of PYY derivatives according to Assay (V) as described herein. The results are shown in Table 7-12.

TABLE 7
Mean cumulative food intake (n = 7-8 per group)

| | Mean cumulative food intake [g] | | |
|---|---|---|---|
| Time after | | SEQ ID NO: 52 | SEQ ID NO: 41 |
| injection | vehicle | 1 μmol/kg | 1 μmol/kg |
| 1 hour | 0.51 | 0.28** | 0.22*** |
| 2 hours | 0.97 | 0.52*** | 0.55*** |
| 3 hours | 1.19 | 0.80*** | 0.89** |
| 4 hours | 1.63 | 1.04*** | 1.10*** |
| 6 hours | 2.26 | 1.42*** | 1.49*** |
| 8 hours | 2.90 | 1.72*** | 1.95*** |
| 12 hours | 3.55 | 2.47* | 2.97 |
| 24 hours | 3.83 | 3.27 | 3.70 |
| 36 hours | 7.44 | 6.56 | 6.65 |
| 48 hours | 7.60 | 7.01 | 7.10 |

*p < 0.05, **p < 0.01, ***p < 0.001 (ANOVA, Dunnetts post hoc)

TABLE 8
Mean cumulative food intake (n = 7-8 per group)

| | Mean cumulative food intake [g] | | |
|---|---|---|---|
| Time after | | SEQ ID NO: 35 | SEQ ID NO: 51 |
| injection | vehicle | 1 μmol/kg | 1 μmol/kg |
| 1 hour | 0.90 | 0.42*** | 0.52*** |
| 2 hours | 1.30 | 0.80** | 0.96* |
| 3 hours | 1.80 | 1.17*** | 1.42** |
| 4 hours | 2.04 | 1.49** | 1.62* |
| 6 hours | 2.58 | 1.89*** | 2.13** |
| 8 hours | 2.96 | 2.42** | 2.75 |
| 12 hours | 3.76 | 3.46 | 3.56 |
| 24 hours | 4.15 | 4.23 | 3.97 |
| 36 hours | 7.93 | 7.20 | 7.17 |
| 48 hours | 8.16 | 7.65 | 7.53 |

*p < 0.05, **p < 0.01, ***p < 0.001 (ANOVA, Dunnetts post hoc)

TABLE 9
Mean cumulative food intake (n = 6-8 per group)

| | Mean cumulative food intake [g] | | | |
|---|---|---|---|---|
| Time after | | SEQ ID NO: 55 | SEQ ID NO: 61 | SEQ ID NO: 69 |
| injection | vehicle | 1.0 μmol/kg | 1.0 μmol/kg | 1.0 μmol/kg |
| 1 hour | 0.79 | 0.49* | 0.34** | 0.54 |
| 2 hours | 1.21 | 0.69* | 0.78** | 0.79 |
| 3 hours | 1.57 | 0.90*** | 1.16* | 0.92*** |
| 4 hours | 1.94 | 1.09*** | 1.44** | 1.15*** |
| 6 hours | 2.28 | 1.39*** | 2.18 | 1.55*** |
| 8 hours | 2.70 | 1.73*** | 2.42 | 2.00** |
| 12 hours | 3.31 | 2.37*** | 3.20 | 2.59** |
| 24 hours | 3.78 | 3.23 | 4.01 | 2.99* |
| 36 hours | 7.38 | 5.48*** | 7.52 | 5.69** |
| 48 hours | 7.59 | 6.14* | 7.94 | 5.97* |

*p < 0.05, **p < 0.01, ***p < 0.001 (ANOVA, Dunnetts post hoc)

TABLE 10
Mean cumulative food intake (n = 7-8 per group)

| | Mean cumulative food intake [g] | | |
|---|---|---|---|
| Time after | | SEQ ID NO: 62 | SEQ ID NO: 59 |
| injection | vehicle | 1 μmol/kg | 1 μmol/kg |
| 1 hour | 0.49 | 0.31 | 0.27* |
| 2 hours | 0.92 | 0.47*** | 0.56*** |
| 3 hours | 1.24 | 0.63*** | 0.77*** |
| 4 hours | 1.58 | 0.76*** | 0.96*** |
| 6 hours | 2.16 | 1.10*** | 1.51* |
| 8 hours | 2.57 | 1.49*** | 2.06* |
| 12 hours | 3.49 | 2.17*** | 3.04 |
| 24 hours | 3.74 | 3.17 | 3.57 |
| 36 hours | 7.30 | 5.94 | 7.26 |
| 48 hours | 7.45 | 6.21 | 7.68 |

*p < 0.05, **p < 0.01, ***p < 0.001 (ANOVA, Dunnetts post hoc)

TABLE 11
Mean cumulative food intake (n = 7-8 per group)

| | Mean cumulative food intake [g] | |
|---|---|---|
| Time after | | SEQ ID NO: 84 |
| injection | vehicle | 1 μmol/kg |
| 1 hour | 0.49 | 0.31 |
| 2 hours | 0.94 | 0.60** |
| 3 hours | 1.20 | 0.91 |
| 4 hours | 1.58 | 1.17* |
| 6 hours | 2.22 | 1.46** |
| 8 hours | 2.71 | 1.72*** |
| 12 hours | 3.60 | 2.51*** |
| 24 hours | 3.69 | 2.87** |
| 36 hours | 7.21 | 6.15 |
| 48 hours | 7.32 | 6.73 |

*p < 0.05, **p < 0.01, ***p < 0.001 (ANOVA, Dunnetts post hoc)

TABLE 12
Mean cumulative food intake (n = 7-8 per group)

| | Mean cumulative food intake [g] | |
|---|---|---|
| Time after | | SEQ ID NO: 77 |
| injection | vehicle | 1 μmol/kg |
| 1 hour | 0.75 | 0.47* |
| 2 hours | 1.16 | 0.85* |
| 3 hours | 1.45 | 1.08* |
| 4 hours | 1.78 | 1.21** |
| 6 hours | 2.35 | 1.61*** |
| 8 hours | 2.87 | 2.10*** |
| 12 hours | 3.55 | 2.76* |
| 24 hours | 3.95 | 3.49 |
| 36 hours | 7.33 | 5.82* |
| 48 hours | 7.62 | 6.12* |

*p < 0.05, **p < 0.01, ***p < 0.001 (ANOVA, Dunnetts post hoc)

### Example 98

### Food Intake in Pigs (Assay (VII))

The effect of PYY derivatives on food intake was determined in pigs according to Assay (VII) described above. The results are shown in Table 13.

TABLE 13
Average food intake (kg) in young growing LYD pigs
after single dose (s.c. 100 nmol/kg)

| Time after | Average food intake [kg] | | |
|---|---|---|---|
| injection | vehicle | SEQ ID NO: 6 | SEQ ID NO: 35 |
| 1 day | 2.32 | 1.54** | 1.44*** |
| 2 days | 2.45 | 1.74** | 1.55*** |
| 3 days | 2.54 | 1.80** | 1.80** |
| 4 days | 2.56 | 1.86** | 1.83** |

*p. < 0.05, **p < 0.01, ***p < 0.001 (Two-way ANOVA, Bonferroni post test)

### Example 99

### Body Weight in Mice (Assay (VI))

The effect of PYY derivatives on body weight was determined ob/ob mice after subchronic administration according to Assay (VI) described above. The results are shown in Table 14.

TABLE 14
Actual body weight (g) and mean body weight change (g) in ob/ob mice after
subchronic administration (n = 7-8 per group, data are mean ± SE)

| | | | SEQ ID NO: 6 | | SEQ ID NO: 6 | |
|---|---|---|---|---|---|---|
| | vehicle | | 0.1 μmol/kg | | 1 μmol/kg | |
| Days of | Actual body | Change of body | Actual body | Change of body | Actual body | Change of body |
| treatment | weight (g) | weight (g) | weight (g) | weight (g) | weight (g) | weight (g) |
| 0 | 54.2 ± 1.3 | n.a. | 53.3 ± 1.1 | n.a. | 54.6 ± 1.5 | n.a. |
| 6 | 54.4 ± 1.4 | 0.19 ± 0.16 | 51.1 ± 1.1 | −2.2 ± 0.2*** | 48.4 ± 1.0** | −6.1 ± 0.3*** |
| 12 | 54.6 ± 1.5 | 0.36 ± 0.31 | 51.2 ± 1.2 | −2.1 ± 0.3*** | 48.5 ± 1.1** | −5.9 ± 0.4*** |
| 17 | 54.9 ± 1.6 | 0.68 ± 0.29 | 51.6 ± 1.2 | −1.7 ± 0.3*** | 48.6 ± 1.1** | −5.8 ± 0.4*** |

**p < 0.01,
***p < 0.001 (Two-way ANOVA, Bonferroni post test),
n.a. not applicable

### Example 100

### Physical Stability

The level of peptide recovery is an indicator of physical stability, wherein higher peptide recovery indicates better physical stability. An increase in peptide recovery indicates a more physically stable PYY derivative.

The test compounds were formulated in 10 mM NaCl and 10 mM phosphate buffer pH 7.4 at a concentration of approx. 2 mM. Sample aliquots of 200 μL in replica of two to four were transferred to a Perkin Elmer OptiPlate™-96, white polystyrene microtiter plate. The well plate was covered by an adhesive tape sheet to avoid sample evaporation, incubated at 37° C. and agitated at 960 rpm with an amplitude of 1 mm for 45 hours in a plate reader (Fluoroskan Ascent FL). After shaking samples for each test compound were transferred from the wells to eppendorf tubes, spun down (at 20000 g for 30 minutes) and filtered through a 0.22 μm filter; the filtered samples are hereafter referred to as the physically stressed samples. Peptide concentration was measured on a sample that was stored under quiescent conditions at 5° C. (start sample) and on the physically stressed sample. Peptide recovery was defined as the concentration of peptide remaining in solution after physical stress compared to the concentration of the sample stored under quiescent conditions.

Peptide concentration was measured using reverse phase HPLC: The concentration of the test compounds was measured on a XBridge™ BEH130 column C18 (3.5 μm, 4.6×50 mm, Waters: part no: 18603567) by using gradient elution of a mixture of A (0.2 mM sodium sulphate, 0.04 M phosphate buffer pH 7.2 with 7.7% (w/w) acetonitrile) and B (acetonitrile 65.5% (w/w)) at 35° C. and detection at 276 nm. The column was operated at 2 ml/min at the following gradient: at 0 min the composition was 80% A and 20% B which over 5.5 min was changed to 50% A and 50% B by a linear gradient. The concentration of the test compound was determined by comparing to a reference compound of known concentration. The concentration of the reference compound was determined using nitrogen detection.

Table 15 shows peptide recovery for several PYY derivatives. SEQ ID NO: 6 and in particular SEQ ID NO: 35 had low peptide recovery, whereas the remaining PYY derivatives had improved physical stability. The serum albumin binding side chain of SEQ ID NO: 82 comprises

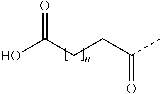

wherein n is 13.

TABLE 15
Recovery of PYY derivatives following 45 hours of shaking

| SEQ ID NO: | Recovery (%) | Substitution(s) in addition to 30K |
|---|---|---|
| 6 | 26 | — |
| 35 | 0 | 4R |
| 46 | 101 | 4E |
| 49 | 93 | 4R, 18E, 22E |
| 54 | 100 | 4V |
| 67 | 93 | NAc, des3, des4 |
| 69 | 96 | NAc, 4R |
| 82 | 99 | NAc |
| 89 | 102 | NAc, 4A |

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

1. A PYY derivative comprising a serum albumin binding side chain, wherein
said derivative has a half-life of at least 7 hours as determined by Assay (IV), and wherein
said serum albumin binding side chain comprises an alkyl chain of at least 14 carbon atoms, and wherein
said alkyl chain comprises a distal carboxylic acid group or a distal tetrazole group, and wherein
said serum albumin binding side chain is attached to the N-terminal amino group or an amino acid in a position selected from the group consisting of position 1, 3, 6, 7, 9, 10, 11, 12, 14, 15, 17, 18, 19, 21, 22, 23 and 30, wherein said position is relative to hPYY(1-36).

2. The PYY derivative according to claim 1, wherein said derivative is not
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY3-36,
N-epsilon13-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys13]hPYY3-36,
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY3-36,
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys11]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY2-36,
N-epsilon4-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY3-36,
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Arg26]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Leu3,Glu4,Val6,Tyr7, Lys13,Arg26]hPYY1-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4, Lys13,Arg26]hPYY3-36,
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4,Tyr7,Lys13,Arg26]hPYY1-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY3-36,
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys4]hPYY3-36,
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys11]hPYY3-36,
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY3-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY18-36
N-epsilon25-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys25]hPYY3-36,
N-epsilon24-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys24]hPYY3-36,
N-epsilon19-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys19]hPYY3-36,
N-epsilon13-[4-(16-(1H-Tetrazol-5-yl)hexadecanoylsulfamoyl)butyl]ethoxy}ethoxy)acetylamino]ethoxy}ethoxy)acetyl][Lys13]PYY3-36,
N-epsilon25-[4-(16-(1H-Tetrazol-5-yl)hexadecanoylsulfamoyl)butyl]ethoxy}ethoxy)acetylamino]ethoxy}ethoxy)acetyl][Lys25]PYY3-36,
N-alpha-[4-(16-(1H-Tetrazol-5-yl)hexadecanoylsulfamoyl)butyl]ethoxy}ethoxy)acetylamino]ethoxy}ethoxy)acetyl]PYY3-36,
N-alpha-[4-(16-(1H-Tetrazol-5-yl)hexadecanoylsulfamoyl)butyl]ethoxy}ethoxy)acetylamino]ethoxy}ethoxy)acetyl]PYY3-36,
N-epsilon18-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys18]PYY3-36,
N-epsilon22-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys22]PYY3-36,
N-epsilon26-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys26]PYY3-36,
N-epsilon29-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys29]PYY3-36,
N-epsilon36-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys36]PYY3-36,
N-epsilon21-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys21]PYY3-36,
N-epsilon30-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys30]PYY3-36,
N-epsilon31-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys31]PYY3-36,
N-epsilon14-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys14]PYY3-36,
N-epsilon15-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys15]PYY3-36,
N-epsilon16-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys16] PYY3-36,
N-epsilon20-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys20]PYY3-36,
N-epsilon28-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys28]PYY3-36,
N-epsilon32-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys32]PYY3-36,
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}-ethoxy)acetylamino]ethoxy}ethoxy)acetyl] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][N-Methyl Arg35] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][His25,N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Aib25,N-Methyl Gln34] PYY(3-36),
N-alpha-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl][Tyr25,N-Methyl Gln34] PYY(3-36),
N-alpha-{2-(2-{2-[2-(2-{2-[4-Carboxy-4-(17-carboxyheptadecanoylamino)butyrylamino]ethoxy}-ethoxy)acetylamino]-ethoxy}ethoxy)acetyl}[Ala20] PYY(3-36),
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[N-methyl Arg35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Ala27] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Agp35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Agb35] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[HomoArg35] PYY(3-36),
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[NArg35] PYY(3-36) or
N-alpha-{2-(2-{2-[2-(2-{2-[4-Carboxy-4-(17-carboxyheptadecanoylamino)butyrylamino]ethoxy}ethoxy)acetylamino]-ethoxy}ethoxy)acetyl}[NGln34] PYY(3-36),
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,N-methyl Tyr36] PYY(3-36),
N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Cα-methyl Tyr36] PYY(3-36),
[N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}] PYY(3-36),
[N-alpha-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[NTyr36]]PYY3-36, or
N-alpha-Acetyl[N-epsilon10-{2-(2-{2-[2-(2-{2-[4-Carboxy-4-(17-carboxyheptadecanoylamino)butyrylamino]ethoxy}ethoxy)-acetylamino]-ethoxy}ethoxy)acetyl}[Lys10,N-methyl Arg35]] PYY(3-36).

3. The PYY derivative according to claim 1, wherein said distal carboxylic acid group has the formula (X) 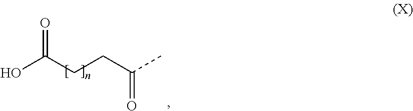
wherein n is at least 13, such as n is 13, 14, 15, 16, 17, 18 or 19.

4. The PYY derivative according to claim 1, wherein said derivative has a Y2 receptor potency of less than 20 nM as determined by Assay (I), and
a) a Y1/Y2 receptor potency ratio which is higher than the Y1/Y2 receptor potency ratio of hPYY(3-36), wherein the Y1 receptor potency and the Y2 receptor potency is determined by Assay (II) and Assay (I), respectively; and/or
b) a Y5/Y2 receptor potency ratio which is higher than the Y5/Y2 receptor potency ratio of hPYY(3-36), and wherein the Y5 receptor potency and the Y2 receptor potency is determined by Assay (III) and Assay (I), respectively.

5. The PYY derivative according to claim 1, wherein said serum albumin binding side chain is attached to the N-terminal amino group or an amino acid in a position selected from the group consisting of position 3, 6, 7, 10, 11, 14, 17, 18, 19, 21, 22 and 30.

6. The PYY derivative according to claim 1, wherein said serum albumin binding side chain is attached to an amino acid in a position selected from the group consisting of position 7, 10, 21, 22 and 30,

7. The PYY derivative according to claim 1, wherein said serum albumin binding side chain is attached to an amino acid in a position selected from the group consisting of position 10, 11, 14, 17, 19, 21 and 30.

8. A PYY derivative according to claim 1, wherein said serum albumin binding side chain is attached to an amino acid in a position selected from the group consisting of position 10, 21 and 30, such as in position 30.

9. The PYY derivative according to claim 1, wherein said derivative has a half-life of at least 8 hours, such as at least 15 hours or at least 30 hours, as determined by Assay (IV).

10. The PYY derivative according to claim 1, wherein said derivative comprises formula (I):
Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅-Xaa₂₆-Xaa₂₇-Xaa₂₈-Xaa₂₉-Xaa₃₀-Xaa₃₁-Xaa₃₂-Xaa₃₃-Xaa₃₄-Xaa₃₅-Xaa₃₆ Formula (I)
wherein
Xaa₁ is Tyr, Phe, Ala, 2,3-diaminopropionic acid, 2,4-diaminobutyric acid, ornitine, Lys or absent;
Xaa₂ is Pro, Ala, Leu, Phe, hydroxyproline, 2,3-diaminopropionic acid, 2,4-diaminobutyric acid, ornitine, Lys or absent;
Xaa₃ is Ile, Val, Leu (1-aminocyclopentyl)carboxylic acid, (1-aminocyclohexyl)carboxylic acid, 1-aminobutyric acid, 2,3-diaminopropionic acid, 2,4-diaminobutyric acid, ornitine, Lys, D-Ile, D-allo-Ile or absent;
Xaa₄ is Lys, Arg, Glu, 2,3-diaminopropionic acid, 2,4-diaminobutyric acid, ornitine, Lys, absent, Ala, Val, Ser or Gly;
Xaa₅ is Pro, 2,3-diaminopropionic acid, 2,4-diaminobutyric acid, ornitine, absent or Lys;
Xaa₆ is Glu, 2,3-diaminopropionic acid, 2,4-diaminobutyric acid, ornithine, absent or Lys;
Xaa₇ is Ala, 2,3-diaminopropionic acid, 2,4-diaminobutyric acid, ornitine, absent or Lys;
Xaa₈ is Pro, Ala, 2,3-diaminopropionic acid, 2,4-diaminobutyric acid, ornitine, absent, Glu or Lys;
Xaa₉ is Gly, 2,3-diaminopropionic acid, 2,4-diaminobutyric acid, ornitine, absent, Glu or Lys;
Xaa₁₀ is Glu, 2,3-diaminopropionic acid, 2,4-diaminobutyric acid, ornitine or Lys;
Xaa₁₁ is Asp, Ala, 2,3-diaminopropionic acid, 2,4-diaminobutyric acid, ornitine or Lys;
Xaa₁₂ is Ala, 2,3-diaminopropionic acid, 2,4-diaminobutyric acid, ornitine or Lys;
Xaa₁₃ is Ser, 2,3-diaminopropionic acid, 2,4-diaminobutyric acid, ornithine, Lys;
Xaa₁₄ is Pro, hydroxyproline or Ala;
Xaa₁₅ is Glu, 2,3-diaminopropionic acid, 2,4-diaminobutyric acid, ornitine or Lys;
Xaa₁₆ is Glu, 2,3-diaminopropionic acid, 2,4-diaminobutyric acid, ornitine or Lys;
Xaa₁₇ is Leu, Val, Ile, homoleucine, norleucine, (1-aminocyclopentyl)carboxylic acid, (1-aminocyclohexyl)carboxylic acid or 1-aminobutyric acid;
Xaa₁₈ is Asn, Ala, Glu, 2,3-diaminopropionic acid, 2,4-diaminobutyric acid, ornitine, Lys, Gln, Asp, D-Asp, IsoAsp or D-IsoAsp;
Xaa₁₉ is Arg, Ala, 2,3-diaminopropionic acid, 2,4-diaminobutyric acid, ornithine, Glu or Lys;
Xaa₂₀ is Tyr, Ala, Phe, 3-pyridylalaine, 2,3-diaminopropionic acid, 2,4-diaminobutyric acid, ornitine or Lys;
Xaa₂₁ is Tyr, Ala, Phe, 3-pyridylalaine, 2,3-diaminopropionic acid, 2,4-diaminobutyric acid, ornitine or Lys;
Xaa₂₂ is Ala, 2,3-diaminopropionic acid, 2,4-diaminobutyric acid, ornithine, Arg, Glu or Lys;
Xaa₂₃ is Ser, 2,3-diaminopropionic acid, 2,4-diaminobutyric acid, ornitine or Lys;
Xaa₂₄ is Leu, Ile, Val, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₂₅ is Arg, Ala, His, aminoisobutyric acid, 2,3-diaminopropionic acid, 2,4-diaminobutyric acid, ornitine or Lys;
Xaa₂₆ is His, Arg, 2,3-diaminopropionic acid, 2,4-diaminobutyric acid, ornitine or Lys;
Xaa₂₇ is Tyr, Ala, Phe, homoPhe or 3-pyridylalanine, 2,3-diaminopropionic acid, 2,4-diaminobutyric acid, ornitine or Lys;
Xaa₂₈ is Leu, Ile, Val, homoleucine, norleucine, (1-aminocyclopentyl)carboxylic acid, (1-aminocyclohexyl)carboxylic acid, aminoisobutyric acid, 1-aminobutyric acid, 2,3-diaminopropionic acid, 2,4-diaminobutyric acid, ornitine or Lys;
Xaa₂₉ is Asn, Gln, 2,3-diaminopropionic acid, 2,4-diaminobutyric acid, ornithine, D-IsoAsp or Lys;
Xaa₃₀ is Leu, Met, Val, Ile, homoleucine, aminoisobutyric acid, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₁ is Val, Leu, Ile, aminoisobutyric acid, homoleucine, norleucine, (1-aminocyclopentyl)carboxylic acid, (1-aminocyclohexyl)carboxylic acid, 1-aminobutyric acid, 2,3-diaminopropionic acid, 2,4-diaminobutyric acid, ornitine or Lys;
Xaa₃₂ is Thr, Ser, 2,3-diaminopropionic acid, 2,4-diaminobutyric acid, ornitine or Lys;
Xaa₃₃ is Arg, N-methyl Arg, methyllysine, dimethyllysine, trimethyllysine, 2-amino-3-guanidino-propionic acid, 2-amino-4-guanidino-butyric acid or monomethylarginine, dimethylarginine, (2-Guanidino-ethylamino)-acetic acid, (3-Guanidino-propylamino)acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)acetic acid;
Xaa₃₄ is Gln, Asn, His, Pro, N-methyl Gln, β-homo Gln, (2-Carbamoyl-ethylamino)-acetic acid, N-methyl Asn or N-methyl His;
Xaa₃₅ is Arg, N-methyl Arg, methyllysine, dimethyllysine, trimethyllysine, 2-amino-3-guanidino-propionic acid, 2-amino-4-guanidino-butyric acid, monomethylarginine, dimethylarginine, (2-Guanidino-ethylamino)-acetic acid, (3-Guanidino-propylamino)acetic acid, (4-Guanidino-butylamino)-acetic acid, 2-Amino-3-(1-carbamimidoyl-pyrrolidin-2-yl)-propionic acid, 2-Amino-4-(2-amino-pyrimidin-4-yl)-butyric acid, 2-Amino-3-(4-guanidino-phenyl)-propionic acid or Amino-(1-carbamimidoyl-piperidin-4-yl)acetic acid; and
Xaa₃₆ is Tyr, Phe, N-methyl Tyr, C-methyl Phe, 3-pyridylalanine or (4-Hydroxybenzylamino)-acetic acid, 4-fluorophenylalanine or 4-pyridylalanine.

11. The PYY derivative according to claim 1, wherein said serum albumin binding side chain is selected from the group consisting of A-B-C-D-, A-C-D-, A-B-C- and A-C-,
wherein A- is 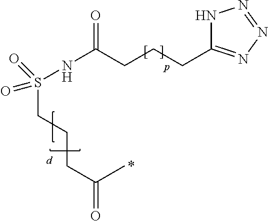
wherein p is selected from the group consisting of 10, 11, 12, 13 and 14, and d is selected from the group consisting of 0, 1, 2, 3, 4 and 5, and
-B- is selected from the group consisting of 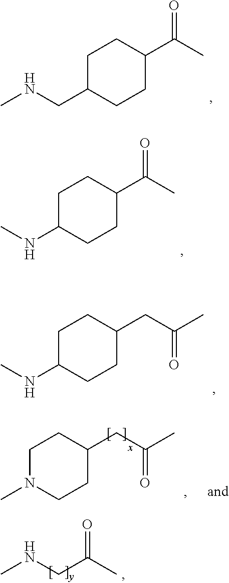
wherein x is selected from the group consisting of 0, 1, 2, 3 and 4, and y is selected from the group consisting of 1, 2, 3, 4, 5, 6, 7, 8, 9, 10, 11 and 12,
or A- is 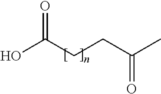
wherein n is selected from the group consisting of 12, 13, 14, 15, 16 17, 18 and 19, and B is selected from the group consisting of 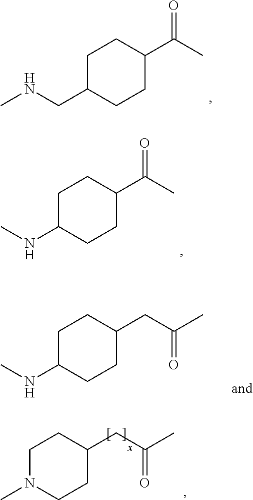
wherein x is selected from the group consisting of 0, 1, 2, 3 and 4, and
-C- is selected from the group consisting of 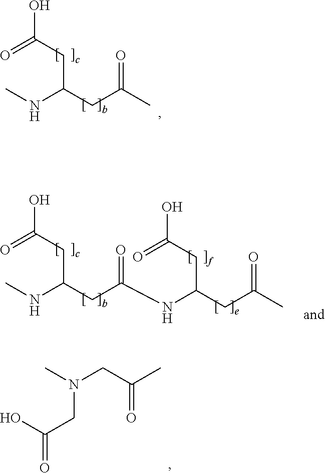
wherein b and e are each independently selected from the group consisting of 0, 1 and 2, and c and f are each independently selected from the group consisting of 0, 1 and 2 with the proviso that b is 1 or 2 when c is 0, or b is 0 when c is 1 or 2, and e is 1 or 2 when f is 0, or e is 0 when f is 1 or 2, and
-D- is attached to said amino acid residue and is a spacer, such as at least one 8-amino-3,6-dioxaoctanoic acid (Oeg) molecule.

12. The PYY derivative according to claim 1, wherein said derivative is selected from the group consisting of
N-epsilon35 [2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys35]hPYY(3-36) (SEQ ID NO: 1);
N-epsilon34[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys34]hPYY(3-36) (SEQ ID NO: 2);
N-epsilon33 [2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys33]hPYY(3-36) (SEQ ID NO: 3);
N-epsilon32[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys32]hPYY(3-36) (SEQ ID NO: 4);
N-epsilon31[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys31]hPYY(3-36) (SEQ ID NO: 5);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(3-36) (SEQ ID NO: 6);
N-epsilon29[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys29]hPYY(3-36) (SEQ ID NO: 7);
N-epsilon28[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys28]hPYY(3-36) (SEQ ID NO: 8);
N-epsilon27[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys27]hPYY(3-36) (SEQ ID NO: 9);
N-epsilon26[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys26]hPYY(3-36) (SEQ ID NO: 10);
N-epsilon25 [2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys25]hPYY(3-36) (SEQ ID NO: 11);
N-epsilon24[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys24]hPYY(3-36) (SEQ ID NO: 12);
N-epsilon23 [2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys23]hPYY(3-36) (SEQ ID NO: 13);
N-epsilon22[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys22]hPYY(3-36) (SEQ ID NO: 14);
N-epsilon21[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys21]hPYY(3-36) (SEQ ID NO: 15);
N-epsilon20 [2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys20]hPYY(3-36) (SEQ ID NO: 16);
N-epsilon19[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys19]hPYY(3-36) (SEQ ID NO: 17);
N-epsilon18[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys18]hPYY(3-36) (SEQ ID NO: 18);
N-epsilon17[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys17]hPYY(3-36) (SEQ ID NO: 19);
N-epsilon16[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys16]hPYY(3-36) (SEQ ID NO: 20);
N-epsilon15[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys15]hPYY(3-36) (SEQ ID NO: 21);
N-epsilon14[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys14]hPYY(3-36) (SEQ ID NO: 22);
N-epsilon13[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys13]hPYY(3-36) (SEQ ID NO: 23);
N-epsilon12[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys12]hPYY(3-36) (SEQ ID NO: 24);
N-epsilon11[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys11]hPYY(3-36) (SEQ ID NO: 25);
N-epsilon10[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys10]hPYY(3-36) (SEQ ID NO: 26);
N-epsilon9[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys9]hPYY(3-36) (SEQ ID NO: 27);
N-epsilon8[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys8]hPYY(3-36) (SEQ ID NO: 28);
N-epsilon7[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys7]hPYY(3-36) (SEQ ID NO: 29);
N-epsilon6[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys6]hPYY(3-36) (SEQ ID NO: 30);
N-epsilon5 [2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys5]hPYY(3-36) (SEQ ID NO: 31);
N-epsilon4[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys4]hPYY(3-36) (SEQ ID NO: 32);
N-epsilon3 [2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys3]hPYY(3-36) (SEQ ID NO: 33);
N-alpha-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl]hPYY(3-36) (SEQ ID NO: 34);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Lys30]hPYY(3-36) (SEQ ID NO: 35);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys30]hPYY(5-36) (SEQ ID NO: 36);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4, Glu18,Lys30]hPYY(3-36) (SEQ ID NO: 37);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu18,Lys30]hPYY(5-36) (SEQ ID NO: 38);
N-epsilon30[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl][Arg4,Glu18,Gln29,Lys30]hPYY(3-36) (SEQ ID NO: 39);
N-epsilon30[2-(2-{2-[2-(2-{2-[(S)-4-Carboxy-4-(hexadecanoylamino)butyrylamino]ethoxy}ethoxy)-acetylamino]ethoxy}ethoxy)acetyl][Lys30]hPYY(3-36) (SEQ ID NO: 40);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Gln18,Lys30]hPYY(3-36) (SEQ ID NO: 41);
N-epsilon1[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Lys1]hPYY(1-36) (SEQ ID NO: 42);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ser4,Lys30]hPYY(3-36) (SEQ ID NO: 43);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][IsoAsp18,Lys30]hPYY(3-36) (SEQ ID NO: 44);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-IsoAsp29,Lys30]hPYY(3-36) (SEQ ID NO: 45);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu4,Lys30]hPYY(3-36) (SEQ ID NO: 46);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][D-IsoAsp18,Lys30]hPYY(3-36) (SEQ ID NO: 47);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,Glu22,Lys30]hPYY(3-36) (SEQ ID NO: 48);
N-epsilon30[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl][Arg4,Glu18,Glu22,Lys30]hPYY(3-36) (SEQ ID NO: 49);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Arg4,D-Asp18,Lys30]hPYY(3-36) (SEQ ID NO: 50);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Asp18,Lys30]hPYY(3-36) (SEQ ID NO: 51);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala14,Lys30]hPYY(3-36) (SEQ ID NO: 52);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala18,Lys30]hPYY(3-36) (SEQ ID NO: 53);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Val4,Lys30]hPYY(3-36) (SEQ ID NO: 54);
N-epsilon30[2-(2-{2-[2-(2-{2-[(S)-4-Carboxy-4-(19-carboxynonadecanoylamino)butyrylamino]ethoxy}ethoxy)acetylamino]ethoxy}ethoxy)acetyl][Lys30]hPYY(3-36) (SEQ ID NO: 55);
N-epsilon30[2-(2-{2-[2-(2-{2-(19-carboxynonadecanoylamino)ethoxy}ethoxy)acetylamino]ethoxy}ethoxy)acetyl][Lys30]hPYY(3-36) (SEQ ID NO: 56);
N-epsilon30[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)-acetylamino]ethoxy}ethoxy)acetyl][Lys30]hPYY(3-36) (SEQ ID NO: 57);
N-epsilon30[2-(2-{2-[2-(2-{2-(17-carboxyheptadecanoylamino)-ethoxy}ethoxy)acetylamino]ethoxy}ethoxy)acetyl][Lys30]hPYY(3-36) (SEQ ID NO: 58);
N-epsilon30[2-(2-{2-[2-(2-{2-[(S)-4-Carboxy-4-(15-carboxypentadecanoylamino) butyrylamino]ethoxy}ethoxy)-acetylamino]ethoxy}ethoxy)acetyl][Lys30]hPYY(3-36) (SEQ ID NO: 59);
N-epsilon30[2-(2-{2-[2-(2-{2-(15-carboxypentadecanoylamino)-ethoxy}ethoxy)acetylamino]ethoxy}ethoxy)acetyl][Lys30]hPYY(3-36) (SEQ ID NO: 60);
N-epsilon30[2-(2-{2-[2-(2-{2-[(S)-4-Carboxy-4-(13-carboxytridecanoylamino) butyrylamino]ethoxy}ethoxy)-acetylamino]ethoxy}ethoxy)acetyl][Lys30]hPYY(3-36) (SEQ ID NO: 61);
N-epsilon30[2-(2-{2-[2-(2-{2-(13-carboxytridecanoylamino)-ethoxy}ethoxy)acetylamino]ethoxy}ethoxy)acetyl][Lys30]hPYY(3-36) (SEQ ID NO: 62);
N-epsilon30[2-(2-{2-[2-(2-{2-[(S)-4-Carboxy-4-(11-carboxyundecanoylamino) butyrylamino]ethoxy}ethoxy)-acetylamino]ethoxy}ethoxy)acetyl][Lys30]hPYY(3-36) (SEQ ID NO: 63);
N-epsilon30[2-(2-{2-[2-(2-{2-(11-carboxyundecanoylamino)-ethoxy}ethoxy)acetylamino]ethoxy}ethoxy)acetyl][Lys30]hPYY(3-36) (SEQ ID NO: 64);
N-epsilon30[2-(2-{2-[2-(2-{2-[(4-(16-(1H-Tetrazol-5-yl)hexadecanoylsulfamoyl)butyryl]-ethoxy}ethoxy)-acetylamino]ethoxy}ethoxy)acetyl][Lys30]hPYY(3-36) (SEQ ID NO: 65);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][D-Asp18,Lys30] hPYY(3-36) (SEQ ID NO: 66);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Lys30]hPYY(5-36) (SEQ ID NO: 67);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Ala4,Asp18,Lys3 0]hPYY(3-36) (SEQ ID NO: 68);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Arg4, Lys30]hPYY(3-36) (SEQ ID NO: 69);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Lys30]hPYY(9-36) (SEQ ID NO: 70);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Gly4, Lys30]hPYY(3-36) (SEQ ID NO: 71);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Arg4, Asp 18,Lys30]hPYY(4-36) (SEQ ID NO: 72);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-succinyl,Arg4, Asp 18,Lys30]hPYY(3-36) (SEQ ID NO: 73);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-isovaleryl,Arg4, Lys30]hPYY(4-36) (SEQ ID NO: 74);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Lys30,4-fluorophenylalanine36]hPYY(3-36) (SEQ ID NO: 75);
N-epsilon30[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl][Lys30,4-pyridylalanine-36]hPYY(3-36) (SEQ ID NO: 76);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu19,Arg22,Lys3 0]hPYY(3-36) (SEQ ID NO: 77);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,D-Ile3,Arg4,Lys30]hPYY(3-36) (SEQ ID NO: 78);
N-epsilon30[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl][N-alpha-acetyl,Gly4,Lys30]hPYY(3-36) (SEQ ID NO: 79);
N-epsilon30[2-(2-{2-[2-(2-{2-[(S)-4-carboxy-[(S)-4-Carboxy-4-(17-carboxyheptadecanoylamino)butyrylamino]butyrylamino)ethoxy}ethoxy)-acetylamino]ethoxy}ethoxy)acetyl][N-alpha-acetyl,Arg4,Lys30]hPYY(3-36) (SEQ ID NO: 80);
N-epsilon30[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl][N-alpha-acetyl,Arg4,Asp18,Lys30]hPYY(3-36) (SEQ ID NO: 81);
N-epsilon30[2-(2-{2-[2-(2-{2-[(S)-4-Carboxy-4-(15-carboxypentadecanoylamino)butyrylamino]ethoxy}ethoxy)acetylamino]ethoxy}ethoxy)acetyl][N-alpha-acetyl,Lys30]hPYY(3-36) (SEQ ID NO: 82);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Ala4,Asp18,Lys30]hPYY(3-36) (SEQ ID NO: 83);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][Arg4,Asp18,Lys30]hPYY(3-36) (SEQ ID NO: 84);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][D-Arg4, Lys30]hPYY(3-36) (SEQ ID NO: 85);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][D-allo-Ile3,Arg4, Lys30]hPYY(3-36) (SEQ ID NO: 86);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][D-Ala4, Lys30]hPYY(3-36) (SEQ ID NO: 87);
N-epsilon30-[(S)-4-Carboxy-4-(17-carboxyheptadecanoylamino)butyrylamino][Lys30]hPYY(3-36) (SEQ ID NO: 88);
N-epsilon30[2-(2-{2-[2-(2-{2-[(S)-4-Carboxy-4-(17-carboxyheptadecanoylamino) butyrylamino]ethoxy}ethoxy)-acetylamino]ethoxy}ethoxy)acetyl][N-alpha-acetyl,Ala4,Lys30]hPYY(3-36) (SEQ ID NO: 89);
N-epsilon30[{(S)-4-Carboxy-4-(17-carboxyheptadecanoylamino)butyrylamino}-Ser-Ser-Gly-Ser-Ser-Gly][Arg4,Lys30]hPYY(3-36) (SEQ ID NO: 90);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu8,Lys30]hPYY(3-36) (SEQ ID NO: 91);
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Glu9,Lys30]hPYY(3-36) (SEQ ID NO: 92); and
N-epsilon30[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Ala4,Lys30]hPYY(3-36) (SEQ ID NO: 93).

13. A composition comprising the PYY derivative according to claim 1 and at least one pharmaceutical excipient.

14. (canceled)

15. A method of treatment of a condition responsive to Y-receptor modulation in a subject in need thereof comprising administering to the subject a pharmaceutical composition comprising the PYY derivative according to claim 1.

16. The method according to claim 15, wherein the condition responsive to Y-receptor modulation is selected from obesity and type 2 diabetes.
